SEQUENCE LISTING (502 sequences)

SEQ ID NO: 1 - protein, 8 aa, Homo sapiens
GFTFSSYS

SEQ ID NO: 2 - protein, 8 aa, Homo sapiens
ISGNSNYI

SEQ ID NO: 3 - protein, 13 aa, Homo sapiens
ARGTIIGAAGYDC

SEQ ID NO: 4 - protein, 6 aa, Homo sapiens
QDISTS

SEQ ID NO: 5 - skipped

SEQ ID NO: 6 - protein, 9 aa, Homo sapiens
QQLKSYPLA

SEQ ID NO: 7 - protein, 120 aa, Homo sapiens
EVQLVESGGGLVKPGGSLRLSCAASGFTFSSYSMNWVRQAPGKGLEWVSSISGNSNYIYYADSVKGRFTISRDNAKNSLYLQTSSLRAEDTALYFCARGTIIGAAGYDCWGQGTLVTVSS

SEQ ID NO: 8 - protein, 107 aa, Homo sapiens
AIQMTQSPSFLSASVGDRVTLTCRASQDISTSLAWYHQKPGKAPERLVYAASTLQSGVPSRFSGSGSGTGFTLTISSLQPEDFGTYYCQQLKSYPLAFGGGTKVEIK

SEQ ID NO: 9 - protein, 468 aa, Homo sapiens
MGWSCIILFLVATATGVHSEVQLVESGGGLVKPGGSLRLSCAASGFTFSSYSMNWVRQAPGKGLEWVSSISGNSNYIYYADSVKGRFTISRDNAKNSLYLQTSSLRAEDTALYFCARGTIIGAAGYDCWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 10 - protein, 233 aa, Homo sapiens
MGWSCIILFLVATATGVHSAIQMTQSPSFLSASVGDRVTLTCRASQDISTSLAWYHQKPGKAPERLVYAASTLQSGVPSRFSGSGSGTGFTLTISSLQPEDFGTYYCQQLKSYPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 57 bp, Homo sapiens
  source 1..57: mol_type = other DNA
atgggatggtcatgtatcatcctttttctagtagcaactgcaaccggtgtacattcc

SEQ ID NO: 12 - DNA, 361 bp, Homo sapiens
  source 1..361: mol_type = other DNA
gaggtgcagctggtggagtctgggggaggcctggtcaagcctggggggtccctgagactctcctgtgcagcctctggattcaccttcagttcctatagcatgaactgggtccgccaggctccagggaaggggctggagtgggtctcatccattagtggcaatagtaattacatatattacgccgactcagtgaagggccgattcaccatctccagagacaacgccaagaactcactctatctgcaaacgagcagcctgagagccgaggacacggctctctatttctgtgcgagggggacaattattggagctgcgggatatgactgctggggacagggaacccttgtcactgtctcctcag

SEQ ID NO: 13 - DNA, 989 bp, Homo sapiens
  source 1..989: mol_type = other DNA
cctccaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 14 - DNA, 1407 bp, Homo sapiens
  source 1..1407: mol_type = other DNA
atgggatggtcatgtatcatcctttttctagtagcaactgcaaccggtgtacattccgaggtgcagctggtggagtctgggggaggcctggtcaagcctggggggtccctgagactctcctgtgcagcctctggattcaccttcagttcctatagcatgaactgggtccgccaggctccagggaaggggctggagtgggtctcatccattagtggcaatagtaattacatatattacgccgactcagtgaagggccgattcaccatctccagagacaacgccaagaactcactctatctgcaaacgagcagcctgagagccgaggacacggctctctatttctgtgcgagggggacaattattggagctgcgggatatgactgctggggacagggaacccttgtcactgtctcctcagcctccaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 15 - DNA, 57 bp, Homo sapiens; identical to SEQ ID NO: 11
  source 1..57: mol_type = other DNA

SEQ ID NO: 16 - DNA, 322 bp, Homo sapiens
  source 1..322: mol_type = other DNA
gccatccagatgacccagtctccatccttcctgtctgcatctgtaggagacagagtcacactcacttgccgggccagtcaggacattagcacttctttagcctggtatcatcagaaaccagggaaagcccctgaacgcctggtctatgctgcatccactttgcaaagtggggtcccatcaaggttcagcggcagtgggtctgggacaggattcactctcacaatcagcagcctgcagcctgaagattttggaacttattactgtcaacaacttaaaagttaccctctcgctttcggcggagggaccaaggtggagatcaaac

SEQ ID NO: 17 - DNA, 323 bp, Homo sapiens
  source 1..323: mol_type = other DNA
gaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 18 - DNA, 702 bp, Homo sapiens
  source 1..702: mol_type = other DNA
atgggatggtcatgtatcatcctttttctagtagcaactgcaaccggtgtacattccgccatccagatgacccagtctccatccttcctgtctgcatctgtaggagacagagtcacactcacttgccgggccagtcaggacattagcacttctttagcctggtatcatcagaaaccagggaaagcccctgaacgcctggtctatgctgcatccactttgcaaagtggggtcccatcaaggttcagcggcagtgggtctgggacaggattcactctcacaatcagcagcctgcagcctgaagattttggaacttattactgtcaacaacttaaaagttaccctctcgctttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 19 - protein, 8 aa, Homo sapiens
GFTFSNYG

SEQ ID NO: 20 - protein, 8 aa, Homo sapiens
ISYDGRNR

SEQ ID NO: 21 - protein, 14 aa, Homo sapiens
AKKILDNGTFQGSY

SEQ ID NO: 22 - protein, 6 aa, Homo sapiens
RGFGNY

SEQ ID NO: 23 - skipped

SEQ ID NO: 24 - protein, 9 aa, Homo sapiens
QKYDNDPWA

SEQ ID NO: 25 - protein, 121 aa, Homo sapiens
EVQLVESGGGVVQPGRSLRLSCTASGFTFSNYGMHWVRQAPGKGLEWVAVISYDGRNRFYAESVKGRFTISRDNSKNTLYLQMNSLRAEDTAVYYCAKKILDNGTFQGSYWGQGTLLTVSS

SEQ ID NO: 26 - protein, 107 aa, Homo sapiens
AIQMTQSPSSLSASVGDRVTITCRASRGFGNYLAWYQQMPGKVPKLLIYGASTLQSGVPSRFSGSGSGTDFSLTISSLQPEDVATYYCQKYDNDPWAFGQGTKVEIK

SEQ ID NO: 27 - protein, 469 aa, Homo sapiens
MGWSCIILFLVATATGVHSEVQLVESGGGVVQPGRSLRLSCTASGFTFSNYGMHWVRQAPGKGLEWVAVISYDGRNRFYAESVKGRFTISRDNSKNTLYLQMNSLRAEDTAVYYCAKKILDNGTFQGSYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 28 - protein, 233 aa, Homo sapiens
MGWSCIILFLVATATGVHSAIQMTQSPSSLSASVGDRVTITCRASRGFGNYLAWYQQMPGKVPKLLIYGASTLQSGVPSRFSGSGSGTDFSLTISSLQPEDVATYYCQKYDNDPWAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 57 bp, Homo sapiens; identical to SEQ ID NO: 11
  source 1..57: mol_type = other DNA

SEQ ID NO: 30 - DNA, 364 bp, Homo sapiens
  source 1..364: mol_type = other DNA
gaggtgcagctggtggagtctgggggaggcgtggtccagcctgggaggtccctgagactctcctgtacagcctctggattcactttcagtaattatggcatgcactgggtccgccaggctccaggcaaggggctggagtgggtggcagttatatcatatgatggaaggaatagattctatgcagagtccgtgaagggccgattcaccatctccagagacaattctaagaacacgctgtatcttcaaatgaacagcctgagagctgaggacacggctgtctactactgtgcgaaaaagatactcgacaacggtacttttcaggggagctattggggccagggaaccctactcaccgtctcctcag

SEQ ID NO: 31 - DNA, 989 bp, Homo sapiens; identical to SEQ ID NO: 13
  source 1..989: mol_type = other DNA

SEQ ID NO: 32 - DNA, 1410 bp, Homo sapiens
  source 1..1410: mol_type = other DNA
atgggatggtcatgtatcatcctttttctagtagcaactgcaaccggtgtacattccgaggtgcagctggtggagtctgggggaggcgtggtccagcctgggaggtccctgagactctcctgtacagcctctggattcactttcagtaattatggcatgcactgggtccgccaggctccaggcaaggggctggagtgggtggcagttatatcatatgatggaaggaatagattctatgcagagtccgtgaagggccgattcaccatctccagagacaattctaagaacacgctgtatcttcaaatgaacagcctgagagctgaggacacggctgtctactactgtgcgaaaaagatactcgacaacggtacttttcaggggagctattggggccagggaaccctactcaccgtctcctcagcctccaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 33 - DNA, 57 bp, Homo sapiens; identical to SEQ ID NO: 11
  source 1..57: mol_type = other DNA

SEQ ID NO: 34 - DNA, 322 bp, Homo sapiens
  source 1..322: mol_type = other DNA
gccatccagatgacccagtctccatcctccctgtctgcatctgtaggagacagagtcaccatcacttgccgggcgagtcggggcttcggtaattatttagcctggtatcagcagatgccagggaaagttcctaagctcctgatctatggtgcatccactttgcaatcaggggtcccatctcgcttcagtggcagtggatctgggacagatttcagtctcaccatcagcagcctgcagcctgaagatgttgcaacttattactgtcaaaagtatgacaatgacccttgggcgttcggccaggggaccaaggtggaaatcaaac

SEQ ID NO: 35 - DNA, 323 bp, Homo sapiens; identical to SEQ ID NO: 17
  source 1..323: mol_type = other DNA

SEQ ID NO: 36 - DNA, 702 bp, Homo sapiens
  source 1..702: mol_type = other DNA
atgggatggtcatgtatcatcctttttctagtagcaactgcaaccggtgtacattccgccatccagatgacccagtctccatcctccctgtctgcatctgtaggagacagagtcaccatcacttgccgggcgagtcggggcttcggtaattatttagcctggtatcagcagatgccagggaaagttcctaagctcctgatctatggtgcatccactttgcaatcaggggtcccatctcgcttcagtggcagtggatctgggacagatttcagtctcaccatcagcagcctgcagcctgaagatgttgcaacttattactgtcaaaagtatgacaatgacccttgggcgttcggccaggggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 37 - protein, 8 aa, Homo sapiens
GFPFSSRW

SEQ ID NO: 38 - protein, 8 aa, Homo sapiens
IDTNGRTT

SEQ ID NO: 39 - protein, 20 aa, Homo sapiens
ARDLPNFDSSDAGWGHGVDV

SEQ ID NO: 40 - protein, 6 aa, Homo sapiens
QTISSH

SEQ ID NO: 41 - skipped

SEQ ID NO: 42 - protein, 9 aa, Homo sapiens
LQTYTSLPT

SEQ ID NO: 43 - protein, 127 aa, Homo sapiens
EVQLVESGGGLVQPGGSLRLSCEASGFPFSSRWIHWVRQGPGKGLVWLSRIDTNGRTTNYADSVNGRFTISKDNGKSTVYLQMNSLRAEDTAVYYCARDLPNFDSSDAGWGHGVDVWGQGTTVIVSS

SEQ ID NO: 44 - protein, 107 aa, Homo sapiens
AIQMTQSPSSLSASVGDRVTITCRARQTISSHLSWYQQKPGKPPKSLIYAASHLQSGVPSRFSGSGSGTDFTLTISRLQPEDSATYYCLQTYTSLPTFGPGTKVEIK

SEQ ID NO: 45 - protein, 475 aa, Homo sapiens
MGWSCIILFLVATATGVHSEVQLVESGGGLVQPGGSLRLSCEASGFPFSSRWIHWVRQGPGKGLVWLSRIDTNGRTTNYADSVNGRFTISKDNGKSTVYLQMNSLRAEDTAVYYCARDLPNFDSSDAGWGHGVDVWGQGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 46 - protein, 233 aa, Homo sapiens
MGWSCIILFLVATATGVHSAIQMTQSPSSLSASVGDRVTITCRARQTISSHLSWYQQKPGKPPKSLIYAASHLQSGVPSRFSGSGSGTDFTLTISRLQPEDSATYYCLQTYTSLPTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - DNA, 57 bp, Homo sapiens; identical to SEQ ID NO: 11
  source 1..57: mol_type = other DNA

SEQ ID NO: 48 - DNA, 381 bp, Homo sapiens
  source 1..381: mol_type = other DNA
gaggtgcagctggtggagtctgggggaggcttagttcagcctggggggtccctgagactctcctgtgaagcctctgggttccccttcagtagtcgctggattcactgggtccgccagggtcccgggaaggggctggtgtggctctcacgtattgacactaatgggaggacaacaaactacgcggactccgtaaatggccgattcaccatctccaaagacaacggcaagagcacggtgtatctgcaaatgaatagcctgagagccgaggacacggctgtgtattattgtgcaagagatttgcccaattttgactcctccgatgcaggatggggccacggtgtggacgtctggggccaagggaccacggtcatcgtctcctca

SEQ ID NO: 49 - DNA, 990 bp, Homo sapiens
  source 1..990: mol_type = other DNA
gcctccaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 50 - DNA, 1428 bp, Homo sapiens
  source 1..1428: mol_type = other DNA
atgggatggtcatgtatcatcctttttctagtagcaactgcaaccggtgtacattccgaggtgcagctggtggagtctgggggaggcttagttcagcctggggggtccctgagactctcctgtgaagcctctgggttccccttcagtagtcgctggattcactgggtccgccagggtcccgggaaggggctggtgtggctctcacgtattgacactaatgggaggacaacaaactacgcggactccgtaaatggccgattcaccatctccaaagacaacggcaagagcacggtgtatctgcaaatgaatagcctgagagccgaggacacggctgtgtattattgtgcaagagatttgcccaattttgactcctccgatgcaggatggggccacggtgtggacgtctggggccaagggaccacggtcatcgtctcctcagcctccaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 51 - DNA, 57 bp, Homo sapiens; identical to SEQ ID NO: 11
  source 1..57: mol_type = other DNA

SEQ ID NO: 52 - DNA, 322 bp, Homo sapiens
  source 1..322: mol_type = other DNA
gccatccagatgacccagtctccatcctccctgtccgcatctgtaggagacagagtcaccatcacttgccgggcaaggcagaccattagtagtcacttaagttggtatcagcagaaaccagggaagccgcctaagtccctgatctatgctgcatcccacttgcaaagtggggtcccatcacggttcagtggcagtggatctgggacagatttcactctcaccatcagccgtctgcaacctgaagattctgctacttattattgtctacagacttatacttcccttcccacattcggccctgggaccaaagtggaaattaaac

SEQ ID NO: 53 - DNA, 323 bp, Homo sapiens
  source 1..323: mol_type = other DNA
gaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 54 - DNA, 702 bp, Homo sapiens
  source 1..702: mol_type = other DNA
atgggatggtcatgtatcatcctttttctagtagcaactgcaaccggtgtacattccgccatccagatgacccagtctccatcctccctgtccgcatctgtaggagacagagtcaccatcacttgccgggcaaggcagaccattagtagtcacttaagttggtatcagcagaaaccagggaagccgcctaagtccctgatctatgctgcatcccacttgcaaagtggggtcccatcacggttcagtggcagtggatctgggacagatttcactctcaccatcagccgtctgcaacctgaagattctgctacttattattgtctacagacttatacttcccttcccacattcggccctgggaccaaagtggaaattaaacgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 55 - protein, 8 aa, Homo sapiens
GYIFSNNG

SEQ ID NO: 56 - protein, 8 aa, Homo sapiens
ISPYTGHT

SEQ ID NO: 57 - protein, 13 aa, Homo sapiens
ATDYQDGVRALAH

SEQ ID NO: 58 - protein, 8 aa, Homo sapiens
NSNIGNNH

SEQ ID NO: 59 - skipped

SEQ ID NO: 60 - protein, 11 aa, Homo sapiens
GTWDTSLNARV

SEQ ID NO: 61 - protein, 120 aa, Homo sapiens
QVQLVQSGADVENPGASVKVSCRSSGYIFSNNGITWVRQVPGQGLEWMGWISPYTGHTNYAQTLQGRVAMTTDTSTSIFYMELRSLRSDDTAVYYCATDYQDGVRALAHWGQGTLVTVSS

SEQ ID NO: 62 - protein, 110 aa, Homo sapiens
QAVVTQEPSMSAAPGQKVTISCSGINSNIGNNHVSWYQQLPGTAPKLLIYDDYKRPSGIPDRFSGSKSGTSATLGITGLQTGDEGDYYCGTWDTSLNARVFGGGTKLTVL

SEQ ID NO: 63 - protein, 469 aa, Homo sapiens
MGWSCIILFLVATATGVHSQVQLVQSGADVENPGASVKVSCRSSGYIFSNNGITWVRQVPGQGLEWMGWISPYTGHTNYAQTLQGRVAMTTDTSTSIFYMELRSLRSDDTAVYYCATDYQDGVRALAHWGQGTLVTVSSASPTSPKSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 64 - protein, 235 aa, Homo sapiens
MGWSCIILFLVATATGSWAQAVVTQEPSMSAAPGQKVTISCSGINSNIGNNHVSWYQQLPGTAPKLLIYDDYKRPSGIPDRFSGSKSGTSATLGITGLQTGDEGDYYCGTWDTSLNARVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 65 - DNA, 57 bp, Homo sapiens; identical to SEQ ID NO: 11
  source 1..57: mol_type = other DNA

SEQ ID NO: 66 - DNA, 361 bp, Homo sapiens
  source 1..361: mol_type = other DNA
caggtgcagctggtgcagtctggggctgacgtggagaaccctggggcctcagtgaaagtctcctgcaggtcttctggttatatttttagcaacaatggcatcacctgggtgcgacaggtccctggacaaggccttgagtggatggggtggatcagcccttacactggtcacacaaactatgcacagacgctccagggcagagtcgccatgaccacagacacatccacgagtatattctacatggagctgaggagcctgaggtctgacgacacggccgtctattactgcgcgaccgattaccaggatggtgtccgagcattggcccactggggccagggaaccctggtcaccgtctcctcag

SEQ ID NO: 67 - DNA, 992 bp, Homo sapiens
  source 1..992: mol_type = other DNA
catccccgaccagccccaagtcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 68 - DNA, 1410 bp, Homo sapiens
  source 1..1410: mol_type = other DNA
atgggatggtcatgtatcatcctttttctagtagcaactgcaaccggtgtacattcccaggtgcagctggtgcagtctggggctgacgtggagaaccctggggcctcagtgaaagtctcctgcaggtcttctggttatatttttagcaacaatggcatcacctgggtgcgacaggtccctggacaaggccttgagtggatggggtggatcagcccttacactggtcacacaaactatgcacagacgctccagggcagagtcgccatgaccacagacacatccacgagtatattctacatggagctgaggagcctgaggtctgacgacacggccgtctattactgcgcgaccgattaccaggatggtgtccgagcattggcccactggggccagggaaccctggtcaccgtctcctcagcatccccgaccagccccaagtcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 69 - DNA, 57 bp, Homo sapiens
  source 1..57: mol_type = other DNA
atgggatggtcatgtatcatcctttttctagtagcaactgcaaccggttcctgggcc

SEQ ID NO: 70 - DNA, 330 bp, Homo sapiens
  source 1..330: mol_type = other DNA
caggctgtggtgactcaggagccctcaatgtctgcggccccaggacagaaggtcaccatctcctgctctggaatcaactccaacattggaaataatcatgtttcctggtatcagcagctcccaggaacagcccccaaactcctcatttatgacgattataagcgaccctcagggattcctgaccgattctctggctccaagtctggcacgtcagccaccctgggcatcaccggactccagactggggacgagggcgattattattgcgggacatgggataccagcctgaatgcccgggtgttcggcggagggaccaagctgaccgtccta

SEQ ID NO: 71 - DNA, 321 bp, Homo sapiens
  source 1..321: mol_type = other DNA
agtcagcccaaggctgccccctcggtcactctgttcccgccctcgagtgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatag

SEQ ID NO: 72 - DNA, 708 bp, Homo sapiens
  source 1..708: mol_type = other DNA
atgggatggtcatgtatcatcctttttctagtagcaactgcaaccggttcctgggcccaggctgtggtgactcaggagccctcaatgtctgcggccccaggacagaaggtcaccatctcctgctctggaatcaactccaacattggaaataatcatgtttcctggtatcagcagctcccaggaacagcccccaaactcctcatttatgacgattataagcgaccctcagggattcctgaccgattctctggctccaagtctggcacgtcagccaccctgggcatcaccggactccagactggggacgagggcgattattattgcgggacatgggataccagcctgaatgcccgggtgttcggcggagggaccaagctgaccgtcctaagtcagcccaaggctgccccctcggtcactctgttcccgccctcgagtgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatag

SEQ ID NO: 73 - protein, 8 aa, Homo sapiens
GFTFTKTW

SEQ ID NO: 74 - protein, 10 aa, Homo sapiens
IKSKIDGETT

SEQ ID NO: 75 - protein, 10 aa, Homo sapiens
TSRVLTTNDY

SEQ ID NO: 76 - protein, 9 aa, Homo sapiens
SSDVGGYDY

SEQ ID NO: 77 - skipped

SEQ ID NO: 78 - protein, 10 aa, Homo sapiens
CSFTTSGTFV

SEQ ID NO: 79 - protein, 119 aa, Homo sapiens
EVQLVESGGGLVKPGGSLRLSCAASGFTFTKTWMNWVRQAPGKGLEWLGRIKSKIDGETTDYAAPVKGRFTISRDDSKNTVYLQMNSLGTEDTALYYCTSRVLTTNDYWGQGTLVTVSS

SEQ ID NO: 80 - protein, 110 aa, Homo sapiens
QSALTQPASVSGSPGQSIAISCTGTSSDVGGYDYVSWYQQHPGKVPKHMIYDVSNRPSGVSNRFSGSKSGNTASLTISGLQPEDEADYYCCSFTTSGTFVFGTGTKVTVL

SEQ ID NO: 81 - protein, 468 aa, Homo sapiens
MGWSCIILFLVATATGVHSEVQLVESGGGLVKPGGSLRLSCAASGFTFTKTWMNWVRQAPGKGLEWLGRIKSKIDGETTDYAAPVKGRFTISRDDSKNTVYLQMNSLGTEDTALYYCTSRVLTTNDYWGQGTLVTVSSASPTSPKSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 82 - protein, 235 aa, Homo sapiens
MGWSCIILFLVATATGSWAQSALTQPASVSGSPGQSIAISCTGTSSDVGGYDYVSWYQQHPGKVPKHMIYDVSNRPSGVSNRFSGSKSGNTASLTISGLQPEDEADYYCCSFTTSGTFVFGTGTKVTVLGQPKANPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 83 - DNA, 57 bp, Homo sapiens; identical to SEQ ID NO: 11
  source 1..57: mol_type = other DNA

SEQ ID NO: 84 - DNA, 358 bp, Homo sapiens
  source 1..358: mol_type = other DNA
gaggtgcagctggtggagtctgggggaggcttggtaaagcctggggggtcccttagactctcctgtgcggcctctggattcactttcactaagacctggatgaactgggtccgccaggctccagggaaggggctggagtggcttggtcgtattaaaagcaaaattgatggtgagacaacagactacgctgcacccgtgaaaggcagattcaccatctcaagagatgattcaaaaaacacggtgtatctgcaaatgaacagcctgggaaccgaggacacagccctatattactgtacctctcgagtcctgactacgaatgactactggggccagggaaccctggtcaccgtctcctcag

SEQ ID NO: 85 - DNA, 992 bp, Homo sapiens; identical to SEQ ID NO: 67
  source 1..992: mol_type = other DNA

SEQ ID NO: 86 - DNA, 1407 bp, Homo sapiens
  source 1..1407: mol_type = other DNA
atgggatggtcatgtatcatcctttttctagtagcaactgcaaccggtgtacattccgaggtgcagctggtggagtctgggggaggcttggtaaagcctggggggtcccttagactctcctgtgcggcctctggattcactttcactaagacctggatgaactgggtccgccaggctccagggaaggggctggagtggcttggtcgtattaaaagcaaaattgatggtgagacaacagactacgctgcacccgtgaaaggcagattcaccatctcaagagatgattcaaaaaacacggtgtatctgcaaatgaacagcctgggaaccgaggacacagccctatattactgtacctctcgagtcctgactacgaatgactactggggccagggaaccctggtcaccgtctcctcagcatccccgaccagccccaagtcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 87 - DNA, 57 bp, Homo sapiens; identical to SEQ ID NO: 69
  source 1..57: mol_type = other DNA

SEQ ID NO: 88 - DNA, 329 bp, Homo sapiens
  source 1..329: mol_type = other DNA
cagtctgccctgactcagcctgcctccgtgtctgggtctcctggacagtcgatcgccatctcctgcactggaacgagcagtgacgttggtggttatgactatgtctcctggtaccaacaacaccccggcaaagtccccaaacacatgatttatgatgtcagtaatcggccctcaggggtttctaatcgcttctctggctccaagtctggcaacacggcctccctgaccatctctggactccagcctgaggacgaggctgattattactgctgctcatttacaaccagcggaacttttgtcttcggaactgggaccaaggtcaccgtcct

SEQ ID NO: 89 - DNA, 322 bp, Homo sapiens
  source 1..322: mol_type = other DNA
tggtcagcccaaggccaaccccactgtcactctgttcccaccctcgagtgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatag

SEQ ID NO: 90 - DNA, 708 bp, Homo sapiens
  source 1..708: mol_type = other DNA
atgggatggtcatgtatcatcctttttctagtagcaactgcaaccggttcctgggcccagtctgccctgactcagcctgcctccgtgtctgggtctcctggacagtcgatcgccatctcctgcactggaacgagcagtgacgttggtggttatgactatgtctcctggtaccaacaacaccccggcaaagtccccaaacacatgatttatgatgtcagtaatcggccctcaggggtttctaatcgcttctctggctccaagtctggcaacacggcctccctgaccatctctggactccagcctgaggacgaggctgattattactgctgctcatttacaaccagcggaacttttgtcttcggaactgggaccaaggtcaccgtccttggtcagcccaaggccaaccccactgtcactctgttcccaccctcgagtgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatag

SEQ ID NO: 91 - protein, 8 aa, Homo sapiens
GFTFSNTW

SEQ ID NO: 92 - protein, 10 aa, Homo sapiens
IKRKVDGETT

SEQ ID NO: 93 - protein, 10 aa, Homo sapiens
TSRVLTTNDH

SEQ ID NO: 94 - protein, 9 aa, Homo sapiens
SSDVGRYDY

SEQ ID NO: 95 - skipped

SEQ ID NO: 96 - protein, 10 aa, Homo sapiens
CSFTGGEIFV

SEQ ID NO: 97 - protein, 117 aa, Homo sapiens
EVQLVESGGGLVKPGGSLRLSCAASGFTFSNTWMNWVRQAPGKGLEYLGRIKRKVDGETTHYAAPAKGRFTISRDDSKNTLYLQLSSLGTEDTALYYCTSRVLTTNDHWGQGTLVTV

SEQ ID NO: 98 - protein, 110 aa, Homo sapiens
QSALTQPASVSGSPGQSITISCTGTSSDVGRYDYVSWYQQHPGKVPKLLIYDVSNRPSGGSNRFSGSKSGNTASLTISGLQPEDEADYYCCSFTGGEIFVFGTGTKVTVL

SEQ ID NO: 99 - protein, 469 aa, Homo sapiens
MGWSCIILFLVATATGVHSEVQLVESGGGLVKPGGSLRLSCAASGFTFSNTWMNWVRQAPGKGLEYLGRIKRKVDGETTHYAAPAKGRFTISRDDSKNTLYLQLSSLGTEDTALYYCTSRVLTTNDHWGQGTLVTVHSSVRSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 100 - protein, 235 aa, Homo sapiens
MGWSCIILFLVATATGSWAQSALTQPASVSGSPGQSITISCTGTSSDVGRYDYVSWYQQHPGKVPKLLIYDVSNRPSGGSNRFSGSKSGNTASLTISGLQPEDEADYYCCSFTGGEIFVFGTGTKVTVLGQPKANPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 101 - DNA, 57 bp, Homo sapiens; identical to SEQ ID NO: 11
  source 1..57: mol_type = other DNA

SEQ ID NO: 102 - DNA, 350 bp, Homo sapiens
  source 1..350: mol_type = other DNA
gaggtgcagctggtggagtctgggggaggcttggtaaagcctggggggtcccttagactctcctgtgcggcctctggattcactttcagtaacacctggatgaactgggtccgccaggctccagggaaggggctggagtaccttggtcgtattaaaaggaaagttgatggtgagacaacacactacgctgcacccgcgaaaggcagattcaccatctcaagagatgattccaaaaacactctgtatctgcaactgagcagcctgggaaccgaggacacagccctatattattgcacctctcgagtcctgactaccaatgaccactggggccagggaaccctggtcaccgt

SEQ ID NO: 103 - DNA, 1003 bp, Homo sapiens
  source 1..1003: mol_type = other DNA
acactcgagcgtacggtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 104 - DNA, 1410 bp, Homo sapiens
  source 1..1410: mol_type = other DNA
atgggatggtcatgtatcatcctttttctagtagcaactgcaaccggtgtacattccgaggtgcagctggtggagtctgggggaggcttggtaaagcctggggggtcccttagactctcctgtgcggcctctggattcactttcagtaacacctggatgaactgggtccgccaggctccagggaaggggctggagtaccttggtcgtattaaaaggaaagttgatggtgagacaacacactacgctgcacccgcgaaaggcagattcaccatctcaagagatgattccaaaaacactctgtatctgcaactgagcagcctgggaaccgaggacacagccctatattattgcacctctcgagtcctgactaccaatgaccactggggccagggaaccctggtcaccgtacactcgagcgtacggtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 105 - DNA, 57 bp, Homo sapiens; identical to SEQ ID NO: 69
  source 1..57: mol_type = other DNA

SEQ ID NO: 106 - DNA, 329 bp, Homo sapiens
  source 1..329: mol_type = other DNA
cagtctgccctgactcagcctgcctccgtgtctgggtctcctggacagtcgatcaccatctcctgcactggaaccagcagtgacgttggtcgttatgactatgtctcctggtaccaacagcacccaggcaaagtccccaaattgttgatttatgatgtcagtaatcggccctcagggggatccaatcgcttctctggctccaagtctggcaacacggcctccctgaccatctctggactccagcctgaggacgaggccgattattactgctgctcatttactggcggcgaaatttttgtcttcggaactgggaccaaggtcactgtcct

SEQ ID NO: 107 - DNA, 322 bp, Homo sapiens; identical to SEQ ID NO: 89
  source 1..322: mol_type = other DNA

SEQ ID NO: 108 - DNA, 708 bp, Homo sapiens
  source 1..708: mol_type = other DNA
atgggatggtcatgtatcatcctttttctagtagcaactgcaaccggttcctgggcccagtctgccctgactcagcctgcctccgtgtctgggtctcctggacagtcgatcaccatctcctgcactggaaccagcagtgacgttggtcgttatgactatgtctcctggtaccaacagcacccaggcaaagtccccaaattgttgatttatgatgtcagtaatcggccctcagggggatccaatcgcttctctggctccaagtctggcaacacggcctccctgaccatctctggactccagcctgaggacgaggccgattattactgctgctcatttactggcggcgaaatttttgtcttcggaactgggaccaaggtcactgtccttggtcagcccaaggccaaccccactgtcactctgttcccaccctcgagtgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatag

SEQ ID NO: 109-126 - skipped (18 entries)

SEQ ID NO: 127 - protein, 8 aa, Homo sapiens
EFTFSSYA

SEQ ID NO: 128 - protein, 8 aa, Homo sapiens
ISTGGDRT

SEQ ID NO: 129 - protein, 18 aa, Homo sapiens
AKSLESGSIPTRVRALDY

SEQ ID NO: 130 - protein, 6 aa, Homo sapiens
QGITNF

SEQ ID NO: 131 - skipped

SEQ ID NO: 132 - protein, 9 aa, Homo sapiens
QKYNSAPWT

SEQ ID NO: 133 - protein, 125 aa, Homo sapiens
EVQLVESGGDLVQPGGSLRLSCAASEFTFSSYAMSWVRQAPGKGLEWVAVISTGGDRTYYADSVKGRFTISRDNSKNTLYLQMNSLRVEDTAVYYCAKSLESGSIPTRVRALDYWGQGTLVTVSS

SEQ ID NO: 134 - protein, 107 aa, Homo sapiens
AIQMTQSPSSLSASVGDRVTITCRASQGITNFLAWYQQKPGKVPKLLIYAASTLQSGVPSRFSGSGSGTDFTLTISSLQPEDVATYYCQKYNSAPWTFGQGTKVEIK

SEQ ID NO: 135 - protein, 473 aa, Homo sapiens
MGWSCIILFLVATATGVHSEVQLVESGGDLVQPGGSLRLSCAASEFTFSSYAMSWVRQAPGKGLEWVAVISTGGDRTYYADSVKGRFTISRDNSKNTLYLQMNSLRVEDTAVYYCAKSLESGSIPTRVRALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 136 - protein, 233 aa, Homo sapiens
MGWSCIILFLVATATGVHSAIQMTQSPSSLSASVGDRVTITCRASQGITNFLAWYQQKPGKVPKLLIYAASTLQSGVPSRFSGSGSGTDFTLTISSLQPEDVATYYCQKYNSAPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - DNA, 57 bp, Homo sapiens; identical to SEQ ID NO: 11
  source 1..57: mol_type = other DNA

SEQ ID NO: 138 - DNA, 376 bp, Homo sapiens
  source 1..376: mol_type = other DNA
gaggtgcagctggtggagtctgggggagacttggtacagcctggggggtccctgagactctcctgtgcagcctctgaattcacctttagcagctatgccatgagctgggtccgccaggctccagggaaggggctggagtgggtcgcagttattagtactggtggtgataggacatactacgcagactccgtgaagggccggttcaccatctccagagacaattccaagaacacgttgtatctgcaaatgaacagcctgagagtcgaggacacggccgtatattactgtgcgaaatccctcgaaagtggttcgataccgactcgggtccgcgctttggactactggggccagggaaccctggtcaccgtctcctcag

SEQ ID NO: 139 - DNA, 989 bp, Homo sapiens
  source 1..989: mol_type = other DNA
cttccaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 140 - DNA, 1422 bp, Homo sapiens
  source 1..1422: mol_type = other DNA
atgggatggtcatgtatcatcctttttctagtagcaactgcaaccggtgtacattccgaggtgcagctggtggagtctgggggagacttggtacagcctggggggtccctgagactctcctgtgcagcctctgaattcacctttagcagctatgccatgagctgggtccgccaggctccagggaaggggctggagtgggtcgcagttattagtactggtggtgataggacatactacgcagactccgtgaagggccggttcaccatctccagagacaattccaagaacacgttgtatctgcaaatgaacagcctgagagtcgaggacacggccgtatattactgtgcgaaatccctcgaaagtggttcgataccgactcgggtccgcgctttggactactggggccagggaaccctggtcaccgtctcctcagcttccaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 141 - DNA, 57 bp, Homo sapiens; identical to SEQ ID NO: 11
  source 1..57: mol_type = other DNA

SEQ ID NO: 142 - DNA, 322 bp, Homo sapiens
  source 1..322: mol_type = other DNA
gccatccagatgacccagtctccatcctccctgtctgcatctgtaggagaccgagtcaccatcacttgccgggcgagtcagggcattaccaattttttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtataacagtgccccgtggacgttcggccaagggaccaaggtggaaatcaaac

SEQ ID NO: 143 - DNA, 323 bp, Homo sapiens; identical to SEQ ID NO: 17
  source 1..323: mol_type = other DNA

SEQ ID NO: 144 - DNA, 702 bp, Homo sapiens
  source 1..702: mol_type = other DNA
atgggatggtcatgtatcatcctttttctagtagcaactgcaaccggtgtacattccgccatccagatgacccagtctccatcctccctgtctgcatctgtaggagaccgagtcaccatcacttgccgggcgagtcagggcattaccaattttttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtataacagtgccccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 145 - protein, 8 aa, Homo sapiens
GFSFSDYA

SEQ ID NO: 146 - protein, 8 aa, Homo sapiens
IDNNGANT

SEQ ID NO: 147 - protein, 6 aa, Homo sapiens
VRGTTT

SEQ ID NO: 148 - protein, 11 aa, Homo sapiens
QSLVHSDGNTY

SEQ ID NO: 149 - skipped

SEQ ID NO: 150 - protein, 10 aa, Homo sapiens
LQATHFPHGT

SEQ ID NO: 151 - protein, 113 aa, Homo sapiens
QVQLVQSGGGLVQPGGSLRLSCSASGFSFSDYAVHWVRQPPGKGLEYLSAIDNNGANTFYVDSVKGRFTISRDNSKNMLYLQMSGLRVDDTAVYYCVRGTTTWGQGTLVTVSS

SEQ ID NO: 152 - protein, 113 aa, Homo sapiens
DIVMTQSPLSSSVTLGQPASISCRSSQSLVHSDGNTYLSWLQQRPGQPPRLLISEISKRFSGVPDRFSGSGAGTDFTLKISRVEAEDVGVYYCLQATHFPHGTFGQGTKLEIK

SEQ ID NO: 153 - protein, 461 aa, Homo sapiens
MGWSCIILFLVATATGVHSQVQLVQSGGGLVQPGGSLRLSCSASGFSFSDYAVHWVRQPPGKGLEYLSAIDNNGANTFYVDSVKGRFTISRDNSKNMLYLQMSGLRVDDTAVYYCVRGTTTWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 154 - protein, 239 aa, Homo sapiens
MGWSCIILFLVATATGVHSDIVMTQSPLSSSVTLGQPASISCRSSQSLVHSDGNTYLSWLQQRPGQPPRLLISEISKRFSGVPDRFSGSGAGTDFTLKISRVEAEDVGVYYCLQATHFPHGTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - DNA, 57 bp, Homo sapiens; identical to SEQ ID NO: 11
  source 1..57: mol_type = other DNA

SEQ ID NO: 156 - DNA, 340 bp, Homo sapiens
  source 1..340: mol_type = other DNA
caggtgcagctggtgcagtctgggggcggcttggtccagccgggggggtccctgagactctcctgttcagcctctggattcagcttcagtgactatgctgtgcactgggtccgccagcctccagggaagggactggaatacctttcagctattgataataatggggctaacacattctacgtagactccgtgaagggcagattcaccatctccagagacaattccaagaacatgttgtatcttcagatgagtgggctgagagttgacgacaccgctgtgtattactgtgtgagggggactaccacctggggccagggaaccctagtcaccgtctcctcag

SEQ ID NO: 157 - DNA, 989 bp, Homo sapiens; identical to SEQ ID NO: 139
  source 1..989: mol_type = other DNA

SEQ ID NO: 158 - DNA, 1386 bp, Homo sapiens
  source 1..1386: mol_type = other DNA
atgggatggtcatgtatcatcctttttctagtagcaactgcaaccggtgtacattcccaggtgcagctggtgcagtctgggggcggcttggtccagccgggggggtccctgagactctcctgttcagcctctggattcagcttcagtgactatgctgtgcactgggtccgccagcctccagggaagggactggaatacctttcagctattgataataatggggctaacacattctacgtagactccgtgaagggcagattcaccatctccagagacaattccaagaacatgttgtatcttcagatgagtgggctgagagttgacgacaccgctgtgtattactgtgtgagggggactaccacctggggccagggaaccctagtcaccgtctcctcagcttccaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 159 - DNA, 57 bp, Homo sapiens; identical to SEQ ID NO: 11
  source 1..57: mol_type = other DNA

SEQ ID NO: 160 - DNA, 340 bp, Homo sapiens
  source 1..340: mol_type = other DNA
gatattgtgatgactcagtctccactctcctcatctgtcacccttggacagccggcctccatctcctgcaggtctagtcaaagcctcgtacacagtgatggaaacacttacttgagttggcttcagcagaggccaggccagccaccaagactcctaatttctgagatttctaagcggttctctggggtcccagacagattcagtggcagtggggcagggacagatttcacactgaaaatcagcagggtggaggctgaagatgtcggggtttattattgcttgcaagctacacattttcctcatgggacttttggccaggggaccaagctggagatcaaac

SEQ ID NO: 161 - DNA, 323 bp, Homo sapiens; identical to SEQ ID NO: 17
  source 1..323: mol_type = other DNA

SEQ ID NO: 162 - DNA, 720 bp, Homo sapiens
  source 1..720: mol_type = other DNA
atgggatggtcatgtatcatcctttttctagtagcaactgcaaccggtgtacattccgatattgtgatgactcagtctccactctcctcatctgtcacccttggacagccggcctccatctcctgcaggtctagtcaaagcctcgtacacagtgatggaaacacttacttgagttggcttcagcagaggccaggccagccaccaagactcctaatttctgagatttctaagcggttctctggggtcccagacagattcagtggcagtggggcagggacagatttcacactgaaaatcagcagggtggaggctgaagatgtcggggtttattattgcttgcaagctacacattttcctcatgggacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 163 - protein, 8 aa, Homo sapiens
GFTFSNHW

SEQ ID NO: 164 - protein, 8 aa, Homo sapiens
INPDGTYT

SEQ ID NO: 165 - protein, 20 aa, Homo sapiens
ARDLPRSDPPGVGWGSGMDV

SEQ ID NO: 166 - protein, 6 aa, Homo sapiens
QTISTY

SEQ ID NO: 167 - skipped

SEQ ID NO: 168 - protein, 9 aa, Homo sapiens
QQSYSTPPT

SEQ ID NO: 169 - protein, 127 aa, Homo sapiens
EVQLVESGGGLVQPGGSLRLSCAASGFTFSNHWMHWVRQGPGKGLVWVSRINPDGTYTSYADSVSGRFTIARDNAKNTLYLHMNSLRDEDTAVYYCARDLPRSDPPGVGWGSGMDVWGQGTRVTVSS

SEQ ID NO: 170 - protein, 107 aa, Homo sapiens
AIQMTQSPSSLSASAGDRVTITCRASQTISTYLSWYQQKPGKAPKMLIYASSSLQSGVPSRFSGSGSGTDFTLTISRLQPEDFATYYCQQSYSTPPTFGPGTKVEIK

SEQ ID NO: 171 - protein, 475 aa, Homo sapiens
MGWSCIILFLVATATGVHSEVQLVESGGGLVQPGGSLRLSCAASGFTFSNHWMHWVRQGPGKGLVWVSRINPDGTYTSYADSVSGRFTIARDNAKNTLYLHMNSLRDEDTAVYYCARDLPRSDPPGVGWGSGMDVWGQGTRVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 172 - protein, 233 aa, Homo sapiens
MGWSCIILFLVATATGVHSAIQMTQSPSSLSASAGDRVTITCRASQTISTYLSWYQQKPGKAPKMLIYASSSLQSGVPSRFSGSGSGTDFTLTISRLQPEDFATYYCQQSYSTPPTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - DNA, 57 bp, Homo sapiens; identical to SEQ ID NO: 11
  source 1..57: mol_type = other DNA

SEQ ID NO: 174 - DNA, 381 bp, Homo sapiens
  source 1..381: mol_type = other DNA
gaggtgcagctggtggagtctgggggaggcttagttcagccgggggggtccctgagactctcctgtgcagcctctggattcaccttcagtaaccactggatgcactgggtccgccaaggtccagggaagggactggtgtgggtctcacgaattaatcctgatgggacttacacaagttacgcggactccgtgagtggccgattcaccatcgccagagacaacgccaagaacaccctgtacctgcacatgaacagtctgagagacgaggacacggctgtatattactgtgcaagagacttgcccagatctgacccccccggtgtgggatggggctccggtatggacgtctggggccaagggaccagggtcaccgtctcctca

SEQ ID NO: 175 - DNA, 990 bp, Homo sapiens; identical to SEQ ID NO: 49
  source 1..990: mol_type = other DNA

SEQ ID NO: 176 - DNA, 1428 bp, Homo sapiens
  source 1..1428: mol_type = other DNA
atgggatggtcatgtatcatcctttttctagtagcaactgcaaccggtgtacattccgaggtgcagctggtggagtctgggggaggcttagttcagccgggggggtccctgagactctcctgtgcagcctctggattcaccttcagtaaccactggatgcactgggtccgccaaggtccagggaagggactggtgtgggtctcacgaattaatcctgatgggacttacacaagttacgcggactccgtgagtggccgattcaccatcgccagagacaacgccaagaacaccctgtacctgcacatgaacagtctgagagacgaggacacggctgtatattactgtgcaagagacttgcccagatctgacccccccggtgtgggatggggctccggtatggacgtctggggccaagggaccagggtcaccgtctcctcagcctccaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 177 - DNA, 57 bp, Homo sapiens; identical to SEQ ID NO: 11
  source 1..57: mol_type = other DNA

SEQ ID NO: 178 - DNA, 321 bp, Homo sapiens
  source 1..321: mol_type = other DNA
gccatccagatgacccagtctccatcctccctgtctgcatctgcaggagacagagtcaccatcacttgccgggcaagtcagaccattagcacctatttaagttggtatcagcaaaaaccagggaaagcccctaagatgttgatctatgcttcatccagtttgcaaagtggggtcccatcaaggttcagtggcagtggatctgggacagatttcactctcaccatcagccgtctgcaacctgaagactttgcaacttactactgtcaacagagttacagtacccctcccactttcggccctgggaccaaagtggagatcaaa

SEQ ID NO: 179 - DNA, 324 bp, Homo sapiens
  source 1..324: mol_type = other DNA
agaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 180 - DNA, 702 bp, Homo sapiens
  source 1..702: mol_type = other DNA
atgggatggtcatgtatcatcctttttctagtagcaactgcaaccggtgtacattccgccatccagatgacccagtctccatcctccctgtctgcatctgcaggagacagagtcaccatcacttgccgggcaagtcagaccattagcacctatttaagttggtatcagcaaaaaccagggaaagcccctaagatgttgatctatgcttcatccagtttgcaaagtggggtcccatcaaggttcagtggcagtggatctgggacagatttcactctcaccatcagccgtctgcaacctgaagactttgcaacttactactgtcaacagagttacagtacccctcccactttcggccctgggaccaaagtggagatcaaaagaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 181 - protein, 8 aa, Homo sapiens
GFTFSNYV

SEQ ID NO: 182 - protein, 8 aa, Homo sapiens
ISTTGYTT

SEQ ID NO: 183 - protein, 12 aa, Homo sapiens
GTLGTTARDFDY

SEQ ID NO: 184 - protein, 6 aa, Homo sapiens
QDIRND

SEQ ID NO: 185 - skipped

SEQ ID NO: 186 - protein, 9 aa, Homo sapiens
LQHNSHPFA

SEQ ID NO: 187 - protein, 119 aa, Homo sapiens
EVQLVESGGGLVQPGGSLRLSCAASGFTFSNYVMSWVRQAPGKGLEWVSAISTTGYTTPYANSVKGRFTISRDNSKNTLYLQMNSLRAEDTALYFCGTLGTTARDFDYWGQGTLVTVSS

SEQ ID NO: 188 - protein, 107 aa, Homo sapiens
AIQMTQSPSSLSASVGDRVTITCRASQDIRNDLGWYQQKPGKAPKRLIYAASNLQSGVPSRFSGSGSGTEFTLTISSLQPEDFATYYCLQHNSHPFAFGPGTKVDIK

SEQ ID NO: 189 - protein, 467 aa, Homo sapiens
MGWSCIILFLVATATGVHSEVQLVESGGGLVQPGGSLRLSCAASGFTFSNYVMSWVRQAPGKGLEWVSAISTTGYTTPYANSVKGRFTISRDNSKNTLYLQMNSLRAEDTALYFCGTLGTTARDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 190 - protein, 233 aa, Homo sapiens
MGWSCIILFLVATATGVHSAIQMTQSPSSLSASVGDRVTITCRASQDIRNDLGWYQQKPGKAPKRLIYAASNLQSGVPSRFSGSGSGTEFTLTISSLQPEDFATYYCLQHNSHPFAFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 191 - DNA, 57 bp, Homo sapiens; identical to SEQ ID NO: 11
  source 1..57: mol_type = other DNA

SEQ ID NO: 192 - DNA, 358 bp, Homo sapiens
  source 1..358: mol_type = other DNA
gaggtgcagctggtggagtctgggggaggcttggtacagcctggggggtccctgagactctcctgtgcagcctctggtttcacctttagtaactatgtcatgagctgggtccgccaggctccagggaaggggctggagtgggtctcagcaattagtactactggttataccacaccctacgcaaactctgtgaagggccggttcaccatctccagagacaattccaagaacacgctgtatctgcaaatgaacagcctgagagccgaggacacggccctttatttctgtgggaccctggggacaacagcacgcgattttgactactggggccagggaaccctggtcaccgtctcctcag

SEQ ID NO: 193 - DNA, 989 bp, Homo sapiens; identical to SEQ ID NO: 139
  source 1..989: mol_type = other DNA

SEQ ID NO: 194 - DNA, 1404 bp, Homo sapiens
  source 1..1404: mol_type = other DNA
atgggatggtcatgtatcatcctttttctagtagcaactgcaaccggtgtacattccgaggtgcagctggtggagtctgggggaggcttggtacagcctggggggtccctgagactctcctgtgcagcctctggtttcacctttagtaactatgtcatgagctgggtccgccaggctccagggaaggggctggagtgggtctcagcaattagtactactggttataccacaccctacgcaaactctgtgaagggccggttcaccatctccagagacaattccaagaacacgctgtatctgcaaatgaacagcctgagagccgaggacacggccctttatttctgtgggaccctggggacaacagcacgcgattttgactactggggccagggaaccctggtcaccgtctcctcagcttccaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 195 - DNA, 57 bp, Homo sapiens; identical to SEQ ID NO: 11
  source 1..57: mol_type = other DNA

SEQ ID NO: 196 - DNA, 322 bp, Homo sapiens
  source 1..322: mol_type = other DNA
gccatccagatgacccagtctccatcctccctgtctgcatctgtcggagacagagtcaccatcacttgccgggcaagtcaggacattagaaatgacttaggctggtatcagcagaaaccagggaaagcccctaagcgcctgatctatgctgcatccaatttgcaaagtggggtcccatcaaggttcagcggcagtggatctgggacagaattcactctcacaatcagcagcctgcagcctgaagattttgcaacttattactgtctacaacataatagtcacccattcgctttcggccctgggaccaaagtggatatcaaac

SEQ ID NO: 197 - DNA, 323 bp, Homo sapiens; identical to SEQ ID NO: 17
  source 1..323: mol_type = other DNA

SEQ ID NO: 198 - DNA, 702 bp, Homo sapiens
  source 1..702: mol_type = other DNA
atgggatggtcatgtatcatcctttttctagtagcaactgcaaccggtgtacattccgccatccagatgacccagtctccatcctccctgtctgcatctgtcggagacagagtcaccatcacttgccgggcaagtcaggacattagaaatgacttaggctggtatcagcagaaaccagggaaagcccctaagcgcctgatctatgctgcatccaatttgcaaagtggggtcccatcaaggttcagcggcagtggatctgggacagaattcactctcacaatcagcagcctgcagcctgaagattttgcaacttattactgtctacaacataatagtcacccattcgctttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 199 - protein, 8 aa, Homo sapiens
GFTFSSYG

SEQ ID NO: 200 - protein, 8 aa, Homo sapiens
IWYDGSNK

SEQ ID NO: 201 - protein, 16 aa, Homo sapiens
ASEYYYGSSGHLPIDC

SEQ ID NO: 202 - protein, 6 aa, Homo sapiens
QSVSSY

SEQ ID NO: 203 - skipped

SEQ ID NO: 204 - protein, 9 aa, Homo sapiens
QQRSNWPRT

SEQ ID NO: 205 - protein, 123 aa, Homo sapiens
EVQLVESGGGVVQPGRSLRLSCVASGFTFSSYGMHWVRQAPGKGLEWVAIIWYDGSNKYYADSVKGRFTISRDNSKNTLYLQMNSLRAEDTAVYYCASEYYYGSSGHLPIDCWGQGTLVTVSS

SEQ ID NO: 206 - protein, 107 aa, Homo sapiens
EIVLTQSPATLSLSPGERATLSCRASQSVSSYLAWYQQKPGQAPRLVIYDASNRATGIPARFSGSGSGTDFTLTISSLEPEDFAVYYCQQRSNWPRTFGQGTKVEIK

SEQ ID NO: 207 - protein, 471 aa, Homo sapiens
MGWSCIILFLVATATGVHSEVQLVESGGGVVQPGRSLRLSCVASGFTFSSYGMHWVRQAPGKGLEWVAIIWYDGSNKYYADSVKGRFTISRDNSKNTLYLQMNSLRAEDTAVYYCASEYYYGSSGHLPIDCWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 208 - protein, 233 aa, Homo sapiens
MGWSCIILFLVATATGVHSEIVLTQSPATLSLSPGERATLSCRASQSVSSYLAWYQQKPGQAPRLVIYDASNRATGIPARFSGSGSGTDFTLTISSLEPEDFAVYYCQQRSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 209 - DNA, 57 bp, Homo sapiens; identical to SEQ ID NO: 11
  source 1..57: mol_type = other DNA

SEQ ID NO: 210 - DNA, 370 bp, Homo sapiens
  source 1..370: mol_type = other DNA
gaggtgcagctggtggagtctgggggaggcgtggtccagcctgggaggtccctgagactctcctgtgtagcgtctggattcaccttcagtagttatggcatgcactgggtccgccaggctccaggcaaggggctggagtgggtggcaattatatggtatgatggaagtaataaatactatgcagactccgtgaagggccgattcaccatctccagagacaattccaagaacacgctgtatctgcaaatgaacagcctgagagccgaggacacggctgtctattattgtgcgagcgagtattactatggtagtagtggtcatttacccattgactgttggggccagggaaccctggtcaccgtctcctcag

SEQ ID NO: 211 - DNA, 989 bp, Homo sapiens; identical to SEQ ID NO: 139
  source 1..989: mol_type = other DNA

SEQ ID NO: 212 - DNA, 1416 bp, Homo sapiens
  source 1..1416: mol_type = other DNA
atgggatggtcatgtatcatcctttttctagtagcaactgcaaccggtgtacattccgaggtgcagctggtggagtctgggggaggcgtggtccagcctgggaggtccctgagactctcctgtgtagcgtctggattcaccttcagtagttatggcatgcactgggtccgccaggctccaggcaaggggctggagtgggtggcaattatatggtatgatggaagtaataaatactatgcagactccgtgaagggccgattcaccatctccagagacaattccaagaacacgctgtatctgcaaatgaacagcctgagagccgaggacacggctgtctattattgtgcgagcgagtattactatggtagtagtggtcatttacccattgactgttggggccagggaaccctggtcaccgtctcctcagcttccaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 213 - DNA, 57 bp, Homo sapiens; identical to SEQ ID NO: 11
  source 1..57: mol_type = other DNA

SEQ ID NO: 214 - DNA, 322 bp, Homo sapiens
  source 1..322: mol_type = other DNA
gaaattgtgttgacacagtctccagccaccctgtctttgtctccaggggaaagagccaccctctcctgcagggccagtcagagtgttagcagttacttagcctggtaccaacagaaacctggccaggctcccaggctcgtcatctatgatgcatccaacagggccactggcatcccagccaggttcagtggcagtgggtctgggacagacttcactctcaccatcagcagcctagagcctgaagattttgcagtttattactgtcagcagcgtagcaactggcctcggacgttcggccaagggaccaaggtggaaatcaaac

SEQ ID NO: 215 - DNA, 323 bp, Homo sapiens; identical to SEQ ID NO: 17
  source 1..323: mol_type = other DNA

SEQ ID NO: 216 - DNA, 702 bp, Homo sapiens
  source 1..702: mol_type = other DNA
atgggatggtcatgtatcatcctttttctagtagcaactgcaaccggtgtacattccgaaattgtgttgacacagtctccagccaccctgtctttgtctccaggggaaagagccaccctctcctgcagggccagtcagagtgttagcagttacttagcctggtaccaacagaaacctggccaggctcccaggctcgtcatctatgatgcatccaacagggccactggcatcccagccaggttcagtggcagtgggtctgggacagacttcactctcaccatcagcagcctagagcctgaagattttgcagtttattactgtcagcagcgtagcaactggcctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 217 - protein, 8 aa, Homo sapiens
GFTFSSHA

SEQ ID NO: 218 - protein, 8 aa, Homo sapiens
ISGTGDRT

SEQ ID NO: 219 - protein, 12 aa, Homo sapiens
ARAPIGTFYYDN

SEQ ID NO: 220 - protein, 7 aa, Homo sapiens
QSVGTRN

SEQ ID NO: 221 - skipped

SEQ ID NO: 222 - protein, 9 aa, Homo sapiens
QQYGISPRT

SEQ ID NO: 223 - protein, 119 aa, Homo sapiens
EVQLVESGGGLVQPGGSLRLSCAASGFTFSSHAMSWVRQAPGKGLEWVSAISGTGDRTDDTGSVRGRFTISRDNSKNTVYLQMFFLTVEDTALYYCARAPIGTFYYDNWGQGTLVTVSS

SEQ ID NO: 224 - protein, 108 aa, Homo sapiens
ETTLTQSPGTLSLSPGERATLSCRASQSVGTRNLAWYQQKPGQSPRLLIYGASSRATGIPDRFSGGGSGTDFTLTISRLEPEDFAVYYCQQYGISPRTFGQGTKVEIK

SEQ ID NO: 225 - protein, 467 aa, Homo sapiens
HGMVMYHPFSSTTATGVHSEVQLVESGGGLVQPGGSLRLSCAASGFTFSSHAMSWVRQAPGKGLEWVSAISGTGDRTDDTGSVRGRFTISRDNSKNTVYLQMFFLTVEDTALYYCARAPIGTFYYDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 226 - protein, 234 aa, Homo sapiens
MGWSCIILFLVATATGVHSETTLTQSPGTLSLSPGERATLSCRASQSVGTRNLAWYQQKPGQSPRLLIYGASSRATGIPDRFSGGGSGTDFTLTISRLEPEDFAVYYCQQYGISPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 227 - DNA, 57 bp, Homo sapiens
  source 1..57: mol_type = other DNA
catgggatggtcatgtatcatcctttttctagtacaactgcaaccggtgtacattcc

SEQ ID NO: 228 - DNA, 358 bp, Homo sapiens
  source 1..358: mol_type = other DNA
gaggtgcagctggtggagtctgggggaggcttggtacagcctggggggtccctgagactctcctgtgcagcctctggattcacctttagtagtcatgccatgagctgggtccgccaggctccagggaaggggctggagtgggtctcagccataagtggcactggtgatagaacagacgacacaggctccgtgaggggccggttcaccatctccagagacaattccaagaacacggtgtatctgcaaatgttttttttgacagtcgaggacacggccctatattactgtgcgagagcccctatcggaactttctactatgacaactggggccagggaaccctggtcaccgtctcatcag

SEQ ID NO: 229 - DNA, 989 bp, Homo sapiens; identical to SEQ ID NO: 13
  source 1..989: mol_type = other DNA

SEQ ID NO: 230 - DNA, 1404 bp, Homo sapiens
  source 1..1404: mol_type = other DNA
catgggatggtcatgtatcatcctttttctagtacaactgcaaccggtgtacattccgaggtgcagctggtggagtctgggggaggcttggtacagcctggggggtccctgagactctcctgtgcagcctctggattcacctttagtagtcatgccatgagctgggtccgccaggctccagggaaggggctggagtgggtctcagccataagtggcactggtgatagaacagacgacacaggctccgtgaggggccggttcaccatctccagagacaattccaagaacacggtgtatctgcaaatgttttttttgacagtcgaggacacggccctatattactgtgcgagagcccctatcggaactttctactatgacaactggggccagggaaccctggtcaccgtctcatcagcctccaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 231 - DNA, 57 bp, Homo sapiens; identical to SEQ ID NO: 11
  source 1..57: mol_type = other DNA

SEQ ID NO: 232 - DNA, 325 bp, Homo sapiens
  source 1..325: mol_type = other DNA
gaaacgacactcacgcagtctccaggcaccctgtctctgtctccaggggaaagagccaccctctcctgcagggccagtcagagtgttggcaccaggaacttagcctggtaccagcagaaacctggccagtctcccaggctcctcatctatggtgcatccagcagggccactggcatcccagacaggttcagtggcggtgggtctgggacagacttcactctcaccatcagcagactggagcctgaagattttgcagtgtattactgtcagcagtatggtatctcacctcgcacgttcggccaagggaccaaggtggaaatcaaac

SEQ ID NO: 233 - DNA, 323 bp, Homo sapiens; identical to SEQ ID NO: 17
  source 1..323: mol_type = other DNA

SEQ ID NO: 234 - DNA, 705 bp, Homo sapiens
  source 1..705: mol_type = other DNA
atgggatggtcatgtatcatcctttttctagtagcaactgcaaccggtgtacattccgaaacgacactcacgcagtctccaggcaccctgtctctgtctccaggggaaagagccaccctctcctgcagggccagtcagagtgttggcaccaggaacttagcctggtaccagcagaaacctggccagtctcccaggctcctcatctatggtgcatccagcagggccactggcatcccagacaggttcagtggcggtgggtctgggacagacttcactctcaccatcagcagactggagcctgaagattttgcagtgtattactgtcagcagtatggtatctcacctcgcacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 235 - protein, 8 aa, Homo sapiens
GLTFSSHS

SEQ ID NO: 236 - protein, 8 aa, Homo sapiens
ISGDSDHI

SEQ ID NO: 237 - protein, 11 aa, Homo sapiens
TNWGKFAVVSY

SEQ ID NO: 238 - protein, 11 aa, Homo sapiens
QSLVFRDGNIY

SEQ ID NO: 239 - skipped

SEQ ID NO: 240 - protein, 9 aa, Homo sapiens
MQGSHWPYS

SEQ ID NO: 241 - protein, 118 aa, Homo sapiens
EVQLVESGGGLVQPGGSLRLSCAASGLTFSSHSMGWVRQAPGKGLEWVSIISGDSDHIYYADSVKGRFTISRDNSQNTVDLLMSGLRAEDTAVYYCTNWGKFAVVSYCGQGTLVTVSS

SEQ ID NO: 242 - protein, 112 aa, Homo sapiens
DIVMTQSPLSLPVTLGQPASISCRSSQSLVFRDGNIYLNWFQQRPGQSPRRLIYQISKRDSGVPDRFSGSGSGTDFTLQISRVEAEDVGVYYCMQGSHWPYSFGQGTKLEIK

SEQ ID NO: 243 - protein, 466 aa, Homo sapiens
MGWSCIILFLVATATGVHSEVQLVESGGGLVQPGGSLRLSCAASGLTFSSHSMGWVRQAPGKGLEWVSIISGDSDHIYYADSVKGRFTISRDNSQNTVDLLMSGLRAEDTAVYYCTNWGKFAVVSYCGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 244 - protein, 238 aa, Homo sapiens
MGWSCIILFLVATATGVHSDIVMTQSPLSLPVTLGQPASISCRSSQSLVFRDGNIYLNWFQQRPGQSPRRLIYQISKRDSGVPDRFSGSGSGTDFTLQISRVEAEDVGVYYCMQGSHWPYSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 245 - DNA, 57 bp, Homo sapiens; identical to SEQ ID NO: 11
  source 1..57: mol_type = other DNA

SEQ ID NO: 246 - DNA, 355 bp, Homo sapiens
  source 1..355: mol_type = other DNA
gaggtgcagctggtggagtctgggggaggcttggtacagcctggggggtccctgagactctcctgtgcagcctctggactcacctttagcagccatagcatgggttgggtccgccaggctccagggaaggggctggagtgggtctcaattattagtggagatagtgatcatatatactacgcagattccgtgaagggccgattcaccatctccagagacaattcccagaacaccgtggatctgctaatgagcggcctgagagccgaggacacggccgtatattactgtacgaactggggaaagttcgcagttgtatcatactgcggccagggaaccctggtcaccgtctcttcag

SEQ ID NO: 247 - DNA, 989 bp, Homo sapiens; identical to SEQ ID NO: 13
  source 1..989: mol_type = other DNA

SEQ ID NO: 248 - DNA, 1401 bp, Homo sapiens
  source 1..1401: mol_type = other DNA
atgggatggtcatgtatcatcctttttctagtagcaactgcaaccggtgtacattccgaggtgcagctggtggagtctgggggaggcttggtacagcctggggggtccctgagactctcctgtgcagcctctggactcacctttagcagccatagcatgggttgggtccgccaggctccagggaaggggctggagtgggtctcaattattagtggagatagtgatcatatatactacgcagattccgtgaagggccgattcaccatctccagagacaattcccagaacaccgtggatctgctaatgagcggcctgagagccgaggacacggccgtatattactgtacgaactggggaaagttcgcagttgtatcatactgcggccagggaaccctggtcaccgtctcttcagcctccaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 249 - DNA, 57 bp, Homo sapiens; identical to SEQ ID NO: 11
  source 1..57: mol_type = other DNA

SEQ ID NO: 250 - DNA, 337 bp, Homo sapiens
  source 1..337: mol_type = other DNA
gatattgtgatgactcagtctccactctccctgcccgtcacccttggacagccggcctccatctcctgcaggtctagtcaaagcctcgtattccgtgatggaaacatctacttaaattggtttcagcagaggccaggccaatctccaaggcgcctaatttatcagatttctaagcgggactctggggtcccagacagattcagcggcagtggatcaggcactgatttcacactgcaaatcagcagggtggaggctgaggatgttggggtttattactgcatgcaaggttcacactggccgtacagttttggccaggggaccaagctggagatcaaac

SEQ ID NO: 251 - DNA, 323 bp, Homo sapiens; identical to SEQ ID NO: 17
  source 1..323: mol_type = other DNA

SEQ ID NO: 252 - DNA, 717 bp, Homo sapiens
  source 1..717: mol_type = other DNA
atgggatggtcatgtatcatcctttttctagtagcaactgcaaccggtgtacattccgatattgtgatgactcagtctccactctccctgcccgtcacccttggacagccggcctccatctcctgcaggtctagtcaaagcctcgtattccgtgatggaaacatctacttaaattggtttcagcagaggccaggccaatctccaaggcgcctaatttatcagatttctaagcgggactctggggtcccagacagattcagcggcagtggatcaggcactgatttcacactgcaaatcagcagggtggaggctgaggatgttggggtttattactgcatgcaaggttcacactggccgtacagttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 253 - protein, 8 aa, Homo sapiens
GFTFSSYS

SEQ ID NO: 254 - protein, 8 aa, Homo sapiens
IDGSSTFI

SEQ ID NO: 255 - protein, 14 aa, Homo sapiens
ATRGSHSITNGFDI

SEQ ID NO: 256 - protein, 6 aa, Homo sapiens
QSVSSS

SEQ ID NO: 257 - skipped

SEQ ID NO: 258 - protein, 10 aa, Homo sapiens
QQRSNWPPYS

SEQ ID NO: 259 - protein, 121 aa, Homo sapiens
EVQLVESGGGLVKPGGSLRLSCAASGFTFSSYSMHWVRQAPGKGLEWVSSIDGSSTFIYYADSVKGRFTISRDNAKNSLYLQMNSLRAEDTAVFYCATRGSHSITNGFDIWGHGTMVTVSS

SEQ ID NO: 260 - protein, 108 aa, Homo sapiens
EIVLTQSPATLSLSPGERATLSCRASQSVSSSLAWYQQKPGQAPRLLIYDASNRATGIPARFSGSGSGTDFTLTISSLEPEDFAVYYCQQRSNWPPYSFGQGTKLEIK

SEQ ID NO: 261 - protein, 469 aa, Homo sapiens
MGWSCIILFLVATATGVHSEVQLVESGGGLVKPGGSLRLSCAASGFTFSSYSMHWVRQAPGKGLEWVSSIDGSSTFIYYADSVKGRFTISRDNAKNSLYLQMNSLRAEDTAVFYCATRGSHSITNGFDIWGHGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 262 - protein, 234 aa, Homo sapiens
MGWSCIILFLVATATGVHSEIVLTQSPATLSLSPGERATLSCRASQSVSSSLAWYQQKPGQAPRLLIYDASNRATGIPARFSGSGSGTDFTLTISSLEPEDFAVYYCQQRSNWPPYSFGQGTKLEIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 263 - DNA, 57 bp, Homo sapiens; identical to SEQ ID NO: 11
  source 1..57: mol_type = other DNA

SEQ ID NO: 264 - DNA, 364 bp, Homo sapiens
  source 1..364: mol_type = other DNA
gaggtgcagctggtggagtctgggggaggcctggtcaagcctggggggtccctgagactctcctgtgcagcctctggattcaccttcagtagttatagcatgcactgggtccgccaggctccagggaaggggctggagtgggtctcatccattgatggtagtagtactttcatatactacgcagactcagtgaagggccgattcaccatctccagagacaacgccaagaactcactgtatctgcaaatgaacagcctgagggccgaggacacggctgtgttttactgtgcgactcggggtagtcattcgatcacaaatggttttgatatctggggccatgggacaatggtcaccgtctcttcag

SEQ ID NO: 265 - DNA, 989 bp, Homo sapiens; identical to SEQ ID NO: 13
  source 1..989: mol_type = other DNA

SEQ ID NO: 266 - DNA, 1410 bp, Homo sapiens
  source 1..1410: mol_type = other DNA
atgggatggtcatgtatcatcctttttctagtagcaactgcaaccggtgtacattccgaggtgcagctggtggagtctgggggaggcctggtcaagcctggggggtccctgagactctcctgtgcagcctctggattcaccttcagtagttatagcatgcactgggtccgccaggctccagggaaggggctggagtgggtctcatccattgatggtagtagtactttcatatactacgcagactcagtgaagggccgattcaccatctccagagacaacgccaagaactcactgtatctgcaaatgaacagcctgagggccgaggacacggctgtgttttactgtgcgactcggggtagtcattcgatcacaaatggttttgatatctggggccatgggacaatggtcaccgtctcttcagcctccaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 267 - DNA, 57 bp, Homo sapiens; identical to SEQ ID NO: 11
  source 1..57: mol_type = other DNA

SEQ ID NO: 268 - DNA, 324 bp, Homo sapiens
  source 1..324: mol_type = other DNA
gaaattgtgttgacacagtctccagccaccctgtctttgtctccaggggaaagagccaccctctcctgcagggccagtcagagtgttagcagctccttagcctggtaccaacagaaacctggccaggctcccaggctcctcatctatgatgcatccaacagggccactggcatcccagccaggttcagtggcagtgggtctgggacagacttcactctcaccatcagcagcctagagccggaagattttgcagtttattactgtcagcagcgtagcaactggcctccgtacagttttggccaggggaccaagctggagatcaaa

SEQ ID NO: 269 - DNA, 324 bp, Homo sapiens
  source 1..324: mol_type = other DNA
ggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 270 - DNA, 705 bp, Homo sapiens
  source 1..705: mol_type = other DNA
atgggatggtcatgtatcatcctttttctagtagcaactgcaaccggtgtacattccgaaattgtgttgacacagtctccagccaccctgtctttgtctccaggggaaagagccaccctctcctgcagggccagtcagagtgttagcagctccttagcctggtaccaacagaaacctggccaggctcccaggctcctcatctatgatgcatccaacagggccactggcatcccagccaggttcagtggcagtgggtctgggacagacttcactctcaccatcagcagcctagagccggaagattttgcagtttattactgtcagcagcgtagcaactggcctccgtacagttttggccaggggaccaagctggagatcaaaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 271 - protein, 8 aa, Homo sapiens
GFTFSTYA

SEQ ID NO: 272 - protein, 8 aa, Homo sapiens
FSGNSDDT

SEQ ID NO: 273 - protein, 15 aa, Homo sapiens
VRLWGGGTYMNRGDY

SEQ ID NO: 274 - protein, 6 aa, Homo sapiens
QGISNY

SEQ ID NO: 275 - skipped

SEQ ID NO: 276 - protein, 9 aa, Homo sapiens
QQYKSYPLT

SEQ ID NO: 277 - protein, 122 aa, Homo sapiens
EVQLVESGGDLVHPGGSLRLSCAASGFTFSTYAMSWVRQAPGKGLDWVSTFSGNSDDTFYADSVKGRFTISRDNSKNTLYLQMNSLRAEDTAVYFCVRLWGGGTYMNRGDYWGQGTLVTVSS

SEQ ID NO: 278 - protein, 107 aa, Homo sapiens
AIQMTQSPSSLSASVGDRVTITCRASQGISNYLAWFQQKPGKAPKSLIYAASSLQSGVPSKFSGSGSGTDFTLTISSLEPEDFATYYCQQYKSYPLTFGGGTKVEIK

SEQ ID NO: 279 - protein, 471 aa, Homo sapiens
MGWSCIILFLVATATGVHSEVQLVESGGDLVHPGGSLRLSCAASGFTFSTYAMSWVRQAPGKGLDWVSTFSGNSDDTFYADSVKGRFTISRDNSKNTLYLQMNSLRAEDTAVYFCVRLWGGGTYMNRGDYWGQGTLVTVSSASPTSPKSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 280 - protein, 233 aa, Homo sapiens
MGWSCIILFLVATATGVHSAIQMTQSPSSLSASVGDRVTITCRASQGISNYLAWFQQKPGKAPKSLIYAASSLQSGVPSKFSGSGSGTDFTLTISSLEPEDFATYYCQQYKSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 281 - DNA, 57 bp, Homo sapiens; identical to SEQ ID NO: 11
  source 1..57: mol_type = other DNA

SEQ ID NO: 282 - DNA, 367 bp, Homo sapiens
  source 1..367: mol_type = other DNA
gaggtgcagctggtggagtctgggggagacttggtacaccctggggggtccctgagactctcttgtgcagcctctggattcacctttagtacctatgccatgagctgggtccgccaggctccagggaaggggctggactgggtctcaacttttagtggcaatagtgatgacacattctacgcagactccgtgaagggccggttcaccatctccagagacaattccaagaacacgctgtatctgcaaatgaacagcctgagagccgaggacaccgccgtatatttctgtgtgagattgtggggtggtgggacctacatgaaccggggggactactggggccagggaaccctggtcaccgtctcctcag

SEQ ID NO: 283 - DNA, 992 bp, Homo sapiens; identical to SEQ ID NO: 67
  source 1..992: mol_type = other DNA

SEQ ID NO: 284 - DNA, 1416 bp, Homo sapiens
  source 1..1416: mol_type = other DNA
atgggatggtcatgtatcatcctttttctagtagcaactgcaaccggtgtacattccgaggtgcagctggtggagtctgggggagacttggtacaccctggggggtccctgagactctcttgtgcagcctctggattcacctttagtacctatgccatgagctgggtccgccaggctccagggaaggggctggactgggtctcaacttttagtggcaatagtgatgacacattctacgcagactccgtgaagggccggttcaccatctccagagacaattccaagaacacgctgtatctgcaaatgaacagcctgagagccgaggacaccgccgtatatttctgtgtgagattgtggggtggtgggacctacatgaaccggggggactactggggccagggaaccctggtcaccgtctcctcagcatccccgaccagccccaagtcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 285 - DNA, 57 bp, Homo sapiens; identical to SEQ ID NO: 11
  source 1..57: mol_type = other DNA

SEQ ID NO: 286 - DNA, 322 bp, Homo sapiens
  source 1..322: mol_type = other DNA
gccatccagatgacccagtctccatcctcactgtctgcatctgtaggagacagagtcaccatcacttgtcgggcgagtcagggcattagcaattatttagcctggtttcagcagaaaccagggaaagcccctaagtccctgatctatgctgcatccagtttgcaaagtggggtcccatcaaaattcagcggcagtggatctgggacagatttcactctcaccatcagcagcctggagcctgaagattttgcaacttattactgccaacagtataaaagttacccgctcactttcggcggagggaccaaggtggagatcaaac

SEQ ID NO: 287 - DNA, 323 bp, Homo sapiens; identical to SEQ ID NO: 17
  source 1..323: mol_type = other DNA

SEQ ID NO: 288 - DNA, 702 bp, Homo sapiens
  source 1..702: mol_type = other DNA
atgggatggtcatgtatcatcctttttctagtagcaactgcaaccggtgtacattccgccatccagatgacccagtctccatcctcactgtctgcatctgtaggagacagagtcaccatcacttgtcgggcgagtcagggcattagcaattatttagcctggtttcagcagaaaccagggaaagcccctaagtccctgatctatgctgcatccagtttgcaaagtggggtcccatcaaaattcagcggcagtggatctgggacagatttcactctcaccatcagcagcctggagcctgaagattttgcaacttattactgccaacagtataaaagttacccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 289 - protein, 8 aa, Homo sapiens
GISVSNIY

SEQ ID NO: 290 - protein, 7 aa, Homo sapiens
IYSSGGI

SEQ ID NO: 291 - protein, 14 aa, Homo sapiens
VRDVRGTCGGDCYK

SEQ ID NO: 292 - protein, 11 aa, Homo sapiens
QSLVHSDGNTY

SEQ ID NO: 293 - skipped

SEQ ID NO: 294 - protein, 8 aa, Homo sapiens
MQGAQWWT

SEQ ID NO: 295 - protein, 120 aa, Homo sapiens
EVQLVESGGGLVQPGGSLRLSCAVSGISVSNIYMTWVRQAPGRGLEWVSVIYSSGGIEYPDSVKGRFTISRDNSKNTLYLQMNSLRAEDTAVYYCVRDVRGTCGGDCYKWGQGTLVTVSS

SEQ ID NO: 296 - protein, 111 aa, Homo sapiens
DIVMTQTPLSLPVTLGQPASISCRSSQSLVHSDGNTYLSWFQQRPGQSPRRLIYKVSNRDSGVPDRFSGSGSGSEFTLKISRVEAEDVGVYYCMQGAQWWTFGQGTRVEIK

SEQ ID NO: 297 - protein, 469 aa, Homo sapiens
MGWSCIILFLVATATGVHSEVQLVESGGGLVQPGGSLRLSCAVSGISVSNIYMTWVRQAPGRGLEWVSVIYSSGGIEYPDSVKGRFTISRDNSKNTLYLQMNSLRAEDTAVYYCVRDVRGTCGGDCYKWGQGTLVTVSSASPTSPKSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 298 - protein, 237 aa, Homo sapiens
MGWSCIILFLVATATGVHSDIVMTQTPLSLPVTLGQPASISCRSSQSLVHSDGNTYLSWFQQRPGQSPRRLIYKVSNRDSGVPDRFSGSGSGSEFTLKISRVEAEDVGVYYCMQGAQWWTFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 299 - DNA, 57 bp, Homo sapiens; identical to SEQ ID NO: 11
  source 1..57: mol_type = other DNA

SEQ ID NO: 300 - DNA, 361 bp, Homo sapiens
  source 1..361: mol_type = other DNA
gaggtgcagctggtggagtctgggggaggcttggtccagccgggggggtccctcagactctcctgtgcagtctctggaatcagcgtcagtaacatctatatgacatgggtccgccaggctccagggagggggctggagtgggtctcagtgatttacagtagtggtggtatagagtacccagactccgtgaagggcagattcaccatctccagagacaattccaagaacacgctgtatcttcaaatgaacagcctgagagccgaggacacggctgtatattactgtgtgagagatgtgcgggggacgtgtggtggtgactgctacaagtggggccagggaaccctggtcaccgtctcctcag

SEQ ID NO: 301 - DNA, 992 bp, Homo sapiens; identical to SEQ ID NO: 67
  source 1..992: mol_type = other DNA

SEQ ID NO: 302 - DNA, 1410 bp, Homo sapiens
  source 1..1410: mol_type = other DNA
atgggatggtcatgtatcatcctttttctagtagcaactgcaaccggtgtacattccgaggtgcagctggtggagtctgggggaggcttggtccagccgggggggtccctcagactctcctgtgcagtctctggaatcagcgtcagtaacatctatatgacatgggtccgccaggctccagggagggggctggagtgggtctcagtgatttacagtagtggtggtatagagtacccagactccgtgaagggcagattcaccatctccagagacaattccaagaacacgctgtatcttcaaatgaacagcctgagagccgaggacacggctgtatattactgtgtgagagatgtgcgggggacgtgtggtggtgactgctacaagtggggccagggaaccctggtcaccgtctcctcagcatccccgaccagccccaagtcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 303 - DNA, 57 bp, Homo sapiens; identical to SEQ ID NO: 11
  source 1..57: mol_type = other DNA

SEQ ID NO: 304 - DNA, 334 bp, Homo sapiens
  source 1..334: mol_type = other DNA
gatattgtgatgacccagactccactctccctgcccgtcacccttggacagccggcctccatctcctgcaggtctagtcaaagcctcgtacacagtgatggaaacacctacttgagttggtttcagcagaggccaggccaatctccaaggcgcctaatttataaggtgtctaaccgggactctggggtcccagacagattcagcggcagtgggtcaggcagtgaattcacactgaaaatcagcagggtggaggctgaggatgttggggtctattactgcatgcaaggcgcacagtggtggacgttcggccaagggaccagggtggaaatcaaac

SEQ ID NO: 305 - DNA, 323 bp, Homo sapiens; identical to SEQ ID NO: 17
  source 1..323: mol_type = other DNA

SEQ ID NO: 306 - DNA, 714 bp, Homo sapiens
  source 1..714: mol_type = other DNA
atgggatggtcatgtatcatcctttttctagtagcaactgcaaccggtgtacattccgatattgtgatgacccagactccactctccctgcccgtcacccttggacagccggcctccatctcctgcaggtctagtcaaagcctcgtacacagtgatggaaacacctacttgagttggtttcagcagaggccaggccaatctccaaggcgcctaatttataaggtgtctaaccgggactctggggtcccagacagattcagcggcagtgggtcaggcagtgaattcacactgaaaatcagcagggtggaggctgaggatgttggggtctattactgcatgcaaggcgcacagtggtggacgttcggccaagggaccagggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 307 - protein, 8 aa, Homo sapiens
GFTFNGFA

SEQ ID NO: 308 - protein, 8 aa, Homo sapiens
ISDSGDHR

SEQ ID NO: 309 - protein, 13 aa, Homo sapiens
AKHVVWGQWYFDS

SEQ ID NO: 310 - protein, 7 aa, Homo sapiens
QTVTSNY

SEQ ID NO: 311 - skipped

SEQ ID NO: 312 - protein, 12 aa, Homo sapiens
QQYGSSVTSGPA

SEQ ID NO: 313 - protein, 120 aa, Homo sapiens
EVQLVESGGGLVQPGGSLILSCAVSGFTFNGFAMTWVRRAPGKGLEWVSTISDSGDHRFYADSVKGRFTISRDNSKNTLYLQMISLRAEDTAVYSCAKHVVWGQWYFDSWGQGTLVTVSS

SEQ ID NO: 314 - protein, 111 aa, Homo sapiens
EIVMTQSPGTLSLSPGERATLSCRASQTVTSNYLAWYQQKPGQAPRLLISGASSRATGIPDRFSGSGSGTDFTLTISRLEPEDFVVYYCQQYGSSVTSGPAFGGGTKVEIK

SEQ ID NO: 315 - protein, 468 aa, Homo sapiens
MGWSCIILFLVATATGVHSEVQLVESGGGLVQPGGSLILSCAVSGFTFNGFAMTWVRRAPGKGLEWVSTISDSGDHRFYADSVKGRFTISRDNSKNTLYLQMISLRAEDTAVYSCAKHVVWGQWYFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 316 - protein, 237 aa, Homo sapiens
MGWSCIILFLVATATGVHSEIVMTQSPGTLSLSPGERATLSCRASQTVTSNYLAWYQQKPGQAPRLLISGASSRATGIPDRFSGSGSGTDFTLTISRLEPEDFVVYYCQQYGSSVTSGPAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 317 - DNA, 57 bp, Homo sapiens; identical to SEQ ID NO: 11
  source 1..57: mol_type = other DNA

SEQ ID NO: 318 - DNA, 361 bp, Homo sapiens
  source 1..361: mol_type = other DNA
gaggtgcagctggtggagtctgggggaggcttggtacagcctggggggtccctgatactctcctgtgcagtctctggattcacctttaatggctttgccatgacctgggtccgccgggctccagggaaggggctggagtgggtctcaactattagtgatagtggtgatcacagattctacgcagactccgtgaagggccggttcaccatctccagagacaattccaagaacaccctgtatctgcaaatgatcagcctgagagccgaggacacggccgtatattcctgtgcgaaacacgtagtctggggacagtggtactttgactcctggggccagggaaccctggtcaccgtctcctcag

SEQ ID NO: 319 - DNA, 989 bp, Homo sapiens; identical to SEQ ID NO: 139
  source 1..989: mol_type = other DNA

SEQ ID NO: 320 - DNA, 1407 bp, Homo sapiens
  source 1..1407: mol_type = other DNA
atgggatggtcatgtatcatcctttttctagtagcaactgcaaccggtgtacattccgaggtgcagctggtggagtctgggggaggcttggtacagcctggggggtccctgatactctcctgtgcagtctctggattcacctttaatggctttgccatgacctgggtccgccgggctccagggaaggggctggagtgggtctcaactattagtgatagtggtgatcacagattctacgcagactccgtgaagggccggttcaccatctccagagacaattccaagaacaccctgtatctgcaaatgatcagcctgagagccgaggacacggccgtatattcctgtgcgaaacacgtagtctggggacagtggtactttgactcctggggccagggaaccctggtcaccgtctcctcagcttccaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 321 - DNA, 57 bp, Homo sapiens; identical to SEQ ID NO: 11
  source 1..57: mol_type = other DNA

SEQ ID NO: 322 - DNA, 334 bp, Homo sapiens
  source 1..334: mol_type = other DNA
gaaattgtgatgacgcagtctccaggcaccctgtctttgtctccaggggaaagagccaccctctcctgcagggccagtcagactgttaccagcaactacttagcctggtaccagcagaaacctggccaggctcccaggctcctcatctctggtgcatccagcagggccactggcatcccagacaggttcagtggcagtgggtctgggacagacttcactctcaccatcagcagactggagcctgaagatttcgtagtgtattactgtcagcagtatggtagctcagtcacctctggtcccgctttcggcggagggaccaaggtggagatcaaac

SEQ ID NO: 323 - DNA, 323 bp, Homo sapiens; identical to SEQ ID NO: 17
  source 1..323: mol_type = other DNA

SEQ ID NO: 324 - DNA, 714 bp, Homo sapiens
  source 1..714: mol_type = other DNA
atgggatggtcatgtatcatcctttttctagtagcaactgcaaccggtgtacattccgaaattgtgatgacgcagtctccaggcaccctgtctttgtctccaggggaaagagccaccctctcctgcagggccagtcagactgttaccagcaactacttagcctggtaccagcagaaacctggccaggctcccaggctcctcatctctggtgcatccagcagggccactggcatcccagacaggttcagtggcagtgggtctgggacagacttcactctcaccatcagcagactggagcctgaagatttcgtagtgtattactgtcagcagtatggtagctcagtcacctctggtcccgctttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 325 - protein, 8 aa, Homo sapiens
GFTFSSYS

SEQ ID NO: 326 - protein, 8 aa, Homo sapiens
ISGTGGTK

SEQ ID NO: 327 - protein, 12 aa, Homo sapiens
AKRTTNSGAFDV

SEQ ID NO: 328 - protein, 7 aa, Homo sapiens
QSVTGSH

SEQ ID NO: 329 - skipped

SEQ ID NO: 330 - protein, 10 aa, Homo sapiens
QQYGSSPLFS

SEQ ID NO: 331 - protein, 119 aa, Homo sapiens
EVQLVESGGGLVQPGGSLRLSCAASGFTFSSYSMIWVRQASGKGLEWVSHISGTGGTKNYADSVKGRFTTSRDSSKNTLYLQMNSLRDEDTAVYYCAKRTTNSGAFDVWGQGTMVTVSS

SEQ ID NO: 332 - protein, 109 aa, Homo sapiens
ETTLTQSPGTLSLSPGERATLSCRASQSVTGSHLAWYQQKPGQAPRLLIYDASSRATGIPDRFSGSGSGTDFTLTISRLEPEDFAVYYCQQYGSSPLFSFGQGTKLEIK

SEQ ID NO: 333 - protein, 467 aa, Homo sapiens
MGWSCIILFLVATATGVHSEVQLVESGGGLVQPGGSLRLSCAASGFTFSSYSMIWVRQASGKGLEWVSHISGTGGTKNYADSVKGRFTTSRDSSKNTLYLQMNSLRDEDTAVYYCAKRTTNSGAFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 334 - protein, 235 aa, Homo sapiens
MGWSCIILFLVATATGVHSETTLTQSPGTLSLSPGERATLSCRASQSVTGSHLAWYQQKPGQAPRLLIYDASSRATGIPDRFSGSGSGTDFTLTISRLEPEDFAVYYCQQYGSSPLFSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 335 - DNA, 57 bp, Homo sapiens; identical to SEQ ID NO: 11
  source 1..57: mol_type = other DNA

SEQ ID NO: 336 - DNA, 358 bp, Homo sapiens
  source 1..358: mol_type = other DNA
gaggtgcagctggtggagtctgggggaggcttggtgcagcctggggggtccctgagactctcctgtgcagcctctggattcaccttcagcagctattccatgatctgggtccgccaggcttcagggaaggggctggagtgggtctcacatattagtggtactggtggtaccaaaaactacgcagactccgtgaagggccgcttcaccacctccagagacagctccaagaacacgctgtatctgcaaatgaacagcctgagagacgaggacacggccgtctattactgtgcgaaacggacgacgaattctggtgcttttgatgtttggggccaagggacaatggtcaccgtctcttcag

SEQ ID NO: 337 - DNA, 989 bp, Homo sapiens; identical to SEQ ID NO: 13
  source 1..989: mol_type = other DNA

SEQ ID NO: 338 - DNA, 1404 bp, Homo sapiens
  source 1..1404: mol_type = other DNA
atgggatggtcatgtatcatcctttttctagtagcaactgcaaccggtgtacattccgaggtgcagctggtggagtctgggggaggcttggtgcagcctggggggtccctgagactctcctgtgcagcctctggattcaccttcagcagctattccatgatctgggtccgccaggcttcagggaaggggctggagtgggtctcacatattagtggtactggtggtaccaaaaactacgcagactccgtgaagggccgcttcaccacctccagagacagctccaagaacacgctgtatctgcaaatgaacagcctgagagacgaggacacggccgtctattactgtgcgaaacggacgacgaattctggtgcttttgatgtttggggccaagggacaatggtcaccgtctcttcagcctccaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 339 - DNA, 57 bp, Homo sapiens; identical to SEQ ID NO: 11
  source 1..57: mol_type = other DNA

SEQ ID NO: 340 - DNA, 328 bp, Homo sapiens
  source 1..328: mol_type = other DNA
gaaacgacactcacgcagtctccaggcaccctgtctttgtctccaggggaaagagccaccctctcctgcagggccagtcagagtgttaccggcagccacttagcctggtaccagcagaaacctggccaggctcccaggctcctcatctatgatgcatccagcagggccactggcatcccagacaggttcagtggcagtgggtctgggacagacttcactctcaccatcagcagactggagcctgaagattttgcagtgtattactgtcagcagtatggtagctcacctctgttcagttttggccaggggaccaagctggagatcaaac

SEQ ID NO: 341 - DNA, 323 bp, Homo sapiens; identical to SEQ ID NO: 17
  source 1..323: mol_type = other DNA

SEQ ID NO: 342 - DNA, 708 bp, Homo sapiens
  source 1..708: mol_type = other DNA
atgggatggtcatgtatcatcctttttctagtagcaactgcaaccggtgtacattccgaaacgacactcacgcagtctccaggcaccctgtctttgtctccaggggaaagagccaccctctcctgcagggccagtcagagtgttaccggcagccacttagcctggtaccagcagaaacctggccaggctcccaggctcctcatctatgatgcatccagcagggccactggcatcccagacaggttcagtggcagtgggtctgggacagacttcactctcaccatcagcagactggagcctgaagattttgcagtgtattactgtcagcagtatggtagctcacctctgttcagttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 343 - protein, 8 aa, Homo sapiens
GFSVSRSW

SEQ ID NO: 344 - protein, 8 aa, Homo sapiens
IQSDDNKI

SEQ ID NO: 345 - protein, 11 aa, Homo sapiens
ARDRGWGLFDI

SEQ ID NO: 346 - protein, 11 aa, Homo sapiens
QSLLQSDGKTY

SEQ ID NO: 347 - skipped

SEQ ID NO: 348 - protein, 9 aa, Homo sapiens
VHHLPLPLT

SEQ ID NO: 349 - protein, 118 aa, Homo sapiens
EVQLVESGGGLVQPGGSLRLSCAASGFSVSRSWMNWVRQAPGKGLVWVSSIQSDDNKIGYADSVRGRFAVSGDNAKNTVFLQMNSLRAEDTAVYYCARDRGWGLFDIWGQGTLVTVSS

SEQ ID NO: 350 - protein, 112 aa, Homo sapiens
DIVMTQSPLSLSVTPGQPASISCKSSQSLLQSDGKTYLHWYLQKPGQSPQPLIYEVSNRFSGVPDRFSGSGSGTDFTLKISRVEAEDVGVYYCVHHLPLPLTFGGGTKVEIK

SEQ ID NO: 351 - protein, 467 aa, Homo sapiens
MGWSCIILFLVATATGVHSEVQLVESGGGLVQPGGSLRLSCAASGFSVSRSWMNWVRQAPGKGLVWVSSIQSDDNKIGYADSVRGRFAVSGDNAKNTVFLQMNSLRAEDTAVYYCARDRGWGLFDIWGQGTLVTVSSASPTSPKSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 352 - protein, 238 aa, Homo sapiens
MGWSCIILFLVATATGVHSDIVMTQSPLSLSVTPGQPASISCKSSQSLLQSDGKTYLHWYLQKPGQSPQPLIYEVSNRFSGVPDRFSGSGSGTDFTLKISRVEAEDVGVYYCVHHLPLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 353 - DNA, 57 bp, Homo sapiens; identical to SEQ ID NO: 11
  source 1..57: mol_type = other DNA

SEQ ID NO: 354 - DNA, 355 bp, Homo sapiens
  source 1..355: mol_type = other DNA
gaggtgcagctggtggagtctgggggaggcttagttcagccgggggggtccctgagactctcctgtgcagcctctggattcagcgtcagtaggtcctggatgaattgggtccgccaagctccagggaaggggctggtgtgggtctcatctattcaaagtgatgacaataagataggctacgcggactccgtgaggggccgattcgccgtctccggagacaacgccaagaatacggtgtttctgcaaatgaacagtctgagagccgaggatacggctgtctattactgcgcaagagatcggggctggggactttttgatatctggggccaagggacattggtcaccgtctcttcag

SEQ ID NO: 355 - DNA, 992 bp, Homo sapiens; identical to SEQ ID NO: 67
  source 1..992: mol_type = other DNA

SEQ ID NO: 356 - DNA, 1404 bp, Homo sapiens
  source 1..1404: mol_type = other DNA
atgggatggtcatgtatcatcctttttctagtagcaactgcaaccggtgtacattccgaggtgcagctggtggagtctgggggaggcttagttcagccgggggggtccctgagactctcctgtgcagcctctggattcagcgtcagtaggtcctggatgaattgggtccgccaagctccagggaaggggctggtgtgggtctcatctattcaaagtgatgacaataagataggctacgcggactccgtgaggggccgattcgccgtctccggagacaacgccaagaatacggtgtttctgcaaatgaacagtctgagagccgaggatacggctgtctattactgcgcaagagatcggggctggggactttttgatatctggggccaagggacattggtcaccgtctcttcagcatccccgaccagccccaagtcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 357 - DNA, 57 bp, Homo sapiens; identical to SEQ ID NO: 11
  source 1..57: mol_type = other DNA

SEQ ID NO: 358 - DNA, 337 bp, Homo sapiens
  source 1..337: mol_type = other DNA
gatattgtgatgactcagtctccactctctctgtccgtcacccctggacagccggcctccatctcctgcaagtctagtcagagcctcctgcaaagtgatggaaagacctatttgcattggtacctgcagaagccaggccagtctccacagcccctgatctatgaagtttccaaccggttctctggagtgccagataggttcagtggcagcgggtcagggacagatttcacactgaaaatcagccgggtggaggctgaggatgttggggtttattactgcgtgcaccatttgccccttcctctcacattcggcggagggaccaaggttgagatcaaac

SEQ ID NO: 359 - DNA, 323 bp, Homo sapiens; identical to SEQ ID NO: 17
  source 1..323: mol_type = other DNA

SEQ ID NO: 360 - DNA, 717 bp, Homo sapiens
  source 1..717: mol_type = other DNA
atgggatggtcatgtatcatcctttttctagtagcaactgcaaccggtgtacattccgatattgtgatgactcagtctccactctctctgtccgtcacccctggacagccggcctccatctcctgcaagtctagtcagagcctcctgcaaagtgatggaaagacctatttgcattggtacctgcagaagccaggccagtctccacagcccctgatctatgaagtttccaaccggttctctggagtgccagataggttcagtggcagcgggtcagggacagatttcacactgaaaatcagccgggtggaggctgaggatgttggggtttattactgcgtgcaccatttgccccttcctctcacattcggcggagggaccaaggttgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 361 - protein, 8 aa, Homo sapiens
GITFNTHS

SEQ ID NO: 362 - protein, 8 aa, Homo sapiens
IDGSSSFI

SEQ ID NO: 363 - protein, 14 aa, Homo sapiens
ATGCPSVVNQGLEN

SEQ ID NO: 364 - protein, 6 aa, Homo sapiens
QGIGND

SEQ ID NO: 365 - skipped

SEQ ID NO: 366 - protein, 9 aa, Homo sapiens
LQHHSYPIT

SEQ ID NO: 367 - protein, 121 aa, Homo sapiens
EVQLVESGGGFVQPGGSLRLSCAASGITFNTHSMAWVRQAPGKGLEWVSSIDGSSSFIYYTDSVRGRFTISRDNSKNTLYLQLTSLRAEDTAVYYCATGCPSVVNQGLENWGQGSLVTVSS

SEQ ID NO: 368 - protein, 107 aa, Homo sapiens
AIQMTQSPSSLSASAGDRVTITCRASQGIGNDLGWYQQKPGKAPKRLIYGASSLQSGVPSRFSGSGSETEFTLTISSLQPEDVATYYCLQHHSYPITFGQGTRLEIK

SEQ ID NO: 369 - protein, 470 aa, Homo sapiens
MGWSCIILFLVATATGVHSEVQLVESGGGFVQPGGSLRLSCAASGITFNTHSMAWVRQAPGKGLEWVSSIDGSSSFIYYTDSVRGRFTISRDNSKNTLYLQLTSLRAEDTAVYYCATGCPSVVNQGLENWGQGSLVTVSSASPTSPKSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 370 - protein, 233 aa, Homo sapiens
MGWSCIILFLVATATGVHSAIQMTQSPSSLSASAGDRVTITCRASQGIGNDLGWYQQKPGKAPKRLIYGASSLQSGVPSRFSGSGSETEFTLTISSLQPEDVATYYCLQHHSYPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 371 - DNA, 57 bp, Homo sapiens; identical to SEQ ID NO: 11
  source 1..57: mol_type = other DNA

SEQ ID NO: 372 - DNA, 364 bp, Homo sapiens
  source 1..364: mol_type = other DNA
gaggtgcagctggtggagtctgggggaggctttgtacagccaggggggagcctgcgactctcctgtgcagcctcaggaatcacttttaacacccattccatggcctgggtccgccaggctccagggaaggggctggagtgggtctcgtctattgatggcagtagtagtttcatttactacacagactccgtgaggggccggttcaccatctccagagacaattccaagaacaccctgtatctgcaactgaccagcctgagagccgaggacacggccgtatattactgtgcgacaggttgtccttcggtggtaaatcaggggttggagaactggggccagggatccctggtcaccgtctcctcag

SEQ ID NO: 373 - DNA, 992 bp, Homo sapiens; identical to SEQ ID NO: 67
  source 1..992: mol_type = other DNA

SEQ ID NO: 374 - DNA, 1413 bp, Homo sapiens
  source 1..1413: mol_type = other DNA
atgggatggtcatgtatcatcctttttctagtagcaactgcaaccggtgtacattccgaggtgcagctggtggagtctgggggaggctttgtacagccaggggggagcctgcgactctcctgtgcagcctcaggaatcacttttaacacccattccatggcctgggtccgccaggctccagggaaggggctggagtgggtctcgtctattgatggcagtagtagtttcatttactacacagactccgtgaggggccggttcaccatctccagagacaattccaagaacaccctgtatctgcaactgaccagcctgagagccgaggacacggccgtatattactgtgcgacaggttgtccttcggtggtaaatcaggggttggagaactggggccagggatccctggtcaccgtctcctcagcatccccgaccagccccaagtcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 375 - DNA, 57 bp, Homo sapiens; identical to SEQ ID NO: 11
  source 1..57: mol_type = other DNA

SEQ ID NO: 376 - DNA, 322 bp, Homo sapiens
  source 1..322: mol_type = other DNA
gccatccagatgacccagtctccatcctccctgtctgcatctgcaggagacagagtcaccatcacttgccgggcaagtcagggcattggaaatgatttaggctggtatcagcagaaaccagggaaagcccctaagcgcctgatctatggtgcatccagtttgcaaagtggggtcccatcaaggttcagcggcagtggatctgagactgaattcactctcacaatcagcagcctgcagcctgaagatgttgcaacttattactgtctacagcatcatagttacccgatcaccttcggccaagggacacgactggagattaaac

SEQ ID NO: 377 - DNA, 323 bp, Homo sapiens; identical to SEQ ID NO: 17
  source 1..323: mol_type = other DNA

SEQ ID NO: 378 - DNA, 702 bp, Homo sapiens
  source 1..702: mol_type = other DNA
atgggatggtcatgtatcatcctttttctagtagcaactgcaaccggtgtacattccgccatccagatgacccagtctccatcctccctgtctgcatctgcaggagacagagtcaccatcacttgccgggcaagtcagggcattggaaatgatttaggctggtatcagcagaaaccagggaaagcccctaagcgcctgatctatggtgcatccagtttgcaaagtggggtcccatcaaggttcagcggcagtggatctgagactgaattcactctcacaatcagcagcctgcagcctgaagatgttgcaacttattactgtctacagcatcatagttacccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 379 - protein, 8 aa, Homo sapiens
GFSVSRSW

SEQ ID NO: 380 - protein, 8 aa, Homo sapiens
IQSDDNKI

SEQ ID NO: 381 - protein, 11 aa, Homo sapiens
ARDRGWGLFDI

SEQ ID NO: 382 - protein, 11 aa, Homo sapiens
QSLLQSDGKTY

SEQ ID NO: 383 - skipped

SEQ ID NO: 384 - protein, 9 aa, Homo sapiens
VHHLPLPLT

SEQ ID NO: 385 - protein, 118 aa, Homo sapiens; identical to SEQ ID NO: 349

SEQ ID NO: 386 - protein, 112 aa, Homo sapiens
DIVMTQTPLSLSVTPGQPASISCKSSQSLLQSDGKTYLHWYLQKPGQSPQPLIYEVSNRFSGVPDRFSGNGSGTDFTLKISRVEAEDVGVYYCVHHLPLPLTFGGGTKVEIK

SEQ ID NO: 387 - protein, 467 aa, Homo sapiens; identical to SEQ ID NO: 351

SEQ ID NO: 388 - protein, 238 aa, Homo sapiens
MGWSCIILFLVATATGVHSDIVMTQTPLSLSVTPGQPASISCKSSQSLLQSDGKTYLHWYLQKPGQSPQPLIYEVSNRFSGVPDRFSGNGSGTDFTLKISRVEAEDVGVYYCVHHLPLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 389 - DNA, 57 bp, Homo sapiens; identical to SEQ ID NO: 11
  source 1..57: mol_type = other DNA

SEQ ID NO: 390 - DNA, 355 bp, Homo sapiens; identical to SEQ ID NO: 354
  source 1..355: mol_type = other DNA

SEQ ID NO: 391 - DNA, 992 bp, Homo sapiens; identical to SEQ ID NO: 67
  source 1..992: mol_type = other DNA

SEQ ID NO: 392 - DNA, 1404 bp, Homo sapiens; identical to SEQ ID NO: 356
  source 1..1404: mol_type = other DNA

SEQ ID NO: 393 - DNA, 57 bp, Homo sapiens; identical to SEQ ID NO: 11
  source 1..57: mol_type = other DNA

SEQ ID NO: 394 - DNA, 337 bp, Homo sapiens
  source 1..337: mol_type = other DNA
gatattgtgatgacccagactccactctctctgtccgtcacccctggacagccggcctccatctcctgcaagtctagtcagagcctcctgcaaagtgatggaaagacctatttgcattggtacctgcagaagccaggccagtctccacagcccctgatctatgaagtttccaaccggttctctggagtgccagataggttcagtggcaacgggtcagggacagatttcacactgaaaatcagccgggtggaggctgaggatgttggggtttattactgcgtgcaccatttgccccttcctctcacattcggcggagggaccaaggttgagatcaaac

SEQ ID NO: 395 - DNA, 323 bp, Homo sapiens; identical to SEQ ID NO: 17
  source 1..323: mol_type = other DNA

SEQ ID NO: 396 - DNA, 717 bp, Homo sapiens
  source 1..717: mol_type = other DNA
atgggatggtcatgtatcatcctttttctagtagcaactgcaaccggtgtacattccgatattgtgatgacccagactccactctctctgtccgtcacccctggacagccggcctccatctcctgcaagtctagtcagagcctcctgcaaagtgatggaaagacctatttgcattggtacctgcagaagccaggccagtctccacagcccctgatctatgaagtttccaaccggttctctggagtgccagataggttcagtggcaacgggtcagggacagatttcacactgaaaatcagccgggtggaggctgaggatgttggggtttattactgcgtgcaccatttgccccttcctctcacattcggcggagggaccaaggttgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 397 - protein, 8 aa, Homo sapiens
GFTFSSYT

SEQ ID NO: 398 - protein, 8 aa, Homo sapiens
ISSSRSSI

SEQ ID NO: 399 - protein, 12 aa, Homo sapiens
ARGGRSDSFFDH

SEQ ID NO: 400 - protein, 11 aa, Homo sapiens
QSLVFSDGDTY

SEQ ID NO: 401 - skipped

SEQ ID NO: 402 - protein, 9 aa, Homo sapiens
MQGALWPLT

SEQ ID NO: 403 - protein, 119 aa, Homo sapiens
EVQLVESGGGLVKSGGSLRLSCAASGFTFSSYTMNWVRQAPGKGLEWVSSISSSRSSIFYAGSVKGRFTISRDNAKNSLYLQMNSLRVEDTAVYFCARGGRSDSFFDHWGQGTLVTVSS

SEQ ID NO: 404 - protein, 112 aa, Homo sapiens
DIVMTQTPLSLPVTLGQPASISCRSSQSLVFSDGDTYLNWFQQRPGQSPRRLIYKVSNRDSGVPDRFSGSGSGTDFTLKISRVEAEDVGVYYCMQGALWPLTFGQGTKVEIK

SEQ ID NO: 405 - protein, 468 aa, Homo sapiens
MGWSCIILFLVATATGVHSEVQLVESGGGLVKSGGSLRLSCAASGFTFSSYTMNWVRQAPGKGLEWVSSISSSRSSIFYAGSVKGRFTISRDNAKNSLYLQMNSLRVEDTAVYFCARGGRSDSFFDHWGQGTLVTVSSASPTSPKSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 406 - protein, 238 aa, Homo sapiens
MGWSCIILFLVATATGVHSDIVMTQTPLSLPVTLGQPASISCRSSQSLVFSDGDTYLNWFQQRPGQSPRRLIYKVSNRDSGVPDRFSGSGSGTDFTLKISRVEAEDVGVYYCMQGALWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 407 - DNA, 57 bp, Homo sapiens; identical to SEQ ID NO: 11
  source 1..57: mol_type = other DNA

SEQ ID NO: 408 - DNA, 358 bp, Homo sapiens
  source 1..358: mol_type = other DNA
gaggtgcagctggtggagtctgggggaggcctggtcaagtctggggggtccctgagactctcctgtgcagcctctggattcaccttcagtagttataccatgaattgggtccgccaggctccagggaaggggctggagtgggtctcatccatcagtagtagccgtagttccatattctacgcaggctcagtgaagggccgattcaccatctccagagacaatgccaagaactcactgtatctgcaaatgaacagcctgagagtcgaggacacggctgtatatttctgtgcgagaggagggcggtcagattccttctttgaccactggggccagggaaccctggtcaccgtctcctcag

SEQ ID NO: 409 - DNA, 992 bp, Homo sapiens; identical to SEQ ID NO: 67
  source 1..992: mol_type = other DNA

SEQ ID NO: 410 - DNA, 1407 bp, Homo sapiens
  source 1..1407: mol_type = other DNA
atgggatggtcatgtatcatcctttttctagtagcaactgcaaccggtgtacattccgaggtgcagctggtggagtctgggggaggcctggtcaagtctggggggtccctgagactctcctgtgcagcctctggattcaccttcagtagttataccatgaattgggtccgccaggctccagggaaggggctggagtgggtctcatccatcagtagtagccgtagttccatattctacgcaggctcagtgaagggccgattcaccatctccagagacaatgccaagaactcactgtatctgcaaatgaacagcctgagagtcgaggacacggctgtatatttctgtgcgagaggagggcggtcagattccttctttgaccactggggccagggaaccctggtcaccgtctcctcagcatccccgaccagccccaagtcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 411 - DNA, 57 bp, Homo sapiens
  source 1..57: mol_type = other DNA
atgggatggtcatgtatcatcctttttctagtagcaactgcaactggtgtacattcc

SEQ ID NO: 412 - DNA, 337 bp, Homo sapiens
  source 1..337: mol_type = other DNA
gatattgtgatgacccagactccactctccctgcccgtcacccttggacagccggcctccatctcctgcaggtctagtcaaagcctcgtattcagtgatggagacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtgcactctggcctctaacgttcggccaagggaccaaggtagaaatcaaac

SEQ ID NO: 413 - DNA, 323 bp, Homo sapiens; identical to SEQ ID NO: 17
  source 1..323: mol_type = other DNA

SEQ ID NO: 414 - DNA, 717 bp, Homo sapiens
  source 1..717: mol_type = other DNA
atgggatggtcatgtatcatcctttttctagtagcaactgcaactggtgtacattccgatattgtgatgacccagactccactctccctgcccgtcacccttggacagccggcctccatctcctgcaggtctagtcaaagcctcgtattcagtgatggagacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtgcactctggcctctaacgttcggccaagggaccaaggtagaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 415 - protein, 8 aa, Homo sapiens
GFTFSSYS

SEQ ID NO: 416 - protein, 8 aa, Homo sapiens
ISETSYHI

SEQ ID NO: 417 - protein, 15 aa, Homo sapiens
AAHVPCGGTCNGIDY

SEQ ID NO: 418 - protein, 6 aa, Homo sapiens
QGIGND

SEQ ID NO: 419 - skipped

SEQ ID NO: 420 - protein, 9 aa, Homo sapiens
LQHNAYPRT

SEQ ID NO: 421 - protein, 122 aa, Homo sapiens
EVQLVESGGGLVKPGGSLRLSCAASGFTFSSYSMNWVRQAPGKGLEWVSSISETSYHIYYADSVKGRFTISRDNARNSLYLQMNSLRAEDTGIYFCAAHVPCGGTCNGIDYWGQGTLVTVSS

SEQ ID NO: 422 - protein, 106 aa, Homo sapiens
AIQMTQSPSSLSASVGDRVTITCRASQGIGNDLGWYQHKPGMAPKRLIYGASTLQSGVPSRISGSGSGTEFTLTISSLQPEDFATYYCLQHNAYPRTFGQGTRLEI

SEQ ID NO: 423 - protein, 471 aa, Homo sapiens
MGWSCIILFLVATATGVHSEVQLVESGGGLVKPGGSLRLSCAASGFTFSSYSMNWVRQAPGKGLEWVSSISETSYHIYYADSVKGRFTISRDNARNSLYLQMNSLRAEDTGIYFCAAHVPCGGTCNGIDYWGQGTLVTVSSASPTSPKSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 424 - protein, 233 aa, Homo sapiens
MGWSCIILFLVATATGVHSAIQMTQSPSSLSASVGDRVTITCRASQGIGNDLGWYQHKPGMAPKRLIYGASTLQSGVPSRISGSGSGTEFTLTISSLQPEDFATYYCLQHNAYPRTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 425 - DNA, 57 bp, Homo sapiens; identical to SEQ ID NO: 11
  source 1..57: mol_type = other DNA

SEQ ID NO: 426 - DNA, 367 bp, Homo sapiens
  source 1..367: mol_type = other DNA
gaggtgcagctggtggagtctgggggaggcctggtcaagcctggggggtccctgagactctcctgtgcagcctctggattcacgttcagtagctacagcatgaactgggtccgccaggctccagggaagggactggagtgggtctcatccatcagtgagactagttatcacatatactacgcagactcagtgaagggccgattcaccatctccagagacaacgccaggaactcactttatctacaaatgaacagcctgagagccgaggacacgggtatatatttctgtgcggcacatgtaccgtgtggcggcacctgtaacgggattgactactggggccagggaaccctggtcaccgtctcctcag

SEQ ID NO: 427 - DNA, 992 bp, Homo sapiens; identical to SEQ ID NO: 67
  source 1..992: mol_type = other DNA

SEQ ID NO: 428 - DNA, 1416 bp, Homo sapiens
  source 1..1416: mol_type = other DNA
atgggatggtcatgtatcatcctttttctagtagcaactgcaaccggtgtacattccgaggtgcagctggtggagtctgggggaggcctggtcaagcctggggggtccctgagactctcctgtgcagcctctggattcacgttcagtagctacagcatgaactgggtccgccaggctccagggaagggactggagtgggtctcatccatcagtgagactagttatcacatatactacgcagactcagtgaagggccgattcaccatctccagagacaacgccaggaactcactttatctacaaatgaacagcctgagagccgaggacacgggtatatatttctgtgcggcacatgtaccgtgtggcggcacctgtaacgggattgactactggggccagggaaccctggtcaccgtctcctcagcatccccgaccagccccaagtcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 429 - DNA, 57 bp, Homo sapiens; identical to SEQ ID NO: 11
  source 1..57: mol_type = other DNA

SEQ ID NO: 430 - DNA, 320 bp, Homo sapiens
  source 1..320: mol_type = other DNA
gccatccagatgacccagtctccatcctccctgtctgcatctgtaggagacagagtcaccatcacttgccgggcaagtcagggcataggaaatgatttaggctggtatcagcacaaaccagggatggcccctaagcgcctgatctatggtgcatccactttgcaaagtggggtcccatcaaggatcagcggcagtgggtctgggacagaattcactctcacaatcagcagcctgcagcctgaggattttgcaacttattactgtctacaacataatgcttaccctcgaacgttcggccaagggaccaggctggaaatcaa

SEQ ID NO: 431 - DNA, 325 bp, Homo sapiens
  source 1..325: mol_type = other DNA
gcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 432 - DNA, 702 bp, Homo sapiens
  source 1..702: mol_type = other DNA
atgggatggtcatgtatcatcctttttctagtagcaactgcaaccggtgtacattccgccatccagatgacccagtctccatcctccctgtctgcatctgtaggagacagagtcaccatcacttgccgggcaagtcagggcataggaaatgatttaggctggtatcagcacaaaccagggatggcccctaagcgcctgatctatggtgcatccactttgcaaagtggggtcccatcaaggatcagcggcagtgggtctgggacagaattcactctcacaatcagcagcctgcagcctgaggattttgcaacttattactgtctacaacataatgcttaccctcgaacgttcggccaagggaccaggctggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 433 - protein, 8 aa, Homo sapiens
GFAFSNYF

SEQ ID NO: 434 - protein, 8 aa, Homo sapiens
INQDGSDK

SEQ ID NO: 435 - protein, 11 aa, Homo sapiens
ARASLSSAADY

SEQ ID NO: 436 - protein, 11 aa, Homo sapiens
QSLVYSDGNTN

SEQ ID NO: 437 - skipped

SEQ ID NO: 438 - protein, 9 aa, Homo sapiens
MQSTHWPHT

SEQ ID NO: 439 - protein, 118 aa, Homo sapiens
EVQLVESGGGLVQPEGSLRLSCAASGFAFSNYFMNWVRQAPGKGLEWVANINQDGSDKRYVDSVKGRFTISRDNAKNSLYLEMNSLRADDTAVYYCARASLSSAADYWGQGTLVTVSS

SEQ ID NO: 440 - protein, 112 aa, Homo sapiens
EIVLTQSPLSLPVTLGQPASISCWSSQSLVYSDGNTNLNWYQQRPGQSPRRLIYRVSNRDSGVPDRFSGSGSGSDFTLKISRVEAEDVGIYYCMQSTHWPHTFGQGTKLEIK

SEQ ID NO: 441 - protein, 467 aa, Homo sapiens
MGWSCIILFLVATATGVHSEVQLVESGGGLVQPEGSLRLSCAASGFAFSNYFMNWVRQAPGKGLEWVANINQDGSDKRYVDSVKGRFTISRDNAKNSLYLEMNSLRADDTAVYYCARASLSSAADYWGQGTLVTVSSASPTSPKSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLFPG

SEQ ID NO: 442 - protein, 238 aa, Homo sapiens
MGWSCIILFLVATATGVHSEIVLTQSPLSLPVTLGQPASISCWSSQSLVYSDGNTNLNWYQQRPGQSPRRLIYRVSNRDSGVPDRFSGSGSGSDFTLKISRVEAEDVGIYYCMQSTHWPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 443 - DNA, 57 bp, Homo sapiens; identical to SEQ ID NO: 11
  source 1..57: mol_type = other DNA

SEQ ID NO: 444 - DNA, 355 bp, Homo sapiens
  source 1..355: mol_type = other DNA
gaggtgcagctggtggagtctgggggaggcttggtccagcctgaagggtccctgagactctcctgtgcagcctctggattcgcctttagtaactattttatgaactgggtccgccaggctccagggaaggggctggagtgggtggccaacataaaccaagatggaagtgacaaacgttatgtggactcggtgaagggccgattcaccatctccagagacaacgccaagaactcactgtacttggaaatgaacagcctgagagccgatgacacggctgtgtattactgtgcgagagcctccctttcatccgccgctgactactggggccagggaaccctggtcaccgtctcctcag

SEQ ID NO: 445 - DNA, 992 bp, Homo sapiens; identical to SEQ ID NO: 67
  source 1..992: mol_type = other DNA

SEQ ID NO: 446 - DNA, 1404 bp, Homo sapiens
  source 1..1404: mol_type = other DNA
atgggatggtcatgtatcatcctttttctagtagcaactgcaaccggtgtacattccgaggtgcagctggtggagtctgggggaggcttggtccagcctgaagggtccctgagactctcctgtgcagcctctggattcgcctttagtaactattttatgaactgggtccgccaggctccagggaaggggctggagtgggtggccaacataaaccaagatggaagtgacaaacgttatgtggactcggtgaagggccgattcaccatctccagagacaacgccaagaactcactgtacttggaaatgaacagcctgagagccgatgacacggctgtgtattactgtgcgagagcctccctttcatccgccgctgactactggggccagggaaccctggtcaccgtctcctcagcatccccgaccagccccaagtcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggcgctctgcacaaccactacacgcagaagagcctctccctgttccccgggtaa

SEQ ID NO: 447 - DNA, 57 bp, Homo sapiens; identical to SEQ ID NO: 11
  source 1..57: mol_type = other DNA

SEQ ID NO: 448 - DNA, 337 bp, Homo sapiens
  source 1..337: mol_type = other DNA
gaaattgtgttgacacagtctccactctccctgcccgtcacccttggacagccggcctccatctcctgctggtctagccaaagcctcgtatatagtgatggaaacactaacttgaattggtatcagcagaggccaggccaatctccaaggcgcctaatttatagggtttctaaccgggactctggggtcccagacagattcagcggcagtgggtcaggctctgatttcacactgaaaatcagcagggtggaggctgaggatgttgggatttattactgcatgcaaagtacacactggccgcacacttttggccaggggaccaagctggagatcaaac

SEQ ID NO: 449 - DNA, 323 bp, Homo sapiens; identical to SEQ ID NO: 17
  source 1..323: mol_type = other DNA

SEQ ID NO: 450 - DNA, 717 bp, Homo sapiens
  source 1..717: mol_type = other DNA
atgggatggtcatgtatcatcctttttctagtagcaactgcaaccggtgtacattccgaaattgtgttgacacagtctccactctccctgcccgtcacccttggacagccggcctccatctcctgctggtctagccaaagcctcgtatatagtgatggaaacactaacttgaattggtatcagcagaggccaggccaatctccaaggcgcctaatttatagggtttctaaccgggactctggggtcccagacagattcagcggcagtgggtcaggctctgatttcacactgaaaatcagcagggtggaggctgaggatgttgggatttattactgcatgcaaagtacacactggccgcacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 451 - DNA, 24 bp, synthetic construct
  source 1..24: mol_type = other DNA
cactcccaggtgcagctggtgcag

SEQ ID NO: 452 - DNA, 24 bp, synthetic construct
  source 1..24: mol_type = other DNA
tgggtcttrtcccaggtcaccttg

SEQ ID NO: 453 - DNA, 23 bp, synthetic construct
  source 1..23: mol_type = other DNA
aaggtgtccagtgtsaggtgcag

SEQ ID NO: 454 - DNA, 24 bp, synthetic construct
  source 1..24: mol_type = other DNA
gtcctgtcccaggtgcagctgcag

SEQ ID NO: 455 - DNA, 24 bp, synthetic construct
  source 1..24: mol_type = other DNA
gagtctgttccgaggtgcagctgg

SEQ ID NO: 456 - DNA, 21 bp, synthetic construct
  source 1..21: mol_type = other DNA
gtgccagggggaagaccgatg

SEQ ID NO: 457 - DNA, 21 bp, synthetic construct
  source 1..21: mol_type = other DNA
gcmgaggctcagcgggaagac

SEQ ID NO: 458 - DNA, 22 bp, synthetic construct
  source 1..22: mol_type = other DNA
gagacgagggggaaaagggttg

SEQ ID NO: 459 - DNA, 23 bp, synthetic construct
  source 1..23: mol_type = other DNA
caggtgccagatgtghcatccag

SEQ ID NO: 460 - DNA, 26 bp, synthetic construct
  source 1..26: mol_type = other DNA
ctggatccagtgsggatattgtgatg

SEQ ID NO: 461 - DNA, 25 bp, synthetic construct
  source 1..25: mol_type = other DNA
cccagataccaccggagaaattgtg

SEQ ID NO: 462 - DNA, 25 bp, synthetic construct
  source 1..25: mol_type = other DNA
ctctggtgcctacggggacatcgtg

SEQ ID NO: 463 - DNA, 24 bp, synthetic construct
  source 1..24: mol_type = other DNA
ctgataccagggcagaaacgacac

SEQ ID NO: 464 - DNA, 28 bp, synthetic construct
  source 1..28: mol_type = other DNA
gaacactctcccctgttgaagctctttg

SEQ ID NO: 465 - DNA, 23 bp, synthetic construct
  source 1..23: mol_type = other DNA
ggtcctgggcccagtctgtgctg

SEQ ID NO: 466 - DNA, 23 bp, synthetic construct
  source 1..23: mol_type = other DNA
ggtcctgggcccagtctgccctg

SEQ ID NO: 467 - DNA, 23 bp, synthetic construct
  source 1..23: mol_type = other DNA
tctgtgrcctcctatgagctgac

SEQ ID NO: 468 - DNA, 23 bp, synthetic construct
  source 1..23: mol_type = other DNA
ctctcgcagcctgtgctgactca

SEQ ID NO: 469 - DNA, 23 bp, synthetic construct
  source 1..23: mol_type = other DNA
gttcttgggccaattttatgctg

SEQ ID NO: 470 - DNA, 23 bp, synthetic construct
  source 1..23: mol_type = other DNA
ggtccaattctcaggctgtggtg

SEQ ID NO: 471 - DNA, 23 bp, synthetic construct
  source 1..23: mol_type = other DNA
gagtggattctcagactgtggtg

SEQ ID NO: 472 - DNA, 24 bp, synthetic construct
  source 1..24: mol_type = other DNA
gtcagtggtccaggcagggctgac

SEQ ID NO: 473 - DNA, 21 bp, synthetic construct
  source 1..21: mol_type = other DNA
gtgctcccttcatgcgtgacc

SEQ ID NO: 474 - DNA, 66 bp, synthetic construct
  source 1..66: mol_type = other DNA
gtatcatcctttttctagtagcaactgcaaccggtgtacattcccaggtgcagctggtgcagtctg

SEQ ID NO: 475 - DNA, 69 bp, synthetic construct
  source 1..69: mol_type = other DNA
gtatcatcctttttctagtagcaactgcaaccggtgtacattcccaggtcaccttgaaggagtctggtc

SEQ ID NO: 476 - DNA, 72 bp, synthetic construct
  source 1..72: mol_type = other DNA
gtatcatcctttttctagtagcaactgcaaccggtgtacattccgaggtgcagctggtggagtctgggggag

SEQ ID NO: 477 - DNA, 67 bp, synthetic construct
  source 1..67: mol_type = other DNA
gtatcatcctttttctagtagcaactgcaaccggtgtacattcccaggtgcagctgcaggagtcggg

SEQ ID NO: 478 - DNA, 67 bp, synthetic construct
  source 1..67: mol_type = other DNA
gtatcatcctttttctagtagcaactgcaaccggtgtacattcccaggtgcagctgcagcagtgggg

SEQ ID NO: 479 - DNA, 48 bp, synthetic construct
  source 1..48: mol_type = other DNA
cttggaggagggtgccagggggaagaccgatgggcccttggtggargc

SEQ ID NO: 480 - DNA, 48 bp, synthetic construct
  source 1..48: mol_type = other DNA
cttggaggagggtgccagggggaagaccgacttggggctggtcgggga

SEQ ID NO: 481 - DNA, 48 bp, synthetic construct
  source 1..48: mol_type = other DNA
cttggaggagggtgccagggggaagaccgatggggcggatgcactccc

SEQ ID NO: 482 - DNA, 70 bp, synthetic construct
  source 1..70: mol_type = other DNA
gtatcatcctttttctagtagcaactgcaaccggtgtacattccgccatccagatgacccagtctccatc

SEQ ID NO: 483 - DNA, 73 bp, synthetic construct
  source 1..73: mol_type = other DNA
gtatcatcctttttctagtagcaactgcaaccggtgtacattccgatattgtgatgacccagactccactctc

SEQ ID NO: 484 - DNA, 73 bp, synthetic construct
  source 1..73: mol_type = other DNA
gtatcatcctttttctagtagcaactgcaaccggtgtacattccgatattgtgatgactcagtctccactctc

SEQ ID NO: 485 - DNA, 69 bp, synthetic construct
  source 1..69: mol_type = other DNA
gtatcatcctttttctagtagcaactgcaaccggtgtacattccgaaattgtgttgacacagtctccag

SEQ ID NO: 486 - DNA, 69 bp, synthetic construct
  source 1..69: mol_type = other DNA
gtatcatcctttttctagtagcaactgcaaccggtgtacattccgaaattgtgatgacgcagtctccag

SEQ ID NO: 487 - DNA, 69 bp, synthetic construct
  source 1..69: mol_type = other DNA
gtatcatcctttttctagtagcaactgcaaccggtgtacattccgacatcgtgatgacccagtctccag

SEQ ID NO: 488 - DNA, 69 bp, synthetic construct
  source 1..69: mol_type = other DNA
gtatcatcctttttctagtagcaactgcaaccggtgtacattccgaaacgacactcacgcagtctccag

SEQ ID NO: 489 - DNA, 58 bp, synthetic construct
  source 1..58: mol_type = other DNA
gatttcaactgctcatcagatggcgggaagatgaagacagatggtgcagccacagttc

SEQ ID NO: 490 - DNA, 72 bp, synthetic construct
  source 1..72: mol_type = other DNA
gtatcatcctttttctagtagcaactgcaaccggttcctgggcccagtctgtgctgactcagccgccctcag

SEQ ID NO: 491 - DNA, 72 bp, synthetic construct
  source 1..72: mol_type = other DNA
gtatcatcctttttctagtagcaactgcaaccggttcctgggcccagtctgccctgactcagcctgcctccg

SEQ ID NO: 492 - DNA, 66 bp, synthetic construct
  source 1..66: mol_type = other DNA
gtatcatcctttttctagtagcaactgcaaccggttcctgggcctcctatgagctgacacagccac

SEQ ID NO: 493 - DNA, 66 bp, synthetic construct
  source 1..66: mol_type = other DNA
gtatcatcctttttctagtagcaactgcaaccggttcctgggcctcctatgagctgactcaggacc

SEQ ID NO: 494 - DNA, 72 bp, synthetic construct
  source 1..72: mol_type = other DNA
gtatcatcctttttctagtagcaactgcaaccggttcctgggcccagcctgtgctgactcaatcgtcctctg

SEQ ID NO: 495 - DNA, 70 bp, synthetic construct
  source 1..70: mol_type = other DNA
gtatcatcctttttctagtagcaactgcaaccggttcctgggcccagcctgtgctgactcagccracttc

SEQ ID NO: 496 - DNA, 70 bp, synthetic construct
  source 1..70: mol_type = other DNA
gtatcatcctttttctagtagcaactgcaaccggttcctgggccaattttatgctgactcagccccactc

SEQ ID NO: 497 - DNA, 70 bp, synthetic construct
  source 1..70: mol_type = other DNA
gtatcatcctttttctagtagcaactgcaaccggttcctgggcccaggctgtggtgactcaggagccctc

SEQ ID NO: 498 - DNA, 70 bp, synthetic construct
  source 1..70: mol_type = other DNA
gtatcatcctttttctagtagcaactgcaaccggttcctgggcccagactgtggtgacccaggagccatc

SEQ ID NO: 499 - DNA, 72 bp, synthetic construct
  source 1..72: mol_type = other DNA
gtatcatcctttttctagtagcaactgcaaccggttcctgggcccaggcagggctgactcagccaccctcgg

SEQ ID NO: 500 - DNA, 50 bp, synthetic construct
  source 1..50: mol_type = other DNA
gtgtggccttgttggcttgaagctcctcactcgagggygggaacagagtg

SEQ ID NO: 501 - DNA, 20 bp, synthetic construct
  source 1..20: mol_type = other DNA
ttaggcaccccaggctttac

SEQ ID NO: 502 - DNA, 20 bp, synthetic construct
  source 1..20: mol_type = other DNA
agatggttctttccgcctca

(19) United States
(12) Patent Application Publication
(10) Pub. No.: US 2026/0226141 A1
(43) Pub. Date: Aug. 6, 2026

(54) ANTIBODIES AGAINST MULTIDRUG-RESISTANT KLEBSIELLA PNEUMONIAE

(71) Applicant: FONDAZIONE TOSCANA LIFE SCIENCES, Siena (IT)

(72) Inventors: Soraya Bosch, Siena (IT); Vittoria Zucconi, Siena (IT); Ida Paciello, Ogliastro Cilento (IT); Emanuele Roscioli, Siena (IT); Giampiero Batani, Siena (IT); Dario Cardamone, Siena (IT); Giuseppe Maccari, Siena (IT); Anna Kabanova, Siena (IT); Claudia Sala, Veduggio con Colzano (IT)

(73) Assignee: FONDAZIONE TOSCANA LIFE SCIENCES, Siena, SI (IT)

(21) Appl. No.: 19/150,284

(22) PCT Filed: Jan. 19, 2024
(86) PCT No.: PCT/IB2024/050528

§ 371 Date: Jul. 23, 2025

(30) Foreign Application Priority Data
Jan. 23, 2023 (IT) 102023000000924

(51) Int. Cl. C07K 16/1228; A61K 39/00

(52) U.S. Cl. CPC C07K 16/1228; A61K 2039/505; C07K 2317/21; C07K 2317/33; C07K 2317/52; C07K 2317/73

(57) ABSTRACT

The present invention relates to monoclonal antibodies or antigen-binding portion thereof that have a potent antibacterial activity against Klebsiella pneumoniae, in particular against multi-drug resistant Klebsiella pneumoniae strains. The invention relates also to the use of such monoclonal antibodies or antigen-binding portion thereof in therapy, prophylaxis, and diagnosis of Klebsiella pneumoniae infections, in particular Klebsiella pneumoniae dependent diseases.

### TECHNICAL FIELD OF THE INVENTION

The present invention relates to monoclonal antibodies or antigen-binding portion thereof that have a potent bactericidal activity against multidrug-resistant Klebsiellapneumoniae, in particular against metallo-β-lactamase (NDM)-producing strains of Klebsiella pneumoniae. The invention relates also to the use of such monoclonal antibodies or antigen-binding portion thereof in therapy, prophylaxis, and diagnosis of Klebsiellapneumoniae infections, in particular Klebsiellapneumoniae dependent diseases.

### State of the Art

Antimicrobial resistance (AMR) has been enlisted by the World Health Organization and the European Medicines Agency as one of the top 10 global health priorities, due to its impact on human health and socio-economic welfare worldwide. Among human pathogens that are increasingly acquiring resistance to antibiotics, a phenomenon known as multi-drug resistance (MDR), Klebsiellapneumoniae (herein abbreviated also as “Kp”) is by large the most common MDR agent. Kp causes both nosocomial infections (i.e., urinary tract infections, pneumonia, wound and surgical site infections, sepsis) and invasive community-acquired diseases, such as pyogenic liver abscess, endophthalmitis, and meningitis. Treatment is often limited by the increasing frequency of MDR Kp isolates producing carbapenemases, a class of enzymes conferring resistance to carbapenems, a subclass of β-lactam antibiotics. Among carbapenemases, the New Delhi Metallo-β-lactamases (NDM) are the most threatening enzymes since they confer resistance also to new β-lactamase inhibitor combinations (i.e., ceftazidime-avibactam, imipenem-relebactam, and meropenem-vaborbactam), which are considered the last line of defence. NDMs are encoded by bla_{NDM} genes located on large multi-resistance plasmids that have been spreading across high-risk MDR Kp strains throughout all continents.

NDM-producing strains are of notable concern since their emergence has been linked to specific genetic features endowing these strains with traits of hypervirulence, thus increasing the probability of Kp spread. Notably, NDM-1-positive sequence type ST147 clone has been causing a nosocomial outbreak in Tuscany, Italy, ongoing since 2018. Overall, ST147 is widely spread in India and South-Eastern Asiatic region and has been recently classified as pan-drug resistant. Genomic surveillance data indicated that Tuscany isolates of ST147 carry a highly diversified plasmid content, encoding both MDR and hypervirulence genes. Similar genetic rearrangements had been already observed in Russia (2017), United Kingdom (2018-2019) and Egypt (2019), where different Kp STs (ST15, ST147, ST395, and ST874) carrying hybrid plasmids coharbouring virulence genes and bla_{NDM} were reported. The same was observed in the 2019 outbreak in Germany where the hybrid element in hypermucoviscous bla_{NDM-1}/bla_{OXA-48}—carrying ST307 isolates was detected. Hence, convergence of genetic elements conferring MDR and hypervirulence traits in these epidemic clones represents an 10 alarming evolutionary development that underscores the potential of similar clones to cause even larger epidemics and urges the search for alternative treatment strategies.

Human monoclonal antibodies (mAbs) may represent a new powerful tool that can rapidly progress to innovative prophylactic or therapeutic solutions against AMR. mAbs are unique in their ability to target a virtually indefinite repertoire of antigens specific to a particular pathogen, in having intrinsically good safety profiles and avoiding depletion of the host microbiota.

However, there remains an urgent need for potent, broad spectrum antibody therapeutics for use in therapy, prophylaxis, and diagnosis of Kp infections, in particular MDR Kp-dependent diseases.

### SUMMARY OF THE INVENTION

To identify potent human mAbs against Kp, and particularly multi-drug resistant strains of Kp, the inventors developed a powerful method of unbiased mAb selection, not bound to a particular predetermined antigen, that allowed to isolate human mAbs from patients that 25 experienced Kp bloodstream infection during the Tuscan outbreak of 2018, that underwent functionality screening.

This strategy, disclosed in detail in the examples, allowed identification of Kp-specific mAbs that have potent in vitro bactericidal activity in the picomolar range of concentrations, with the top candidate being protective in the in vivo bacteremia model against multi-drug resistant Kp.

Notably, as will be clearly shown in the examples, the authors of the invention found that selected mAbs targeting the capsule of ST147_{NDM-1} were able to promote bacterial uptake in phagocytosis assays and trigger enchained bacterial growth which correlated with their ability to protect against bloodstream infection in vivo.

In certain aspects, the invention thus provides a human monoclonal antibody or antigen-binding portion thereof that specifically binds to a surface antigen of Kp.

In certain aspects, the invention provides a human monoclonal antibody or antigen-binding portion that specifically binds to a surface antigen of a drug resistant or multi-drug resistant strain of Kp, in particular to a New Delhi Metallo-β-lactamase (NDM)-producing strain of Kp.

In certain aspects, the invention provides a human monoclonal antibody or antigen-binding portion thereof that specifically binds to a surface antigen of at least one Klebsiella pneumoniae capsular type K64 strain.

In certain aspects, the invention provides a human monoclonal antibody or antigen-binding portion thereof that specifically binds to a surface antigen of Klebsiella pneumoniae Sequence Type 147 (ST147). In certain aspects, the invention provides a human monoclonal antibody or antigen-binding portion that specifically binds to capsular antigen (also herein indicated as K-antigen) or O-antigen of at least one Kp strain.

In certain aspects, the invention provides a human monoclonal antibody or antigen-binding portion thereof that specifically binds to capsular-antigen of and is bactericidal against at least one of the following strains: NDM-1 positive Klebsiella pneumoniae Sequence Type 147 (ST147) strain and NDM-9 positive Klebsiella pneumoniae Sequence Type 147 (ST147) strain.

In certain aspects, the invention provides a human monoclonal antibody or antigen-binding portion thereof that specifically binds to capsular-antigen of and is bactericidal against NDM-1 positive Klebsiellapneumoniae Sequence Type 147 (ST147) strain.

In certain aspects, the invention provides a human monoclonal antibody or antigen-binding portion thereof that specifically binds to a surface antigen of Klebsiella Pneumoniae comprising the light chain variable domain (VL) and heavy chain variable domain (VH) of a monoclonal antibody selected from the group consisting of: SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, SBJ11-J13.

In certain aspects, the invention provides a human monoclonal antibody or antigen-binding portion thereof that specifically binds to a region of a surface antigen of Klebsiella Pneumoniae comprising the CDRs of a monoclonal antibody selected from the group consisting of. SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, SBJ11-J13.

In certain aspects, the invention provides a human monoclonal antibody or antigen-binding portion thereof that specifically binds to a region of a surface antigen of Kp comprising the VL and VH domains that are at least 85%, 90%, 95%, 97%, 98% or 99% identical in amino acid sequence to the VL and VH domains, respectively, of a monoclonal antibody selected from the group consisting of: SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, SBJ11-J13.

In certain aspects, the invention provides human monoclonal antibodies or antigen-binding portion thereof that compete for the surface antigen of Kp with any of the antibodies herein disclosed.

In certain aspects, the invention provides human monoclonal antibody or an antigen-binding portion according to any embodiments herein disclosed, for use in a prophylactic or therapeutic treatment of a Kp infection or conditions or disorders resulting from such infection, preferably an infection of a Klebsiella pneumoniae capsular type K64 strain according to any of the variants disclosed herein.

In certain aspects, the invention provides human monoclonal antibody or an antigen-binding portion according to any embodiments herein disclosed, for use in a prophylactic or therapeutic treatment of a Kp infection or conditions or disorders resulting from such infection preferably an infection of a Klebsiella pneumoniae capsular type K64 strain according to any of the variants disclosed herein.

In certain aspects, the invention provides a method of preventing or treating a Kp infection or conditions or disorders resulting from such infection, comprising administering a human monoclonal antibody or an antigen-binding portion according to any embodiments herein disclosed, to a subject in need thereof. The invention further provides human monoclonal antibody or an antigen-binding portion according to any embodiments herein disclosed for use in the diagnosis, prophylaxis and/or treatment of a subject having, or at risk of developing, a Kp infection preferably an infection of a Klebsiella pneumoniae capsular type K64 strain according to any of the variants disclosed herein. Furthermore, the invention pertains to the use of the human binding molecules and/or the nucleic acid molecules of the invention in the diagnosis/detection of such infections.

In certain aspects, the invention provides a pharmaceutical composition comprising at least one or more human monoclonal antibodies or antigen-binding portions thereof according to any one of the embodiments herein disclosed and a pharmaceutically acceptable carrier and its use in the prevention and/or treatment of a Kp infection or conditions or disorders resulting from such infection, preferably an infection of a Klebsiellapneumoniae capsular type K64 strain according to any of the variants disclosed herein.

In certain aspects, the invention provides an isolated cell line that produces the antibody or antigen-binding portion thereof according to any one of the embodiments herein disclosed.

In certain aspects, the invention provides an isolated nucleic acid molecule comprising a nucleotide sequence that encodes the antibody or antigen-binding portion thereof according to any one of the embodiments herein disclosed.

In certain aspects, the invention provides a vector comprising the nucleic acid molecule encoding the antibody or antigen-binding portion thereof embodiments according to any one of the embodiments herein disclosed, wherein the vector optionally comprises an expression control sequence operably linked to the nucleic acid molecule.

In certain aspects, the invention provides a non-human transgenic animal or transgenic plant comprising the nucleic acid according to any one of the preceding embodiments, wherein the non-human transgenic animal or transgenic plant expresses said nucleic acid. In certain embodiments, said non-human transgenic animal is a mammal.

In certain aspects, the invention provides the use of the human monoclonal antibody or an antigen-binding portion thereof according to any one of the embodiments herein disclosed in the diagnosis of a Kp infection.

In certain aspects, the invention provides an in vitro method for revealing the presence of Klebsiellapneumoniae in a sample comprising the following steps: i) Contacting the antibody or an antigen-binding portion thereof according to any one of the embodiments herein disclosed;
ii) Detecting the binding of said antibody or an antigen-binding portion thereof to a surface antigen of Kp, preferably detecting the binding of said antibody or an antigen-binding portion thereof with capsular antigen of Klebsiella pneumoniae, more preferably capsular antigen of the K64 type.

In certain aspects, the invention provides an in vitro method for the diagnosis of a Kp infection in a subject comprising the following steps:
i) Contacting the antibody or an antigen-binding portion thereof according to any one of the embodiments herein disclosed with a biological sample of said subject;
ii) Detecting the binding of said antibody or an antigen-binding portion thereof to a surface antigen of Kp, preferably detecting the binding of said antibody or an antigen-binding portion thereof with capsular antigen of Klebsiella pneumoniae, more preferably capsular antigen of the K64 type.

The invention contemplates combinations of any of the foregoing aspects and embodiments of the invention.

### BRIEF DESCRIPTION OF THE DRAWINGS

FIG. 1. Isolation and selection of Kp-specific mAbs against ST147NDM-1 Tuscany outbreak strain(s). A) Schematic workflow of anti-Kp antibody isolation. B) ELISA screening of supernatants of 18′390 single cell-sorted MBCs isolated from 7 convalescent patients. Assay was performed to detect IgG/IgA against surface antigens of ST147_{NDM-1} c.i. 1 and ST147_{NDM-1} c.i.2. 214 supernatants showed a signal at least 2-fold superior to the blank of OD₄₀₅ and were selected for further analysis. C) L-SBA on recombinant mAbs against ST147_{NDM-1} c.i.1. 30% reduction in bacterial viability was used as a cut-off to select 20 bactericidal mAbs.

FIG. 2. Binding properties of 20 bactericidal ST147_{NDM-1}-specific mAbs A) Heatmap representing high-throughput flow cytometry screening of 20 functional mAbs against genetically diverse Kp panel. MFI was normalized to controls and transformed in logarithmic scale. B) Heatmap of ELISA with purified K-(capsule) and O-antigens. ELISA plates were coated with 100 μg/ml different Kp-specific O-Antigens (i.e. LPS O2a, in-house purified O2a from ST147_{NDM-1}, LPS O2afg, in-house purified O1v2 (v2: O2afg) from ST130_{OXA-48}, in-house purified capsule type 64 from ST147_{NDM-1}). Unrelated E. co/i LPS 0111:B4 was used as negative control and total bacterial lysate as a positive control. Values at least 3-fold superior to the blank of OD₄₀₅ were considered as positive. C-E) Representative immunoblot profiles of selected anti-Kp mAbs probed against total sugar extracts of indicated strains. O8O09 (panel C) results show a smeared, high molecular weight band in presence of total sugar extracts of ST147 Kp species, indicating recognition of KL64 capsule. O5N02 (panel D) results show a wide ladder-like profile against a broad panel of Kp STs, bearing both O2- and O1-type of O-antigen. O5D08 (panel E) shows medium molecular weight ladder-like signal for O2 carrying-Kp strains.

FIG. 3. Microscopy characterization of anti-Kp mAb binding on ST147NDM1 strain. A) Images show ST147 NDM1 expressing sfmCherry stained with O8O09, 05D08 and O5N02 mAbs labelled with anti-human A488 conjugated secondary antibody (green). Bacterial DNA is stained with dapi (blue). Scale bar 2 mm. B) Scatter plot showing the quantification of A488 intensity at the single bacterium level (n= . . . - . . . objects analysed). C) Scatter plot displaying the measurement of the area of individual A488 spots. D) Representative images showing ST147 ND_{M}1 stained with O8009-A488, 05N02-A555 and O5D08-A647 conjugated mAbs. Scale bar 2 mm.

FIG. 4. Functional in vitro characterization of 20 bactericidal ST147_{NDM-1}-targeting mAbs. A) Heatmap representing IC₅₀ values resulting from F-SBA screening of 20 anti-Kp mAbs against the panel of indicated bacterial strains. B) Bacterial fold uptake by THP1 macrophages measured in phagocytosis assay in the presence of anti-Kp mAbs from cluster 1. C) Bacterial fold uptake by THP1 cells measured in samples treated with anti-Kp mAbs from cluster 2. D) Panels show still frames from time-lapse imaging of bacteria expressing sfmCherry grown in 100 mg/mL of indicated mAbs. Scale bar 5 mm.

FIG. 5. Evaluation of in vivo protective properties of O8O09, 05D08 and O5N02 mAbs in immunocompetent ST147_{NDM-1} bacteremia model. A) Scheme of prophylaxis (PRO) study. Survival analysis of O8O09 (B), O5N02 (C) and O5D08 (D) PRO regimens and sham control (n=10 mice per group). (E) Survival analysis of O8O09 PRO regimens administered as a single dose of 5 mg/kg (n=10 mice per group). (F) Scheme of treatment (THR) study. (G) Survival analysis of O8O09 THR regimens and sham control (n=10 mice per group). (H) Scheme of prophylaxis plus treatment (PRO+THR). (I) Survival analysis of O8O09 PRO regimens and PRO+THR regiments, at 1 mg/kg and 5 mg/kg single doses (n=10 animals per group).

FIG. 6. Gating strategy for single cell sorting of total memory B cells. Flow cytometry plots reporting gating strategy to identify as follows: live cells, lymphocyte population, single cells, CD19-positive cells (B cells), CD19⁺ CD27T IgD— cells (memory B cells), CD19⁺ CD27⁺ IgD⁻ IgM⁻ B cells (memory B cells expressing IgG, IgA or IgE).

FIG. 7. Heatmap of ELISA results of 134 recombinant mAbs against ST147_{NDM-1} clinical isolates. ELISA against two different ST147_{NDM-1} clinical isolates was performed to confirm mAb binding to Kp surface. Values at least 3-fold superior to the blank of OD₄₀₅ were considered as positive hits.

FIG. 8. Heatmap representing the summary of high-throughput flow cytometry screening of 20 functional anti-Kp mAbs against a selected panel of different Klebsiella species, as well as commensals. MFI was normalized on controls and transformed in logarithmic scale. 20 FIG. 9. Purification of capsule type KL64. A) SEC-HPLC profile of purified KL64 capsule from ST147_{NDM-1}. Three arrows indicate molecular weight standards of 410, 80 and 12 kDa.

ST147_{NDM-1} capsular polysaccharide has been purified as reported in Materials and Methods. B) Theoretical structure of capsule type 64.

FIG. 10. Purification of different O antigen subtypes. SEC-HPLC profiles of purified β-antigens, (A) O2v1 version purified from ST147_{NDM-1} and (B) Olv2 version purified from ST13-_{OXA48}. Three arrows indicate molecular weight standards of 410, 80 and 12 kDa. C) Molecular structures of O antigens of O1, 02a, and O2afg type.

FIG. 11. Polysaccharide characterization of β-antigen deficient strain ST147_{NDM-9}. A) SEC-HPLC analysis of total sugar content after hydrolysis with acetic acid. Three peaks correspond to capsule, O antigen and the core. Three arrows indicate molecular weight standards of 410, 80 and 12 kDa. B) Silver staining analysis of total sugar extract from Kp strains used in this work. ST147_{NDM-9} lacks LPS ladder-like signal (25-50 kDa range).

FIG. 12. ROI definition and spot detection for mAb binding characterization. Images show on the left the binding pattern of A488-labelled anti-Kp mAbs on ST147_{NDM-1} Kp, in the middle the ROI where A488 signal was detected and on the right the morphology of A488 spot. Scale bar 2 μm.

FIG. 13. Sequence analysis of 20 selected mAbs. A) Frequency of IGHV and IGHJ genes usage (left), IGKV and IGKJ genes usage (right). B) IGHV and IGHJ genes pairing heatmap (left), IGKV and IGKJ genes pairing heatmap (right). C) IGHV and IGKV genes pairing heatmap (left), IGHJ and IGKJ genes pairing heatmap (right). D) CDR3 length distribution, heavy chains (left) and light chains (right). E) Percentage of variable chain identity with respect to the inferred germline.

FIG. 14. F-SBA profiling of 20 functional mAbs against complement-sensitive pathogenic Kp strains. F-SBA of 20 bactericidal anti-Kp mAbs against ST147_{NDM-1} c.i.1 (A), ST147_{NDM-1} c.i.2 (B), ST147_{NDM-9} (C), and ST307_{NDM-5} (D). Single experiments were normalized to negative control (no mAb). For each mAb, the bactericidal curve and the associated IC50 were obtained using the [Inhibitor]vs. normalized response, variable slope analysis on GraphPad Prism. IC₅₀ values are reported in FIG. 4A.

FIG. 15. Complement-dependent killing efficacy of 20 functional mAbs against four pathogenic Kp strains. Heatmap displays the percentage of reduction in resazurin fluorescence as a readout of bacterial viability, normalized to controls (no mAb). Results were extrapolated from F-SBA with single mAbs.

FIG. 16. Evaluation of in vivo protective properties of O8O09, 05D08 and O5N02 mAbs in immunocompetent ST147_{NDM-1} bacteremia model. (A) Survival analysis for groups of mice infected with ST147 isolate 30 at four tested inoculums (in log₁₀ CFU/mL). (B) O8O09 prophylaxis (PRO) regimens log₁₀ CFU/spleen at endpoint compared with controls. (C) 08O09 treatment (THR) regimens log₁₀ CFU/spleen at endpoint compared with controls. (D) Dose response comparison of survival time over 96 h in mice infected and receiving mAbs 08O09 treatment (THR) and prophylaxis plus treatment (PRO+THR) at 1 mg/kg (E) and 5 mg/kg single doses. In each curve 10 animals were tested.

FIG. 17. Pharmacokinetic experiments in THR and PRO studies. The concentration of human mAbs in mouse plasma was measured by quantitative ELISA of O8O09 administered in a single dose of 5 mg/kg by IV or IP. Values were obtained after 20 min measurement of of absorbance at 405 nm and interpolated from a sigmoidal curve in the presence of mice plasma by GraphPad.

### DETAILED DESCRIPTION OF THE INVENTION

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclature used in connection with, and techniques of, cell and tissue culture, molecular biology, immunology, microbiology, genetics and protein and nucleic acid chemistry and hybridization described herein are those well-known and commonly used in the art.

The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, second ed., Cold Spring Harbor Laboratory Press, Cold Spring Harbor, N.Y. (1989) and Ausubel et al, Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990), incorporated herein by reference.

The following terms, unless otherwise indicated, shall be understood to have the following meanings: The term “polypeptide” encompasses native or artificial proteins, protein fragments and polypeptide analogues of a protein sequence. A polypeptide may be monomeric or polymeric. The term “isolated protein”, “isolated polypeptide” or “isolated antibody” is a protein, polypeptide or antibody that by virtue of its origin or source of derivation (1) is not associated with naturally associated components that accompany it in its native state, (2) is free of other proteins from the same species, (3) is expressed by a cell from a different species, or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art. Examples of isolated antibodies include an anti-Kp antibody that has been affinity purified using Kp whole bacteria or a portion thereof, in particular a surface antigen thereof, an anti-Kp antibody that has been synthesized by a hybridoma or other cell line in vitro, and a human anti-Kp antibody derived from a transgenic animal. A protein or polypeptide is “substantially pure”, “substantially homogeneous”, or “substantially purified” when at least about 60 to 75% of a sample exhibits a single polypeptide. The polypeptide or protein may be monomeric or multimeric. A substantially pure polypeptide or protein will typically comprise about 50%, 60%, 70%, 80% or 90% W/W of a protein sample, more usually about 95%, and preferably will be over 99% pure.

Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification. The term “polypeptide fragment” as used herein refers to a polypeptide that has an amino-terminal and/or carboxy-terminal deletion, but where the remaining amino acid sequence is identical to the corresponding positions in the naturally occurring sequence. In some embodiments, fragments are at least 5, 6, 8 or 10 amino acids long. In other embodiments, the fragments are at least 14, at least 20, at least 50, or at least 70, 80, 90, 100, 150 or 200 amino acids long.

The term “polypeptide analogue” as used herein refers to a polypeptide that comprises a segment that has substantial identity to a portion of an amino acid sequence and that has at least one of the following properties: (1) specific binding to a surface antigen of Kp according to any of the embodiments disclosed herein under suitable binding conditions, (2) bactericidal activity against Kp. Typically, polypeptide analogues comprise a conservative amino acid substitution (or insertion or deletion) with respect to the native sequence.

Analogues typically are at least 20 or 25 amino acids long, preferably at least 50, 60, 70, 80, 90, 100, 150 or 200 amino acids long or longer, and can often be as long as a full-length polypeptide. Some embodiments of the invention include polypeptide fragments or polypeptide analogue antibodies with 1, 2, 3, 4, 5, 6, 7, 8, 9, 10, 11, 12, 13, 14, 15, 16 or 17 substitutions from the germline amino acid sequence. In certain embodiments, amino acid substitutions to an anti-Kp antibody or antigen-binding portion thereof are those which: (1) reduce susceptibility to proteolysis, (2) reduce susceptibility to oxidation, (3) alter binding affinity to form protein complexes, and (4) confer or modify other physicochemical or functional properties of such analogues, but still retain specific binding to Kp surface antigens. Analogues can include various muteins of a sequence other than the normally occurring peptide sequence. For example, single or multiple amino acid substitutions, preferably conservative amino acid substitutions, may be made in the normally occurring sequence, preferably in the portion of the polypeptide outside the domain(s) forming intermolecular contacts. A conservative amino acid substitution should not substantially change the structural characteristics of the parent sequence, e.g., a replacement amino acid should not alter the anti-parallel [beta]-sheet that makes up the immunoglobulin binding domain that occurs in the parent sequence or disrupt other types of secondary structure that characterizes the parent sequence. In general, glycine and proline would not be used in an anti-parallel [beta]-sheet. Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et ai, Nature 354:105 (1991), incorporated herein by reference.

As used herein, the term “Klebsiella pneumoniae”, also abbreviated throughout the specification as “Kp”, refers to the species of Gram-negative, non-motile, encapsulated, lactose-fermenting, facultative anaerobic, rod-shaped bacterium. It appears as a mucoid lactose fermenter on MacConkey agar. In the clinical setting, it is the most significant member of the genus Klebsiella of the Enterobacteriaceae. In recent years, Klebsiella species have become important pathogens in nosocomial infections.

Although found in the normal flora of the mouth, skin, and intestines, Kp can cause destructive changes to human and animal lungs if aspirated, specifically to the alveoli resulting in bloody, brownish or yellow coloured jelly like sputum. Notably, Kp causes both nosocomial infections (i.e., urinary tract infections, pneumonia, wound and surgical site infections, sepsis) and invasive community-acquired diseases, such as pyogenic liver abscess, endophthalmitis, and meningitis.

Kp also naturally occurs in the soil, and about 30% of strains can fix nitrogen in anaerobic conditions. As a free-living diazotroph, its nitrogen-fixation system has been much-studied, and is of agricultural interest, as Kp has been demonstrated to increase crop yields in agricultural conditions.

Where an “antibody” is referred to herein with respect to the invention, it is normally understood that an antigen-binding portion thereof may also be used. An antigen-binding portion competes with the intact antibody for specific binding. See generally, Fundamental Immunology, Ch. 7 (Paul, W., ed., second ed. Raven Press, N.Y. (1989)) (incorporated by reference in its entirety for all purposes). Antigen-binding portions may be produced by recombinant DNA techniques or by enzymatic or chemical cleavage of intact antibodies.

In some embodiments, antigen-binding portions include Fab, Fab′, F(ab′)₂, Fd, Fv, dAb, and complementarity determining region (CDR) fragments, single-chain antibodies (scFv), chimeric antibodies, diabodies, nanobodies and any polypeptides that contain at least a portion of an antibody that is sufficient to confer specific antigen binding to the polypeptide.

From N-terminus to C-terminus, both the mature light and heavy chain variable domains comprise the regions FR1, CDR1, FR2, CDR2, FR3, CDR3 and FR4. The assignment of amino acids to each domain herein is in accordance with the definitions of IMGT convention described in Lefranc et al. (2003), Developmental & Comparative Immunology 27.1 (2003): 55-77.

As used herein, an antibody that is referred to by number is the same as a monoclonal antibody that is obtained from the human peripheral blood mononuclear cells (PBMCs) isolated from the donor of the same number. For example, monoclonal antibody SBJ08-009 (also herein denoted as J08009) is the same antibody as one obtained from PBMCs isolated from subject identified by the code O8, or a subclone thereof. As used herein, an antibody or antigen-binding portion thereof which specifically binds to a surface antigen of Kp according to any of the embodiments disclosed in the present specification and in the claims is also denoted as an anti-Kp antibody or antigen-binding portion thereof.

As used herein, a Fd fragment means an antibody fragment that consists of the VH and CH 1 domains; an Fv fragment consists of the VL and VH domains of a single arm of an antibody; and a dAb fragment (Ward et al, Nature 341:544-546 (1989)) consists of a VH domain.

In some embodiments, the antibody is a single-chain antibody (scFv) in which a VL and VH domains are paired to form a monovalent molecule via a synthetic linker that enables them to be made as a single protein chain. (Bird et al, Science 242:423-426 (1988) and Huston et al, Proc. Natl Acad. ScL USA 85:5879-5883 (1988)). In some embodiments, the antibodies are diabodies, i.e., are bivalent antibodies in which VH and VL domains are expressed on a single polypeptide chain but using a linker that is too short to allow for pairing between the two domains on the same chain, thereby forcing the domains to pair with complementary domains of another chain and creating two antigen binding sites. (See e.g., Holliger P. et al, Proc. Natl. Acad. ScL USA 90:6444-6448 (1993), and Poljak R. J. et al, Structure 2:1121-1123 (1994)). In such embodiments, the CDR(s) may be incorporated as part of a larger polypeptide chain, may be covalently linked to another polypeptide chain, or may be incorporated noncovalently. In embodiments having one or more binding sites, the binding sites may be identical to one another or may be different.

As used herein, the term “human antibody” means any antibody in which the variable and constant domain sequences are human sequences or any of the CDRs of the variable domain sequences are human sequences. The term encompasses antibodies with sequences derived from human genes, but which have been changed, e.g., to decrease possible immunogenicity, increase affinity, eliminate cysteines that might cause undesirable folding, etc. The term encompasses such antibodies produced recombinantly in non-human cells, which might impart glycosylation not typical of human cells. The term “chimeric antibody” as used herein means an antibody that comprises regions from two or more different antibodies.

The term “epitope” includes any protein determinant capable of specific binding to an immunoglobulin or T-cell receptor or otherwise interacting with a molecule. Epitopes or antigenic determinants generally consist of chemically active surface groupings of molecules such as amino acids or carbohydrate or sugar side chains and generally have specific three-dimensional structural characteristics, as well as specific charge characteristics. An epitope may be “linear” or “conformational.” In a linear epitope, all of the points of interaction between the protein and the interacting molecule (such as an antibody) occur linearly along the primary amino acid sequence of the protein. In a conformational epitope, the points of interaction occur across amino acid residues on the protein that are separated from one another.

An antibody with “bactericidal activity” or “anti-bacterial activity” as used herein means an antibody that exhibits the capability of destroying and/or inhibiting the action of its target, namely one or more Kp strains according to any of the embodiments disclosed herein, or else an antibody that exhibits the capability of killing one or more Kp strains according to any of the embodiments disclosed herein.

In particular, an antibody or antigen-binding portion thereof according to the present invention displays “functional activity” against a Kp organism when the antibody or antigen-binding portion thereof exhibits complement-mediated bactericidal activity against Kp as determined using any of the standard assays described herein, e.g. a fluorescence-based serum bactericidal assay using resazurin staining as readout of cell-viability.

An antibody with “bactericidal activity” or “anti-bacterial activity”, in particular with complement-mediated bactericidal or anti-bacterial activity as used herein is referred for example to an antibody or antigen-binding portion thereof showing a 50% inhibitory concentration (IC50) of less than 100 ng/ml, preferably less than 10 ng/ml, more preferably less than 5 ng/ml, when tested by an in vitro fluorescence-based serum bactericidal assay (F-SBA) against Kp, performed for example as disclosed in the present specification and in the examples.

The term “polynucleotide” as referred to herein means a polymeric form of nucleotides of at least 10 bases in length, either ribonucleotides or deoxyribonucleotides or a modified form of either type of nucleotide. The term includes single and double stranded forms.

The term “isolated polynucleotide” as used herein means a polynucleotide of genomic, cDNA, or synthetic origin or some combination thereof, which by virtue of its origin the “isolated polynucleotide” (1) is not associated with all or a portion of a polynucleotides with which the “isolated polynucleotide” is found in nature, (2) is operably linked to a polynucleotide to which it is not linked in nature, or (3) does not occur in nature as part of a larger sequence.

The term “naturally occurring nucleotides” as used herein includes deoxyribonucleotides and ribonucleotides. The term “modified nucleotides” as used herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al., Nucl. Acids Res. 14:9081 (1986); Stec et al, J. Am. Chem. Soc. 106:6077 (1984); Stein et al., Nucl. Acids Res. 16:3209 (1988); Zon et al., Anti-Cancer Drug Design 6:539 (1991); Zon et al.,

Oligonucleotides and Analogues: A Practical Approach, pp. 87-108 (F. Eckstein, Ed., Oxford University Press, Oxford England (1991)); U.S. Pat. No. 5,151,510; Uhlmann and Peyman, Chemical Reviews 90:543 (1990), the disclosures of which are hereby incorporated by reference. An oligonucleotide can include a label for detection, if desired. “Operably linked” sequences include both expression control sequences that are contiguous with the gene of interest and expression control sequences that act in trans or at a distance to control the gene of interest. The term “expression control sequence” as used herein means polynucleotide sequences that are necessary to affect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing and can also include additional components whose presence is advantageous, for example, leader sequences and fusion partner sequences. The term “vector”, as used herein, means a nucleic acid molecule capable of transporting another nucleic acid to which it has been linked. In some embodiments, the vector is a plasmid, i.e., a circular double stranded piece of DNA into which additional DNA segments may be ligated. In some embodiments, the vector is a viral vector, wherein additional DNA segments may be ligated into the viral genome. In some embodiments, the vectors are capable of autonomous replication in a host cell into which they are introduced (e.g., bacterial vectors having a bacterial origin of replication and episomal mammalian vectors). In other embodiments, the vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”).

The term “recombinant host cell” (or simply “host cell”), as used herein, means a cell into which a recombinant expression vector has been introduced. It should be understood that “recombinant host cell” and “host cell” mean not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein.

The term “percent sequence identity” in the context of nucleotide or aminoacidic sequences means the residues in two sequences that are the same when aligned for maximum correspondence. The length of sequence identity comparison may be over a stretch of at least about nine nucleotides, usually at least about 18 nucleotides, more usually at least about 24 nucleotides, typically at least about 28 nucleotides, more typically at least about 32 nucleotides, and preferably at least about 36, 48 or more nucleotides. There are a number of different algorithms known in the art which can be used to measure nucleotide sequence identity. For instance, polynucleotide sequences can be compared using FASTA, Gap or Bestfit, which are programs available, provides alignments and percent sequence identity of the regions of the best overlap between the query and search sequences (Pearson, Methods Enzymol. 183:63-98 (1990); Pearson, Methods MoI. Biol. 132:185-219 (2000); Pearson, Methods Enzymol. 266:227-258 (1996); Pearson, J MoI. Biol 276:71-84 (1998); incorporated herein by reference). The term “substantial similarity” or “substantial sequence similarity,” when referring to a nucleic acid or fragment thereof, or aminoacidic means that when optimally aligned with appropriate nucleotide insertions or deletions with another nucleic acid (or its complementary strand), there is nucleotide sequence identity in at least about 85%, preferably at least about 90%, and more preferably at least about 95%, 96%, 97%, 98% or 99% of the nucleotide bases, as measured by any well-known algorithm of sequence identity, such as FASTA, BLAST or Gap, as discussed above. As applied to polypeptides, the term “substantial identity” means that two peptide sequences, when optimally aligned, such as by the programs GAP or BESTFIT using default gap weights as supplied with the programs, share at least 70%, 75% or 80% sequence identity, preferably at least 90% or 95% sequence identity, and more preferably at least 97%, 98% or 99% sequence identity. In certain embodiments, residue positions that are not identical differ by conservative amino acid substitutions. A “conservative amino acid substitution” is one in which an amino acid residue is substituted by another amino acid residue having a side chain R group with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity may be adjusted upwards to correct for the conservative nature of the substitution. Means for making this adjustment are well-known to those of skill in the art. See, e.g., Pearson, Methods MoI. Biol. 243:307-31 (1994).

Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulphur-containing side chains: cysteine and methionine. Conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al, Science 256:1443-45 (1992), incorporated herein by reference. A “moderately conservative” replacement is any change having a nonnegative value in the PAM250 log-likelihood matrix. Sequence identity for polypeptides is typically measured using sequence analysis software. Protein analysis software matches sequences using measures of similarity assigned to various substitutions, deletions and other modifications, including conservative amino acid substitutions. For instance, GCG contains programs such as “Gap” and “Bestfit” which can be used with default parameters as specified by the programs to determine sequence homology or sequence identity between closely related polypeptides, such as homologous polypeptides from different species of organisms or between a wild type protein and a mutein thereof.

As used herein, the terms “label” or “labelled” refers to incorporation of another molecule in the antibody. In one embodiment, the label is a detectable marker, e.g., incorporation of a radiolabelled amino acid or attachment to a polypeptide of biotinyl moieties that can be detected by marked avidin (e.g., streptavidin containing a fluorescent marker or enzymatic activity that can be detected by optical or colorimetric methods). In another embodiment, the label or marker can be therapeutic, e.g., a drug conjugate or toxin. Various methods of labelling polypeptides and glycoproteins are known in the art and may be used.

The expressions “drug resistant” and “multi-drug resistant” (abbreviated as MDR) as applied herein to a bacterium define a bacterium which is resistant to at least one class or else to two or more classes of drugs, respectively. In particular, the expression “multi-drug resistant” is used to define a bacterium which is resistant to two or more classes of antibiotics, including but not limited to β-lactam antibiotics (herein indicated also as beta-lactam antibiotics), in particular carbapenems.

The terms “capsular-antigen” or “K-antigen” as used throughout the present specification as synonyms refer to antigen(s) belonging to the bacterial capsule polysaccharide (CPS) of Kp according to any of the variants herein disclosed. Unless otherwise defined, the terms “capsular-antigen” or “K-antigen” as used herein encompass capsular polysaccharide of said Kp or any portion or fragment thereof. The term “O-antigen” as used herein refers to the somatic antigen on the cell surface of Kp. The O-antigen is the most surface exposed part of the bacterial lipopolysaccharide. It is composed of repetitive units of oligosaccharides of different lengths with usually 2 to 7 sugar moieties per unit.

As used herein, the expression “carbapenemase-producing strain” is referred to any strain of Kp producing carbapenemases, a class of beta-lactamase enzymes conferring resistance to carbapenems, a subclass of β-lactam antibiotics. In terms of structure, the carbapenems are very similar to the penicillins (penams), but the sulfur atom in position 1 of the structure has been replaced with a carbon atom, and an unsaturation has been introduced-hence the name of the group, the carbapenems.

Carbapenemases confer a large antibiotic-resistance spectrum to Kp strains because these enzymes can hydrolyze and inactivate not only carbapenem antibiotics but also broad-spectrum penicillins, oxymino-cephalosporins, and cephamycins.

As used herein, the expression “New Delhi Metallo-β-lactamase (NDM)-producing strain” is referred to any strain of Kp producing New Delhi Metallo-β-lactamases (NDMs, herein indicated also as New Delhi Metallo-beta-lactamases), which are specific members of the large gene family that encodes the β-lactamase enzymes called carbapenemases. NDMs are capable of conferring resistance not only to a broad range of β-lactam antibiotics but also to new β-lactamase inhibitor combinations, including but not limited to ceftazidime-avibactam, imipenem-relebactam, and meropenem-vaborbactam combinations.

NDMs are encoded by bla_{NDM} genes located on large multi-resistance plasmids that have been spreading across high-risk MDR Kp strains throughout all continents (di Pilato V. et al. 2022 Mar. 1; 3(3): e224-34; Martin M J, et al. PNAS 2021; 118:1-8). NDMs comprise several variants encoded by corresponding bla_{NDM} genes, which share different properties.

As used herein, the expressions “NDM-1 positive strain(s)”, “NDM-5 positive strain(s)” or “NDM-9 positive strain(s)” (herein abbreviated also as NDM-1⁺, NDM-5⁺ and NDM-9⁺) are referred to any strain of Kp capable of producing the following enzymes: New Delhi metallo-β-lactamase 1, New Delhi metallo-β-lactamase 5, and New Delhi metallo-β-lactamase 9, respectively, each encoded by the corresponding gene, i.e. bla_{NDM-1}, bla_{NDM-5} and bla_{NDM-9}.

The NDM-1 enzyme was originally named after New Delhi, the capital city of India, as it was first described by Yong et al. in December 2009 in a Swedish national who fell ill with an antibiotic-resistant bacterial infection that he acquired in India (Yong D, et al. 2009 Antimicrob. Agents Chemother. 53 (12): 5046-5054). The infection was identified as a carbapenem-resistant Klebsiella pneumoniae strain bearing the novel gene bla_{NDM-1}.

In the context of the present specification, an NDM-producing strain of Kp belonging to a specific Sequence Type (ST) can be denoted by the following nomenclature: “STX_{NDMY}” or “STX_{NDM-Y}” or “STX_{NDMY}+”, wherein X represents the specific ST number of the Kp strain and Y represents the specific variant of NDM that is produced by said strain.

As used herein, the expression “Klebsiella pneumoniae capsular type K64 strain” refers to any strain of Kp bearing gene(s) from the capsule type 64 biosynthetic locus, namely gene(s) encoding for enzyme(s) responsible or involved in the biosynthesis of capsular polysaccharide (CPS) of the K64 type.

In other words, the expression “Klebsiella pneumoniae capsular type K64 strain” as used herein refers to any strain or clonal lineage of Klebsiella pneumoniae bearing a capsular polysaccharide (CPS) of the K64 type.

In the context of the present specification the terms “K64”, “KL64”, “K-64”, “KL-64” all referring to the capsular type 64 of Klebsiella pneumoniae, are used interchangeably.

The term “IC₅₀” as used herein refers to concentration of an antibody which reduces Kp cell viability by 50% as measured for example by a fluorescence-based serum bactericidal assay, in particular resazurin-based serum bactericidal assay as disclosed herein.

### Human Anti-Kp Antibodies and Characterization Thereof

In one embodiment, the invention provides a human monoclonal antibody or antigen-binding portion thereof that specifically binds to a surface antigen of Kp.

Preferably, in one embodiment the invention provides a human monoclonal antibody or antigen-binding portion thereof that specifically binds to a surface antigen of at least one drug resistant or multi-drug resistant strain of Kp, more preferably at least one carbapenemase-producing strain of Kp, even more preferably at least one NDM-producing strain of Kp.

Preferably, in one embodiment the invention provides a human monoclonal antibody or antigen-binding portion thereof that specifically binds to a surface antigen of at least one Klebsiella pneumoniae capsular type K64 strain.

In one preferred embodiment, said strain of Kp comprises one or more strains of Kp selected from the following: NDM-1 positive strains, NDM-9 positive strains, and NDM-5 positive strains.

In one preferred embodiment, a strain of Kp according to any of the variants disclosed herein comprises one or more strains or clinical isolates of Kp selected from the strains termed or clonal lineages defined by Sequence Type 147 (ST147), Sequence Type 258 (ST258), Sequence Type 493 (ST493), Sequence Type 307 (ST307), Sequence Type 13 (ST13), and Sequence Type 512 (ST512), in particular from ST147, ST 258.

In one embodiment, the present invention particularly provides a human monoclonal antibody or antigen-binding portion thereof that specifically binds to a surface antigen of at least one NDM-producing strain of Kp selected from the following Sequence Types: ST147, ST258, ST493, ST307, and ST13, in particular from ST147, ST 258, ST307 and ST493.

Preferably, said Kp comprises at least one NDM-producing strain of Kp selected from the group consisting of: ST147_{NDM-1}, ST147_{NDM-9}, and ST307_{NDM-5}.

In some aspects, the human monoclonal antibody or antigen-binding portion thereof according to any of the embodiments disclosed in the present specification and in the claims specifically binds to a surface antigen of at least one strain, at least two strains, at least three strains, at least four strains or at least five strains of Kp according to any of the variants disclosed in the present specification and in the claims.

According to a more preferred embodiment, the invention provides a human monoclonal antibody or antigen-binding portion thereof that specifically binds to a surface antigen of the ST147_{NDM-1} strain of Kp, which has been causing a nosocomial outbreak in Tuscany, Italy, ongoing since 2018. Overall, ST147 is widely spread in India and South-Eastern Asiatic region and has been recently classified as pan-drug resistant.

In one preferred embodiment said Kp is one, more or all of ST147_{NDM-1}, and ST147_{NDM-9} strains, more preferably is one or all of ST147_{NDM-1} and ST147NDM-₉ strains, and said human monoclonal antibody or antigen-binding portion thereof is selected from SBJ08-009, SBJ08-D18, SBJ03-F18, SBJ08-H10, SBJ08-N23, SBJ08-K19, SBJ08-M13, SBJ03-L02, SBJ08-J10, SBJ10-H18, SBJ08-003, SBJ05-K07, SBJ09-I10, and SBJ11-C06.

In another preferred embodiment said Kp is one or more, preferably comprises all of strains ST147_{NDM-1}, ST307_{NDM-5}, ST258 and ST493, and said human monoclonal antibody or antigen-binding portion thereof is selected from SBJ05-D08, SBJ05-C11, SBJ05-D14, SBJ11-J13, SBJ05-M13, SBJ09-G14, SBJ05-N02, SBJ09-M06, SBJ08-F04, and SBJ05-B17.

In another preferred embodiment, said Kp is one or more, preferably comprises all of strains ST147_{NDM-1}, and ST307_{NDM-5}, and said human monoclonal antibody or antigen-binding portion thereof is selected from SBJ05-D08, SBJ05-C11, SBJ05-D14, SBJ11-J13, and SBJ05-M13.

In another preferred embodiment, said Kp is one or more, preferably comprises all of strains ST147_{NDM-1}, ST307_{NDM-5}, and ST493, more preferably is a ST493 strain, and said human monoclonal antibody or antigen-binding portion thereof is selected from SBJ09-G14, SBJ05-N02, SBJ09-M06, SBJ08-F04, and SBJ05-B17.

As used in the present specification and in the claims, the term “surface antigen” encompasses any antigens displayed on the bacterial surface of Kp, in particular of a strain of Kp according to any of the variants herein disclosed.

In one embodiment, said surface antigen is selected from i) capsular-antigen (K-antigen) and/or the (ii) O-antigen of at least one strain of Kp according to any of the variants disclosed herein.

According to a more preferred embodiment, the invention provides a human monoclonal antibody or antigen-binding portion thereof that specifically binds to capsular antigen of and is bactericidal against at least one of the following strains: NDM-1 positive Klebsiella pneumoniae Sequence Type 147 (ST 147) strain and NDM-9 positive Klebsiellapneumoniae Sequence Type 147 (ST147) strain.

In particular, the invention provides a human monoclonal antibody or antigen-binding portion thereof that specifically binds to capsular antigen of and is bactericidal against NDM-1 positive Klebsiellapneumoniae Sequence Type 147 (ST147) strain.

In one preferred embodiment, said capsular-antigen is capsular polysaccharide, more preferably is capsular polysaccharide of the K64 type.

In one embodiment, said O-antigen is the O1-type antigen and/or the O2-type antigen of at least one strain of Kp according to any of the variants disclosed herein.

In another embodiment, said O2-type antigen is selected from O2a and O2afg antigen.

In one preferred embodiment said surface antigen is capsular-antigen and said human monoclonal antibody or antigen-binding portion thereof is selected from SBJ08-009, SBJ08-D18, SBJ03-F18, SBJ08-H10, SBJ08-N23, SBJ08-K19, SBJ08-M13, SBJ03-L02, SBJ08-J10, SBJ10-H18, SBJ08-003, SBJ05-K07, SBJ09-I10, and SBJ11-C06, preferably is SBJ08-009.

Preferably, when said surface antigen is capsular-antigen, in particular a capsular-antigen from a KL64 type capsule, said Kp is one or more of, preferably comprises all of strains ST147_{NDM-1}, and ST147_{NDM-9}, more preferably is one or all of ST147_{NDM-1} and ST147_{NDM-9} strains.

In another preferred embodiment said surface antigen is O-antigen and said human monoclonal antibody or antigen-binding portion thereof is selected from SBJ05-D08, SBJ05-C11, SBJ05-D14, SBJ11-J13, SBJ05-M13, SBJ09-G14, SBJ05-N02, SBJ09-M06, SBJ08-F04, and SBJ05-B17.

Preferably, when said surface antigen is the O-antigen, said Kp is one or more of, preferably comprises all of strains ST147_{NDM-1}, ST307N_{DM}-5, ST258 and ST493.In another preferred embodiment, said surface antigen is O2-type antigen and said human monoclonal antibody or antigen-binding portion thereof is selected from SBJ05-D08, SBJ05-C11, SBJ05-D14, SBJ11-J13, and SBJ05-M13.

Preferably, when said surface antigen is the O2-type antigen, said Kp is one or more of, preferably comprises all of strains ST147_{NDM-1}, and ST307_{NDM-5}.

In another preferred embodiment, said surface antigen is an epitope shared by O1-type and 02-type of O-antigens and said human monoclonal antibody or antigen-binding portion thereof is selected from SBJ09-G14, SBJ05-N02, SBJ09-M06, SBJ08-F04, and SBJ05-B17.

Preferably, when said surface antigen is the O1-type antigen, said Kp is a ST493 strain.

In certain aspects, the human monoclonal antibody or antigen-binding portion thereof that specifically binds to a surface antigen of Kp according to any of the embodiments disclosed herein, exhibits bactericidal or anti-bacterial activity, in particular exhibits complement-mediated bactericidal or anti-bacterial activity.

Complement-mediated bactericidal activity of the antibodies can be evaluated using standard assays, such as luminescence-based or fluorescence-based serum bactericidal assays known to the person skilled in the art. Merely by way of example, in these standard assays, Kp is reacted with a complement source as well as with the antibody to be tested. Bacterial counts are determined at various sampling times. Those antibodies that demonstrate complement-mediated bactericidal activity, as demonstrated by dose-dependent reduction in viable bacterial cell counts to a minimum of 50% determined after incubation with antibody and complement, as compared to colony counts at time zero, are considered to exhibit bactericidal activity for purposes of the present invention and are suitable for further use.

In one embodiment, the invention provides a human monoclonal antibody or antigen-binding portion that specifically binds to a surface antigen of Kp according to any of the embodiments disclosed herein, wherein said antibody or antigen-binding portion thereof provides a percentage of bacterial killing, in particular complement-dependent bacterial killing greater than 30%, preferably greater than 60%, preferably greater than 70%, more preferably greater than 80%, even more preferably greater than 85%, 90%, 95%, 98% or 99%, as measured by an in vitro luminescence-based serum bactericidal assay (L-SBA) and/or by an in vitro fluorescence-based serum bactericidal assay (F-SBA) performed for example as disclosed in the examples.

In a preferred embodiment, bacterial killing is measured by an L-SBA assay performed by incubating at least one strain of Kp according to any of the variants herein disclosed with at least four serial dilutions of mAbs in the presence of an appropriate amount of baby rabbit complement (BRC). Upon addition of luciferase, the luminescence signal generated by turnover of luciferin in the presence of bacterial ATP is measured as a proxy of Kp viability, thus allowing for rapid and sensitive quantification of bacterial survival. In each experiment, luminescence values obtained from all mAbs can be used to calculate the median value for each dilution tested. The difference between the luminescence of each single mAb to the median value can used as a readout of bacterial viability. The proper amount or concentration of BRC used in the assay can be optimized by performing a complement sensitivity screening of the selected strain(s) of Kp, for instance as disclosed in the examples.

According to a preferred aspect, a human monoclonal antibody or antigen-binding portion thereof of the invention shows a 50% inhibitory concentration (IC50) of less than 100 ng/ml, preferably less than 50, 25, 20, 10, 8, 6, 5, 4, 3, 2, or 1 ng/ml, when tested by an in vitro fluorescence-based serum bactericidal assay (F-SBA) against Kp, for example against the ST147_{NDM-1} strain, the ST147_{NDM-9} strain and/or the ST307NDM-₅ strain of Kp, performed for instance as disclosed in the examples.

In a preferred embodiment, said F-SBA assay is performed by incubating at least one strain of Kp according to any of the variants herein disclosed with at least three serial dilutions of mAbs in the presence of an appropriate amount of baby rabbit complement (BRC) for instance as disclosed in the examples, in particular using resazurin staining as a fluorescent readout of bacterial viability. In case resazurin is used the F-SBA assay is also herein abbreviated as “R-SBA”.

As disclosed in more details in the Examples, the inventors found that the most potent bactericidal antibodies SBJ08-009, SBJ08-D18, and SBJ03-F18, surprisingly exhibit bactericidal activity against all of ST147_{NDM-1}, ST147_{NDM-9} strains.

In certain aspects, the invention provides a human monoclonal antibody or antigen-binding portion thereof that specifically binds a surface antigen of Kp according to any of the embodiments disclosed herein, in particular a surface antigen of at least one strain of Kp according to any of the variants disclosed herein, comprising: (a) a heavy chain variable domain amino acid sequence that comprises the amino acid sequence of the heavy chain variable domain of an antibody selected from: SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, and SBJ11-J13; (b) a light chain variable domain amino acid sequence that comprises the amino acid sequence of the light chain variable domain of an antibody selected from: SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, and SBJ11-J13; (c) a heavy chain variable domain of (a) and a light chain variable domain of (b); or (d) heavy chain and light chain variable domain amino acid sequences comprising the heavy chain and light chain variable domain amino acid sequences, respectively, from the same antibody selected from: SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, and SBJ11-J13. 10 The nucleic acids encoding the full-length, or variable domain-comprising portions, of heavy and light chains, and the corresponding deduced amino acid sequences of the antibodies of the invention can be found in the sequence listing herein enclosed in the description.

In certain aspects, the invention provides a monoclonal antibody or an antigen-binding portion thereof that specifically binds a surface antigen of Kp according to any of the embodiments disclosed herein, in particular a surface antigen of at least one strain of Kp according to any of the variants disclosed herein, comprising: (a) a heavy chain variable domain amino acid sequence that comprises the heavy chain CDR1, CDR2 and CDR3 amino acid sequences of an antibody selected from: SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, and SBJ11-J13; (b) a light chain variable domain amino acid sequence that comprises the light chain CDR1, CDR2 and CDR3 amino acid sequences of an antibody selected from: SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, and SBJ11-J13; (c) a heavy chain variable domain of (a) and a light chain variable domain of (b); or (d) the heavy chain variable domain and light chain variable domain of (c), comprising heavy chain and light chain CDR amino acid sequences from the same antibody selected from: SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, and SBJ11-J13.

In certain aspects, the invention provides a monoclonal antibody that specifically binds a surface antigen of Kp according to any of the embodiments disclosed herein, in particular a surface antigen of at least one strain of Kp according to any of the variants disclosed herein, wherein said antibody comprises a heavy chain of an antibody selected from the group consisting of. SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, and SBJ11-J13. In certain aspects, the invention provides a monoclonal antibody that specifically binds a surface antigen of Kp according to any of the embodiments disclosed herein, in particular a surface antigen of at least one strain of Kp according to any of the variants disclosed herein, wherein said antibody comprises a light chain of an antibody selected from the group consisting of SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, and SBJ11-J13. In certain aspects, the invention provides a monoclonal antibody that specifically binds a surface antigen of Kp according to any of the embodiments disclosed herein, in particular a surface antigen of at least one strain of Kp according to any of the variants disclosed herein, wherein said antibody comprises a heavy chain and a light chain of the same antibody which is selected from the group consisting of SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, and SBJ11-J13.

In certain aspects, the invention provides a human monoclonal antibody or antigen-binding portion thereof that specifically binds a surface antigen of Kp according to any of the embodiments disclosed herein, in particular a surface antigen of at least one strain of Kp 30 according to any of the variants disclosed herein, comprising VL and VH domains that are at least 85%, 90%, 95%, 97%, 98% or 99% identical in amino acid sequence to the VL and VH domains, respectively, of a monoclonal antibody selected from the group consisting of SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, and SBJ11-J13.

In certain aspects, the invention provides a human monoclonal antibody or antigen-binding portion thereof that specifically binds a surface antigen of Kp according to any of the embodiments disclosed herein, in particular a surface antigen of at least one strain of Kp according to any of the variants disclosed herein, comprising the light chain and the heavy chain that are at least 85%, 90%, 95%, 97%, 98% or 99% identical in amino acid sequence to the light chain and the heavy chain, respectively, of a monoclonal antibody selected from the group consisting of SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, and SBJ11-J13.

One type of amino acid substitution that may be made is to change one or more cysteines in the antibody, which may be chemically reactive, to another residue, such as, without limitation, alanine or serine. In one embodiment, there is a substitution of a non-canonical cysteine. The substitution can be made in a CDR or framework region of a variable domain or in the constant domain of an antibody. In some embodiments, the cysteine is canonical.

Another type of amino acid substitution that may be made is to change any potential proteolytic sites in the antibody. Such sites may occur in a CDR or framework region of a variable domain or in the constant domain of an antibody. Substitution of cysteine residues and removal of proteolytic sites may decrease the risk of any heterogeneity in the antibody product and thus increase its homogeneity. Another type of amino acid substitution is to eliminate asparagine-glycine pairs, which form potential deamidation sites, by altering one or both of the residues. In some embodiments, the C-terminal lysine of the heavy chain of the anti-Kp antibody of the invention is cleaved. In various embodiments of the invention, the heavy and light chains of the anti-Kp antibodies may optionally include a signal sequence.

The class and subclass of anti-Kp antibodies may be determined by any method known in the art. In general, the class and subclass of an antibody may be determined using antibodies that are specific for a particular class and subclass of antibody. Such antibodies are commercially available. The class and subclass can be determined by ELISA, or Western blot (immunoblot) as well as other techniques. Alternatively, the class and subclass may be determined by sequencing all or a portion of the constant domains of the heavy and/or light chains of the antibodies, comparing their amino acid sequences to the known amino acid sequences of various class and subclasses of immunoglobulins, and determining the class and subclass of the antibodies.

In some embodiments, the human monoclonal antibody of the invention is an IgG, an IgM, an IgE, an IgA, or an IgD molecule. In one embodiment, the human monoclonal antibody is an IgG and is an IgG1, IgG2, IgG3, IgG4 subclass. In still another embodiment, the human antibody subclass is IgG1.

### Binding Affinity of Anti-Kp Antibodies to Kp Surface Antigens.

In some embodiments of the invention, the anti-Kp antibodies bind to a surface antigen of a Kp according to any of the embodiments disclosed herein with high affinity. In some 15 embodiments, the anti-Kp antibodies bind with high affinity to the capsular antigen or polysaccharide of at least one strain of Kp according to any of the variants disclosed herein, preferably to capsular antigen of the K64 type. In some embodiments, the anti-Kp antibodies bind to the O-antigen of at least one strain of Kp according to any of the variants disclosed herein, in particular to the O1-antigen and/or the O2-antigen. The binding affinity and dissociation rate of an anti-Kp antibody to a Kp surface antigen according to any of the embodiments disclosed herein can be determined by methods known in the art. The binding affinity can be measured by ELISAs, RIAs, flow cytometry, surface plasmon resonance, such as BIACORE™. The dissociate rate can be measured by surface plasmon resonance. Preferably, the binding affinity and dissociation rate is measured by surface plasmon resonance. More preferably, the binding affinity and dissociation rate are measured using BIACORE™. One can determine whether an antibody has substantially the same KD as an anti-Kp antibody by using methods known in the art.

The invention provides a human anti-Kp monoclonal antibody that binds to a surface antigen of Kp according to any of the embodiments disclosed herein, in particular a surface antigen of at least one strain of Kp according to any of the variants disclosed herein, and competes or cross-competes with and/or binds the same epitope as an antibody selected from SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, and SBJ11-J13. If two antibodies reciprocally compete with each other for binding to Kp surface antigen, they are said to cross-compete.

One can determine whether an antibody binds to the same epitope or cross competes for binding with an anti-Kp antibody by using methods known in the art. In one embodiment, one allows the anti-Kp antibody of the invention to bind to Kp surface antigens under saturating conditions and then measures the ability of the test antibody to bind to Kp surface antigens. If the test antibody is able to bind to Kp surface antigens at the same time as the anti-Kp antibody, then the test antibody binds to a different epitope as the anti-Kp antibody.

However, if the test antibody is not able to bind to the Kp surface antigen at the same time, then the test antibody binds to the same epitope, an overlapping epitope, or an epitope that is in close proximity to the epitope bound by the human anti-Kp antibody, or the binding of the human anti-Kp antibody may induce a conformational change in the surface antigen that prevents or reduces binding of the test antibody. This experiment can be performed using ELISA, RIA, BIACORE™, flow cytometry or other methods known in the art.

To test whether an anti-Kp antibody cross-competes with another anti-Kp antibody, one may use the competition method described above in two directions i.e. determining if the reference antibody blocks the test antibody and vice versa. In one embodiment, the experiment is performed using ELISA. Methods of determining KD are discussed further below.

In another embodiment, the invention provides an anti-Kp antibody that destroys, inhibits, blocks, neutralizes or decreases Kp action or function, in particular is bactericidal against or kills Kp cells.

In another embodiment, the invention provides an anti-Kp antibody that is capable of competent-dependent killing of cells of Kp, in particular cells of at least one strain of Kp according to any of the variants disclosed herein.

In certain embodiments, the invention provides an anti-Kp antibody that inhibits, blocks, or decreases in severity for a day, a week, a month, 6 months, a year, or for the remainder of the subjects' life symptoms or conditions resulting from a Kp infection, in particular an infection by a MDR Kp strain, and particularly one or more NDM-producing strains according to any of the variants disclosed herein, by 25, 30, 35, 40, 45, 50, 55, 60, 65, 70, 75, 80, 85, 90, 95, or 100%. In certain embodiments, the invention provides an anti-Kp antibody that may perform any combination of the preceding embodiments.

In certain embodiments, in order to increase complement-dependent killing of Kp, mutations that enhance the hexamerization process that naturally occurs between Fc regions of antibodies after bacterial opsonization will be introduced in the Fc part of the IgG1 antibodies as previously described, preferably the E430G mutation as described in de Jong R N et al. “A Novel Platform for the Potentiation of Therapeutic Antibodies Based on Antigen-Dependent Formation of IgG Hexamers at the Cell Surface.” PLoS Biol. 2016 Jan. 6; 14(1).

All of these modifications may be carried out by means of site-directed mutagenesis, for example using the Agilent Quick-Change II Site-Directed Mutagenesis Kit, according to the manufacturer's recommendations.

In certain embodiments, the antibody comprises the variable regions of an antibody selected from SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, and SBJ11-J13 and a mutant IgG1 constant region backbone containing the E430G mutation in the Fc domain (as in de Jong R N et al. PLoS Biol. 2016 Jan. 6; 14(1)).

### Nucleic Acids, Vectors, Host Cells, and Recombinant Methods of Making Antibodies Nucleic Acids

The present invention also encompasses nucleic acid molecules encoding anti-Kp antibodies or antigen-binding portions thereof. In some embodiments, different nucleic acid molecules encode a heavy chain and a light chain of an anti-Kp immunoglobulin. In other embodiments, the same nucleic acid molecule encodes a heavy chain and a light chain of an anti-Kp immunoglobulin. In one embodiment, the nucleic acid encodes a Kp antibody, or antigen-binding portion thereof, of the invention. In some embodiments, the nucleic acid molecule comprises a nucleotide sequence that encodes a VL amino acid sequence comprising 1, 2, 3, 4, 5, 6, 7, 8, 9, or 10 conservative amino acid substitutions and/or 1, 2, or 3 non- conservative substitutions compared to germline. Substitutions may be in the CDR regions, the framework regions, or in the constant domain. In some embodiments, the nucleic acid molecule encodes a VL amino acid sequence comprising one or more variants compared to germline sequence that are identical to the variations found in the VL of one of the antibodies selected from SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, and SBJ11-J13.

In some embodiments, the nucleic acid molecule encodes at least three amino acid substitutions compared to the germline sequence found in the VL of one of the antibodies selected from SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, and SBJ11-J13.

In some embodiments, the nucleic acid molecule comprises a nucleotide sequence that encodes the VL amino acid sequence of SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, SBJ11-J13 or a variant or portion thereof. In some embodiments, the nucleic acid encodes an amino acid sequence comprising the light chain CDRs of one of said above-listed antibodies. In some embodiments, said portion is a contiguous portion comprising CDR1-CDR3. In some embodiments, the nucleic acid encodes the amino acid sequence of the light chain CDRs of said antibody. In some embodiments, said portion encodes a contiguous region from CDR1-CDR3 of the light chain of an anti-Kp antibody.

In some embodiments, the nucleic acid molecule encodes a VL amino acid sequence that is at least 70%, 75%, 80%, 85%, 90%, 95%, 97%, 98% or 99% identical to a VL amino acid sequence of a VL region of any one of antibodies SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, SBJ11-J13.

Nucleic acid molecules of the invention include nucleic acids that hybridize under highly stringent conditions, such as those described above, to a nucleotide sequence encoding the amino acid sequence of a VL region.

In another embodiment, the nucleic acid encodes a full-length light chain of an antibody selected SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, SBJ11-J13 or a light chain comprising a mutation, such as one disclosed herein.

In still another embodiment, the nucleic acid molecule encodes the variable domain of the heavy chain (VH) that comprises a human VH1, VH3 or VH4 family gene sequence or a sequence derived therefrom. In some embodiments, the nucleic acid molecule encodes one or more amino acid mutations compared to the germline sequence that are identical to amino acid mutations found in the VH of one of monoclonal antibodies SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, SBJ11-J13.

In some embodiments, the nucleic acid molecule comprises a nucleotide sequence that encodes at least a portion of the VH amino acid sequence of a monoclonal antibody selected from monoclonal antibodies SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, SBJ11-J13 all three CDR regions, a contiguous portion including CDR1-CDR3, or the entire VH region, with or without a signal sequence. In some embodiments, the nucleic acid molecule comprises a nucleotide sequence that encodes the amino acid sequence of one of SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, SBJ11-J13 or said sequence lacking the signal sequence. In some preferred embodiments, the nucleic acid molecule comprises at least a portion of the nucleotide sequence of SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, SBJ11-J13 or said sequence lacking the signal sequence. In some embodiments, said portion encodes the VH region (with or without a signal sequence), a CDR3 region, all three CDR regions, or a contiguous region including CDR1-CDR3.

In some embodiments, the nucleic acid molecule encodes a VH amino acid sequence that is at least 70%, 75%, 80%, 85%, 90%, 95%, 97%, 98% or 99% identical to the VH amino acid sequences of any one of SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, SBJ11-J13.

Nucleic acid molecules of the invention include nucleic acids that hybridize under highly stringent conditions, such as those described above, to a nucleotide sequence encoding the amino acid sequence of SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, SBJ11-J13 or that encodes a VH region thereof.

In another embodiment, the nucleic acid encodes a full-length heavy chain of an antibody selected from SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, SBJ11-J13 or a heavy chain having the amino acid sequence of SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, SBJ11-J13 with or without a signal sequence, or a heavy chain comprising a mutation, such as one of the variants discussed herein. Further, the nucleic acid may comprise the nucleotide sequence of SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, SBJ11-J13 with or without a signal sequence, or a nucleic acid molecule encoding a heavy chain comprising a mutation, such as one of the variants discussed herein.

A nucleic acid molecule encoding the heavy or light chain of an anti-Kp antibody or portions thereof can be isolated from any source that produces such antibody. In various embodiments, the nucleic acid molecules are isolated from a B cell isolated from an animal immunized with surface antigens or from an immortalized cell derived from such a B cell that expresses or encodes an anti-Kp antibody. Methods of isolating mRNA encoding an antibody are well known in the art. See, e.g., Sambrook et al. The mRNA may be used to produce cDNA for use in the polymerase chain reaction (PCR) or cDNA cloning of antibody genes. In one embodiment, the nucleic acid molecule is isolated from a hybridoma that has as one of its fusion partners a human immunoglobulin-producing cell from a non-human transgenic animal. In an even more preferred embodiment, the human immunoglobulin producing cell is isolated from a XENOMOUSE animal. In another embodiment, the human immunoglobulin-producing cell is from a non-human, non-mouse transgenic animal, as described above. In another embodiment, the nucleic acid is isolated from a non-human, non-transgenic animal. The nucleic acid molecules isolated from a non-human, non-transgenic animal may be used, e.g., for humanized antibodies. In some embodiments, a nucleic acid encoding a heavy chain of an anti-Kp antibody of the invention can comprise a nucleotide sequence encoding a VH domain of the invention joined in-frame to a nucleotide sequence encoding a heavy chain constant domain from any source. Similarly, a nucleic acid molecule encoding a light chain of an anti-Kp antibody of the invention can comprise a nucleotide sequence encoding a VL domain of the invention joined in-frame to a nucleotide sequence encoding a light chain constant domain from any source. In a further aspect of the invention, nucleic acid molecules encoding the variable domain of the heavy (VH) and/or light (VL) chains are “converted” to full-length antibody genes. In one embodiment, nucleic acid molecules encoding the VH or VL domains are converted to full-length antibody genes by insertion into an expression vector already encoding heavy chain constant (CH) or light chain constant (CL) domains, respectively, such that the VH segment is operatively linked to the CH segment(s) within the vector, and/or the VL segment is operatively linked to the CL segment within the vector. In another embodiment, nucleic acid molecules encoding the VH and/or VL domains are converted into full-length antibody genes by linking, e.g., ligating, a nucleic acid molecule encoding a VH and/or VL domains to a nucleic acid molecule encoding a CH and/or CL domain using standard molecular biological techniques.

Nucleotide sequences of human heavy and light chain immunoglobulin constant domain genes are known in the art. See, e.g., Kabat et al., Sequences of Proteins of Immunological Interest, 5th Ed., NIH Publ. No. 91-3242, 1991. Nucleic acid molecules encoding the full-length heavy and/or light chains may then be expressed from a cell into which they have been introduced and the anti-Kp antibody isolated.

The nucleic acid molecules may be used to recombinantly express large quantities of anti-Kp antibodies. The nucleic acid molecules also may be used to produce chimeric antibodies, bispecific antibodies, single chain antibodies, immunoadhesins, diabodies, mutated antibodies and antibody derivatives, as described further below. If the nucleic acid molecules are derived from a non-human, non-transgenic animal, the nucleic acid molecules may be used for antibody humanization, also as described below.

In another embodiment, a nucleic acid molecule of the invention is used as a probe or PCR primer for a specific antibody sequence. For instance, the nucleic acid can be used as a probe in diagnostic methods or as a PCR primer to amplify regions of DNA that could be used, inter alia, to isolate additional nucleic acid molecules encoding variable domains of anti-Kp antibodies. In some embodiments, the nucleic acid molecules are oligonucleotides. In some embodiments, the oligonucleotides are from highly variable domains of the heavy and light chains of the antibody of interest. In some embodiments, the oligonucleotides encode all or a part of one or more of the CDRs of antibodies SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, SBJ11-J13 or variants thereof as described herein. The nucleic acid molecules herein disclosed may be DNA or RNA molecules.

### Vectors

The invention provides vectors comprising nucleic acid molecules that encode the heavy chain of an anti-Kp antibody of the invention or an antigen-binding portion thereof. The invention also provides vectors comprising nucleic acid molecules that encode the light chain of such antibodies or antigen-binding portion thereof. The invention further provides vectors comprising nucleic acid molecules encoding fusion proteins, modified antibodies, antibody fragments, and probes thereof. In some embodiments, the anti-Kp antibodies or antigen-binding portions of the invention are expressed by inserting DNAs encoding partial or full-length light and heavy chains, obtained as described above, into expression vectors such that the genes are operatively linked to necessary expression control sequences such as transcriptional and translational control sequences. Expression vectors include plasmids, retroviruses, adenoviruses, adeno-associated viruses (AAV), plant viruses such as cauliflower mosaic virus, tobacco mosaic virus, cosmids, YACs, EBV derived episomes, and the like. The antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vectors. In one embodiment, both genes are inserted into the same expression vector. The antibody genes are inserted into the expression vector by standard methods (e.g., ligation of complementary restriction sites on the antibody gene fragment and vector, or blunt end ligation if no restriction sites are present). A convenient vector is one that encodes a functionally complete human CH or CL immunoglobulin sequence, with appropriate restriction sites engineered so that any VH or VL sequence can easily be inserted and expressed, as described above. In such vectors, splicing usually occurs between the splice donor site in the inserted J region and the splice acceptor site preceding the human C domain, and also at the splice regions that occur within the human CH exons. Polyadenylation and transcription termination occur at native chromosomal sites downstream of the coding regions. The recombinant expression vector also can encode a signal peptide that facilitates secretion of the antibody chain from a host cell. The antibody chain gene may be cloned into the vector such that the signal peptide is linked in-frame to the amino terminus of the immunoglobulin chain. The signal peptide can be an immunoglobulin signal peptide or a heterologous signal peptide (i.e. a signal peptide from a non-immunoglobulin protein). In addition to the antibody chain genes, the recombinant expression vectors of the invention carry regulatory sequences that control the expression of the antibody chain genes in a host cell. It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g. the adenovirus major late promoter (AdMLP)), polyoma and strong mammalian promoters such as native immunoglobulin and actin promoters. For further description of viral regulatory elements, and sequences thereof, see e.g., U.S. Pat. Nos. 5,168,062, 4,510,245 and 4,968,615. Methods for expressing antibodies in plants, including a description of promoters and vectors, as well as transformation of plants is known in the art. See, e.g., U.S. Pat. No. 6,517,529, incorporated herein by reference. Methods of expressing polypeptides in bacterial cells or fungal cells, e.g., yeast cells, are also well known in the art. In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, incorporated herein by reference). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr- host cells with methotrexate selection/amplification), the neo gene (for G418 selection), and the glutamate synthetase gene.

In some embodiments said vector is a selected from RNA virus vectors, DNA virus vectors, plasmid viral vectors, adenovirus vectors, adenovirus associated virus vectors, herpes virus vectors and retrovirus vectors.

In some aspects, the invention provides compositions (e.g., pharmaceutical compositions), methods, kits and reagents, comprising one or more isolated nucleic acid molecules or vectors according to any one of the embodiments herein disclosed for use in the prevention and/or treatment of a Kp infections, in particular in humans and other mammals. In some embodiments such nucleic acid molecules and vectors are formulated in a nanoparticle, for example in lipid nanoparticle, cationic lipid nanoparticle, examples of such formulations can be found in US2020197510 herein incorporated by reference.

### Non-Hybridoma Host Cells and Methods of Recombinantly Producing Protein

Nucleic acid molecules encoding anti-Kp antibodies and vectors comprising these nucleic acid molecules can be used for transfection or transformation of a suitable mammalian, plant, bacterial or yeast host cell. Transfection or transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors. Methods of transforming cells are well known in the art (see, e.g., U.S. Pat. Nos. 4,399,216, 4,912,040, 4,740,461, and 4,959,455, incorporated herein by reference). Methods for transforming plant cells are well known in the art, including, e.g., Agrobacterium-mediated transformation, biolistic transformation, direct injection, electroporation and viral transformation. Methods for transforming bacterial and yeast cells are also well known in the art. Mammalian cell lines available as hosts for expression are well known in the art and include many immortalized cell lines available from the American Type Culture Collection (ATCC). These include, inter alia, Chinese hamster ovary (CHO) cells, N50 cells, SP2 cells, HEK-293T cells, NIH-3T3 cells, HeLa cells, baby hamster kidney (BHK) cells, African green monkey kidney cells (COS), human hepatocellular carcinoma cells (e.g., Hep G2), A549 cells, and a number of other cell lines. Cell lines of particular preference are selected through determining which cell lines have high expression levels. Other cell lines that may be used are insect cell lines, such as Sf9 or Sf21 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods. Plant host cells include, e.g., Nicotiana, Arabidopsis, duckweed, corn, wheat, potato, etc. Bacterial host cells include E. coli and Streptomyces species. Yeast host cells include Schizosaccharomyces pombe, Saccharomyces cerevisiae and Pichia pastoris. Further, expression of antibodies of the invention from production cell lines can be enhanced using a number of known techniques. For example, the glutamine synthetase gene expression system (the GS system) is a common approach for enhancing expression under certain conditions. The GS system is discussed in whole or part in connection with European Patent Nos. O216 846, 0 256 055, 0 323 997 and O338 841. It is likely that antibodies expressed by different cell lines or in transgenic animals will have different glycosylation from each other. However, all antibodies encoded by the nucleic acid molecules provided herein, or comprising the amino acid sequences provided herein are part of the instant invention, regardless of the glycosylation of the antibodies.

### Transgenic Animals and Plants

Anti-Kp antibodies of the invention also can be produced transgenically through the generation of a mammal or plant that is transgenic for the immunoglobulin heavy and light chain sequences of interest and production of the antibody in a recoverable form therefrom. In connection with the transgenic production in mammals, anti-Kp antibodies can be produced in, and recovered from, the milk of goats, cows, or other mammals. See, e.g., U.S. Pat. Nos. 5,827,690, 5,756,687, 5,750,172, and 5,741,957, incorporated herein by reference. In some embodiments, non- human transgenic animals that comprise human immunoglobulin loci are immunized with Kp whole cells or an immunogenic portion thereof, such as one or more surface antigens as described above. Methods for making antibodies in plants are described, e.g., in U.S. Pat. Nos. 6,046,037 and 5,959,177, incorporated herein by reference.

In some embodiments, non-human transgenic animals or plants are produced by introducing one or more nucleic acid molecules encoding an anti-Kp antibody of the invention into the animal or plant by standard transgenic techniques. See Hogan and U.S. Pat. No. 6,417,429, supra. The transgenic cells used for making the transgenic animal can be embryonic stem cells or somatic cells or a fertilized egg. The transgenic non-human organisms can be chimeric, nonchimeric heterozygotes, and nonchimeric homozygotes. See, e.g., Hofian et al. Manipulating the Mouse Embryo: A Laboratory Manual second ed., Cold Spring Harbor Press (1999); Jackson et al, Mouse Genetics and Transgenics: A Practical Approach, Oxford University Press (2000); and Pinkert, Transgenic Animal Technology: A Laboratory Handbook, Academic Press (1999), all incorporated herein by reference. In some embodiments, the transgenic non-human animals have a targeted disruption and replacement by a targeting construct that encodes a heavy chain and/or a light chain of interest. In one embodiment, the transgenic animals comprise and express nucleic acid molecules encoding heavy and light chains that specifically bind to Kp, and preferably to (i) the capsular polysaccharide or (ii) the O-antigen of a Kp strain according to any of the embodiments disclosed herein; or (iii) both (i) and (ii). In one embodiment, the transgenic animals comprise and express nucleic acid molecules encoding heavy and light chains that specifically bind to a surface antigen of at least one NDM producing Kp strain according to any of the variants disclosed herein, preferably to at least two, at least three, at least four or at least five strains. In some embodiments, the transgenic animals comprise nucleic acid molecules encoding a modified antibody such as a single-chain antibody, a chimeric antibody or a humanized antibody. The anti-Kp antibodies may be made in any transgenic animal. In one embodiment, the non-human animals are mice, rats, sheep, pigs, goats, cattle or horses. The non-human transgenic animal expresses said encoded polypeptides in blood, milk, urine, saliva, tears, mucus and other bodily fluids.

### Class Switching

Another aspect of the invention provides a method for converting the class or subclass of an anti-Kp antibody to another class or subclass. In some embodiments, a nucleic acid molecule encoding a VL or VH that does not include sequences encoding CL or CH is isolated using methods well-known in the art. The nucleic acid molecule then is operatively linked to a nucleotide sequence encoding a CL or CH from a desired immunoglobulin class or subclass. This can be achieved using a vector or nucleic acid molecule that comprises a CL or CH chain, as described above. For example, an anti- Kp antibody that was originally IgM can be class switched to an IgG. Further, the class switching may be used to convert one IgG subclass to another, e.g., from IgG1 to IgG2. Another method for producing an antibody of the invention comprising a desired isotype comprises the steps of isolating a nucleic acid encoding a heavy chain of an anti-Kp antibody and a nucleic acid encoding a light chain of an anti- Kp antibody, isolating the sequence encoding the VH region, ligating the VH sequence to a sequence encoding a heavy chain constant domain of the desired isotype, expressing the light chain gene and the heavy chain construct in a cell, and collecting the anti-Kp antibody with the desired isotype.

### Modified Antibodies

According to one embodiment, the invention provides an immunospecific polypeptide comprising at least one variable domain, in particular at least a heavy chain variable domain (VH) and a light chain variable domain (VL) of a human monoclonal antibody as defined in any one of the embodiments disclosed herein, or the CDRs as defined in any one of the embodiments disclosed herein wherein said immunospecific polypeptide is for example a multispecific antibody, a bispecific antibody, a three-specific antibody, a diabody, a triabody, a tetrabody, a minibody, a linear antibody, a monoclonal antibody, a chelating recombinant antibody, a tribody, a bibody, an intrabody, a nanobody, a binding-domain immunoglobulin fusion protein, a fusion antibody, an immunoadhesin or a an antigen binding portion thereof.

In one particular embodiment, said antibody or antigen binding fragment is an Fab, an F(ab′)2, an Fv or a single-chain antibody fragment.

In certain embodiments the antibodies or antigen binding fragments thereof of any of the embodiments disclosed herein are bispecific, three-specific or multispecific. Bi-, three- or multi-specific antibodies of the invention may be formed using methods well known in the art, e.g., chemical conjugation of one or more antibodies or antigen binding variable domains disclosed herein to each other and/or to differing epitope binding polypeptides, wherein the binding domains of the bi-, three- or multi-specific molecule exhibit affinity for at least two different antigens. For example, an antibody according to the present invention may comprise a first and a second VL domain, or a first and second VH domain, wherein said first and second domain have differing binding specificities (i.e., bind to different antigens). According to one preferred embodiment, an antibody or antigen binding fragment thereof according to the present invention comprises a first and a second VL domain, or a first and second VH domain according to any of the embodiments disclosed in the present specification and in the claims, wherein said first and second domain have differing binding specificities for Kp (i.e., bind to different antigens).

According to one object of the invention, the immunospecific polypeptides of the invention comprise one VL, or one VH domain according to any of the embodiments disclosed in the present specification and in the claims, and one antigen binding polypeptide, wherein the VL domain, or VH domain, and said polypeptide exhibit differing binding specificities. In certain embodiments of the invention, at least one antigen binding domain of the bi-, three-or multispecific antibody or antigen binding fragment of the invention immunospecifically binds to Kp. In other embodiments of the invention, at least one antigen binding domain of the bi-, three- or multispecific molecule of the invention immunospecifically binds to a surface antigen of Kp.

In another embodiment, the invention provides a fusion antibody or immunoadhesin made that comprises all or a portion of at least an anti-Kp antibody of the invention according to any of the embodiments disclosed herein, linked to another polypeptide. In one embodiment, only the variable domains of at least one, at least two or at least three anti-Kp antibodies according to any of the embodiments disclosed herein are linked to the polypeptide. In still another embodiment, the VH domain of an anti-Kp antibody is linked to a first polypeptide, while the VL domain of an anti-Kp antibody is linked to a second polypeptide that associates with the first polypeptide in a manner such that the VH and VL domains can interact with one another to form an antigen binding site. In still another embodiment, the VH domain is separated from the VL domain by a linker such that the VH and VL domains can interact with one another (see below under Single Chain Antibodies). The VH-linker-VL antibody is then linked to the polypeptide of interest. The fusion antibody is useful for directing a polypeptide to a Kp surface antigen-expressing cell or tissue. The polypeptide may be a therapeutic agent, such as a toxin, chemokine or other regulatory protein, or may be a diagnostic agent, such as an enzyme that may be easily visualized, such as horseradish peroxidase. In addition, fusion antibodies can be created in which two (or more) single-chain antibodies are linked to one another.

This is useful if one wants to create a divalent or polyvalent antibody on a single polypeptide chain, or if one wants to create a bispecific antibody or nanobody.

According to one preferred embodiment, said antibody or antigen binding fragment is a single chain antibody (scFv). To create a single chain antibody, (scFv) the VH- and VL-encoding DNA fragments according to any of the embodiments disclosed herein are operatively linked to another fragment encoding a flexible linker, e.g., encoding the amino acid sequence (GIy4-Ser)3, such that the VH and VL sequences can be expressed as a contiguous single-chain protein, with the VL and VH domains joined by the flexible linker. See, e.g., Bird et al, Science 242:423-426 (1988); Huston et al, Proc. Natl. Acad. ScL USA 85:5879-5883 (1988); McCafferty et al., Nature 348:552-554 (1990).

The single chain antibody may be monovalent, if only a single VH and VL are used, bivalent, if two VH and VL are used, or polyvalent, if more than two VH and VL are used.

Bispecific or polyvalent antibodies may be generated that bind specifically to a Kp surface antigen and to another molecule. Bispecific antibodies or antigen-binding fragments can be produced by a variety of methods including fusion of hybridomas or linking of Fab′ fragments. See, e.g., Songsivilai & Lachmann, Clin. Exp. Immunol. 79: 315-321 (1990), Kostelny et al, J. Immunol. 148:1547-1553 (1992).

In addition, bispecific antibodies may be formed as “diabodies” or “Janusins”. In some embodiments, the bispecific antibody binds to two different epitopes of a Kp surface antigen according to any of the variants disclosed herein. In some embodiments, the bispecific antibody has a first heavy chain and a first light chain from monoclonal antibody SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, SBJ11-J13 and an additional antibody heavy chain and light chain. In some embodiments, the additional light chain and heavy chain also are from one of the above-identified monoclonal antibodies, but are different from the first heavy and light chains. In some embodiments, the modified antibodies described above are prepared using one or more of the variable domains or CDR regions from a human anti-Kp monoclonal antibody provided herein. It forms part of the present invention also the use of one or more antigen binding regions or variable domains of a human monoclonal antibody according to any of the embodiments disclosed in the present specification and in the claims for the production of bi-, three- or multispecific antibodies or nanobodies according to any of the variants herein disclosed.

The immunospecific polypeptides such as multispecific antibodies, diabodies, triabodies, tetrabodies, minibodies, linear antibodies, chelating recombinant antibodies, tribodies, bibodies, monoclonal antibody, intrabodies, nanobodies, binding-domain immunoglobulin fusion proteins, fusion antibodies, immunoadhesins or and antigen binding fragment thereof according to any of the embodiments disclosed in the present specification and in the claims can be used in a prophylactic or therapeutic treatment of a Klebsiella pneumoniae infection or conditions or disorders resulting from such infection, in particular for use in the prevention and/or treatment of an infection of a drug resistant or multi-drug resistant strain of Klebsiella pneumoniae, in particular according to any of the embodiments herein disclosed.

The invention hence provides also pharmaceutical compositions comprising an immunospecific polypeptides such as one or more multispecific antibodies, diabodies, triabodies, tetrabodies, minibodies, linear antibodies, monoclonal antibodies, chelating recombinant antibodies, tribodies, bibodies, intrabodies, nanobodies, binding-domain immunoglobulin fusion proteins, fusion antibodies, immunoadhesins or and antigen binding fragment thereof according to any of the embodiments disclosed in the present specification and in the claims, and at least one pharmaceutically acceptable carrier. Said compositions are useful in the prevention and/or treatment of a Kp infection according to any of the embodiments disclosed herein, preferably an infection of a Klebsiellapneumoniae capsular type K64 strain according to any of the variants disclosed herein.

The multispecific antibodies, diabodies, triabodies, tetrabodies, minibodies, linear antibodies, chelating recombinant antibodies, tribodies, bibodies, intrabodies, nanobodies, binding-domain immunoglobulin fusion proteins, fusion antibodies, immunoadhesins or and antigen binding fragment thereof according to any of the embodiments disclosed in the present specification and in the claims can also be used in an in vitro method for the diagnosis of Kp or else for the design of a vaccine against Kp according to any of the variants disclosed in the present specification and in the claims, preferably against a Klebsiellapneumoniae capsular type K64 strain according to any of the variants disclosed herein.

### Derivatized and Labelled Antibodies

An anti-Kp antibody or antigen-binding portion of the invention can be derivatized or linked to another molecule (e.g., another peptide or protein). In general, the antibodies or portion thereof are derivatized such that the binding with a Kp surface antigen according to any of the embodiments disclosed herein is not affected adversely by the derivatization or labelling. Accordingly, the antibodies and antibody portions of the invention are intended to include both intact and modified forms of the human anti-Kp antibodies described herein. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detection agent, a cytotoxic agent, a pharmaceutical agent, and/or a protein or peptide that can mediate association of the antibody or antibody portion with another molecule (such as a streptavidin core region or a polyhistidine tag). One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N- hydroxysuccinimide ester) or homobifunctional {e.g., disuccinimidyl suberate). Such linkers are available from Pierce Chemical Company, Rockford, II. [O179]. Another type of derivatized antibody is a labelled antibody. Useful detection agents with which an antibody or antigen-binding portion of the invention may be derivatized include fluorescent compounds, including fluorescein, fluorescein isothiocyanate, rhodamine, phycoerythrin, 5-dimethylamine-1-napthalenesulfonyl chloride, lanthanide phosphors and the like. An antibody can also be labelled with enzymes that are useful for detection, such as horseradish peroxidase, [beta]-galactosidase, luciferase, alkaline phosphatase, glucose oxidase and the like. When an antibody is labelled with a detectable enzyme, it is detected by adding additional reagents that the enzyme uses to produce a reaction product that can be discerned. For example, when the agent horseradish peroxidase is present, the addition of hydrogen peroxide and diaminobenzidine leads to a coloured reaction product, which is detectable. An antibody can also be labelled with biotin, and detected through indirect measurement of avidin or streptavidin binding. An antibody can also be labelled with a predetermined polypeptide epitope recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. An anti-Kp antibody can also be labelled with a radiolabelled amino acid. The radiolabel can be used for both diagnostic and therapeutic purposes. Further, the radiolabel can be used therapeutically as a toxin for cancerous cells or tumours. In some embodiments, the anti-Kp antibody can be labelled with a paramagnetic, radioactive or florigenic ion that is detectable upon imaging. In some embodiments, the paramagnetic ion is chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III). In other embodiments, the radioactive ion is iodine 123, technetium 99, indium 111, rhenium 188, rhenium 186, copper 67, iodine 131, yttrium90, iodine 125, astatine 211, and gallium 67. In other embodiments, the anti-Kp antibody is labelled with an X-ray imaging agent such as lanthanum (III), gold (III) lead (II) and bismuth (III).

### Compositions and Kits

The invention relates to compositions comprising the human anti-Kp antibody of the invention and one or more pharmaceutical acceptable excipients and/or carriers.

In certain embodiments, the composition may comprise one or more antibodies or a binding portion thereof of any of the preceding embodiments.

In one preferred embodiment, the composition comprises one or more antibodies selected from SBJ08-009, SBJ08-D18, SBJ03-F18, SBJ08-H10, SBJ08-N23, SBJ08-K19, SBJ08-M13, SBJ03-L02, SBJ08-J10, SBJ10-H18, SBJ08-003, SBJ05-K07, SBJ09-I10, and SBJ11-C06, preferably comprises all these antibodies.

In another preferred embodiment, the composition comprises one or more antibodies selected from SBJ05-D08, SBJ05-C11, SBJ05-D14, SBJ11-J13, SBJ05-M13, SBJ09-G14, SBJ05-N02, SBJ09-M06, SBJ08-F04, and SBJ05-B17, preferably comprises all these antibodies.

In another preferred embodiment, the composition comprises one or more antibodies selected from SBJ05-D08, SBJ05-C11, SBJ05-D14, SBJ11-J13, and SBJ05-M13, preferably comprises all these antibodies.

In another preferred embodiment, the composition comprises one or more antibodies selected from SBJ09-G14, SBJ05-N02, SBJ09-M06, SBJ08-F04, and SBJ05-B17, preferably comprises all these antibodies.

In one embodiment, the compositions can further comprise one or more multispecific antibodies, diabodies, triabodies, tetrabodies, minibodies, linear antibodies, chelating recombinant antibodies, tribodies, bibodies, intrabodies, nanobodies, binding-domain immunoglobulin fusion proteins, fusion antibodies, immunoadhesins or and antigen binding fragment thereof according to any of the embodiments disclosed in the present specification and in the claims.

The compositions according to any of the embodiments disclosed herein are particularly for use in the prevention and/or treatment of a Kp infections. In some embodiments, the subject of treatment is a human. In other embodiments, the subject is a veterinary subject. In some embodiments, an antagonist anti-Kp antibody that binds to capsular polysaccharide and one that binds to the O-antigen or antigen-binding portions of either or both, are both administered to a subject, either together or separately. In certain embodiments the antibodies are in a composition comprising a pharmaceutically acceptable carrier. In another embodiment, one or more of the antagonist Kp antibodies of the invention are administered in combination with one or more additional antagonistic antibodies that bind different surface antigens of Kp or different epitopes on the surface antigen of Kp and/or that bind the surface antigen from different isolates of Kp.

As used herein, “pharmaceutically acceptable carrier” means any and all solvents, dispersion media, coatings, antibacterial and antifungal agents, isotonic and absorption delaying agents, and the like that are physiologically compatible. Some examples of pharmaceutically acceptable carriers are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

The compositions of this invention may be in a variety of forms, for example, liquid, semi-solid and solid dosage forms, such as liquid solutions (e.g., injectable and infusible solutions), dispersions or suspensions, tablets, pills, powders, liposomes and suppositories.

The preferred form depends on the intended mode of administration and therapeutic application. Typical preferred compositions are in the form of injectable or infusible solutions, such as compositions similar to those used for passive immunization of humans.

The preferred mode of administration is parenteral (e.g., intravenous, subcutaneous, intraperitoneal, intramuscular). In one embodiment, the antibody is administered by intravenous infusion or injection. In still another embodiment, the antibody is administered by intramuscular or subcutaneous injection. Therapeutic compositions are typically sterile and stable under the conditions of manufacture and storage. The composition can be formulated as a solution, microemulsion, dispersion, liposome, or other ordered structure suitable to high drug concentration. Sterile injectable solutions can be prepared by incorporating the anti-Kp antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yield a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin. The antibodies of the present invention can be administered by a variety of methods known in the art, although for many therapeutic applications, the preferred route/mode of administration is subcutaneous, intramuscular, or intravenous infusion. As will be appreciated by the skilled artisan, the route and/or mode of administration will vary depending upon the desired results. Other modes of administration include intraperitoneal, intrabronchial, transmucosal, intraspinal, intrasynovial, intraaortic, intranasal, ocular, otic, topical and buccal. In certain embodiments, the active compound of the antibody compositions may be prepared with a carrier that will protect the antibody against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems (J. R. Robinson, ed., Marcel Dekker, Inc., New York, 1978). The invention also provides compositions suitable for administration by inhalation, which comprise the anti-Kp antibodies described herein. The anti-Kp antibodies may be conveniently delivered to a subject in the form of an aerosol spray presentation from pressurized packs or from a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch. Dellamary et al. (2004) J Control Release.;95(3): 489-500 describes formulations for the pulmonary delivery of antibodies. The invention also provides compositions, suitable for administration through the oral mucosa, which comprise the anti-Kp antibody described herein. Oral transmucosal delivery refers to the delivery of a delivery vehicle across a mucous membrane in the oral cavity, pharyngeal cavity, or esophagus, and may be contrasted, for example, with traditional oral delivery, in which absorption of a drug occurs in the intestine. Accordingly, routes of administration in which the anti-Kp antibodies are absorbed through the buccal, sublingual, gingival, pharyngeal, and/or esophageal mucosa are all encompassed within “oral transmucosal delivery,” as that term is used herein. For administration through the transmucosal mucosa, the anti-Kp antibody may be formulated, for example, into chewing gums (see U.S. Pat. No. 5,711,961) or buccal patches (see e.g. U.S. Pat. No. 5,298,256). The invention also provides compositions suitable for administration through the vaginal mucosa, which comprise the anti-Kp antibodies described herein. The anti-Kp antibodies of the invention may be formulated into a vaginal suppository, foam, cream, tablet, capsule, ointment, or gel. In certain embodiments, the compositions comprising the anti-Kp antibodies are formulated with permeants appropriate to the transmucosal barrier to be permeated. Such penetrants are generally known in the art, and include, for example, for trans mucosal administration bile salts and fusidic acid derivatives. In certain embodiments, an anti-Kp antibody of the invention can be orally administered, for example, with an inert diluent or an assailable edible carrier. The compound (and other ingredients, if desired) can also be enclosed in a hard- or soft-shell gelatine capsule, compressed into tablets, or incorporated directly into the subject's diet. For oral therapeutic administration, the anti-Kp antibodies can be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation. Additional active compounds also can be incorporated into the compositions. In certain embodiments, an inhibitory anti-Kp antibody of the invention is co-formulated with and/or co-administered with one or more additional therapeutic agents, particularly anti-microbial agents, more particularly one or more β-lactamase inhibitors.

These therapeutic agents include, without limitation, antibodies that bind other targets, photosensitizers, androgen, oestrogen, nonsteroidal anti-inflammatory agents, antihypertensive agents, analgesic agents, antidepressants, antibiotics, anticancer agents, anaesthetics, antiemetics, anti-infectants, contraceptives, antidiabetic agents, steroids, anti-allergy agents, chemotherapeutic agents, anti-migraine agents, agents for smoking cessation, anti-viral agents, immunosuppressants, thrombolytic agent, cholesterol-lowering agents and anti-obesity agents. Therapeutic agents also include peptide analogues that inhibit or kill Kp cells, and agents that inhibit Kp function or activity. In one embodiment, the additional agents that inhibit are well-known in the art.

Such combination therapies may require lower dosages of the inhibitory anti-Kp antibody as well as the co-administered agents, thus avoiding possible toxicities or complications associated with the various monotherapies. In certain specific embodiments, the therapeutic agent(s) that is co-formulated with and/or co-administered with an inhibitory anti-Kp antibody of the invention is an antimicrobial agent. Antimicrobial agents include antibiotics (e.g. antibacterial), antiviral agents, antifungal agents, and anti-protozoan agents. Non-limiting examples of antimicrobial agents are sulfonamides, trimethoprim-sulfamethoxazole, quinolones, penicillins, and cephalosporins.

The compositions of the invention may include a “therapeutically effective amount” or a “prophylactically effective amount” of an antibody or antigen-binding portion of the invention. A “therapeutically effective amount” refers to an amount effective, at dosages and for periods of time necessary, to achieve the desired therapeutic result. A therapeutically effective amount of the antibody or antibody portion may vary according to factors such as the disease state, age, sex, and weight of the individual, and the ability of the antibody or antibody portion to elicit a desired response in the individual. A therapeutically effective amount is also one in which any toxic or detrimental effects of the antibody or antibody portion are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

Dosage regimens can be adjusted to provide the optimum desired response (e.g., a therapeutic or prophylactic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the anti-Kp antibody or portion thereof and the particular therapeutic or prophylactic effect to be achieved, and (b) the limitations inherent in the art of compounding such an antibody for the treatment of sensitivity in individuals.

In one preferred embodiment, the pharmaceutical composition comprising equal or less than 400 mg for dosage unit of the human monoclonal antibody or antigen-binding portion thereof, more preferably less than 400, 350, 300, 250, 200, 150, 100, 50, 25, 10 mg for dosage unit.

The pharmaceutical composition comprising such dosage unit is preferably for parental administration, for example for intravenous, subcutaneous, intraperitoneal or intramuscular administration. In one preferred embodiment, the pharmaceutical composition is in the liquid form in a concentration between 20 and 200 mg/ml, more preferably between 40 and 80 mg/ml.

An exemplary, non-limiting range for a therapeutically or prophylactically-effective amount of an antibody or antibody portion of the invention is 0.025 to 50 mg/kg, more preferably 0.1 to 5 mg/kg, more preferably 0.1-5, 0.1 to 4 or 0.25 to 3 mg/kg.

In one embodiment, the invention provides human monoclonal antibody or an antigen-binding portion according to any one of the embodiments herein disclosed, for use in a method for prophylactic or therapeutic treatment of Kp infection or conditions or disorders resulting from such infection, in particular infection from at least one multi-drug resistant strain of Kp, preferably an infection of a Klebsiella pneumoniae capsular type K64 strain according to any of the variants disclosed herein, more preferably from at least an NDM-producing strain of Kp according to any of the variants disclosed in the present specification, wherein said method comprising the step of administering to a patient a therapeutically effective amount of said antibody or antigen-binding portion thereof, preferably between 0.025 to 50 mg/kg, more preferably 0.1 to 5 mg/kg, more preferably 0.1-5, 0.1 to 4 or 0.25 to 3 mg/kg once a day, for example for at least one, two, three, four, five, six, seven, eight, nine, ten, eleven days. Such patient is a mammal, preferably a human.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Another aspect of the present invention provides kits comprising an anti-Kp, or antigen-binding portion, of the invention or a composition comprising such an antibody or antigen-binding fragment. A kit may include, in addition to the antibody or composition, diagnostic or therapeutic agents. A kit can also include instructions for use in a diagnostic or therapeutic method, as well as packaging material such as, but not limited to, ice, dry ice, styrofoam, foam, plastic, cellophane, shrink wrap, bubble wrap, cardboard and starch peanuts. In one embodiment, the kit includes the antibody or a composition comprising it and a diagnostic agent that can be used in a method described below. In still another embodiment, the kit includes the antibody or a composition comprising it and one or more therapeutic agents that can be used in a method described below.

In one embodiment, the antibodies or binding portion thereof or composition comprising such antibodies according to any one of the embodiments herein disclosed are for use in the prevention or the treatment of patients infected with Kp, in particular infected with at least one strain of a drug resistant or multi-drug resistant strain of KP, preferably infected with a Klebsiella pneumoniae capsular type K64 strain according to any of the variants disclosed herein, for example infected with an NDM-producing strain of Kp, in particular an NDM-1 positive strain of Kp or any other strain disclosed in the present specification and in the claims.

The use of such antibodies and compositions of Kp-specific mAbs include, but are not limited to passive immunization in persons at risk of contracting the infection (e.g. professionally exposed personnel, people living in endemic areas) and therapy of acute cases, either hospitalized or not. The invention also relates to compositions for inhibiting Kp infection, and in particular infection from at least one drug resistant or multi-drug resistant strain of Kp, more in particular infection from an NDM-1 producing strain of Kp such as the ST147_{NDM-1} strain, in a mammal comprising an amount of an antibody of the invention optionally in combination with an amount of beta-lactamase inhibitor, wherein the amounts of the anti-Kp antibody and of the inhibitor are together effective in bacterial killing.

### Diagnostic Methods of Use

The antibodies according to the invention may use also as diagnostic tools for rapid detection of Kp infection, in particular infection from at least one drug resistant or multi-drug resistant strain of Kp, preferably an infection from a Klebsiellapneumoniae capsular type K64 strain according to any of the variants disclosed herein, more in particular infection from an NDM producing strain of Kp, or any other strain of Kp according to any of the variants disclosed in the present specification. In another aspect, the invention provides diagnostic methods. The anti-Kp antibodies can be used to detect Kp in a biological sample in vitro or in vivo. In one embodiment, the invention provides a method for diagnosing the presence or location of Kp cells in a subject in need thereof. The anti-Kp antibodies can be used in a conventional immunoassay, including, without limitation, an ELISA, an RIA, flow cytometry, tissue immunohistochemistry, Western blot (immunoblot) or immunoprecipitation. The anti-Kp antibodies of the invention can be used to detect Kp from humans. The invention provides a method for detecting Kp cells in a biological sample comprising contacting the biological sample with an anti-Kp antibody of the invention and detecting the bound antibody. In one embodiment, the anti-Kp antibody is directly labelled with a detectable label. In another embodiment, the anti-Kp antibody (the first antibody) is unlabelled and a second antibody or other molecule that can bind the anti-Kp antibody is labelled. As is well known to one of skill in the art, a second antibody is chosen that is able to specifically bind the particular species and class of the first antibody. For example, if the anti-Kp antibody is a human IgG, then the secondary antibody could be an anti-human-IgG. Other molecules that can bind to antibodies include, without limitation, Protein A and Protein G, both of which are available commercially, e.g., from Pierce Chemical Co. Example of biological samples to use in the diagnostic methods herein disclosed are urine, stool, blood, saliva, biopsies, cerebrospinal fluid, nasopharyngeal and oropharyngeal wash, sputum, endotracheal aspirate, bronchoalveolar lavage or other biological samples obtainable from a human subject.

Suitable labels for the antibody or secondary antibody have been disclosed supra, and include various enzymes, prosthetic groups, fluorescent materials, luminescent materials and radioactive materials. Examples of suitable enzymes include horseradish peroxidase, alkaline phosphatase, [beta]-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol. In other embodiments, Kp cells can be assayed in a biological sample by a competition immunoassay utilizing Kp or Kp surface antigens standards labelled with a detectable substance and an unlabelled anti-Kp antibody.

In this assay, the biological sample, the labelled Kp or Kp surface antigen standards and the anti-Kp antibody are combined and the amount of labelled Kp or Kp antigen standard bound to the unlabelled antibody is determined. The amount of Kp cells or Kp surface antigen in the biological sample is inversely proportional to the amount of labelled Kp or Kp antigen standard bound to the anti-Kp antibody. One can use the immunoassays disclosed above for a number of purposes. For example, the anti-Kp antibodies can be used to detect Kp cells in cultured cells samples or as a diagnostic assay in samples from a subject. The diagnostic methods according to any embodiments herein disclosed may be followed by a further step of the administration in the positive subject of an anti-Kp drugs, for example according to any of the Therapeutic Methods herein disclosed.

### Therapeutic Methods of Use

In another embodiment, the invention provides a method for neutralizing or inhibiting or killing Kp cells by administering one or more anti-Kp antibodies according to any of the embodiments disclosed herein to a patient in need thereof. Any of the types of antibodies described herein may be used therapeutically. In various embodiments, the anti-Kp antibody is a human antibody. In some embodiments, the antibody, or antigen-binding portion thereof, binds to the capsular antigen or polysaccharide and/or O-antigen of at least one Kp strain according to any of the variants disclosed in the present specification. In some embodiments, the patient is a human patient. Alternatively, the patient may be a mammal infected with Kp. In one embodiment, the invention provides methods of treating, aiding in the treatment, preventing or aiding in the prevention of, Kp infection and conditions or disorders resulting from such infection, in particular infection from at least one drug resistant or multi-drug resistant strain of Kp, preferably an infection of a Klebsiellapneumoniae capsular type K64 strain according to any of the variants disclosed herein, more in particular infection from an NDM producing strain of Kp, or any other strain of Kp according to any of the variants disclosed in the present specification, in a subject by administering to the subject a therapeutically-effective or prophylactically effective amount of one or more anti-Kp antibody of the invention. Antibodies and antigen-binding fragments thereof which are antagonists of Kp or a surface antigen of Kp can be used as therapeutics for Kp infection.

The antibody may be administered locally or systemically. The therapeutic compositions comprising anti-Kp antibodies may be administered to the subject, for example, orally, nasally, vaginally, buccally, rectally, via the eye, or via the pulmonary route, in a variety of pharmaceutically acceptable dosing forms, which will be familiar to those skilled in the art.

For example, before administration, a formulation containing the anti- Kp antibodies may be conveniently contained in a two-compartment unit dose container, one compartment containing a freeze-dried anti-Kp antibody preparation and the other compartment containing normal saline. The serum concentration of the antibody may be measured by any method known in the art.

In some aspects, the method according to any of the embodiments disclosed herein comprises administering one or more antibodies selected from SBJ08-009, SBJ08-D18, SBJ03-F18, SBJ08-H10, SBJ08-N23, SBJ08-K19, SBJ08-M13, SBJ03-L02, SBJ08-J10, SBJ10-H18, SBJ08-003, SBJ05-K07, SBJ09-I10, and SBJ11-C06.

In another preferred embodiment, the method comprises administering one or more antibodies selected from SBJ05-D08, SBJ05-C11, SBJ05-D14, SBJ11-J13, SBJ05-M13, SBJ09-G14, SBJ05-N02, SBJ09-M06, SBJ08-F04, and SBJ05-B17.

In another preferred embodiment, said method comprises administering one or more antibodies selected from SBJ05-D08, SBJ05-C11, SBJ05-D14, SBJ11-J13, and SBJ05-M13.

In another preferred embodiment, said method comprises administering one or more antibodies selected from SBJ09-G14, SBJ05-N02, SBJ09-M06, SBJ08-F04, and SBJ05-B17.

In another embodiment, the antibodies of the present invention are administered to the subject in combination with other therapeutic agents. In one embodiment, the additional therapeutic agents may be treating the symptoms of Kp infection on their own, and may optionally synergize with the effects of the antibodies. The additional agent that is administered may be selected by one skilled in the art for treating the infection. Co-administration of the antibody with an additional therapeutic agent (combination therapy) encompasses administering a composition comprising the anti-Kp antibody and the additional therapeutic agent as well as administering two or more separate compositions, one comprising the anti-Kp antibody and the other(s) comprising the additional therapeutic agent(s). Further, although co-administration or combination therapy generally means that the antibody and additional therapeutic agents are administered at the same time as one another, it also encompasses instances in which the antibody and additional therapeutic agents are administered at different times. For instance, the antibody may be administered once every three days, while the additional therapeutic agent is administered once daily.

Alternatively, the antibody may be administered prior to or subsequent to treatment with the additional therapeutic agent, for example after a patient has failed therapy with the additional agent. Similarly, administration of the anti-Kp antibody may be administered prior to or subsequent to other therapy.

The antibody and one or more additional therapeutic agents (the combination therapy) may be administered once, twice or at least the period of time until the condition is treated, palliated or cured. Preferably, the combination therapy is administered multiple times. The combination therapy may be administered from three times daily to once every six months.

The administering may be on a schedule such as three times daily, twice daily, once daily, once every two days, once every three days, once weekly, once every two weeks, once every month, once every two months, once every three months and once every six months, or may be administered continuously via a minipump. The combination therapy may be administered via an oral, mucosal, buccal, intranasal, inhalable, intravenous, subcutaneous, intramuscular, or parenteral.

In certain aspects, the invention provides a method for treating, preventing or alleviating the symptoms of a Kp mediated disorder in a subject in need thereof, in particular an infection from at least one drug resistant or multi-drug resistant strain of Kp, preferably an infection of a Klebsiella pneumoniae capsular type K64 strain according to any of the variants disclosed herein, more in particular infection from an NDM producing strain of Kp, or any other strain of Kp according to any of the variants disclosed in the present specification, comprising the step of administering to said subject an antibody or antigen-binding portion according to any one of the preceding embodiments, further comprising administering at least one additional therapeutic agent selected from the group consisting of. (a) one or more antibodies from the group consisting of: SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, SBJ11-J13; in particular
(b) one or more antibodies that specifically bind a surface antigen of a plurality of Kp strains; and/or
(c) one or more neutralizing antibodies that do not bind a surface antigen of Kp; and/or (d) one or more antimicrobial agents and/or
(e) one or more β-lactamase inhibitors.

In certain aspects, the invention provides a kit for treating, preventing or alleviating the symptoms of a Kp mediated disorder in a subject in need thereof, in particular an infection from at least one drug resistant or multi-drug resistant strain of Kp, preferably an infection of a Klebsiella pneumoniae capsular type K64 strain according to any of the variants disclosed herein, more in particular infection from an NDM producing strain of Kp, or any other strain of Kp according to any of the variants disclosed in the present specification, comprising a) one or more antibodies from the group consisting of. SBJ08-009, SBJ08-D18, SBJ03-F18, SBJ03-L02, SBJ05-B17, SBJ05-C11, SBJ05-D08, SBJ05-D14, SBJ05-K07, SBJ05-M13, SBJ05-N02, SBJ08-F04, SBJ08-H10, SBJ08-J10, SBJ08-K19, SBJ08-M13, SBJ08-N23, SBJ08-003, SBJ09-G14, SBJ09-I10, SBJ09-M06, SBJ10-H18, SBJ11-C06, SBJ11-J13; in particular
(b) one or more of these antibodies that specifically bind a surface antigen of a plurality of Kp strains; and/or
(c) one or more neutralizing antibodies that do not bind a surface antigen of Kp; and/or (d) one or more antimicrobial agents and/or
(e) one or more β-lactamase inhibitors.

In one preferred embodiment, said kit comprises or consists of one or more antibodies selected from SBJ08-009, SBJ08-D18, SBJ03-F18, SBJ08-H10, SBJ08-N23, SBJ08-K19, SBJ08-M13, SBJ03-L02, SBJ08-J10, SBJ10-H18, SBJ08-003, SBJ05-K07, SBJ09-I10, and SBJ11-C06, preferably comprises or consists of all these antibodies.

In another preferred embodiment, said kit comprises or consists of one or more antibodies selected from SBJ05-D08, SBJ05-C11, SBJ05-D14, SBJ11-J13, SBJ05-M13, SBJ09-G14, SBJ05-N02, SBJ09-M06, SBJ08-F04, and SBJ05-B17, preferably comprises or consists of all these antibodies.

In another preferred embodiment, said kit comprises or consists of one or more antibodies selected from SBJ05-D08, SBJ05-C11, SBJ05-D14, SBJ11-J13, and SBJ05-M13, preferably comprises or consists of all these antibodies.

In another preferred embodiment, said kit comprises or consists of one or more antibodies selected from SBJ09-G14, SBJ05-N02, SBJ09-M06, SBJ08-F04, and SBJ05-B17, preferably comprises or consists of all these antibodies.

The human monoclonal antibody or antigen-binding portion thereof herein disclosed may also be used advantageously as a diagnostic reagent in an in vitro method for detecting in a biological sample previously obtained from a patient (such as for example a serum, plasma, blood sample or any other suitable biological material, obtained from the patient, preferably a human being) anti-Kp antibodies. These antibodies may be found in the biological sample obtained from the patient for instance as a result of a previous exposure to the virus, or because a monoclonal antibody of the invention had been previously administered to the patient for therapeutic or prophylactic or research purposes. Thus, a diagnostic kit comprising the human monoclonal antibody or antigen-binding portion thereof herein disclosed of the invention, as a specific reagent, also falls within the scope of the invention, said kit being in particular designed for the detection and/or quantification, in a biological 20 sample previously obtained from a patient, of anti-Kp antibodies.

According to one embodiment, the kit can further comprise one or more multispecific antibodies, diabodies, triabodies, tetrabodies, minibodies, linear antibodies, chelating recombinant antibodies, tribodies, bibodies, intrabodies, nanobodies, binding-domain immunoglobulin fusion proteins, fusion antibodies, immunoadhesins or and antigen binding fragment thereof according to any of the embodiments disclosed in the present specification and in the claims.

The human monoclonal antibody or antigen-binding portion thereof herein disclosed may also be used advantageously for the design of a vaccine against Kp, in particular against at least one drug resistant or multi-drug resistant strain of Kp, preferably against a Klebsiella pneumoniae capsular type K64 strain according to any of the variants disclosed herein, more particularly against at least one NDM-producing strain of Kp or against a strain of Kp according to any of the variants disclosed in the present specification and in the claims. As disclosed in Rappuoli, Rino et al. “Reverse vaccinology 2.0: Human immunology instructs vaccine antigen design.” The Journal of experimental medicine vol. 213,4 (2016): 469-81. doi:10.1084jem.20151960”, human mAb may be used to identify protective antigens/epitopes. Structural characterization of the Ab-antigen complex may be used to instruct antigen design. Thus, also a method or the use of the human monoclonal antibody or antigen-binding portion thereof herein disclosed for the design of a vaccine against Kp, in particular against an NDM-producing strain of Kp, is within the scope of the invention. The human monoclonal antibody or antigen-binding portion thereof herein disclosed may be used for the preparation of mimotopes, such as for example anti-idiotype antibodies, peptides, S-protein truncated or artificial forms or others, endowed with the ability of evoking the antibodies herein disclosed. Among these, the anti-idiotype antibodies are preferred. The anti-idiotype antibodies are antibodies specifically directed against the idiotype of the neutralizing antibodies used for the manufacture thereof, and thus are able to mimic the key epitopes that they recognize. The manufacture of anti-idiotype antibodies is carried out by per se known methodologies that do not need further detailed explanations here. Thus, also mimotopes, preferably anti-idiotype antibodies, directed against an antibody of the invention fall within the scope of the invention. The human monoclonal antibody or antigen-binding portion thereof herein disclosed may be used for the manufacture of anti-idiotype antibodies according to methods per se known. Anti-idiotype antibodies are antibodies specifically directed towards the idiotype of the broad-range neutralizing antibodies used to prepare them, and as such are able to mimic the key epitopes they recognize. Therefore, anti-idiotype antibodies directed against a monoclonal antibody of the invention are also included in the scope of the invention.

### Preferred Embodiments of the Invention

Embodiment 1. A first embodiment of the invention is directed to a human monoclonal antibody or an antigen-binding portion thereof that specifically binds to a surface antigen of Klebsiella pneumoniae.
Embodiment 2. Herein provided is also the human monoclonal antibody or an antigen-binding portion thereof according to embodiment 1, which specifically binds to a surface antigen of a drug resistant or multi-drug resistant strain of Klebsiellapneumoniae.
Embodiment 3. Herein provided is also the human monoclonal antibody or an antigen-binding portion thereof according to embodiments 1 or 2, which specifically binds to a surface antigen of a carbapenemase-producing strain of Klebsiella pneumoniae, preferably a New Delhi Metallo-β-lactamase (NDM)-producing strain, more preferably at least a Klebsiella pneumoniae capsular type K64 strain according to any of the variants disclosed herein.
Embodiment 4. Herein provided is also the human monoclonal antibody or an antigen-binding portion thereof according to embodiment 3, wherein said strain is selected from NDM-1 positive strains, NDM-9 positive strains, and NDM-5 positive strains.
Embodiment 5. Herein provided is also the human monoclonal antibody or an antigen-binding portion thereof according to embodiments 3 or 4, wherein said strain is selected from ST147, ST258, ST493, ST307, and ST13.
Embodiment 6. Herein provided is also the human monoclonal antibody or an antigen-binding portion thereof according to any one of the embodiments from 1 to 5, wherein said surface antigen is selected from capsular polysaccharide or O-antigen.
Embodiment 7. Herein provided is also the human monoclonal antibody or an antigen-binding portion thereof according to embodiment 6, wherein said O-antigen is the O1-antigen or the 02-antigen.
Embodiment 8. Herein provided is also the human monoclonal antibody or an antigen-binding portion thereof according to any one of the embodiments from 1 to 7, wherein said antibody or antigen-binding portion thereof provides a percentage of bacterial killing greater than 30%, preferably greater than 60%, more preferably greater than 80%, as measured by an in vitro fluorescence-based serum bactericidal assay (F-SBA).
Embodiment 9. Herein provided is also the human monoclonal antibody or an antigen-binding portion thereof according to any one of the embodiments from 1 to 8, wherein said antibody or an antigen-binding portion thereof shows 50% inhibitory concentration (IC50) of less than 100 ng/ml, preferably less than 10 ng/ml, more preferably less than 5 ng/ml, when tested by an in vitro fluorescence-based serum bactericidal assay (F-SBA).
Embodiment 10. Herein provided is also the human monoclonal antibody or an antigen-binding portion thereof according to embodiment 9, wherein said antibody or antigen-binding portion is tested in an in vitro F-SBA assay against the ST147_{NDM-1} strain, the ST147NDM-₉ strain and/or the ST307_{NDM-5} strain of Klebsiellapneumoniae.
Embodiment 11. Herein provided is also the human monoclonal antibody or an antigen-binding portion thereof according to any one of the embodiments from 1 to 10, comprising a heavy chain variable domain (VH) and a light chain variable domain (VL), wherein said VH and VL comprise the following complementarity-determining regions (CDRs):
CDR1 of VH having SEQ ID NO: 1,
CDR2 of VH having SEQ ID NO: 2,
CDR3 of VH having SEQ ID NO: 3,
CDR1 of VL having SEQ ID NO: 4,
CDR2 of VL having the sequence AAS (Ala-Ala-Ser) and
CDR3 of VL having SEQ ID NO: 6;

or
CDR1 of VH having SEQ ID NO: 19,
CDR2 of VH having SEQ ID NO: 20,
CDR3 of VH having SEQ ID NO: 21,
CDR1 of VL having SEQ ID NO: 22,
CDR2 of VL having the sequence GAS (Gly-Ala-Ser) and
CDR3 of VL having SEQ ID NO: 24;

or
CDR1 of VH having SEQ ID NO: 37,
CDR2 of VH having SEQ ID NO: 38,
CDR3 of VH having SEQ ID NO: 39,
CDR1 of VL having SEQ ID NO: 40,
CDR2 of VL having the sequence AAS (Ala-Ala-Ser) and
CDR3 of VL having SEQ ID NO: 42.

or
CDR1 of VH having SEQ ID NO: 55,
CDR2 of VH having SEQ ID NO: 56,
CDR3 of VH having SEQ ID NO: 57,
CDR1 of VL having SEQ ID NO: 58,
CDR2 of VL having the sequence DDY (Asp-Asp-Tyr) and
CDR3 of VL having SEQ ID NO: 60

or
CDR1 of VH having SEQ ID NO: 73,
CDR2 of VH having SEQ ID NO: 74,
CDR3 of VH having SEQ ID NO: 75,
CDR1 of VL having SEQ ID NO: 76,
CDR2 of VL having the sequence DVS (Asp-Val-Ser) and
CDR3 of VL having SEQ ID NO: 78

or
CDR1 of VH having SEQ ID NO: 91,
CDR2 of VH having SEQ ID NO: 92,
CDR3 of VH having SEQ ID NO: 93,
CDR1 of VL having SEQ ID NO: 94,
CDR2 of VL having the sequence DVS (Asp-Val-Ser) and
CDR3 of VL having SEQ ID NO: 96

or
CDR1 of VH having SEQ ID NO: 127,
CDR2 of VH having SEQ ID NO: 128,
CDR3 of VH having SEQ ID NO: 129,
CDR1 of VL having SEQ ID NO: 130,
CDR2 of VL having the sequence AAS (Ala-Ala-Ser) and
CDR3 of VL having SEQ ID NO: 132

or
CDR1 of VH having SEQ ID NO: 145,
CDR2 of VH having SEQ ID NO: 146,
CDR3 of VH having SEQ ID NO: 147,
CDR1 of VL having SEQ ID NO: 148,
CDR2 of VL having the sequence EIS (Glu-Ile-Ser) and
CDR3 of VL having SEQ ID NO: 150

or
CDR1 of VH having SEQ ID NO: 163,
CDR2 of VH having SEQ ID NO: 164,
CDR3 of VH having SEQ ID NO: 165,
CDR1 of VL having SEQ ID NO: 166,
CDR2 of VL having the sequence ASS (Ala-Ser-Ser) and
CDR3 of VL having SEQ ID NO: 168

or
CDR1 of VH having SEQ ID NO: 181,
CDR2 of VH having SEQ ID NO: 182,
CDR3 of VH having SEQ ID NO: 183,
CDR1 of VL having SEQ ID NO: 184,
CDR2 of VL having the sequence AAS (Ala-Ala-Ser) and
CDR3 of VL having SEQ ID NO: 186

or
CDR1 of VH having SEQ ID NO: 199,
CDR2 of VH having SEQ ID NO: 200,
CDR3 of VH having SEQ ID NO: 201,
CDR1 of VL having SEQ ID NO: 202,
CDR2 of VL having the sequence DAS (Asp-Ala-Ser) and
CDR3 of VL having SEQ ID NO: 204

or
CDR1 of VH having SEQ ID NO: 217,
CDR2 of VH having SEQ ID NO: 218,
CDR3 of VH having SEQ ID NO: 219,
CDR1 of VL having SEQ ID NO: 220,
CDR2 of VL having the sequence GAS (Gly-Ala-Ser) and
CDR3 of VL having SEQ ID NO: 222

or
CDR1 of VH having SEQ ID NO: 235,
CDR2 of VH having SEQ ID NO: 236,
CDR3 of VH having SEQ ID NO: 237,
CDR1 of VL having SEQ ID NO: 238,
CDR2 of VL having the sequence QIS (Gln-Ile-Ser) and
CDR3 of VL having SEQ ID NO: 240

or
CDR1 of VH having SEQ ID NO: 253,
CDR2 of VH having SEQ ID NO: 254,
CDR3 of VH having SEQ ID NO: 255,
CDR1 of VL having SEQ ID NO: 256,
CDR2 of VL having the sequence DAS (Asp-Ala-Ser) and
CDR3 of VL having SEQ ID NO: 258

or
CDR1 of VH having SEQ ID NO: 271,
CDR2 of VH having SEQ ID NO: 272,
CDR3 of VH having SEQ ID NO: 273,
CDR1 of VL having SEQ ID NO: 274,
CDR2 of VL having the sequence AAS (Ala-Ala-Ser) and
CDR3 of VL having SEQ ID NO: 276

or
CDR1 of VH having SEQ ID NO: 289,
CDR2 of VH having SEQ ID NO: 290,
CDR3 of VH having SEQ ID NO: 291,
CDR1 of VL having SEQ ID NO: 292,
CDR2 of VL having the sequence KVS (Lys-Val-Ser) and
CDR3 of VL having SEQ ID NO: 294

or
CDR1 of VH having SEQ ID NO: 307,
CDR2 of VH having SEQ ID NO: 308,
CDR3 of VH having SEQ ID NO: 309,
CDR1 of VL having SEQ ID NO: 310,
CDR2 of VL having the sequence GAS (Gly-Ala-Ser) and
CDR3 of VL having SEQ ID NO: 312

or
CDR1 of VH having SEQ ID NO: 325,
CDR2 of VH having SEQ ID NO: 326,
CDR3 of VH having SEQ ID NO: 327,
CDR1 of VL having SEQ ID NO: 328,
CDR2 of VL having the sequence DAS (Asp-Ala-Ser) and
CDR3 of VL having SEQ ID NO: 330

or
CDR1 of VH having SEQ ID NO: 343,
CDR2 of VH having SEQ ID NO: 344,
CDR3 of VH having SEQ ID NO: 345,
CDR1 of VL having SEQ ID NO: 346,
CDR2 of VL having the sequence EVS (Glu-Val-Ser) and
CDR3 of VL having SEQ ID NO: 348

or
CDR1 of VH having SEQ ID NO: 361,
CDR2 of VH having SEQ ID NO: 362,
CDR3 of VH having SEQ ID NO: 363,
CDR1 of VL having SEQ ID NO: 364,
CDR2 of VL having the sequence GAS (Gly-Ala-Ser) and
CDR3 of VL having SEQ ID NO: 366

or
CDR1 of VH having SEQ ID NO: 379,
CDR2 of VH having SEQ ID NO: 380,
CDR3 of VH having SEQ ID NO: 381,
CDR1 of VL having SEQ ID NO: 382,
CDR2 of VL having the sequence EVS (Glu-Val-Ser) and
CDR3 of VL having SEQ ID NO: 383

or
CDR1 of VH having SEQ ID NO: 397,
CDR2 of VH having SEQ ID NO: 398,
CDR3 of VH having SEQ ID NO: 399,
CDR1 of VL having SEQ ID NO: 400,
CDR2 of VL having the sequence KVS (Lys-Val-Ser) and
CDR3 of VL having SEQ ID NO: 402

or
CDR1 of VH having SEQ ID NO: 415,
CDR2 of VH having SEQ ID NO: 416,
CDR3 of VH having SEQ ID NO: 417,
CDR1 of VL having SEQ ID NO: 418,
CDR2 of VL having the sequence GAS (Gly-Ala-Ser) and
CDR3 of VL having SEQ ID NO: 420

or
CDR1 of VH having SEQ ID NO: 433,
CDR2 of VH having SEQ ID NO: 434,
CDR3 of VH having SEQ ID NO 435,
CDR1 of VL having SEQ ID NO: 436,
CDR2 of VL having the sequence RVS (Arg-Val-Ser) and
CDR3 of VL having SEQ ID NO: 438.
Embodiment 12. Herein provided is also the human monoclonal antibody or an antigen-binding portion thereof according to any one of the embodiments from 1 to 11, comprising a heavy chain variable domain (VH) and a light chain variable domain (VL),
wherein said VH having SEQ ID NO: 7 and said VL having SEQ ID NO: 8; or
wherein said VH having SEQ ID NO: 25 and said VL having SEQ ID NO: 26; or
wherein said VH having SEQ ID NO: 43 and said VL having SEQ ID NO: 44 or
wherein said VH having SEQ ID NO: 61 and said VL having SEQ ID NO: 62 or
wherein said VH having SEQ ID NO: 79 and said VL having SEQ ID NO: 80 or
wherein said VH having SEQ ID NO: 97 and said VL having SEQ ID NO: 98 or
wherein said VH having SEQ ID NO: 133 and said VL having SEQ ID NO: 134 or
wherein said VH having SEQ ID NO: 151 and said VL having SEQ ID NO: 152 or
wherein said VH having SEQ ID NO: 169 and said VL having SEQ ID NO: 170 or
wherein said VH having SEQ ID NO: 187 and said VL having SEQ ID NO: 188 or
wherein said VH having SEQ ID NO: 205 and said VL having SEQ ID NO: 206 or
wherein said VH having SEQ ID NO: 223 and said VL having SEQ ID NO: 224 or
wherein said VH having SEQ ID NO: 241 and said VL having SEQ ID NO: 242 or
wherein said VH having SEQ ID NO: 259 and said VL having SEQ ID NO: 260 or
wherein said VH having SEQ ID NO: 277 and said VL having SEQ ID NO: 278 or
wherein said VH having SEQ ID NO: 295 and said VL having SEQ ID NO: 296 or
wherein said VH having SEQ ID NO: 313 and said VL having SEQ ID NO: 314 or
wherein said VH having SEQ ID NO: 331 and said VL having SEQ ID NO: 332 or
wherein said VH having SEQ ID NO: 349 and said VL having SEQ ID NO: 350 or
wherein said VH having SEQ ID NO: 367 and said VL having SEQ ID NO: 368 or
wherein said VH having SEQ ID NO: 385 and said VL having SEQ ID NO: 386 or
wherein said VH having SEQ ID NO: 403 and said VL having SEQ ID NO: 404 or
wherein said VH having SEQ ID NO: 421 and said VL having SEQ ID NO: 422 or
wherein said VH having SEQ ID NO: 439 and said VL having SEQ ID NO: 440.
Embodiment 13. Herein provided is also the human monoclonal antibody or antigen-binding portion thereof according to any one of the embodiments from 1 to 12, wherein said VL and said VH is at least 85% identical in amino acid sequence, preferably at least 95%, more preferably at least 99% of
VH having SEQ ID NO: 7 and said VL having SEQ ID NO: 8; or
VH having SEQ ID NO: 25 and said VL having SEQ ID NO: 26; or
VH having SEQ ID NO: 43 and said VL having SEQ ID NO: 44 or
VH having SEQ ID NO: 61 and said VL having SEQ ID NO: 62 or
VH having SEQ ID NO: 79 and said VL having SEQ ID NO: 80 or
VH having SEQ ID NO: 97 and said VL having SEQ ID NO: 98 or
VH having SEQ ID NO: 133 and said VL having SEQ ID NO: 134 or
VH having SEQ ID NO: 151 and said VL having SEQ ID NO: 152 or
VH having SEQ ID NO: 169 and said VL having SEQ ID NO: 170 or
VH having SEQ ID NO: 187 and said VL having SEQ ID NO: 188 or
VH having SEQ ID NO: 205 and said VL having SEQ ID NO: 206 or
VH having SEQ ID NO: 223 and said VL having SEQ ID NO: 224 or
VH having SEQ ID NO: 241 and said VL having SEQ ID NO: 242 or
VH having SEQ ID NO: 259 and said VL having SEQ ID NO: 260 or
VH having SEQ ID NO: 277 and said VL having SEQ ID NO: 278 or
VH having SEQ ID NO: 295 and said VL having SEQ ID NO: 296 or
VH having SEQ ID NO: 313 and said VL having SEQ ID NO: 314 or
VH having SEQ ID NO: 331 and said VL having SEQ ID NO: 332 or
VH having SEQ ID NO: 349 and said VL having SEQ ID NO: 350 or
VH having SEQ ID NO: 367 and said VL having SEQ ID NO: 368 or
VH having SEQ ID NO: 385 and said VL having SEQ ID NO: 386 or
VH having SEQ ID NO: 403 and said VL having SEQ ID NO: 404 or
VH having SEQ ID NO: 421 and said VL having SEQ ID NO: 422 or
VH having SEQ ID NO: 439 and said VL having SEQ ID NO: 440.
Embodiment 14. Herein provided is also the human monoclonal antibody according to any one of embodiments from 1 to 13, wherein the heavy chain of said antibody having SEQ ID NO: 9 and the light chain of said antibody having SEQ ID NO: 10; or
the heavy chain of said antibody having SEQ ID NO: 27 and the light chain of said antibody having SEQ ID NO: 28,or
the heavy chain of said antibody having SEQ ID NO: 45 and the light chain of said antibody having SEQ ID NO: 46,or
the heavy chain of said antibody having SEQ ID NO: 63 and the light chain of said antibody having SEQ ID NO: 64,
or the heavy chain of said antibody having SEQ ID NO: 81 and the light chain of said antibody having SEQ ID NO: 82,
or the heavy chain of said antibody having SEQ ID NO: 99 and the light chain of said antibody having SEQ ID NO: 100,or
the heavy chain of said antibody having SEQ ID NO: 135 and the light chain of said antibody having SEQ ID NO: 136,or
the heavy chain of said antibody having SEQ ID NO: 153 and the light chain of said antibody having SEQ ID NO: 154,or
the heavy chain of said antibody having SEQ ID NO: 171 and the light chain of said antibody having SEQ ID NO: 172,or
the heavy chain of said antibody having SEQ ID NO: 189 and the light chain of said antibody having SEQ ID NO: 190,or
the heavy chain of said antibody having SEQ ID NO: 207 and the light chain of said antibody having SEQ ID NO: 208,or
the heavy chain of said antibody having SEQ ID NO: 225 and the light chain of said antibody having SEQ ID NO: 226,or
the heavy chain of said antibody having SEQ ID NO: 243 and the light chain of said antibody having SEQ ID NO: 244,or
the heavy chain of said antibody having SEQ ID NO: 261 and the light chain of said antibody having SEQ ID NO: 262,or
the heavy chain of said antibody having SEQ ID NO: 279 and the light chain of said antibody having SEQ ID NO: 280,or
the heavy chain of said antibody having SEQ ID NO: 297 and the light chain of said antibody having SEQ ID NO: 298, or
the heavy chain of said antibody having SEQ ID NO: 315 and the light chain of said antibody having SEQ ID NO: 316 or
the heavy chain of said antibody having SEQ ID NO: 333 and the light chain of said antibody having SEQ ID NO: 334 or
the heavy chain of said antibody having SEQ ID NO: 351 and the light chain of said antibody having SEQ ID NO: 352 or
the heavy chain of said antibody having SEQ ID NO: 369 and the light chain of said antibody having SEQ ID NO: 370 or
the heavy chain of said antibody having SEQ ID NO: 387 and the light chain of said antibody having SEQ ID NO: 388 or
the heavy chain of said antibody having SEQ ID NO: 405 and the light chain of said antibody having SEQ ID NO: 406 or
the heavy chain of said antibody having SEQ ID NO: 423 and the light chain of said antibody having SEQ ID NO: 424 or
the heavy chain of said antibody having SEQ ID NO: 441 and the light chain of said antibody having SEQ ID NO: 442.
Embodiment 15. Herein provided is also the human monoclonal antibody according to any one of the embodiments from 1 to 14, wherein the fragment crystallizable (Fc) region of IgG1 backbone contains the following mutation E430G.
Embodiment 16. Herein provided is also a human monoclonal antibody or an antigen-binding portion thereof that compete for the binding to a surface antigen of a Klebsiella pneumoniae strain with any one of the antibody or antigen-binding portion according to any one of the embodiments 1 to 15.
Embodiment 17. Herein provided is also the human monoclonal antibody or an antigen-binding portion thereof according to any one of the embodiments from 1 to 16 for use in a prophylactic or therapeutic treatment of a Klebsiella pneumoniae infection or conditions or disorders resulting from such infection, in particular for use in the prevention and/or treatment of an infection of a drug resistant or multi-drug resistant strain of Klebsiella pneumoniae.
Embodiment 18. Herein provided is also the human monoclonal antibody or an antigen-binding portion thereof according to any one of the embodiments from 1 to 16, for use in a prophylactic or therapeutic treatment of an infection of an NDM-producing strain of Klebsiella pneumoniae or conditions or disorders resulting from such infection, in particular an NDM-1, NDM-5 and/or NDM-9 positive strain.
Embodiment 19. Herein provided is also the human monoclonal antibody or an antigen-binding portion thereof according to any one of the embodiments from 1 to 16, for use in a prophylactic or therapeutic treatment of an infection of the NDM-1 positive ST147 strain of Klebsiella pneumoniae or conditions or disorders resulting from such infection.
Embodiment 20. Herein provided is also the human monoclonal antibody or an antigen-binding portion thereof according to any one of the embodiments from 1 to 16, for use in a method for prophylactic or therapeutic treatment of a Klebsiellapneumoniae infection or conditions or disorders resulting from such infection, wherein said method comprising the step of administering to a patient between 0.025 to 5 mg/kg of said human monoclonal antibody or an antigen-binding portion thereof.
Embodiment 21. Herein provided is also the human monoclonal antibody or an antigen-binding portion thereof according to any one of the embodiments from 1 to 16, for use in a method for prophylactic or therapeutic treatment of a Klebsiellapneumoniae infection or conditions or disorders resulting from such infection, wherein said method comprising the step of administering to a patient between 0.1 to 3 mg/kg of said human monoclonal antibody or an antigen-binding portion, preferably 0.25 mg/kg.
Embodiment 22. Herein provided is also the human monoclonal antibody or an antigen-binding portion thereof for use according to any embodiments from 17 to 21, wherein said human monoclonal antibody or an antigen-binding portion thereof is administered by intravenous, subcutaneous, intraperitoneal or intramuscular route.
Embodiment 23. Herein provided is also the human monoclonal antibody or an antigen-binding portion thereof for use according to any one of the embodiments from 17 to 21, wherein said method comprising the step of administering to a patient said human monoclonal antibody or an antigen-binding portion once a day for at least 3 days.
Embodiment 24. Herein provided is also a pharmaceutical composition comprising one or more human monoclonal antibody or antigen-binding portion thereof according to any one of the embodiments from 1 to 16 and a pharmaceutically acceptable carrier.
Embodiment 25. Herein provided is also the pharmaceutical composition according to embodiment 24 comprising equal or less than 400 mg for unit dosage of said human monoclonal antibody or antigen-binding portion thereof
Embodiment 26. Herein provided is also the pharmaceutical composition according to embodiment 24 comprising equal or less than 100 mg for unit dosage of said human monoclonal antibody or antigen-binding portion thereof.
Embodiment 27. Herein provided is also the pharmaceutical composition according to any one of the embodiments from 24 to 26 for intravenous, subcutaneous, intraperitoneal or intramuscular administration.
Embodiment 28. Herein provided is also the composition according to any one of the embodiments from 24 to 27, for use in the prevention and/or treatment of a Klebsiella pneumoniae infection.
Embodiment 29. Herein provided is also an isolated cell line that produces the antibody or antigen-binding portion thereof according to any one of the embodiments from 1 to 16.
Embodiment 30. Herein provided is also an isolated nucleic acid molecule comprising a nucleotide sequence that encodes the antibody or antigen-binding portion thereof according to any one of the embodiments from 1 to 16.
Embodiment 31. Herein provided is also a vector comprising the nucleic acid molecule according to embodiment 30, wherein the vector optionally comprises an expression control sequence operably linked to the nucleic acid molecule.
Embodiment 32. Herein provided is also the vector according to embodiment 31, wherein said vector is a selected from RNA virus vectors, DNA virus vectors, plasmid viral vectors, adenovirus vectors, adenovirus associated virus vectors, herpes virus vectors and retrovirus vectors.
Embodiment 33. Herein provided is also a composition, comprising an isolated nucleic acid molecule according to embodiment 30 or a vector according to embodiment 31 or 32 for use in the prevention and/or treatment of a Klebsiellapneumoniae infection.
Embodiment 34. Herein provided is also the composition according to embodiment 33 wherein said nucleic acid molecule or said vector is formulated in a lipid nanoparticle.
Embodiment 35. Herein provided is also a host cell comprising the vector according to embodiment 29 or the nucleic acid molecule according to embodiment 30.
Embodiment 36. Herein provided is also a non-human transgenic animal or transgenic plant comprising the nucleic acid according to embodiment 30, wherein the non-human transgenic animal or transgenic plant expresses said nucleic acid.
Embodiment 37. Herein provided is also the use of the human monoclonal antibody or an antigen-binding portion thereof according to any one of the embodiments from 1 to 16 in the diagnosis of a Klebsiellapneumoniae infection.
Embodiment 38. Herein provided is also an in vitro method for revealing the presence of a Klebsiella pneumoniae strain in a sample comprising the following steps:
   i) Contacting the antibody or an antigen-binding portion thereof according to any one of the embodiments from 1 to 16 with said sample;
   ii) Detecting the binding of said antibody or an antigen-binding portion thereof with a surface antigen of Klebsiella pneumoniae.
Embodiment 39. Herein provided is also an in vitro method for the diagnosis of a Klebsiella pneumoniae infection in a subject comprising the following steps:
   i) Contacting the antibody or an antigen-binding portion thereof according to any one of the embodiments from 1 to 16 with a biological sample of said subject;
   ii) Detecting the binding of said antibody or an antigen-binding portion thereof with a surface antigen of Klebsiella pneumoniae.
Embodiment 40. Herein provided is also the method in vitro according to embodiment 38 or 39, wherein said method is immunoassay selected from ELISA, RIA, flow cytometry, tissue immunohistochemistry, Western blot (immunoblot), immunoprecipitation or any other equivalent assays.
Embodiment 41. Herein provided is also the in vitro method according to embodiments 38 to 40, wherein said antibody or an antigen-binding portion thereof is directly labelled with a detectable label or wherein said antibody or an antigen-binding portion thereof (the first antibody) is bound to a second labelled antibody or to another labelled molecule.
Embodiment 42. Herein provided is also a diagnostic kit comprising as a specific reagent an antibody or an antigen-binding portion thereof according to any one of the embodiments from 1 to 16, said kit being intended in particular for use in a method for detecting or quantifying, in a biological sample from a patient, anti-Klebsiella pneumoniae antibodies, in particular anti-NMD-producing Klebsiellapneumoniae antibodies.
Embodiment 43. Herein provided is also the use of an antibody or an antigen-binding portion thereof according to any one of the embodiments from 1 to 16 for the design of a vaccine against Klebsiella pneumoniae.
Embodiment 44. Herein provided is also the use of an antibody or an antigen-binding portion thereof according to any one of the embodiments from 1 to 16 for the design of a vaccine against an NMD-producing strain of Klebsiella pneumoniae.
Embodiment 45. Herein provided is also a mimotope specifically directed against the idiotype of an antibody or an antigen-binding portion thereof according to any one of the embodiments from 1 to 16.
Embodiment 46. Herein provided is also an anti-idiotype antibody that is specifically directed against the idiotype of an antibody or an antigen-binding portion thereof according to any one of the embodiments from 1 to 16.
Embodiment 47. Herein provided is also An immunospecific polypeptide comprising at least a variable domain, in particular at least a heavy chain variable domain (VH) and a light chain variable domain (VL), as defined in any one of the embodiments herein disclosed, or any one of the CDRs as herein defined, in particular said immunospecific polypeptide is a multispecific antibody, a bi-specific antibody, a three-specific antibody, a monoclonal antibody, a scFV, a diabody, a triabody, a tetrabody, a minibody, a linear antibody, a chelating recombinant antibody, a tribody, a bibody, an intrabody, a nanobody, a binding-domain immunoglobulin fusion protein, a fusion antibody, an immunoadhesin or and antigen binding fragment thereof. ***

The following experimental section is provided solely by way of illustration and not limitation and does not intend to restrict the scope of the invention as defined in the appended claims. The claims are an integral part of the description.

### Examples

### 1. Results

### 1.1. Isolation of Human Monoclonal Antibodies Recognizing Outbreak Strain ST147_{NDM-1}

To isolate Klebsiella-specific mAbs representing the breadth of Ab repertoire induced by natural Kp infection, we used an antigen-agnostic approach. Peripheral blood mononuclear cells (PBMCs) were collected from seven convalescent patients who recovered from Kp ST147_{NDM-1} bloodstream infection of the Tuscany nosocomial outbreak, and one had gut colonization by ST147_{NDM-1} during the Tuscany outbreak (Table 1).

TABLE 1
Information about the patients from Tuscany
outbreak enrolled in the study.

| | | | | Months | |
|---|---|---|---|---|---|
| Patient | | Date of | Collection | after | |
| ID | Age | Bacteremia | Date | infection | Germ |
| 1 | 67 | 21-08-19 | 21-03-20 | 7 | Klebsiella ST147_{NDM1} |
| 3 | 69 | 16-02-20 | 07-10-20 | 8 | Klebsiella ST147_{NDM1} |
| 5 | 74 | 02-11-19 | 12-11-20 | 12 | Klebsiella ST147_{NDM1} |
| 6 | 77 | 05-11-20 | 04-12-20 | 1 | Klebsiella ST147_{NDM1} |
| 8 | 43 | 27-08-20 | 07-01-21 | 5 | Klebsiella ST147_{NDM1} |
| 9 | 61 | 11/04/20 | 08/01/21 | 8 | Klebsiella ST147_{NDM1} |
| 10 | 74 | 20-04-20 | 25-01-21 | 8 | Klebsiella ST147_{NDM1} |
| 11 | 78 | n/a | 27-11-20 | | Klebsiella ST147_{NDM1} |
| | | (asympto- | | | |
| | | matic | | | |
| | | colonized | | | |
| | | patient) | | | |

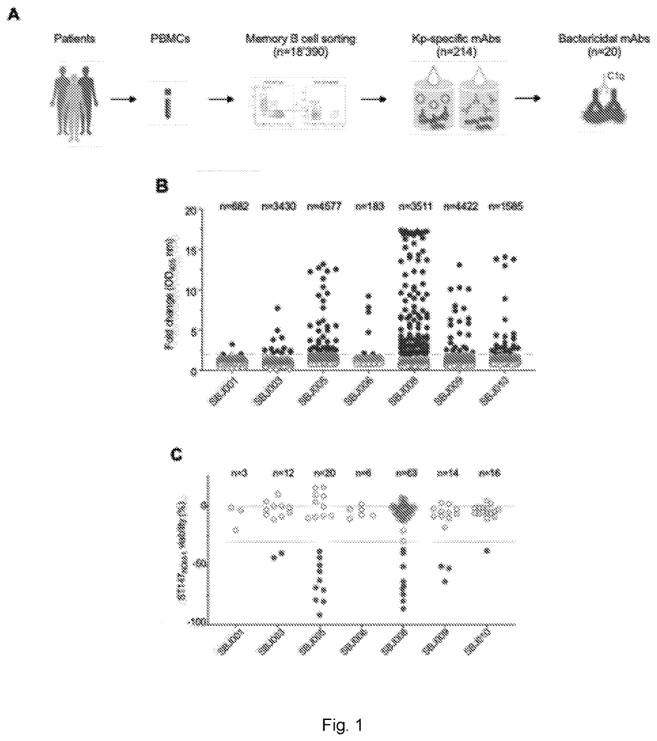

A total of 18′390 CD19⁺ CD27⁺ IgM-IgD— memory B cells (MBCs), either IgG⁺ or IgA⁺-positive, was single-cell sorted and plated together with CD40L-expressing feeder cells, IL-2 and IL-21 to promote MBC activation, proliferation and antibody secretion (FIG. 1A;

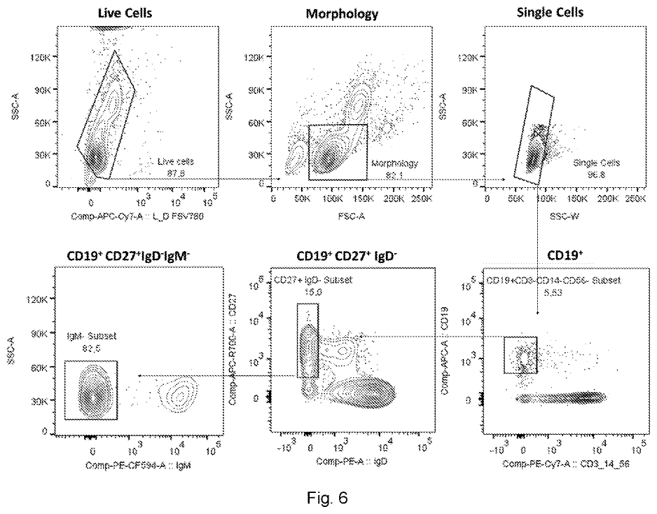

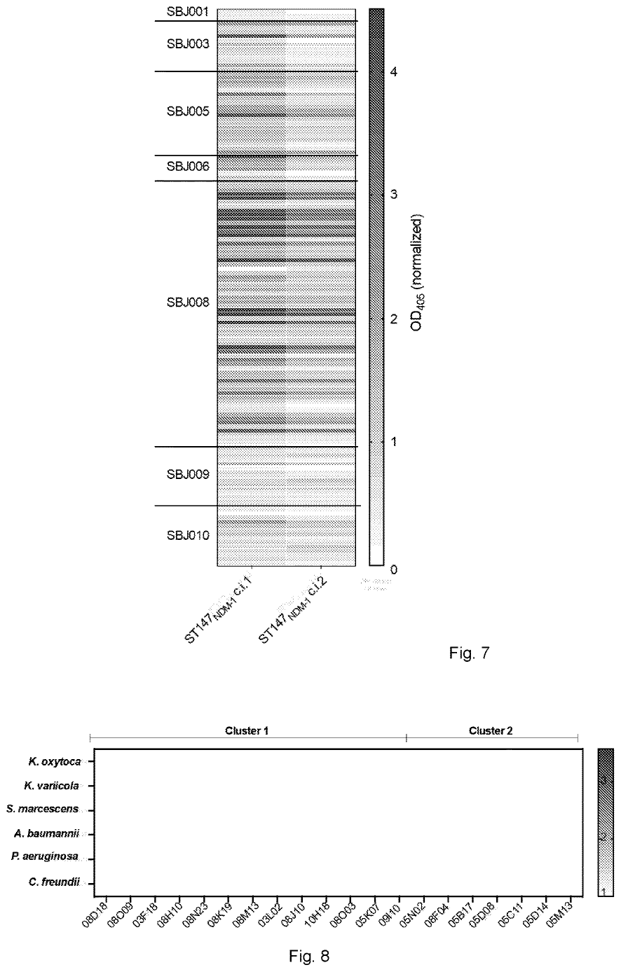

FIG. 6). MBC culture supernatants were screened by enzyme-linked immunosorbent assay (ELISA) against fixed intact ST147_{NDM-1} bacteria to select Kp-reactive mAbs.

MBC culture supernatants were screened by ELISA against fixed intact ST147_{NDM-1} bacteria which allowed to identify 214 mAbs specific for antigens displayed on the bacterial surface (FIG. 1B, Table 2).

TABLE 2
Kp isolates used in the manuscript.

| | | | K | O | O | Carbapenemase |
|---|---|---|---|---|---|---|
| Strain name | Origin | ST | locus | locus | type | gene |
| ST147_{NDM-1} | Bacteremia | 147 | KL64 | O2v1 | O2a | bla_{NDM-1} |
| c.i.1 | | | | | | |
| ST147_{NDM-1} | Bacteremia | 147 | KL64 | O2v1 | O2a | bla_{NDM-1} |
| c.i.2 | | | | | | |
| ST147_{NDM-9} | Bacteremia | 147 | KL64 | O2v1 | O2a | bla_{NDM-9} |
| ST307_{NDM-5} | Abdominal | 307 | KL102 | O2v2 | O2afg | bla_{NDM-5} |
| | drainage | | | | | |
| ST258 | Urinary infection | 258 | KL107 | O2v2 | O2afg | — |
| ST512_{VIM} | n/a | 512 | KL107 | O2v2 | O2a | bla_{VIM} |
| ST13_{OXA-48} | Rectal swab | 13 | KL57 | O1v2 | O1 | bla_{OXA-48} |
| ST493 | ATCC43816 | 493 | KL2 | O1v1 | O1 | — |

Upon molecular cloning of their V_{H} and V_{L} variable portions, sequences of 134 Kp-reactive mAbs were recovered with successful V_{H} and V_{L} pairing. To validate binding properties of isolated mAbs, they were expressed on a small scale and tested by ELISA against outbreak-belonging ST147_{NDM-1} Kp strains (FIG. 1B).

1.2. Selection of Functional ST147_{NDM-1} Kp-Specific mAbs by High-Throughput Luminescence-Based Serum Bactericidal Assays (L-SBA)

Further, mAbs were screened for their ability to induce complement-dependent killing of ST147_{NDM-1} Kp as it is widely acknowledged that pathogens can elicit both functional and non-functional antibodies.

For this purpose, a high-throughput luminescence-based serum bactericidal assay (L-SBA) was developed that utilizes luminescent readout of ATP production as a proxy of Kp viability. Four dilutions were tested per each mAb, and those giving a reduction greater than 30% of bacterial viability, were considered as positive hits. Out of the 134 Kp-binding mAbs screened, 25 candidates showing bactericidal activity against ST147_{NDM-1} were thus selected (FIG. 1C) and expressed at larger scale. Upon excluding five mAbs that failed to re-confirm their activity, we continued evaluating binding and functional properties of 20 bactericidal ST147_{NDM-1}-targetting mAbs.

1.3 Klebsiella Library Profiling Reveals Bactericidal mAbs have Different Degree of Cross-Reactivity and Target ST147_{NDM-1} Capsule and O-Antigen

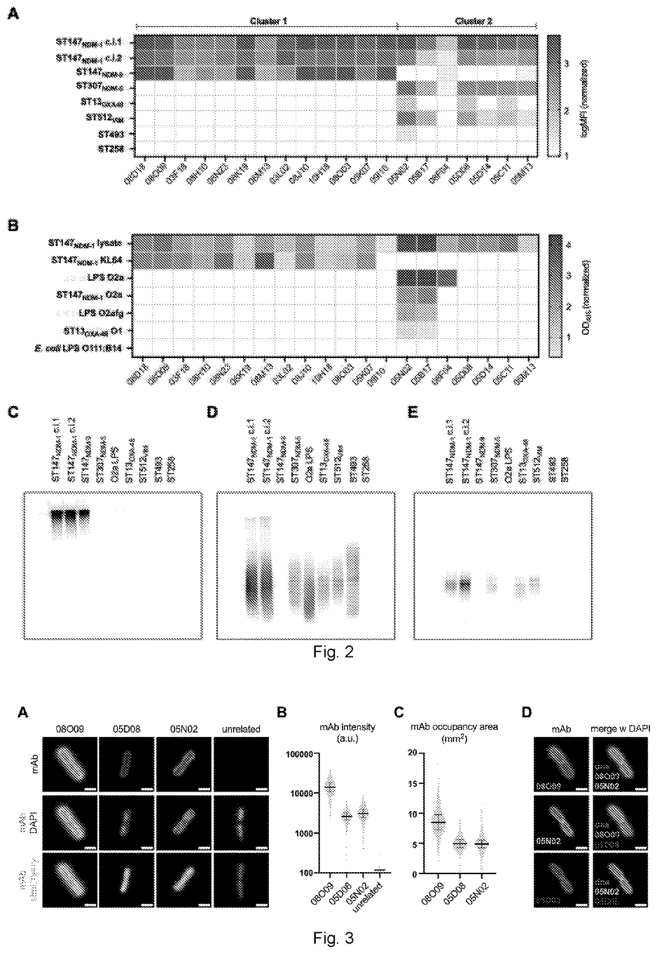

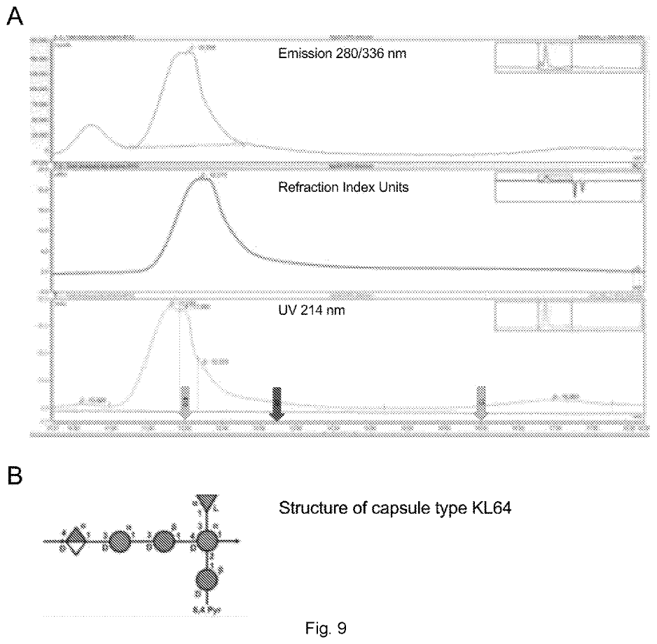

To characterize specificity and cross-reactivity of isolated bactericidal mAbs, flow cytometry was employed to profile their binding against a panel of bacterial strains. Pathogenic Kp belonging to different genetically distant STs were included (Table 2) as well as non-pathogenic Klebsiella species and several commensal strains. mAbs binding to bacterial surface displayed different levels of intensity, ranging from virtually null to high, suggesting differential expression of target antigens (FIG. 2A). Moreover, mAbs showed high specificity to pathogenic sensu stricto Kp strains as they did not react with other Kp phylogroups nor with commensals (FIG. 8). Based on binding profiles, mAbs were grouped into two main clusters (FIG. 2A).

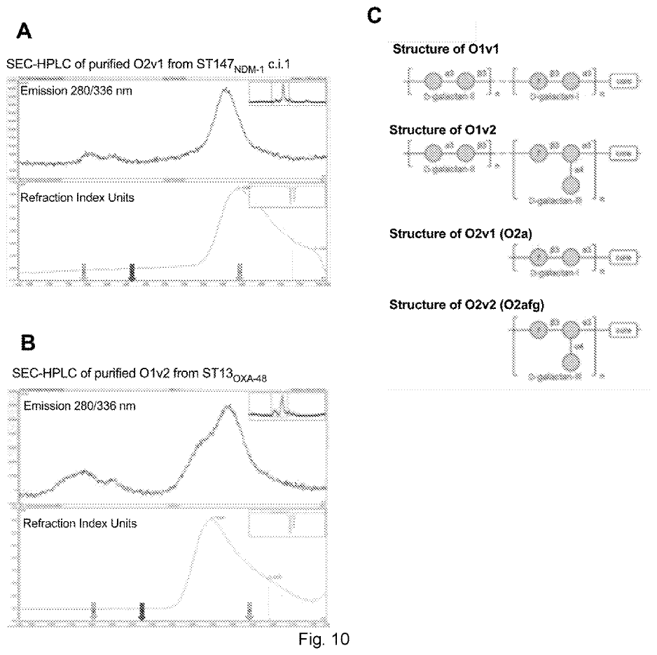

The first cluster contained mAbs with rather restricted binding characteristics, recognising exclusively outbreak ST147 carrying either bla_{NDM-1} or bla_{NDM-9} genes (doi: 10.2807/1560-7917.ES.2020.25.48.2001779). The second group of mAbs displayed a broader binding pattern, being able to recognize up to seven strains of genetically distant pathogenic Kp. Comparative whole genome sequencing analysis of Kp library allowed to predict surface antigens targeted by mAbs. We inferred that cluster 1 mAbs were likely directed against ST147_{NDM-1} capsule (K-antigen), as these mAbs recognized strains bearing genes from the capsule type 64 biosynthetic locus (KL64) (https://pubmed.ncbi.nlm.nih.gov/487363/). Instead, cluster 2 mAbs were likely recognising ST147_{NDM-1}β-antigen, as they bound to the surface of strains bearing genes from O2a locus encoding O-antigen biosynthetic genes (Table S4). To test these assumptions, we assessed mAb binding in ELISA against purified Kp capsule and O-antigens of several types (FIGS. 9 and 10). We observed that KL64 capsule was recognized only by cluster 1 mAbs (FIG. 2B). Immunoblotting of Kp total sugar extract further confirmed that cluster 1 mAbs recognized high molecular weight (MW) molecular species only in KL64-bearing Kp strains (FIG. 2C).

Instead, ELISA against purified O-antigens revealed heterogeneous picture within cluster 2 mAbs. Three mAbs recognized purified O2a O-antigen, with variable degree of cross-reactivity against O2afg and O1 0-antigens (FIG. 2B). Immunoblotting analysis evidenced that these mAbs displayed a ladder-like binding pattern, compatible with typical O-antigen immunoblotting profile (FIG. 2D). Five other mAbs from cluster 2 did not recognize purified O-antigens in ELISA, however they displayed similar ladder-like binding profile in the middle MW range against total sugar extracts of Kp strains (FIG. 2E). Binding was restricted to strains expressing O2a O-antigens. Moreover, no binding was observed against ST147_{NDM-9}, closely related to ST147_{NDM-1} at the genomic level.

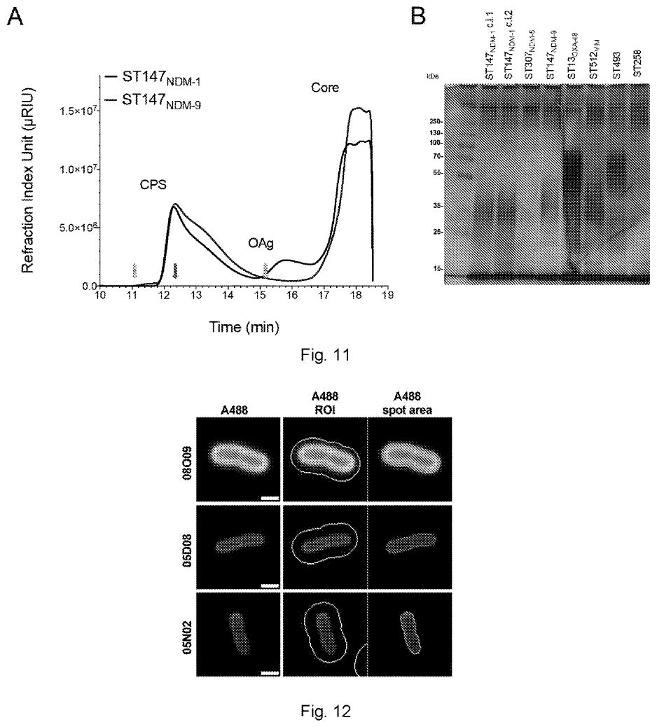

Comparative genetic analysis revealed that, among all genes encoding surface Kp antigens, ST147_{NDM-9} presented a single-point deletion in the wbbO gene, which encodes for a glycosyltransferase essential for O2a biosynthesis (REF). Mutation causes a frame shift and a premature stop, hindering the production of the complete O-antigen. HPLC and SDS-PAGE analysis of total sugar extracts from ST147_{NDM-9} indeed confirmed that ST147_{NDM-9} was a natural O-antigen-deficient mutant (FIG. 11) explaining the lack of binding by cluster 2 mAbs.

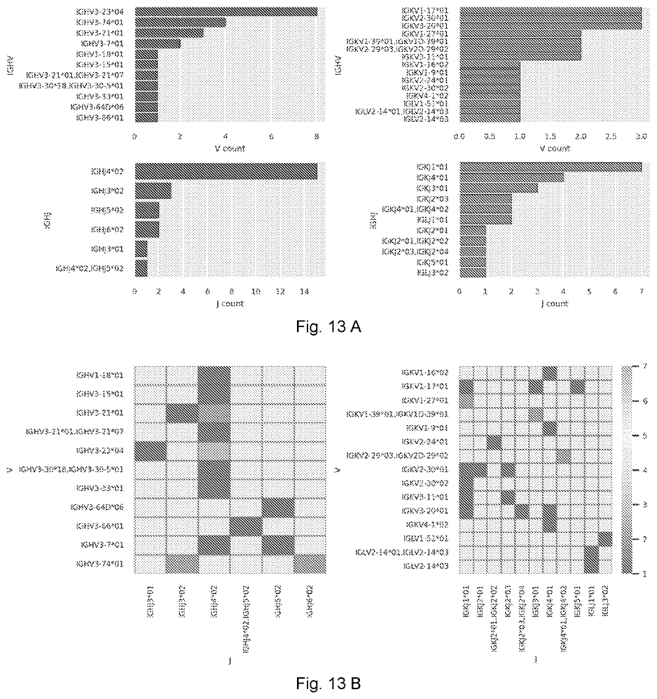

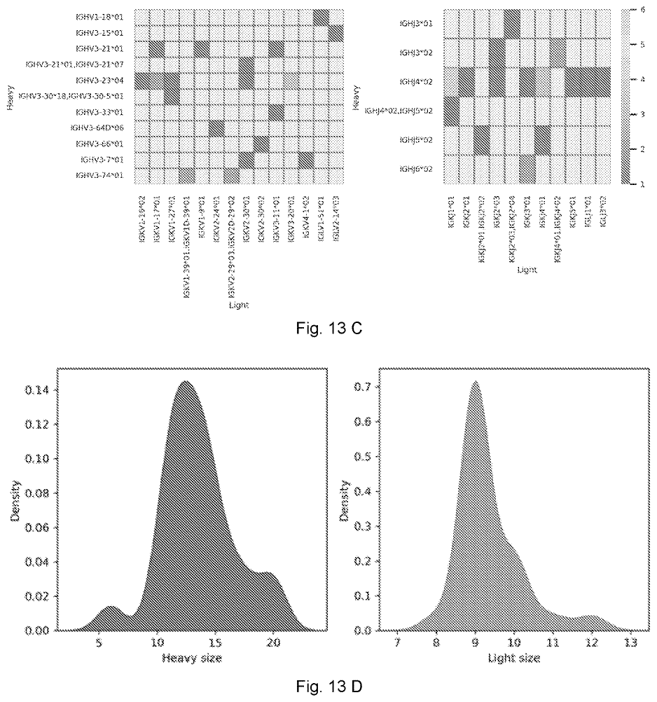

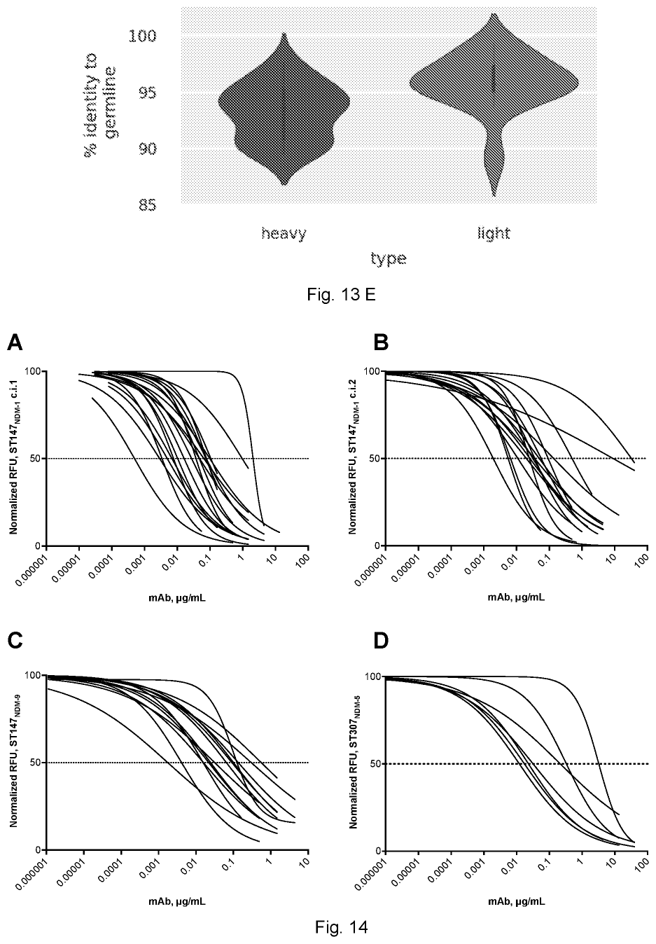

Hence, all together, our analysis allowed us to divide isolated ST147_{NDM-1}-targetting mAbs into KL64-specific and O2a β-antigen specific mAbs, wherein some O-antigen-specific mAbs were cross-reactive also against O1 type O-antigen. High-resolution and high-content analysis of mAb binding to ST147_{NDM-1} surface evidenced that mAbs targeting capsule and O-antigen produced non-overlapping patterns. Anti-capsule mAb O8O09 displayed higher intensity binding which distributed over a larger area with respect to the anti-O-antigen mAbs O5D08 and O5N02 (FIG. 3B, FIG. 3C, FIG. 12). Moreover, O8O09 signal was positioned on the most external layer of the bacteria (FIG. 3D). Analysis of the germline composition of mAbs evidenced a bias in germline usage for anti-capsular mAbs, while 3 clonal pairs were detected among O-antigen-specific mAbs (FIG. 13). Hence, we have collected information about cross-binding properties, specificity and sequence characteristics for 20 bactericidal ST147_{NDM-1}-targetting mAbs.

1.4. Functional Profiling Revealed that Isolated mAbs have Extremely Potent Bactericidal Activity, However Only Anti-Capsular mAbs are Poly-Functional

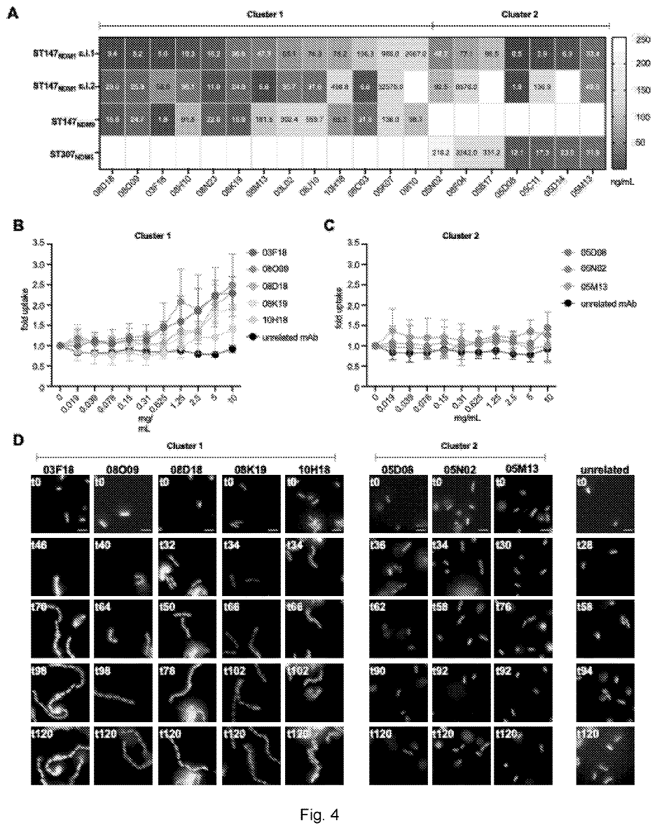

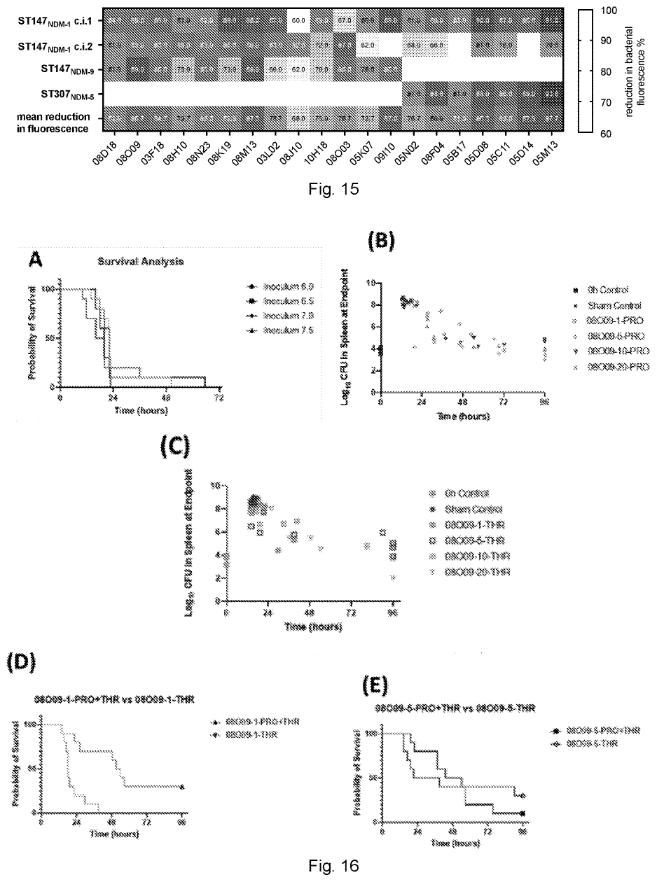

To better assess protective properties of anti-capsule and anti-O-antigen bactericidal antibodies, we used several in vitro assays relevant for antibody-mediated bacterial control in vivo. Bactericidal mAbs were expressed as human IgG1 with E430G mutation in the Fc domain, which enhances the hexamerization process that naturally occurs between Fc domains and thus potentiates antibody effector function. We also improved the processivity of L-SBA by designing a fluorescence-based SBA (F-SBA) in 384-well format and using resazurin staining for fluorescent readout of bacterial viability. F-SBA profiling of mAbs against complement-sensitive pathogenic Kp strains (ST147_{NDM-1}, ST147_{NDM-9}, and ST307_{NDM-5}) revealed that most of the antibodies were functional in the picomolar range of concentrations, with the most potent mAb showing 0.5-6 ng/mL IC₅₀ values (FIG. 4A; FIG. 14). Ability to kill different Kp strains correlated with mAb cross-binding properties, wherein anti-KL64 mAbs were effective against KL64-bearing ST147_{NDM-1} and ST147NDM-9, while anti-O-antigen mAbs were effective against O2 0-antigen-bearing ST147_{NDM-1} and ST307_{NDM-5} (FIG. 4A). F-SBA profiling allowed us to rank mAbs according to cumulative bactericidal potency in each cluster (FIG. 15). The ranking was additionally refined based on maximum ability of the mAb to reduce bacterial viability (FIG. 15).

Antibodies displaying highest bactericidal potency were then evaluated in macrophage-mediated opsonophagocytosis assays since phagocytes play crucial role in Kp clearance during infection (Broug-Holub et al., 1997; Cheung et al., 2000). Super-folded (sf)mCherry-overexpressing recombinant ST147_{NDM-1} was opsonized with escalating doses of mAbs prior to incubation with differentiated THP1 cells. Then fluorescence of internalised Kp was measured as a readout of mAb ability to promote opsonophagocytosis. We found that anti-KL64 mAbs increased Kp uptake into THP-1 cells at doses greater than 0.6 mg/mL, except for 10H18 which showed only a very mild effect (FIG. 4B). However, no Kp uptake was detected by anti-O-antigen mAbs (FIG. 4C). This indicated, on one hand, that anti-capsular but not anti-O-antigen mAbs were able to promote bacterial engulfment. On the other hand, it showed that opsonization with anti-capsular mAbs was able to overcome inhibition of opsonophagocytosis exerted by Kp surface virulence factors (Alvarez et al., 2000; Cortes et al., 2002; Regueiro et al., 2006; March et al., 2013).

Finally, aiming to analyse the effect of Kp culturing in the presence of bactericidal mAbs over time, we performed time-lapse imaging of ST147_{NDM-1}-sfmCherry incubated with increasing doses of mAbs. We found that cluster 1 mAbs induced enchained bacterial growth at 10 and 100 mg/mL (FIG. 4D). Mechanistically, this phenotype was associated with accumulation of concatenated replicating bacteria, without physical division of sister cells. Cluster 2 antibodies instead did not induce enchained growth at any tested dose (FIG. 4D). Bacterial concatenation induced by dimeric IgA has been previously proposed as a protective mechanism restricting pathogen growth and facilitating its clearance from the intestine (Moor et al., 2017; Bansept et al., 2019). Interestingly, we observe this phenomenon here in the presence of monomeric IgG.

Altogether, undertaken multi-layer analysis suggested that isolated mAbs had an extremely potent bactericidal activity and that their functionality may extend beyond opsonophagocytosis and implicate other effector mechanisms, such as enchained Kp growth. However, surprisingly, only anti-capsular mAbs were endowed with such poly-functionality.

TABLE 3
Sequence analysis of 24 functional mAbs.

| | | | | | Variable | |
|---|---|---|---|---|---|---|
| | | | | | region | CDR3 |
| | | | | | germline | aminoacid |
| Sample | Type | V_usage | D_usage | J_usage | identity, % | length |
| SBJ03-F18 | light | IGKV1-9*01 | | IGKJ4*01 | 93,706 | 9 |
| SBJ03-F18 | heavy | IGHV3-21*01 | IGHJ3*01 | IGHJ4*02 | 95,222 | 13 |
| SBJ03-L02 | light | IGKV1-27*01 | | IGKJ1*01 | 95,088 | 9 |
| SBJ03-L02 | heavy | IGHV3-30*18 | IGHJ2*01 | IGHJ4*02 | 95,578 | 14 |
| SBJ05-B17 | heavy | IGHV3-74*01 | IGHJ1*01 | IGHJ6*02 | 90,203 | 20 |
| SBJ05-B17 | light | IGKV1-39*01 | | IGKJ3*01 | 89,895 | 9 |
| SBJ05-C11 | heavy | IGHV1-18*01 | IGHJ1*01 | IGHJ4*02 | 89,384 | 13 |
| SBJ05-C11 | light | IGLV1-51*01 | | IGLJ3*02 | 93,515 | 11 |
| SBJ05-D08 | heavy | IGHV3-15*01 | IGHJ5*01 | IGHJ4*02 | 95,286 | 10 |
| SBJ05-D08 | light | IGLV2-14*03 | | IGLJ1*01 | 95,578 | 10 |
| SBJ05-D14 | light | IGLV2-14*01 | | IGLJ1*01 | 93,75 | 10 |
| SBJ05-D14 | heavy | IGHV3-15*01 | IGHJ5*01 | IGHJ4*02 | 92,256 | 10 |
| SBJ05-K07 | heavy | IGHV3-23*04 | IGHJ5*01 | IGHJ4*02 | 96,259 | 18 |
| SBJ05-K07 | light | IGKV1-27*01 | | IGKJ1*01 | 98,592 | 9 |
| SBJ05-M13 | light | IGKV2-24*01 | | IGKJ2*01 | 95,695 | 10 |
| SBJ05-M13 | heavy | IGHV3-64D*06 | IGHJ4*01 | IGHJ5*02 | 90,753 | 6 |
| SBJ05-N02 | heavy | IGHV3-74*01 | IGHJ1*01 | IGHJ6*02 | 92,905 | 20 |
| SBJ05-NO2 | light | IGKV1-39*01 | | IGKJ3*01 | 95,819 | 9 |
| SBJ08-D18 | heavy | IGHV3-23*04 | IGHJ5*01 | IGHJ4*02 | 93,493 | 12 |
| SBJ08-D18 | light | IGKV1-17*01 | | IGKJ3*01 | 97,535 | 9 |
| SBJ08-F04 | heavy | IGHV3-33*01 | IGHJ6*01 | IGHJ4*02 | 97,635 | 16 |
| SBJ08-F04 | light | IGKV3-11*01 | | IGKJ1*01 | 99,301 | 9 |
| SBJ08-H10 | heavy | IGHV3-23*04 | IGHJ4*01 | IGHJ4*02 | 91,186 | 12 |
| SBJ08-H10 | light | IGKV3-20*01 | | IGKJ1*01 | 94,81 | 9 |
| SBJ08-J10 | heavy | IGHV3-23*04 | IGHJ1*01 | IGHJ4*02 | 90,785 | 11 |
| SBJ08-J10 | light | IGKV2-30*01 | | IGKJ2*03 | 96,321 | 9 |
| SBJ08-K19 | heavy | IGHV3-21*01 | IGHJ2*01 | IGHJ3*02 | 96,918 | 14 |
| SBJ08-K19 | light | IGKV3-11*01 | | IGKJ2*03 | 99,303 | 10 |
| SBJ08-M13 | heavy | IGHV3-23*04 | IGHJ6*03 | IGHJ4*02 | 93,836 | 15 |
| SBJ08-M13 | light | IGKV1-16*02 | | IGKJ4*01 | 98,592 | 9 |
| SBJ08-N23 | heavy | IGHV3-66*01 | IGHJ6*01 | IGHJ4*02 | 92,15 | 14 |
| SBJ08-N23 | light | IGKV2-30*02 | | IGKJ1*01 | 96,296 | 8 |
| SBJ08-003 | heavy | IGHV3-23*04 | IGHJ3*01 | IGHJ4*02 | 93,878 | 13 |
| SBJ08-003 | light | IGKV3-20*01 | | IGKJ4*01 | 97,544 | 12 |
| SBJ08-009 | light | IGKV3-20*01 | | IGKJ2*03 | 96,194 | 10 |
| SBJ08-009 | heavy | IGHV3-23*04 | IGHJ2*01 | IGHJ3*01 | 94,218 | 12 |
| SBJ09-G14 | heavy | IGHV3-74*01 | IGHJ6*01 | IGHJ3*02 | 90,203 | 11 |
| SBJ09-G14 | light | IGKV2-29*03 | | IGKJ4*01 | 95,681 | 9 |
| SBJ09-110 | heavy | IGHV3-23*04 | IGHJ1*01 | IGHJ4*02 | 89,492 | 14 |
| SBJ09-110 | light | IGKV1-17*01 | | IGKJ5*01 | 97,183 | 9 |
| SBJ09-M06 | heavy | IGHV3-74*01 | IGHJ6*01 | IGHJ3*02 | 90,203 | 11 |
| SBJ09-M06 | light | IGKV2-29*03 | | IGKJ4*01 | 96,013 | 9 |
| SBJ10-H18 | heavy | IGHV3-21*01 | IGHJ6*01 | IGHJ4*02 | 95,254 | 12 |
| SBJ10-H18 | light | IGKV2-30*01 | | IGKJ1*01 | 97,674 | 9 |
| SBJ11-C06 | heavy | IGHV3-21*01 | IGHJ1*01 | IGHJ4*02 | 93,814 | 15 |
| SBJ11-C06 | light | IGKV1-17*01 | | IGKJ1*01 | 95,105 | 9 |
| SBJ11-J13 | heavy | IGHV3-7*01 | | IGHJ4*02 | 94,237 | 11 |
| SBJ11-J13 | light | IGKV2-30*01 | | IGKJ2*01 | 95,318 | 9 |

1.5. mAb poly-functionality is required for protection against fulminant ST147_{NDM-1} bloodstream infection in vivo

To answer the fundamental question of whether picomolar bactericidal activity on its own is sufficient to make therapeutic mAbs efficient in vivo or whether they need to be poly-functional, we evaluated protective properties of the most potent mAb candidates (anti-capsular O8009, anti-02 0-antigen specific O5D08, and anti-01/02 0-antigen specific 05N02) in prophylaxis (PRO) and treatment (THR) studies in an immunocompetent murine septicemia model. Fulminant bloodstream infection model was selected to reflect the clinical history of donors from whom mAbs were isolated and was set up to achieve 90-100% mortality within 24 hours of bacterial challenge (FIG. 16A), leaving a narrow time window for mAb therapeutic action.

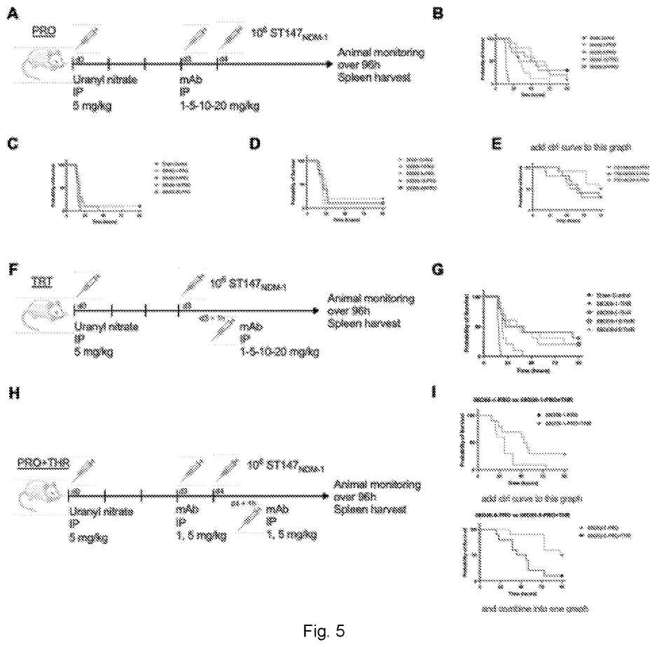

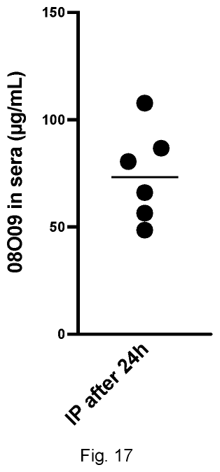

To assess the in vivo PRO activity against ST147_{NDM-1}, three groups of ten mice each were administered with 1, 5, 10, and 20 mg/kg mAb dose 24 hours prior to bacterial inoculation (FIG. 5A). O8O09 at 5 mg/kg and higher dosages resulted in significant survival extension compared to sham control (P<0.05), with 30% survival rate (FIG. 5B). Consistent with this observation, CFU counts in the spleen showed a reduction compare with control (FIG. 16B). Instead, O5D08 and O5N02 mAbs did not significantly prolong animal survival, at none of the doses administered (FIG. 5C-D). The prophylaxis experiment with 5 mg/kg 08O09 was repeated on 30 animals in total, showing consistency and reproducibility, with at least 35 h of mice survival and 50% survived up to 96 h (FIG. 5E). Plasma concentration of O8O09 associated with optimal in vivo efficacy was 74.3 (±21.7) μg/mL (FIG. 17).

Considering these results, we evaluated the treatment (THR) efficacy of O8O09 administered intravenously at 1, 5, 10, and 20 mg/kg one-hour post-bacterial challenge (FIG. 5F). In this case, moderate protection was achieved at 5 mg/kg and higher dosages (FIG. 5G) which was associated with lower CFU counts in the spleen (FIG. 16C). Lastly, we evaluated protective properties of O8O09 in a combinatorial prophylaxis/treatment (PRO+THR) regimen, where the first dose of O8O09 was administered intraperitoneally 24 hours before bacterial inoculation, and the second dose was administered intravenously one hour post-bacterial challenge (FIG. 5H). Interestingly, an advantage of using a combinatorial treatment was observed only for 1 mg/kg dosage, with 30% of survival till 96 h, which was otherwise suboptimal in monotherapy regimen (FIGS. 51 and 16D). However, when O8O09 was administered at 5 mg/kg, prophylaxis monotherapy performed significantly better than either combinatorial treatment or treatment monotherapy (FIGS. 51 and 16E), suggesting that while suboptimal mAb dosing could be compensated by combined prophylaxis-treatment regimen, optimal mAb dosing may perform well as a single monotherapy in a prophylactic regimen. Overall, these experiments demonstrate for the first time a potential use of anti-KL64-specific poly-functional mAb as a therapeutic agent against pandrug-resistant ST147_{NDM-1}.

### 1.6 Discussion

Here we present a panel with 25 potent mAbs which display different characteristics. Cluster 1 mAbs, in particular, O8O09 demonstrates to be the one promoting opsonophagocytosis of Kp, and protective in in vivo models. Despite this mAbs target a particular target as it's the CPS type 64, it is also true that the Tuscany outbreak is still ongoing with not a big diversity in the isolates (data not shown). Moreover, this is the first time, to our knowledge that an anti-K64 mAbs is described.

AMR Kp infections represent a staggering clinical challenge from the treatment point of view. K. pneumoniae has developed virulence factors (e.g., capsule and LPS) which prevent its uptake by phagocytes (Alvarez et al., 2000; Cortes et al., 2002; Regueiro et al., 2006; March et al., 2013). Our rationale was to investigate whether cluster 1 and cluster 2 mAbs could weaken the capsule and LPS protective action and favour macrophage-dependent uptake. We found that anti-capsular mAbs could block the protective action exerted by the capsule itself in preventing phagocytosis and macrophage-mediated killing.

Computational models suggested that enchained bacterial growth, triggered by the host immune response, involves only fast replicating bacteria. Thus, through this mechanism, infected organisms clump bacteria facilitating their clearance and protect their microbiota. It should also be noted that the strain used in the in vivo challenges is a pan drug strain which is spread not only through Italy but also globally, highlighting the potential to use O8O09 against MDR infections.

### 2. Materials and Methods

2.1. Single-cell sorting of memory B cells from convalescent donors

Peripheral blood mononuclear cells (PBMCs) were isolated from heparin-treated whole blood by density gradient centrifugation (Ficoll-Paque PREMIUM, GE Healthcare). After separation, PBMC were stained with Live/Dead FSV780(BD Horizon) in 100 μL final volume diluted 1:1000 at room temperature (RT). After 20 minutes of incubation, cells were washed with phosphate-buffered saline (PBS) 1× and unspecific bindings were saturated with 100 μL of 20% normal rabbit serum (Life Technologies). Following incubation at 4° C. for 30 min, cells were washed with PBS 1×, centrifuged at 1200 rpm for 8 min, and stained with CD19 APC (BD cat #561742), IgM PeCF594 (BD cat #562539), CD27 APCR700 (BD cat #565116), IgD PE (BD cat #562024), CD3 PE-Cy7 (BD cat #560910), CD14 PE-Cy7 (BD cat #560919) and CD56 PECy7 (BD cat #560916) in Staining Buffer (1% Fetal Bovine Serum, in PBS 1×) at 4° C. for 30 min. Following additional washing, cells were resuspended in Sorting Buffer (PBS/EDTA 2.5 mM). Stained memory B cells (MBCs) were single-cell sorted with a FACS Aria III Fusion BD (BD Biosciences) into 384-well plates containing 3T3-CD40L feeder cells and were incubated with IL-2 and IL-21 for 14 days (Andreano et al., 2021).

### ELISA Screening of mAb Binding Against Klebsiellapneumoniae

Supernatants resulting from single cell sorting of MBCs were used as a source of IgG1 and IgA monoclonal antibodies (mAbs) to test their binding to whole bacteria in a high-throughput enzyme-linked immunosorbent assay (ELISA). Pools of ST147_{NDM-1} c.i. 1 and ST147_{NDM-1} c.i. 2 (Table S2) were grown in LB medium, pelleted at exponential phase (OD₆₀₀=0.5), resuspended in PB, and plated into 384-well plastic plates (Greiner, ref. 781101). Plated bacteria were fixed at RT for 30 minutes in 0.5% of paraformaldehyde.

Plates were then blocked in PBS plus 1% Fetal Bovine Serum at RT for 1 h. After incubation, bacteria were incubated for 1 h with MBC supernatants diluted in sample buffer (PBS-BSA 1% - Tween 20 0.05%). Next, anti-human IgG and Anti human IgA secondary antibodies conjugated with Alkaline Phosphatase (Southern Biotech) were added for 45 min. To detect bacteria-mAbs binding, pNPP (β-nitrophenyl phosphate; Sigma-Aldrich) was used as soluble substrate and the final reaction was quantified at a wavelength of 405 nm by using the Varioskan Lux Reader (Thermo Fisher Scientific). After each incubation step, plates were washed three times with 100 μl per well of washing buffer (phosphate-buffered saline and 0.05% Tween-20). Sample buffer was used as a blank and we considered as positive hits those wells showing an OD₄₀₅ value of at least 2-fold superior to blank. Cells corresponding to positive hits were lysed in 25 μL of a buffer containing RNAsi Out 0.2 U/μL, ultrapure BSA 1 mg/mL and H₂O DEPC (ThermoFisher Scientific) and stored at −80° C. for following steps.

2.2. Single-Cell RT-PCR and Nested PCR were Used to Amplify V_{H} and V_{L}

cDNA was synthesized from 5 μL of MBC lysates. Reverse transcription (RT) reaction was performed by adding 25 μL per well of a mix containing 1 μL of random hexamer primers (50 ng/mL),1 μL of dNTPs (10 mM), 2 μL 0.1 μM DTT, 40 U/L RNase OUT, MgCl₂ (25 mM), 5 μL of 5× buffer, 0.25 μL of Superscript IV reverse transcriptase (Invitrogen) and nuclease-free water (DEPC) and RT-PCR conditions were 42° C./10 min, 25° C./10 min, 50° C./60 min and 94° C./5 min. After cDNA synthesis, two additional rounds of PCR were performed to obtain the variable regions of the heavy (V_{H}) and light (V_{L}) chains. Briefly, in the first round of PCR (PCR I) a total volume of 25 μL containing 4 μL of cDNA, 10 μM of VH or 10 μM VL/VK primer mix (Table 4), 0.5 μL of dNTPs (10 mM), 1.5 μL MgCl₂ (25 mM), 5 μL of 5× Kapa Long Range Buffer, and 0.125 μL of Kapa Long Range Polymerase (Sigma) was added in each well and amplified using the following conditions: 95° C./3′, 5 cycles at 95° C./30”, 57° C./30”, 72° C./30” and 30 cycles at 95° C./30”, 60° C./30”, 72° C./30” and 72° C./2′. 3 μL of un-purified PCR I products were used as a template for the nested PCR (PCR II) using the same cycling conditions and primers indicated in Table 5. PCR II products were then purified by Millipore MultiScreen PCR 96 plate according to the manufacturer's instructions. Samples were eluted in 30 μL nuclease-free water pre-warmed at 50° C. and quantified by NanoDrop One (Thermo Fisher Scientific).

TABLE 4
List of primers mix used for PCR I.

| NAME | SEQUENCE | SEQ ID |
|---|---|---|
| L-VH1_VH7 fw | CACTCCCAGGTGCAGCTGGTGCAG | SEQ ID NO: 451 |
| L-VH2 fw | TGGGTCTTRTCCCAGGTCACCTTG | SEQ ID NO: 452 |
| L-VH 3fw | AAGGTGTCCAGTGTSAGGTGCAG | SEQ ID NO: 453 |
| L-VH4_6 fw | GTCCTGTCCCAGGTGCAGCTGCAG | SEQ ID NO: 454 |
| L-VH5 fw | GAGTCTGTTCCGAGGTGCAGCTGG | SEQ ID NO: 455 |
| IgG CH rev | GTGCCAGGGGGAAGACCGATG | SEQ ID NO: 456 |
| IgA CH rev | GCMGAGGCTCAGCGGGAAGAC | SEQ ID NO: 457 |
| IgM CH rev | GAGACGAGGGGGAAAAGGGTTG | SEQ ID NO: 458 |
| L-VK1 fw | CAGGTGCCAGATGTGHCATCCAG | SEQ ID NO: 459 |
| L-VK2 fw | CTGGATCCAGTGSGGATATTGTGATG | SEQ ID NO: 460 |
| L-VK3 fw | CCCAGATACCACCGGAGAAATTGTG | SEQ ID NO: 461 |
| L-VK4 fw | CTCTGGTGCCTACGGGGACATCGTG | SEQ ID NO: 462 |
| L-VK5 fw | CTGATACCAGGGCAGAAACGACAC | SEQ ID NO: 463 |
| CK rev 1st | GAACACTCTCCCCTGTTGAAGCTCTTTG | SEQ ID NO: 464 |
| L-VL1 fw | GGTCCTGGGCCCAGTCTGTGCTG | SEQ ID NO: 465 |
| L-VL2 fw | GGTCCTGGGCCCAGTCTGCCCTG | SEQ ID NO: 466 |
| L-VL3 fw | TCTGTGRCCTCCTATGAGCTGAC | SEQ ID NO: 467 |
| L-VL4_VL5_VL9 fw | CTCTCGCAGCCTGTGCTGACTCA | SEQ ID NO: 468 |
| L-VL6 fw | GTTCTTGGGCCAATTTTATGCTG | SEQ ID NO: 469 |
| L-VL7 fw | GGTCCAATTCTCAGGCTGTGGTG | SEQ ID NO: 470 |
| L-VL8 fw | GAGTGGATTCTCAGACTGTGGTG | SEQ ID NO: 471 |
| L-VL10 fw | GTCAGTGGTCCAGGCAGGGCTGAC | SEQ ID NO: 472 |
| CL rev 1st | GTGCTCCCTTCATGCGTGACC | SEQ ID NO: 473 |

TABLE 5
List of primers mix used for PCR II.

| NAME | SEQUENCE | |
|---|---|---|
| HIFI*_C134_VH1_5_7 | GT ATC ATC CTT TTT CTA GTA GCA ACT GCA ACC GGT | SEQ ID NO: 474 |
| | GTACATTCC CAG GTG CAG CTG GTG CAG TCT G | |
| HIFI*_C134_VH2 | GT ATC ATC CTT TTT CTA GTA GCA ACT GCA ACC GGT | SEQ ID NO: 475 |
| | GTACATTCC CAG GTC ACC TTG AAG GAG TCT GGT C | |
| HIFI*_C134_VH3 | GT ATC ATC CTT TTT CTA GTA GCA ACT GCA ACC GGT | SEQ ID NO: 476 |
| | GTACATTCC GAG GTG CAG CTG GTG GAG TCT GGG GGA G | |
| HIFI*_C134_VH4_6_a | GT ATC ATC CTT TTT CTA GTA GCA ACT GCA ACC GGT | SEQ ID NO: 477 |
| | GTACATTCC CAG GTG CAG CTG CAG GAG TCG GG | |
| HIFI*_C134_VH4_6_b | GT ATC ATC CTT TTT CTA GTA GCA ACT GCA ACC GGT | SEQ ID NO: 478 |
| | GTACATTCC CAG GTG CAG CTG CAG CAG TGG GG | |
| HIFI*_IgG_CH rev | CTT GGA GGA GGG TGC CAG GGG GAA GAC CGA TGG GCC | |
| | CTT GGT GGA RGC | |
| HIFI*_IgA_CH rev | CTT GGA GGA GGG TGC CAG GGG GAA GAC CGA CTT GGG | SEQ ID NO: 480 |
| | GCT GGT CGG GGA | |
| HIFI*_IgM_CH rev | CTT GGA GGA GGG TGC CAG GGG GAA GAC CGA TGG GGC | SEQ ID NO: 481 |
| | GGA TGC ACT CCC | |
| HIFI*_C135_VK1 | GT ATC ATC CTT TTT CTA GTA GCA ACT GCA ACC GGT | SEQ ID NO: 482 |
| | GTACATTCC GCC ATC CAG ATG ACC CAG TCT CCA TC | |
| HIFI*_C135_VK2_a | GT ATC ATC CTT TTT CTA GTA GCA ACT GCA ACC GGT | SEQ ID NO: 483 |
| | GTACATTCC GAT ATT GTG ATG ACC CAG ACT CCA CTC TC | |
| HIFI*_C135_VK2_b | GT ATC ATC CTT TTT CTA GTA GCA ACT GCA ACC GGT | SEQ ID NO: 484 |
| | GTACATTCC GAT ATT GTG ATG ACT CAG TCT CCA CTC TC | |
| HIFI*_C135_VK3_a | GT ATC ATC CTT TTT CTA GTA GCA ACT GCA ACC GGT | SEQ ID NO: 485 |
| | GTACATTCC GAA ATT GTG TTG ACA CAG TCT CCA G | |
| HIFI*_C135_VK3_b | GT ATC ATC CTT TTT CTA GTA GCA ACT GCA ACC GGT | SEQ ID NO: 486 |
| | GTACATTCC GAA ATT GTG ATG ACG CAG TCT CCA G | |
| HIFI*_C135_VK4 | GT ATC ATC CTT TTT CTA GTA GCA ACT GCA ACC GGT | SEQ ID NO: 487 |
| | GTACATTCC GAC ATC GTG ATG ACC CAG TCT CCA G | |
| HIFI*_C135_VK5 | GT ATC ATC CTT TTT CTA GTA GCA ACT GCA ACC GGT | SEQ ID NO: 488 |
| | GTACATTCC GAA ACG ACA CTC ACG CAG TCT CCA G | |
| HIFI*_C080_VK_Rev | GA TTT CAA CTG CTC ATC AGA TGG CGG GAA GAT GAA | SEQ ID NO: 489 |
| | GAC AGA TGG TGC AGC CAC AGT TC | |
| HIFI*_C080_VL1 | GT ATC ATC CTT TTT CTA GTA GCA ACT GCA ACC GGT | SEQ ID NO: 490 |
| | TCCTGGGCC CAG TCT GTG CTG ACT CAG CCG CCC TCA G | |
| HIFI*_C080_VL2 | GT ATC ATC CTT TTT CTA GTA GCA ACT GCA ACC GGT | SEQ ID NO: 491 |
| | TCCTGGGCC CAG TCT GCC CTG ACT CAG CCT GCC TCC G | |
| HIFI*_C080_VL3_a | GT ATC ATC CTT TTT CTA GTA GCA ACT GCA ACC GGT | SEQ ID NO: 492 |
| | TCCTGGGCC TCC TAT GAG CTG ACA CAG CCA C | |
| HIFI*_C080_VL3_b | GT ATC ATC CTT TTT CTA GTA GCA ACT GCA ACC GGT | SEQ ID NO: 493 |
| | TCCTGGGCC TCC TAT GAG CTG ACT CAG GAC C | |
| HIFI*_C080_VL4 | GT ATC ATC CTT TTT CTA GTA GCA ACT GCA ACC GGT | SEQ ID NO: 494 |
| | TCCTGGGCC CAG CCT GTG CTG ACT CAA TCG TCC TCT G | |
| HIFI*_C080_VL5-9 | GT ATC ATC CTT TTT CTA GTA GCA ACT GCA ACC GGT | SEQ ID NO: 495 |
| | TCCTGGGCC CAG CCT GTG CTG ACT CAG CCR ACT TC | |
| HIFI*_C080_VL6 | GT ATC ATC CTT TTT CTA GTA GCA ACT GCA ACC GGT | SEQ ID NO: 496 |
| | TCCTGGGCC AAT TTT ATG CTG ACT CAG CCC CAC TC | |
| HIFI*_C080_VL7 | GT ATC ATC CTT TTT CTA GTA GCA ACT GCA ACC GGT | SEQ ID NO: 497 |
| | TCCTGGGCC CAG GCT GTG GTG ACT CAG GAG CCC TC | |
| HIFI*_C080_VL8 | GT ATC ATC CTT TTT CTA GTA GCA ACT GCA ACC GGT | SEQ ID NO: 498 |
| | TCCTGGGCC CAG ACT GTG GTG ACC CAG GAG CCA TC | |
| HIFI*_C080_VL10 | GT ATC ATC CTT TTT CTA GTA GCA ACT GCA ACC GGT | SEQ ID NO: 499 |
| | TCCTGGGCC CAG GCA GGG CTG ACT CAG CCA CCC TCG G | |
| HIFI*_CL_generic | G TGT GGC CTT GTT GGC TTG AAG CTC CTC ACT CGA GGG | SEQ ID NO: 500 |
| | YGG GAA CAG AGT G | |

2.3. Cloning of V_{H} and V_{L} and Recombinant Antibody Expression by TAP Transfection

Amplified antibody sequences were ligated into human IgG1 expression vectors and used to produce transcriptionally active PCR (TAP) products. Briefly, IgG1, Igκ or Igλ expression vectors were digested with AgeI, SalI and XhoJ restriction enzymes, respectively. 25 ng of linearised plasmids were ligated with 75 ng of purified V_{H} and V_{L} by Gibson Assembly (New England BioLabs). The reaction was performed in a final volume of 5 μL. The ligation product was 10-fold diluted in DEPC water and used as template for transcriptionally active PCR (TAP) reaction which allowed the direct use of linear DNA fragments for in vitro expression. Functional promoter (human CMV) and terminator sequences (SV40) were attached onto PCR 11 products directly by amplification using specific primers (Table 6).

TABLE 6
List of primers mix used for PCR II.

| NAME | SEQUENCE | SEQ ID |
|---|---|---|
| CMV_TAP_FW | TTAGGCACCCCAGGCTTTAC | SEQ ID NO: 501 |
| polyA_TAP_ | AGATGGTTCTTTCCGCCTCA | SEQ ID NO: 502 |
| Rev | | |

TAP-PCR was performed in a total volume of 25 μL containing 0.25 μL of Q5 polymerase (NEB), 5 μL of GC Enhancer (NEB), 5 μL of 5× buffer, 0.5 μL dNTPs (10 mM), 0.125 L of forward/reverse primers and 3 μL of ligation product, and using the following conditions: one step of 98° C./2′, 35 cycles of 98° C./10”, 61° C./20”, 72° C./1′ and an extension of 72° C./5′. Once purified and quantified, the TAP products were transfected into Expi293F cells to allow small scale production of recombinant mAbs. Briefly, cells were transfected with both TAP amplifications in a final volume of 1 ml into 96 deep well plate (Eppendorf), and after 7 days of expression mAb-containing supernatants were harvested by centrifugation.

2.4. Quantification of TAP-Produced mAbs by ELISA

To quantify the concentration of mAbs in each supernatant, ELISA plates were coated with 2μg/ml of goat anti-human IgG (Southern Biotech) at 4° covernight. Plates were washed 3 times with PBS plus Tween20 0.05%, blocked in PBS plus BSA 1% at 37° C. for 1 h. Then samples were washed and incubated with TAP-produced mAbs diluted in sample buffer (PBS 1×- BSA 1%- Tween20 0.05%) at 37° C. for 1 h. Following additional washings and incubation at 37° C. for 1 h with AP-conjugated anti-goat IgG secondary antibody (Southern Biotech), absorbance was read by addition of PNPP. Concentrations were evaluated by linear regression analysis built by potting OD₄₀₅ values against a standard curve generated by titration of a human IgG-unlabelled antibody (Southern Biotech).

2.5. Large Scale Expression and Purification of mAbs

Expi293F cells (Thermo Fisher) were transiently transfected with plasmids carrying the heavy and the light chains of each antibody with a 1:2 weight/weight ratio. Cells were grown for six days at 37° C. at 8% CO₂ and 125 rpm shaking, with an optimized cocktail of enhancers 1 and 2 (ThermoFisher) added on day 1 post-transfection. Two mAb harvests were performed on the third and sixth day by pelleting the cells at 1,100×g for 10 min at RT and supernatants were pooled and clarified by centrifugation (3,000×g for 15 min at 4° C.), followed by 0.45 mm filtration. mAbs were purified at RT by affinity chromatography on the AKTAgo purification system (Cytiva) using the HiTrap Protein G HP column (Cytiva), which binds to the Fc domain. Specifically, the column was equilibrated in 0.02 μM sodium phosphate buffer pH 7 at a flow rate of 1 mL/min, which was used also for the following steps. After sample injection, the column was washed with 10 column volumes (CV), followed by mAb elution with 10 CV of 0.1 μM glycine-HCl, pH 2.7. mAb pool was dialyzed in PBS pH 7.4 using Slide-A-Lyzer G2 Dialysis Cassette 3.5K (Thermo Scientific) overnight at 4° C. For each purified antibody the concentration was determined by measuring absorbance at 280 nm at Nanodrop. All purified antibodies were aliquoted and stored at −80° C.

### 2.6. Serum Bactericidal Assay (SBA)

Two days prior to the assay, glycerol stocks of Kp were streaked on LB-Agar plates and incubated overnight (ON) at 37° C. The following day, a single colony was inoculated in 4 mL of LB and incubated ON at 37° C. Cultures were expanded to 10 ml LB in a 125 ml flask to obtain an OD₆₀₀ of 0.05 and incubated at 37° C. with shaking until the exponential phase (OD₆₀₀ 0.4-0.6) was reached. Bacteria were then 1:10 diluted in PBS.

Appropriate baby rabbit complement (BRC) concentrations to be used in the assay were established through complement sensitivity tests. Briefly, 2×10⁵ bacteria resuspended in PBS were seeded in round bottom 96-well plates, and BRC concentrations were screened starting from 50% in 11 serial 2-fold step dilutions. After 2 h incubation at 37° C., bacteria were pelleted by centrifugation. The supernatant was discarded, and bacteria were resuspended in 30 μl of PBS and transferred into a White Optiplate (Perkin Elmer). 30 μl of BacTiter-Go™ 1× (Promega) was added to each well and luminescence was measured by the Varioskan Lux microplate reader (Thermo Fisher Scientific) with an exposure time of 500 ms. Data were plotted and analysed using GraphPad Prism. 12.5% BRC was established as a universal BRC concentration allowing to achieve complement-based antibody killing without significant toxicity.

For luminescence-based SBA (L-SBA) with TAP-expressed mAbs, 2×10⁶ bacteria/mL of ST 147_{NDM-1} were seeded into a 96-well U-round bottom plate in the presence of 12.5% BRC, with the addition of four serial dilutions of TAP-expressed mAbs (1:10, 1:50: 1:250: 1:1250), in a total volume of 100 μl per well. Upon 2 hours incubation at 37° C., bacteria were pelleted by centrifugation at 4000×g. Bacterial pellets were resuspended in 30 μl of PBS per well and transferred into a white Optiplate (Perkin Elmer). 30 μl of BacTiter-Go™ 1× (Promega) was added to each well and luminescence was measured by the Varioskan Lux microplate reader (Thermo Fisher Scientific) with 500 ms exposure. In each experiment, luminescence values obtained were used to calculate the median value for each dilution factor. Difference between the luminescence signal of each mAb and the median value was measured and plotted as percentage with 30% cut-off.

For fluorescence-based SBA (F-SBA), 2×10⁶ bacteria/mL in PBS were seeded in 384-well black clear-bottom plate (ViewPlate®-384 F TC, PerkinElmer) in the presence of 12.5% BRC, in a final volume of 50 μl per well. Purified recombinant mAbs were added in a 3-fold step serial dilution panel. After 2 hours incubation at 37° C., 40 μl LB and 10 μl of 0.025% resazurin (Sigma-Aldrich) were added to each well. Fluorescence (λ_{Ex} 560 nm and λ_{E}m 590 nm). Exposition: 250 ms. Wide 12 nm) was measured by the Varioskan Lux microplate reader (Thermo Fisher Scientific) upon 2 hours incubation at 37° C. GraphPad Prism was used to plot and analyze data, as well as to extrapolate IC₅₀ values.

### 2.7. Flow Cytometry Analysis of mAb Binding to Bacterial Surface

Binding of mAbs to the panel of bacterial strains (Table S2) was performed on bacteria in the exponential phase of growth in LB (OD₆₀₀ 0.4-0.6). Bacteria were pelleted and resuspended in an equal volume of PBS- BSA 1%. 100 μL of bacteria were plated in each well of a round bottom 96-well plates by centrifuging at 4000×g for 5 min, followed by incubation with 5 μg/ml of purified antibodies at RT for 1 h. Plates were centrifuged, and pellets were washed three times with PBS-BSA 1% and incubated at RT for 30 min in the dark with 1:2000 diluted Alexa488-conjugated α-human IgG secondary antibody. After an additional centrifugation and washing step, bacteria were fixed with 0.5% of paraformaldehyde at RT for 30 min, washed again, resuspended in PBS-BSA 1% to an OD₆₀₀ of 0.05 and read by their fluorescence. Samples were acquired on the BD FACSCanto II (BD Biosciences, USA). Data were analysed by FlowJo software v 10 (BD Biosciences, USA).

### 2.8. Antibody Sequence Analysis

Immunoglobulin genes were identified using a custom python script that employs NCBI IgBlast and IMGT nomenclature (in the time between 2020 and 2022). IGHV gene somatic hypermutations were counted from the start of FWR1 until the end of FWR3. Insertions or deletions were counted as one single mutation.

### 2.9 Genomic Analysis of Kp Isolates.

ST147 strains were sequenced using both short- and long-reads technology. High-throughput-sequencing was performed on the MiSeq platform (Illumina; San Diego, CA, USA) with a paired-end layout of 150 bp. Paired-end short reads were quality-checked and poor-quality reads were filtered using fastp v0.20.1 (REF). The long-read library was prepared with multiplexing and sequenced according to the manufactures' guide using flow cell R9.4.1 (Nanopore). The quality of long reads was controlled by being mapped with their corresponding short reads using Filtlong v0.2.0 (https://github.com/rrwick/Filtlong) with minimum quality and length as Q8 and 2,000 bp respectively. Along with the corresponding clean reads, long reads were fed into the hybrid assembler Unicycler v0.4.8 (REF) and run under the conservative mode. ST and capsule typing prediction was performed using Kleborate v2.0.0 (REF) and Kaptive (REF).

### 2.10 Western Blot Analysis of mAb Binding to Kp Lysates

For sample preparation, bacteria from glycerol stocks were grown overnight on LB-agar plates Single colonies of Kp have been picked from plates and grown overnight with static incubation at 37° C. in LB. The following day, bacteria were grown at 37° C. starting from OD 0.05 at 150 rpm in LBuntil they reached exponential phase (OD 0.4-0.6). Polysaccharide extracts for immunoblotting has been prepared with lipopolysaccharide (LPS) extraction kit (Sigma). Total bacterial lysates were prepared by centrifuging inoculum for 10 min at 2500 g at 4° C., followed by filtration at 14000 g with Nanosep 0.2 μm columns. Part of the sample was treated with proteinase K for 1 hour at 60° C. to eliminate proteins. SDS-PAGE samples were prepared by adding 1:4 loading buffer and 1:10 reducing agent and incubated for 5 minutes at 95° C. SDS-PAGE gel was transferred onto PVDF membrane using the iBlot™ Gel Transfer Device and Stacks (Termofisher). Membranes were blocked for 1 h at room temperature in TBS 1×/0.1% Tween-20/5% milk. Purified mAbs were used at 1μg/mL in TBS 1×/0.1% Tween-20/5% milk. Following an overnight incubation at 4° C., membranes were washed 3 times with TBS 1×/0.1% Tween. Incubation with secondary antibody (goat anti-human Fab) diluted 1:75000 in TBS 1×/0.1% Tween/5% milk was carried out for 1 hour at room temperature. Membranes were washed 3 times in TBS 1×/0.1% Tween and then developed with chemiluminescence readout.

### 2.11 Characterization of mAb Binding by High-Resolution and High-Content Confocal Microscopy

A single colony of bacteria stably expressing super-folder(sf)mCherry was picked from an LB-agar plate and grown ON in LB with 150 g/mL hygromycin. The overnight culture was diluted in LB without antibiotics and grown to ODs 0.025 or 0.05. Then, 50 mL of bacteria were transferred into a 96-well Phenoplate (Perkin Elmer, 6055300) and incubated for 2 h at 37° C. without CO₂ in static conditions. Subsequently, the supernatant was discarded and the adherent bacteria were fixed in 4% paraformaldehyde (PFA)/PBS (Thermo Fisher) or Cytofix (BD) for 15 minutes at RT.

For experiments aimed at staining a single anti-Kp mAb, selected antibodies were diluted in a solution of PBS/1% BSA (Bovine Serum Albumin) at a concentration of 0.5 g/mL and incubated for 30 minutes at RT. Then, samples were washed in PBS and a mixture of goat anti-Human Alexa488 conjugated secondary antibody (diluted 1:2000) and DAPI (diluted 1:2000) in PBS/BSA1% was added for 30 minutes at RT. Finally, samples were washed in PBS and 50 μL of 1% Low Melting (LM) agarose were distributed in each well. Samples were stored at 4° C. and imaging was performed within the following 24 hours.

For experiments in which O8O09, 05N02 and O5D08 mAbs were simultaneously stained, antibodies were conjugated with Alexa488, Alexa555 and Alexa647 fluorophores, respectively, using the Zip Alexa Fluor™ Rapid Antibody Labeling Kits (Thermo Fisher) following manufacturer instructions. After fixation in PFA, samples were first blocked in PBS/1% BSA for 30 minutes at RT and then incubated with a mixture of fluorophore-conjugated mAbs (each one at 0.5 mg/mL in PBS/1% BSA) and DAPI (1:2000). Following 30 minutes incubation at RT, samples were washed in PBS and prepared for imaging by adding 50 μL of 1% LM agarose. On the following day, samples were imaged.

### 2.12 Opsonophagocytosis Assay

THP1 cells (ATCC) were maintained in RPMI 1640 containing GlutaMax (Thermo Fisher) and complemented with 10 mM Hepes (Thermo Fisher), 1 mM Sodium Pyruvate (Thermo Fisher) and 10% fetal bovine serum (FBS) (Thermo Fisher). Three days before the assay, 50.000 cells per well were seeded into a 96-well black-shielded optiplate (Perkin Elmer) in the presence of 20 ng/mL phorbol-12-myristate-13-acetate (PMA) to promote monocyte differentiation into macrophages. The day after, PMA was washed out and cells were maintained in fresh medium for two additional days.

Infection of macrophage was performed using ST147_{NDM1}-sfmCherry expressing Kp. Bacteria were grown ON and re-launched the following morning to reach OD₆₀₀ 0.5. After centrifugation, bacteria were resuspended in phagocytosis media (RPMI 1640+GlutaMax, 10 mM Hepes, 1 mM Sodium Pyruvate) to have a 1:2 dilution of the initial culture volume. To allow bacterial opsonization, mAbs dilutions were prepared in 25 mL of phagocytosis media and incubated with 25 mL of bacteria for 30 minutes at 37° C. in shaking conditions (500 rpm). This mixture was then added to differentiated macrophages and centrifuged for 3 minutes at room temperature to synchronize the infection. Following the incubation of 1 hour at 37° C. in the presence of 5% CO2, samples were treated for an additional hour with 150 mg/mL streptomycin to kill not engulfed bacteria. Finally, samples were incubated for 5 minutes at room temperature with PBS/0.1% X100-Triton to permeabilize cells and allow the release of internalised bacteria in the supernatants. Bacterial fluorescence was read using a Varioskan Lux microplate reader (Thermo Fisher) as a readout of the uptaken bacteria.

### 2.13 Live Bacterial Imaging

ST147_{NDM1}-sfmCherry was grown ON as described before and re-launched until OD₆₀₀ 0.5. The assay was carried out in a final volume of 50 mL containing exponential Kp diluted 1:100, 12.5% BRC and escalating doses of anti-Kp mAbs in PBS. Samples were prepared in 96-well Phenoplate (Perkin Elmer, 6055300) and briefly centrifuged at 1000 rpm to keep bacteria closer to the bottom of the well. Acquisition started immediately after centrifugation and was carried out at 37° C. by acquiring frames every 2 minutes for 2 hours. 2.14 Microscopy and image analysis Imaging was performed using the Opera Phenix platform (Perkin Elmer) and all samples were acquired using a 63× N.A. 1.15 water objective.

For mAb binding experiments carried out in fixed conditions, 20 fields of view (FOV)/well were selected. For each FOV, five z-stacks separated by a z-step of 0.5 m were acquired. Imaging was performed in the confocal mode by exciting the samples with lasers at 425 nm, 488 nm and 561 nm and collecting the emitted light using bandpass filters 435-480 nm, 500-550 nm and 570-630 nm, respectively.

For bacterial live imaging experiments, 3 FOV/well were acquired and three z-stacks separated by a z-step of 0.5 m were imaged. Imaging was performed in the widefield mode by exciting the samples with lasers at 561 nm and collecting the light using the bandpass emission filter 570-630 nm.

Images were analyzed with Harmony (v4.9), provided by Perkin Elmer, by using custom-made image analysis pipeline described in supplementary materials. Individual bacteria were detected using the DAPI channel and filtered according to their morphology based on data available in literature. To measure mAb intensity levels, the mean intensity of the A488 signal was measured within a ROI drawn around each bacterium. To measure mAb occupancy area, A488 spots were detected, and their morphology features were measured. A488 spots displaying an area smaller than 0.5 mm² were discarded from the analysis because they were not representative of the distribution of the mAb signals around the bacteria.

### 2.15 Purification of ST147_{NDM-1} Capsule

For capsule extraction, ST147_{NDM-1} Kp from glycerol stocks was grown at 37° C. overnight in LB in the presence of meropenem. 200 μL of liquid culture were plated on carbohydrate-rich Worfel-Ferguson agar plates to increase Kp capsule production. The Worfel-Ferguson agar plates contain 0.2% yeast extract, 0.2% sodium chloride, 0.1% potassium sulphate, 0.025% magnesium sulphate, 2% sucrose, and 1.5% Agar. Upon overnight incubation at 37° C., bacteria were mechanically collected from the plates and resuspended in LB. The samples were centrifuged for 8 minutes at 4000 rpm, pellets were collected and resuspended in H₂O. After a 6 h incubation at 99° C., samples were centrifuged for 8 minutes at 4000 rpm. Supernatants were collected and filtered with 0.22 μM filters. To allow DNA precipitation, 1% CTAB, 5 mM Na₂SO₄ and 0.24 mM NaCl were added to the solution. Samples were vortexed and incubated at 100° C. with 550 rpm for 4 h, then centrifuged for 15 minutes at 14000 rpm. Supernatants were collected, diluted 1:10 with 5 mM Na₂SO₄, and incubated at 37° C. for 1 h. Upon 15 minutes of centrifugation at 4000×g, the pellet was resuspended in 4 mL of 1M CaCl₂. To remove impurities, 25% of EtOH was added to the sample and left on a tilting plane for 30 minutes at room temperature. The sample was centrifuged for 15 minutes at 4000 xg and the supernatant was collected. To allow capsule precipitation, 80% of EtOH was added to the collected supernatant and left for 1 h at room temperature on a tilting plane. Incubation was followed by centrifugation for 15 minutes at 4000 xg. The obtained pellet was resuspended in 1 mL of 1M NaCl and purified with Amicon 10K filters with at least 10 washing steps in MilliQ H₂O to remove protein contaminants and other impurities. Sugar content was evaluated by phenol-H₂SO₄ assay, protein content was estimated by microBCA (Thermo Fisher) and DNA presence was measured using the Qubit kit (add more info on the kit). Protein and DNA contaminants were <1% compared to the sugar concentration, quality of the estimated molecular size distribution was done by SEC-HPLC, with a Tosoh TSK gel PWXL guard—G3000 PWXL columns connected in series (4.0 cm×6.0 mm; cod. 808033, and 30 cm×7.8 mm; cod. 808021, respectively), as a running buffer, 0.1 μM NaCl, 0.1 μM NaH₂PO₄, pH 7.2

2.16 Purification of β-Antigen from Kp Strains

O-antigen was extracted based on the published protocol (https://pubmed.ncbi.nlm.nih.gov/32959249/) with slight modifications. 300 ml of LB supplemented with 0.4% glucose, were inoculated with Kp, after reaching logarithmic growth, bacteria were pelleting at 4,000 rpm for 15 min. SN was discarded and the pellet was resuspended in 15 ml of 3% acetic acid, solutions were incubated for at least 4 h at 90° C. shaking at 500 rpm, after that SN was collected and neutralized its pH with 100 mM NaOH. Samples were desalted using Disposable PD 10 (Cytiva). 100 mM of citrate buffer pH 2.7 was added with continuous stirring to achieve the final citrate concentration of 20 mM, incubated at RT for 30 min, then centrifuged at 12,000 rpm at 15° C. for 30 min, with the SN a cation-exchange purification was performed with Sartobind MA75 (Sartorius). After neutralization follow by a second desalt step, anion exchange was performed with HiTrap Q FF (Cytiva) by AKTA GO (GE Healthcare Life Sciences). The column was previously equilibrated with 5 CV Buffer A (10 mM Tris-HCl pH 8), the sample was diluted to 100 mL to a final concentration of Buffer A and loaded with the sample pump. The column was washed with 5 CV of Buffer A, and elution was performed with 10 CV of Buffer B (10 mM TRIS pH8+200 mM NaCl). The flow rate for all steps was 5 mL/min. O-antigen was eluted in the flow through, concentrated by Amicon 10K. Total sugar quantification was measured by phenol-sulfuric assay (https://pub.acs.org/doi/10.1021/ac60111a017), and quality of the estimated molecular size distribution was done by SEC-HPLC, with a Tosoh TSK gel PWXL guard—G3000 PWXL columns connected in series (4.0 cm×6.0 mm; cod. 808033, and 30 cm×7.8 mm; cod. 808021, respectively), as a running buffer, 0.1 μM NaCl, 0.1 μM NaH₂PO₄, pH 7.2. Detection was performed with a UV and a refraction index detector. Protein impurities were measured by microBCA (Thermo Fisher).

### 2.17 ELISA on Purified Kp O-Antigen and Capsule

To perform the ELISA, LPS was used with O2a and O2afg antigen, already characterized and kindly provided by Chris Whitfield's group, in addition, internal purified material, capsule and O antigen were used to coat high-binding 384-well plates (Greiner ref 781061) and incubated at 4° C. overnight. The next day, plates were blocked in PBS plus 1% BSA at 37° C. for 1 h. After blocking, plates were incubated with the primary antibody (produced in-house) at a final concentration of 10 μg/ml for 2 h at RT, in the presence of PBS, 1% BSA, and 0.05% Tween-20. Next, anti-human IgG secondary antibodies conjugated with Alkaline Phosphatase (Southern Biotech) were added for 1 h at 37° C. To detect antigen-mAbs binding, pNPP (β-nitrophenyl phosphate; Sigma-Aldrich) was used as a soluble substrate, and the final reaction was quantified at a wavelength of 405 nm using the Varioskan Lux Reader (Thermo Fisher Scientific). After each incubation step, plates were washed three times with 100 μl per well of a washing buffer (PBS plus 0.05% Tween-20). Sample buffer and an unrelated mAb were used as a blank, and we considered positive hits those wells showing an OD₄₀₅ value 3-fold higher than the blank. As a positive control, a 1:100 dilution of a mix of plasma was used.

### 2.18 Immunocompetent ST147_{NDM-1} Bacteraemia Model

This was done in the laboratory of Dr. David P. Nicolau and Dr. Kamilia Abdelraouf, at the Center for Anti-infective Research and Development Hartford Hospital. O8O09, 05N02 and 05D08 were reconstituted with PBS to the concentrations required to deliver 1, 5, 10, and 20 mg/kg doses based on the mean weight of the study mice population. For prophylaxis (PRO) studies, single dose mAb at examined doses was administered through intraperitoneal (IP) route 24 h prior to bacterial inoculation. For treatment (THR) studies, single dose mAb at examined doses was administered through intravenous (IV) route 1 h post bacterial inoculation. For prophylaxis plus treatment (PRO+THR) studies, one dose of mAb was administered IP 24 h prior to bacterial inoculation, and a second dose of the same mAb at the same dose was administered IV 1 h post bacterial inoculation. All dosing solutions were kept on ice while filled syringes with dosing solutions were refrigerated until use. PBS solution with a pH of 7.4 was utilized as the vehicle for dosing control animals throughout the study.

Specific pathogen-free, female ICR mice weighing 20-22 grams were obtained from Charles River Laboratories, Inc., (Wilmington, MA). The animals were allowed to acclimate for a minimum of 48 h before commencement of experimentation and were provided food and water ad libitum. The protocol was reviewed and approved by the Institutional Animal Care and Use Committee at Hartford Hospital. Mice were administered uranyl nitrate 5 mg/kg three days prior to inoculation to produce a controlled degree of renal impairment. In total, 322 mice were used in the experiments described in this report. ST147_{NDM-1} was previously frozen at - 80° C. in skim milk (BD BioSciences, Sparks, MD). Prior to mice inoculation, two transfers of the organisms were performed onto Trypticase Soy Agar plates with 5% sheep blood (TSA II™; Becton, Dickinson & Co.; Sparks, MD) and incubated at 37° C. for approximately 24 h. After 18-24 h of incubation of the second transfer, a bacterial suspension of approximating the target number of colony forming units (CFU)/ml in 5% hog gastric mucin was made for inoculation. Final inoculum concentrations were confirmed by serial dilution and plating techniques. Septicemia was produced by IP injection of 0.5 ml of the inoculum.

### Sequence Listing in the Description

### Sequence of the Antibody Herein Identified as SBJ03-F18

### Amino Acid Sequences of SBJ03-F18:

>CDR 1 of variable domain of Heavy chain of
SBJ03-F18
SEQ ID NO: 1
GFTFSSYS
>CDR 2 of variable domain of Heavy chain of
SBJ03-F18
SEQ ID NO: 2
ISGNSNYI
>CDR 3 of variable domain of Heavy chain of
SBJ03-F18
SEQ ID NO: 3
ARGTIIGAAGYDC
>CDR 1 of variable domain of Light chain of
SBJ03-F18
SEQ ID NO: 4
QDISTS
>CDR 2 of variable domain of Light chain of
SBJ03-F18 this sequence is not included in the
sequence listing because is less than 4 amino
acid
SEQ ID NO: 5
AAS (Ala-Ala-Ser)
>CDR 3 of variable domain of Light chain of
SBJ03-F18
SEQ ID NO: 6
QQLKSYPLA
>variable domain of Heavy chain of SBJ03-F18
SEQ ID NO: 7
EVQLVESGGGLVKPGGSLRLSCAASGFTFSSYSMNWVRQAPGKGLEWVSS
ISGNSNYIYYADSVKGRFTISRDNAKNSLYLQTSSLRAEDTALYFCARGT
IIGAAGYDCWGQGTLVTVSS
>variable domain of Light chain of SBJ03-F18
SEQ ID NO: 8
AIQMTQSPSFLSASVGDRVTLTCRASQDISTSLAWYHQKPGKAPERLVYA
ASTLQSGVPSRFSGSGSGTGFTLTISSLQPEDFGTYYCQQLKSYPLAFGG
GTKVEIK
>Heavy chain of SBJ03-F18
SEQ ID NO: 9
MGWSCIILFLVATATGVHSEVQLVESGGGLVKPGGSLRLSCAASGFTFSS
YSMNWVRQAPGKGLEWVSSISGNSNYIYYADSVKGRFTISRDNAKNSLYL
QTSSLRAEDTALYFCARGTIIGAAGYDCWGQGTLVTVSSASTKGPSVFPL
APSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPP
CPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVM
HGALHNHYTQKSLSLFPG*
>Light chain of SBJ03-F18
SEQ ID NO: 10
MGWSCIILFLVATATGVHSAIQMTQSPSFLSASVGDRVTLTCRASQDIST
SLAWYHQKPGKAPERLVYAASTLQSGVPSRFSGSGSGTGFTLTISSLQPE
DFGTYYCQQLKSYPLAFGGGTKVEIKRTVAAPSVFIFPPSDEQLKSGTAS
VVCLLNNFYPREAKVQWKVDNALQSGNSQESVTEQDSKDSTYSLSSTLTL
SKADYEKHKVYACEVTHQGLSSPVTKSFNRGEC*

### Nucleic Acid Sequences of SBJ03-F18:

> SBJ03-F18_heavy_chain leader sequence
SEQ ID NO: 11
ATGGGATGGTCATGTATCATCCTTTTTCTAGTAGCAACTGCAACCGGTGTACAT
TCC
> SBJ03-F18_heavy_chain variable domain
SEQ ID NO: 12
GAGGTGCAGCTGGTGGAGTCTGGGGGAGGCCTGGTCAAGCCTGGGGGGTCCCT
GAGACTCTCCTGTGCAGCCTCTGGATTCACCTTCAGTTCCTATAGCATGAACTG
GGTCCGCCAGGCTCCAGGGAAGGGGCTGGAGTGGGTCTCATCCATTAGTGGCA
ATAGTAATTACATATATTACGCCGACTCAGTGAAGGGCCGATTCACCATCTCC
AGAGACAACGCCAAGAACTCACTCTATCTGCAAACGAGCAGCCTGAGAGCCG
AGGACACGGCTCTCTATTTCTGTGCGAGGGGGACAATTATTGGAGCTGCGGGA
TATGACTGCTGGGGACAGGGAACCCTTGTCACTGTCTCCTCAG
> SBJ03-F18_heavy_chain constant domain
SEQ ID NO: 13
CCTCCACCAAGGGCCCATCGGTCTTCCCCCTGGCACCCTCCTCCAAGAGCACCT
CTGGGGGCACAGCGGCCCTGGGCTGCCTGGTCAAGGACTACTTCCCCGAACCT
GTGACGGTCTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCC
CTCCAGCAGCTTGGGCACCCAGACCTACATCTGCAACGTGAATCACAAGCCCA
GCAACACCAAGGTGGACAAGAGAGTTGAGCCCAAATCTTGTGACAAAACTCA
CACATGCCCACCGTGCCCAGCACCTGAACTCCTGGGGGGACCGTCAGTCTTCC
TCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAG
CAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCACA
GGTGTACACCCTGCCCCCATCCCGGGAGGAGATGACCAAGAACCAGGTCAGCC
TGACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTC
CGACGGCTCCTTCTTCCTCTATAGCAAGCTCACCGTGGACAAGAGCAGGTGGC
AGCAGGGGAACGTCTTCTCATGCTCCGTGATGCATGGCGCTCTGCACAACCAC
TACACGCAGAAGAGCCTCTCCCTGTTCCCCGGGTAA
> SBJ03-F18_heavy_chain complete
SEQ ID NO: 14
ATGGGATGGTCATGTATCATCCTTTTTCTAGTAGCAACTGCAACCGGTGTACAT
TCCGAGGTGCAGCTGGTGGAGTCTGGGGGAGGCCTGGTCAAGCCTGGGGGGTC
CCTGAGACTCTCCTGTGCAGCCTCTGGATTCACCTTCAGTTCCTATAGCATGAA
CTGGGTCCGCCAGGCTCCAGGGAAGGGGCTGGAGTGGGTCTCATCCATTAGTG
GCAATAGTAATTACATATATTACGCCGACTCAGTGAAGGGCCGATTCACCATC
TCCAGAGACAACGCCAAGAACTCACTCTATCTGCAAACGAGCAGCCTGAGAGC
CGAGGACACGGCTCTCTATTTCTGTGCGAGGGGGACAATTATTGGAGCTGCGG
GATATGACTGCTGGGGACAGGGAACCCTTGTCACTGTCTCCTCAGCCTCCACC
AAGGGCCCATCGGTCTTCCCCCTGGCACCCTCCTCCAAGAGCACCTCTGGGGG
CACAGCGGCCCTGGGCTGCCTGGTCAAGGACTACTTCCCCGAACCTGTGACGG
TCTCGTGGAACTCAGGCGCCCTGACCAGCGGCGTGCACACCTTCCCGGCTGTC
CTACAGTCCTCAGGACTCTACTCCCTCAGCAGCGTGGTGACCGTGCCCTCCAGC
AGCTTGGGCACCCAGACCTACATCTGCAACGTGAATCACAAGCCCAGCAACAC
CAAGGTGGACAAGAGAGTTGAGCCCAAATCTTGTGACAAAACTCACACATGCC
CACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTG
GTGGTGGACGTGAGCCACGAAGACCCTGAGGTCAAGTTCAACTGGTACGTGGA
CGGCGTGGAGGTGCATAATGCCAAGACAAAGCCGCGGGAGGAGCAGTACAAC
AGCACGTACCGTGTGGTCAGCGTCCTCACCGTCCTGCACCAGGACTGGCTGAA
TGGCAAGGAGTACAAGTGCAAGGTCTCCAACAAAGCCCTCCCAGCCCCCATCG
AGAAAACCATCTCCAAAGCCAAAGGGCAGCCCCGAGAACCACAGGTGTACAC
CCTGCCCCCATCCCGGGAGGAGATGACCAAGAACCAGGTCAGCCTGACCTGCC
TGGTCAAAGGCTTCTATCCCAGCGACATCGCCGTGGAGTGGGAGAGCAATGGG
CAGCCGGAGAACAACTACAAGACCACGCCTCCCGTGCTGGACTCCGACGGCTC
CTTCTTCCTCTATAGCAAGCTCACCGTGGACAAGAGCAGGTGGCAGCAGGGGA
ACGTCTTCTCATGCTCCGTGATGCATGGCGCTCTGCACAACCACTACACGCAGA
AGAGCCTCTCCCTGTTCCCCGGGTAA
> SBJ03-F18_light_kappa_chain leader sequence
SEQ ID NO: 15
ATGGGATGGTCATGTATCATCCTTTTTCTAGTAGCAACTGCAACCGGTGTACAT
TCC
> SBJ03-F18_light_kappa_chain variable domain
SEQ ID NO: 16
GCCATCCAGATGACCCAGTCTCCATCCTTCCTGTCTGCATCTGTAGGAGACAGA
GTCACACTCACTTGCCGGGCCAGTCAGGACATTAGCACTTCTTTAGCCTGGTAT
CATCAGAAACCAGGGAAAGCCCCTGAACGCCTGGTCTATGCTGCATCCACTTT
GCAAAGTGGGGTCCCATCAAGGTTCAGCGGCAGTGGGTCTGGGACAGGATTCA
CTCTCACAATCAGCAGCCTGCAGCCTGAAGATTTTGGAACTTATTACTGTCAAC
AACTTAAAAGTTACCCTCTCGCTTTCGGCGGAGGGACCAAGGTGGAGATCAAA
C
> SBJ03-F18_light_kappa_chain constant domain
SEQ ID NO: 17
GAACTGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGT
GTTAG
> SBJ03-F18_light_kappa_chain complete
SEQ ID NO: 18
ATGGGATGGTCATGTATCATCCTTTTTCTAGTAGCAACTGCAACCGGTGTACAT
TCCGCCATCCAGATGACCCAGTCTCCATCCTTCCTGTCTGCATCTGTAGGAGAC
AGAGTCACACTCACTTGCCGGGCCAGTCAGGACATTAGCACTTCTTTAGCCTG
GTATCATCAGAAACCAGGGAAAGCCCCTGAACGCCTGGTCTATGCTGCATCCA
CTTTGCAAAGTGGGGTCCCATCAAGGTTCAGCGGCAGTGGGTCTGGGACAGGA
TTCACTCTCACAATCAGCAGCCTGCAGCCTGAAGATTTTGGAACTTATTACTGT
CAACAACTTAAAAGTTACCCTCTCGCTTTCGGCGGAGGGACCAAGGTGGAGAT
CAAACGAACTGTGGCTGCACCATCTGTCTTCATCTTCCCGCCATCTGATGAGCA
GTTGAAATCTGGAACTGCCTCTGTTGTGTGCCTGCTGAATAACTTCTATCCCAG
AGAGGCCAAAGTACAGTGGAAGGTGGATAACGCCCTCCAATCGGGTAACTCCC
AGGAGAGTGTCACAGAGCAGGACAGCAAGGACAGCACCTACAGCCTCAGCAG
CACCCTGACGCTGAGCAAAGCAGACTACGAGAAACACAAAGTCTACGCCTGC
GAAGTCACCCATCAGGGCCTGAGCTCGCCCGTCACAAAGAGCTTCAACAGGGG
AGAGTGTTAG

### Sequence of the Antibody Herein Identified as SBJ03-L02

### Amino Acid Sequences of SBJ03-L02:

>CDR 1 of variable domain of Heavy chain of
SBJ03-L02
SEQ ID NO: 19
GFTFSNYG
>CDR 2 of variable domain of Heavy
SEQ ID NO: 20
chain of SBJ03-L02
ISYDGRNR
>CDR 3 of variable domain of Heavy
SEQ ID NO: 21
chain of SBJ03-L02
AKKILDNGTFQGSY
>CDR 1 of variable domain of Light
SEQ ID NO: 22
chain of SBJ03-L02
RGFGNY
>CDR 2 of variable domain of Light
chain of SBJ03-L02 this sequence is not included
in the sequence listing because is less than 4
amino acid
SEQ ID NO: 23
GAS (Gly-Ala-Ser)
>CDR 3 of variable domain of Light chain of
SBJ03-L02
SEQ ID NO: 24
QKYDNDPWA
>variable domain of Heavy chain of SBJ03-L02
SEQ ID NO: 25
EVQLVESGGGVVQPGRSLRLSCTASGFTFSNYGMHWVRQAPGKGLEWVAV
ISYDGRNRFYAESVKGRFTISRDNSKNTLYLQMNSLRAEDTAVYYCAKKI
LDNGTFQGSYWGQGTLLTVSS
>variable domain of Light chain of SBJ03-L02
SEQ ID NO: 26
AIQMTQSPSSLSASVGDRVTITCRASRGFGNYLAWYQQMPGKVPKLLIYG
ASTLQSGVPSRFSGSGSGTDFSLTISSLQPEDVATYYCQKYDNDPWAFGQ
GTKVEIK
>Heavy chain of SBJ03-L02
SEQ ID NO: 27
MGWSCIILFLVATATGVHSEVQLVESGGGVVQPGRSLRLSCTASGFTFSN
YGMHWVRQAPGKGLEWVAVISYDGRNRFYAESVKGRFTISRDNSKNTLYL
QMNSLRAEDTAVYYCAKKILDNGTFQGSYWGQGTLLTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSV
MHGALHNHYTQKSLSLFPG*
>Light chain of SBJ03-L02
SEQ ID NO: 28
MGWSCIILFLVATATGVHSAIQMTQSPSSLSASVGDRVTITCRASRGFGN
YLAWYQQMPGKVPKLLIYGASTLQSGVPSRFSGSGSGTDFSLTISSLQPE
DVATYYCQKYDNDPWAFGQGTKVEIKRTVAAPSVFIFPPSDEQLKSGTAS
VVCLLNNFYPREAKVQWKVDNALQSGNSQESVTEQDSKDSTYSLSSTLTL
SKADYEKHKVYACEVTHQGLSSPVTKSFNRGEC*

### Nucleic Acid Sequences of SBJ03-L02:

> SBJ03-L02 heavy chain leader sequence
SEQ ID NO: 29
ATGGGATGGTCATGTATCATCCTTTTTCTAGTAGCAACTGCAACCGGTGTACAT
TCC
> SBJ03-L02_heavy_chain variable domain
SEQ ID NO: 30
GAGGTGCAGCTGGTGGAGTCTGGGGGAGGCGTGGTCCAGCCTGGGAGGTCCCT
GAGACTCTCCTGTACAGCCTCTGGATTCACTTTCAGTAATTATGGCATGCACTG
GGTCCGCCAGGCTCCAGGCAAGGGGCTGGAGTGGGTGGCAGTTATATCATATG
ATGGAAGGAATAGATTCTATGCAGAGTCCGTGAAGGGCCGATTCACCATCTCC
AGAGACAATTCTAAGAACACGCTGTATCTTCAAATGAACAGCCTGAGAGCTGA
GGACACGGCTGTCTACTACTGTGCGAAAAAGATACTCGACAACGGTACTTTTC
AGGGGAGCTATTGGGGCCAGGGAACCCTACTCACCGTCTCCTCAG
> SBJ03-L02_heavy_chain constant domain
SEQ ID NO: 31
CCTCCACCAAGGGCCCATCGGTCTTCCCCCTGGCACCCTCCTCCAAGAGCACCT
CTGGGGGCACAGCGGCCCTGGGCTGCCTGGTCAAGGACTACTTCCCCGAACCT
GTGACGGTCTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCC
CTCCAGCAGCTTGGGCACCCAGACCTACATCTGCAACGTGAATCACAAGCCCA
GCAACACCAAGGTGGACAAGAGAGTTGAGCCCAAATCTTGTGACAAAACTCA
CACATGCCCACCGTGCCCAGCACCTGAACTCCTGGGGGGACCGTCAGTCTTCC
TCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAG
CAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCACA
GGTGTACACCCTGCCCCCATCCCGGGAGGAGATGACCAAGAACCAGGTCAGCC
TGACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTC
CGACGGCTCCTTCTTCCTCTATAGCAAGCTCACCGTGGACAAGAGCAGGTGGC
AGCAGGGGAACGTCTTCTCATGCTCCGTGATGCATGGCGCTCTGCACAACCAC
TACACGCAGAAGAGCCTCTCCCTGTTCCCCGGGTAA
> SBJ03-L02_heavy_chain complete
SEQ ID NO: 32
ATGGGATGGTCATGTATCATCCTTTTTCTAGTAGCAACTGCAACCGGTGTACAT
TCCGAGGTGCAGCTGGTGGAGTCTGGGGGAGGCGTGGTCCAGCCTGGGAGGTC
CCTGAGACTCTCCTGTACAGCCTCTGGATTCACTTTCAGTAATTATGGCATGCA
CTGGGTCCGCCAGGCTCCAGGCAAGGGGCTGGAGTGGGTGGCAGTTATATCAT
ATGATGGAAGGAATAGATTCTATGCAGAGTCCGTGAAGGGCCGATTCACCATC
TCCAGAGACAATTCTAAGAACACGCTGTATCTTCAAATGAACAGCCTGAGAGC
TGAGGACACGGCTGTCTACTACTGTGCGAAAAAGATACTCGACAACGGTACTT
TTCAGGGGAGCTATTGGGGCCAGGGAACCCTACTCACCGTCTCCTCAGCCTCC
ACCAAGGGCCCATCGGTCTTCCCCCTGGCACCCTCCTCCAAGAGCACCTCTGG
GGGCACAGCGGCCCTGGGCTGCCTGGTCAAGGACTACTTCCCCGAACCTGTGA
CGGTCTCGTGGAACTCAGGCGCCCTGACCAGCGGCGTGCACACCTTCCCGGCT
GTCCTACAGTCCTCAGGACTCTACTCCCTCAGCAGCGTGGTGACCGTGCCCTCC
AGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACAT
GCCCACCGTGCCCAGCACCTGAACTCCTGGGGGGACCGTCAGTCTTCCTCTTCC
CCCCAAAACCCAAGGACACCCTCATGATCTCCCGGACCCCTGAGGTCACATGC
GTGGTGGTGGACGTGAGCCACGAAGACCCTGAGGTCAAGTTCAACTGGTACGT
GGACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTGGCT
GAATGGCAAGGAGTACAAGTGCAAGGTCTCCAACAAAGCCCTCCCAGCCCCCA
TCGAGAAAACCATCTCCAAAGCCAAAGGGCAGCCCCGAGAACCACAGGTGTA
CACCCTGCCCCCATCCCGGGAGGAGATGACCAAGAACCAGGTCAGCCTGACCT
GCCTGGTCAAAGGCTTCTATCCCAGCGACATCGCCGTGGAGTGGGAGAGCAAT
GGGCAGCCGGAGAACAACTACAAGACCACGCCTCCCGTGCTGGACTCCGACG
GCTCCTTCTTCCTCTATAGCAAGCTCACCGTGGACAAGAGCAGGTGGCAGCAG
GGGAACGTCTTCTCATGCTCCGTGATGCATGGCGCTCTGCACAACCACTACAC
GCAGAAGAGCCTCTCCCTGTTCCCCGGGTAA
> SBJ03-L02_light_kappa_chain leader sequence
SEQ ID NO: 33
ATGGGATGGTCATGTATCATCCTTTTTCTAGTAGCAACTGCAACCGGTGTACAT
TCC
> SBJ03-L02_light_kappa_chain variable domain
SEQ ID NO: 34
GCCATCCAGATGACCCAGTCTCCATCCTCCCTGTCTGCATCTGTAGGAGACAGA
GTCACCATCACTTGCCGGGCGAGTCGGGGCTTCGGTAATTATTTAGCCTGGTAT
CAGCAGATGCCAGGGAAAGTTCCTAAGCTCCTGATCTATGGTGCATCCACTTT
GCAATCAGGGGTCCCATCTCGCTTCAGTGGCAGTGGATCTGGGACAGATTTCA
GTCTCACCATCAGCAGCCTGCAGCCTGAAGATGTTGCAACTTATTACTGTCAAA
AGTATGACAATGACCCTTGGGCGTTCGGCCAGGGGACCAAGGTGGAAATCAA
AC
> SBJ03-L02_light_kappa_chain constant domain
SEQ ID NO: 35
GAACTGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGT
GTTAG
> SBJ03-L02_light_kappa_chain complete
SEQ ID NO: 36
ATGGGATGGTCATGTATCATCCTTTTTCTAGTAGCAACTGCAACCGGTGTACAT
TCCGCCATCCAGATGACCCAGTCTCCATCCTCCCTGTCTGCATCTGTAGGAGAC
AGAGTCACCATCACTTGCCGGGCGAGTCGGGGCTTCGGTAATTATTTAGCCTG
GTATCAGCAGATGCCAGGGAAAGTTCCTAAGCTCCTGATCTATGGTGCATCCA
CTTTGCAATCAGGGGTCCCATCTCGCTTCAGTGGCAGTGGATCTGGGACAGATT
TCAGTCTCACCATCAGCAGCCTGCAGCCTGAAGATGTTGCAACTTATTACTGTC
AAAAGTATGACAATGACCCTTGGGCGTTCGGCCAGGGGACCAAGGTGGAAAT
CAAACGAACTGTGGCTGCACCATCTGTCTTCATCTTCCCGCCATCTGATGAGCA
GTTGAAATCTGGAACTGCCTCTGTTGTGTGCCTGCTGAATAACTTCTATCCCAG
AGAGGCCAAAGTACAGTGGAAGGTGGATAACGCCCTCCAATCGGGTAACTCCC
AGGAGAGTGTCACAGAGCAGGACAGCAAGGACAGCACCTACAGCCTCAGCAG
CACCCTGACGCTGAGCAAAGCAGACTACGAGAAACACAAAGTCTACGCCTGC
GAAGTCACCCATCAGGGCCTGAGCTCGCCCGTCACAAAGAGCTTCAACAGGGG
AGAGTGTTAG

### Sequence of the Antibody Herein Identified as SBJ05-B17

### Amino Acid Sequences of SBJ05-B17:

>CDR 1 of variable domain of Heavy chain of
SBJ05-B17
SEQ ID NO: 37
GFPFSSRW
>CDR 2 of variable domain of Heavy chain of
SBJ05-B17
SEQ ID NO: 38
IDTNGRTT
>CDR 3 of variable domain of Heavy chain of
SBJ05-B17
SEQ ID NO: 39
ARDLPNFDSSDAGWGHGVDV
>CDR 1 of variable domain of Light chain of
SBJ05-B17
SEQ ID NO: 40
QTISSH
>CDR 2 of variable domain of Light chain of
SBJ05-B17 this sequence is not included in
the sequence listing because is less than 4 amino
acid
SEQ ID NO: 41
AAS (Ala-Ala-Ser)
>CDR 3 of variable domain of Light chain of
SBJ05-B17
SEQ ID NO: 42
LQTYTSLPT
>variable domain of Heavy chain of
SBJ05-B17
SEQ ID NO: 43
EVWLVESGGGLVQPGGSLRLSCEASGFPFSSRWIHWVRQGPGKGLVWLSR
IDINGRTTNYADSVNGRFTISKDNGKSTVYLQMNSLRAEDTAVYYCARDL
PNFDSSDAGWGHGVDVWGQGTTVIVSS
>variable domain of Light chain of
SBJ05-B17
SEQ ID NO: 44
AIQMTQSPSSLSASVGDRVTITCRARQTISSHLSWYQQKPGKPPKSLIYA
ASHLQSGVPSRFSGSGSGTDFTLTISRLQPEDSATYYCLQTYTSLPTFGP
GTKVEIK
>Heavy chain of SBJ05-B17
SEQ ID NO: 45
MGWSCIILFLVATATGVHSEVQLVESGGGLVQPGGSLRLSCEASGFPFSS
RWIHWVRQGPGKGLVWLSRIDINGRTTNYADSVNGRFTISKDNGKSTVYL
QMNSLRAEDTAVYYCARDLPNFDSSDAGWGHGVDVWGQGTTVIVSSASTK
GPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSCD
KTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGN
VFSCSVMHGALHNHYTQKSLSLFPG*
>Light chain of SBJ05-B17
SEQ ID NO: 46
MGWSCIILFLVATATGVHSAIQMTQSPSSLSASVGDRVTITCRARQTISS
HLSWYQQKPGKPPKSLIYAASHLQSGVPSRFSGSGSGTDFTLTISRLQPE
DSATYYCLQTYTSLPTFGPGTKVEIKRTVAAPSVFIFPPSDEQLKSGTAS
VVCLLNNFYPREAKVQWKVDNALQSGNSQESVTEQDSKDSTYSLSSTLTL
SKADYEKHKVYACEVTHQGLSSPVTKSFNRGEC*

### Nucleic Acid Sequences of SBJ05-B17:

> SBJ05-B17_heavy_chain leader sequence
SEQ ID NO: 47
ATGGGATGGTCATGTATCATCCTTTTTCTAGTAGCAACTGCAACCGGTGTACAT
TCC
> SBJ05-B17_heavy_chain variable domain
SEQ ID NO: 48
GAGGTGCAGCTGGTGGAGTCTGGGGGAGGCTTAGTTCAGCCTGGGGGGTCCCT
GAGACTCTCCTGTGAAGCCTCTGGGTTCCCCTTCAGTAGTCGCTGGATTCACTG
GGTCCGCCAGGGTCCCGGGAAGGGGCTGGTGTGGCTCTCACGTATTGACACTA
ATGGGAGGACAACAAACTACGCGGACTCCGTAAATGGCCGATTCACCATCTCC
AAAGACAACGGCAAGAGCACGGTGTATCTGCAAATGAATAGCCTGAGAGCCG
AGGACACGGCTGTGTATTATTGTGCAAGAGATTTGCCCAATTTTGACTCCTCCG
ATGCAGGATGGGGCCACGGTGTGGACGTCTGGGGCCAAGGGACCACGGTCAT
CGTCTCCTCA
> SBJ05-B17_heavy_chain constant domain
SEQ ID NO: 49
GCCTCCACCAAGGGCCCATCGGTCTTCCCCCTGGCACCCTCCTCCAAGAGCAC
CTCTGGGGGCACAGCGGCCCTGGGCTGCCTGGTCAAGGACTACTTCCCCGAAC
CTGTGACGGTCTCGTGGAACTCAGGCGCCCTGACCAGCGGCGTGCACACCTTC
CCGGCTGTCCTACAGTCCTCAGGACTCTACTCCCTCAGCAGCGTGGTGACCGTG
CCCTCCAGCAGCTTGGGCACCCAGACCTACATCTGCAACGTGAATCACAAGCC
CAGCAACACCAAGGTGGACAAGAGAGTTGAGCCCAAATCTTGTGACAAAACT
CACACATGCCCACCGTGCCCAGCACCTGAACTCCTGGGGGGACCGTCAGTCTT
CCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACT
GGTACGTGGACGGCGTGGAGGTGCATAATGCCAAGACAAAGCCGCGGGAGGA
GCAGTACAACAGCACGTACCGTGTGGTCAGCGTCCTCACCGTCCTGCACCAGG
ACTGGCTGAATGGCAAGGAGTACAAGTGCAAGGTCTCCAACAAAGCCCTCCCA
GCCCCCATCGAGAAAACCATCTCCAAAGCCAAAGGGCAGCCCCGAGAACCAC
AGGTGTACACCCTGCCCCCATCCCGGGAGGAGATGACCAAGAACCAGGTCAGC
CTGACCTGCCTGGTCAAAGGCTTCTATCCCAGCGACATCGCCGTGGAGTGGGA
GAGCAATGGGCAGCCGGAGAACAACTACAAGACCACGCCTCCCGTGCTGGAC
TCCGACGGCTCCTTCTTCCTCTATAGCAAGCTCACCGTGGACAAGAGCAGGTG
GCAGCAGGGGAACGTCTTCTCATGCTCCGTGATGCATGGCGCTCTGCACAACC
ACTACACGCAGAAGAGCCTCTCCCTGTTCCCCGGGTAA
> SBJ05-B17_heavy_chain complete
SEQ ID NO: 50
ATGGGATGGTCATGTATCATCCTTTTTCTAGTAGCAACTGCAACCGGTGTACAT
TCCGAGGTGCAGCTGGTGGAGTCTGGGGGAGGCTTAGTTCAGCCTGGGGGGTC
CCTGAGACTCTCCTGTGAAGCCTCTGGGTTCCCCTTCAGTAGTCGCTGGATTCA
CTGGGTCCGCCAGGGTCCCGGGAAGGGGCTGGTGTGGCTCTCACGTATTGACA
CTAATGGGAGGACAACAAACTACGCGGACTCCGTAAATGGCCGATTCACCATC
TCCAAAGACAACGGCAAGAGCACGGTGTATCTGCAAATGAATAGCCTGAGAG
CCGAGGACACGGCTGTGTATTATTGTGCAAGAGATTTGCCCAATTTTGACTCCT
CCGATGCAGGATGGGGCCACGGTGTGGACGTCTGGGGCCAAGGGACCACGGT
CATCGTCTCCTCAGCCTCCACCAAGGGCCCATCGGTCTTCCCCCTGGCACCCTC
CTCCAAGAGCACCTCTGGGGGCACAGCGGCCCTGGGCTGCCTGGTCAAGGACT
ACTTCCCCGAACCTGTGACGGTCTCGTGGAACTCAGGCGCCCTGACCAGCGGC
GTGCACACCTTCCCGGCTGTCCTACAGTCCTCAGGACTCTACTCCCTCAGCAGC
GTGGTGACCGTGCCCTCCAGCAGCTTGGGCACCCAGACCTACATCTGCAACGT
GAATCACAAGCCCAGCAACACCAAGGTGGACAAGAGAGTTGAGCCCAAATCT
TGTGACAAAACTCACACATGCCCACCGTGCCCAGCACCTGAACTCCTGGGGGG
ACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGG
TCAAGTTCAACTGGTACGTGGACGGCGTGGAGGTGCATAATGCCAAGACAAAG
CCGCGGGAGGAGCAGTACAACAGCACGTACCGTGTGGTCAGCGTCCTCACCGT
CCTGCACCAGGACTGGCTGAATGGCAAGGAGTACAAGTGCAAGGTCTCCAACA
AAGCCCTCCCAGCCCCCATCGAGAAAACCATCTCCAAAGCCAAAGGGCAGCCC
CGAGAACCACAGGTGTACACCCTGCCCCCATCCCGGGAGGAGATGACCAAGA
ACCAGGTCAGCCTGACCTGCCTGGTCAAAGGCTTCTATCCCAGCGACATCGCC
GTGGAGTGGGAGAGCAATGGGCAGCCGGAGAACAACTACAAGACCACGCCTC
CCGTGCTGGACTCCGACGGCTCCTTCTTCCTCTATAGCAAGCTCACCGTGGACA
AGAGCAGGTGGCAGCAGGGGAACGTCTTCTCATGCTCCGTGATGCATGGCGCT
CTGCACAACCACTACACGCAGAAGAGCCTCTCCCTGTTCCCCGGGTAA
> SBJ05-B17_light_kappa_chain leader sequence
SEQ ID NO: 51
ATGGGATGGTCATGTATCATCCTTTTTCTAGTAGCAACTGCAACCGGTGTACAT
TCC
> SBJ05-B17_light_kappa_chain variable domain
SEQ ID NO: 52
GCCATCCAGATGACCCAGTCTCCATCCTCCCTGTCCGCATCTGTAGGAGACAG
AGTCACCATCACTTGCCGGGCAAGGCAGACCATTAGTAGTCACTTAAGTTGGT
ATCAGCAGAAACCAGGGAAGCCGCCTAAGTCCCTGATCTATGCTGCATCCCAC
TTGCAAAGTGGGGTCCCATCACGGTTCAGTGGCAGTGGATCTGGGACAGATTT
CACTCTCACCATCAGCCGTCTGCAACCTGAAGATTCTGCTACTTATTATTGTCT
ACAGACTTATACTTCCCTTCCCACATTCGGCCCTGGGACCAAAGTGGAAATTA
AAC
> SBJ05-B17_light_kappa_chain constant domain
SEQ ID NO: 53
GAACGGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGT
GTTAG
> SBJ05-B17_light_kappa_chain complete
SEQ ID NO: 54
ATGGGATGGTCATGTATCATCCTTTTTCTAGTAGCAACTGCAACCGGTGTACAT
TCCGCCATCCAGATGACCCAGTCTCCATCCTCCCTGTCCGCATCTGTAGGAGAC
AGAGTCACCATCACTTGCCGGGCAAGGCAGACCATTAGTAGTCACTTAAGTTG
GTATCAGCAGAAACCAGGGAAGCCGCCTAAGTCCCTGATCTATGCTGCATCCC
ACTTGCAAAGTGGGGTCCCATCACGGTTCAGTGGCAGTGGATCTGGGACAGAT
TTCACTCTCACCATCAGCCGTCTGCAACCTGAAGATTCTGCTACTTATTATTGT
CTACAGACTTATACTTCCCTTCCCACATTCGGCCCTGGGACCAAAGTGGAAATT
AAACGAACGGTGGCTGCACCATCTGTCTTCATCTTCCCGCCATCTGATGAGCAG
TTGAAATCTGGAACTGCCTCTGTTGTGTGCCTGCTGAATAACTTCTATCCCAGA
GAGGCCAAAGTACAGTGGAAGGTGGATAACGCCCTCCAATCGGGTAACTCCCA
GGAGAGTGTCACAGAGCAGGACAGCAAGGACAGCACCTACAGCCTCAGCAGC
ACCCTGACGCTGAGCAAAGCAGACTACGAGAAACACAAAGTCTACGCCTGCG
AAGTCACCCATCAGGGCCTGAGCTCGCCCGTCACAAAGAGCTTCAACAGGGGA
GAGTGTTAG

### Sequence of the Antibody Herein Identified as SBJ05-C11

### Amino Acid Sequences of SBJ05-C11:

>CDR 1 of variable domain of Heavy chain of
SBJ05-C11
SEQ ID NO: 55
GYIFSNNG
>CDR 2 of variable domain of Heavy chain of
SBJ05-C11
SEQ ID NO: 56
ISPYTGHT
>CDR 3 of variable domain of Heavy chain of
SBJ05-C11
SEQ ID NO: 57
ATDYQDGVRALAH
>CDR 1 of variable domain of Light chain of
SBJ05-C11
SEQ ID NO: 58
NSNIGNNH
>CDR 2 of variable domain of Light chain of
SBJ05-C11 this sequence is not included in the
sequence listing because is less than 4 amino
acid
SEQ ID NO: 59
DDY (Asp-Asp-Tyr)
>CDR 3 of variable domain of Light chain of
SBJ05-C11
SEQ ID NO: 60
GTWDTSLNARV
>variable domain of Heavy chain of SBJ05-C11
SEQ ID NO: 61
QVQLVQSGADVENPGASVKVSCRSSGYIFSNNGITWVRQVPGQGLEWMGW
ISPYTGHTNYAQTLQGRVAMTTDTSTSIFYMELRSLRSDDTAVYYCATDY
QDGVRALAHWGQGTLVTVSS
>variable domain of Light chain of SBJ05-C11
SEQ ID NO: 62
QAVVTQEPSMSAAPGQKVTISCSGINSNIGNNHVSWYQQLPGTAPKLLIY
DDYKRPSGIPDRFSGSKSGTSATLGITGLQTGDEGDYYCGTWDTSLNARV
FGGGTKLTVL
>Heavy chain of SBJ05-C11
SEQ ID NO: 63
MGWSCIILFLVATATGVHSQVQLVQSGADVENPGASVKVSCRSSGYIFSN
NGITWVRQVPGQGLEWMGWISPYTGHTNYAQTLQGRVAMTTDTSTSIFYM
ELRSLRSDDTAVYYCATDYQDGVRALAHWGQGTLVTVSSASPTSPKSVFP
LAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSV
MHGALHNHYTQKSLSLFPG*
>Light chain of SBJ05-C11
SEQ ID NO: 64
MGWSCIILFLVATATGSWAQAVVTQEPSMSAAPGQKVTISCSGINSNIGN
NHVSWYQQLPGTAPKLLIYDDYKRPSGIPDRFSGSKSGTSATLGITGLQT
GDEGDYYCGTWDTSLNARVFGGGTKLTVLSQPKAAPSVTLFPPSSEELQA
NKATLVCLISDFYPGAVTVAWKADSSPVKAGVETTTPSKQSNNKYAASSY
LSLTPEQWKSHRSYSCQVTHEGSTVEKTVAPTECS*

### Nucleic Acid Sequences of SBJ05-C11:

> SBJ05-C11_heavy_chain leader sequence
SEQ ID NO: 65
ATGGGATGGTCATGTATCATCCTTTTTCTAGTAGCAACTGCAACCGGTGTACAT
TCC
> SBJ05-C11_heavy_chain variable domain
SEQ ID NO: 66
CAGGTGCAGCTGGTGCAGTCTGGGGCTGACGTGGAGAACCCTGGGGCCTCAGT
GAAAGTCTCCTGCAGGTCTTCTGGTTATATTTTTAGCAACAATGGCATCACCTG
GGTGCGACAGGTCCCTGGACAAGGCCTTGAGTGGATGGGGTGGATCAGCCCTT
ACACTGGTCACACAAACTATGCACAGACGCTCCAGGGCAGAGTCGCCATGACC
ACAGACACATCCACGAGTATATTCTACATGGAGCTGAGGAGCCTGAGGTCTGA
CGACACGGCCGTCTATTACTGCGCGACCGATTACCAGGATGGTGTCCGAGCAT
TGGCCCACTGGGGCCAGGGAACCCTGGTCACCGTCTCCTCAG
> SBJ05-C11_heavy_chain constant domain
SEQ ID NO: 67
CATCCCCGACCAGCCCCAAGTCGGTCTTCCCCCTGGCACCCTCCTCCAAGAGC
ACCTCTGGGGGCACAGCGGCCCTGGGCTGCCTGGTCAAGGACTACTTCCCCGA
ACCTGTGACGGTCTCGTGGAACTCAGGCGCCCTGACCAGCGGCGTGCACACCT
TCCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAAG
CCCAGCAACACCAAGGTGGACAAGAGAGTTGAGCCCAAATCTTGTGACAAAA
CTCACACATGCCCACCGTGCCCAGCACCTGAACTCCTGGGGGGACCGTCAGTC
TTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAA
CTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCA
GGACTGGCTGAATGGCAAGGAGTACAAGTGCAAGGTCTCCAACAAAGCCCTCC
CAGCCCCCATCGAGAAAACCATCTCCAAAGCCAAAGGGCAGCCCCGAGAACC
ACAGGTGTACACCCTGCCCCCATCCCGGGAGGAGATGACCAAGAACCAGGTCA
GCCTGACCTGCCTGGTCAAAGGCTTCTATCCCAGCGACATCGCCGTGGAGTGG
GAGAGCAATGGGCAGCCGGAGAACAACTACAAGACCACGCCTCCCGTGCTGG
ACTCCGACGGCTCCTTCTTCCTCTATAGCAAGCTCACCGTGGACAAGAGCAGG
TGGCAGCAGGGGAACGTCTTCTCATGCTCCGTGATGCATGGCGCTCTGCACAA
CCACTACACGCAGAAGAGCCTCTCCCTGTTCCCCGGGTAA
> SBJ05-C11_heavy_chain complete
SEQ ID NO: 68
ATGGGATGGTCATGTATCATCCTTTTTCTAGTAGCAACTGCAACCGGTGTACAT
TCCCAGGTGCAGCTGGTGCAGTCTGGGGCTGACGTGGAGAACCCTGGGGCCTC
AGTGAAAGTCTCCTGCAGGTCTTCTGGTTATATTTTTAGCAACAATGGCATCAC
CTGGGTGCGACAGGTCCCTGGACAAGGCCTTGAGTGGATGGGGTGGATCAGCC
CTTACACTGGTCACACAAACTATGCACAGACGCTCCAGGGCAGAGTCGCCATG
ACCACAGACACATCCACGAGTATATTCTACATGGAGCTGAGGAGCCTGAGGTC
TGACGACACGGCCGTCTATTACTGCGCGACCGATTACCAGGATGGTGTCCGAG
CATTGGCCCACTGGGGCCAGGGAACCCTGGTCACCGTCTCCTCAGCATCCCCG
ACCAGCCCCAAGTCGGTCTTCCCCCTGGCACCCTCCTCCAAGAGCACCTCTGGG
GGCACAGCGGCCCTGGGCTGCCTGGTCAAGGACTACTTCCCCGAACCTGTGAC
GGTCTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCA
GCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATG
CCCACCGTGCCCAGCACCTGAACTCCTGGGGGGACCGTCAGTCTTCCTCTTCCC
CCCAAAACCCAAGGACACCCTCATGATCTCCCGGACCCCTGAGGTCACATGCG
TGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACA
ACAGCACGTACCGTGTGGTCAGCGTCCTCACCGTCCTGCACCAGGACTGGCTG
AATGGCAAGGAGTACAAGTGCAAGGTCTCCAACAAAGCCCTCCCAGCCCCCAT
CGAGAAAACCATCTCCAAAGCCAAAGGGCAGCCCCGAGAACCACAGGTGTAC
ACCCTGCCCCCATCCCGGGAGGAGATGACCAAGAACCAGGTCAGCCTGACCTG
CCTGGTCAAAGGCTTCTATCCCAGCGACATCGCCGTGGAGTGGGAGAGCAATG
GGCAGCCGGAGAACAACTACAAGACCACGCCTCCCGTGCTGGACTCCGACGGC
TCCTTCTTCCTCTATAGCAAGCTCACCGTGGACAAGAGCAGGTGGCAGCAGGG
GAACGTCTTCTCATGCTCCGTGATGCATGGCGCTCTGCACAACCACTACACGCA
GAAGAGCCTCTCCCTGTTCCCCGGGTAA
> SBJ05-C11_light_kappa_chain leader sequence
SEQ ID NO: 69
ATGGGATGGTCATGTATCATCCTTTTTCTAGTAGCAACTGCAACCGGTTCCTGG
GCC
> SBJ05-C11_light_kappa_chain variable domain
SEQ ID NO: 70
CAGGCTGTGGTGACTCAGGAGCCCTCAATGTCTGCGGCCCCAGGACAGAAGGT
CACCATCTCCTGCTCTGGAATCAACTCCAACATTGGAAATAATCATGTTTCCTG
GTATCAGCAGCTCCCAGGAACAGCCCCCAAACTCCTCATTTATGACGATTATA
AGCGACCCTCAGGGATTCCTGACCGATTCTCTGGCTCCAAGTCTGGCACGTCA
GCCACCCTGGGCATCACCGGACTCCAGACTGGGGACGAGGGCGATTATTATTG
CGGGACATGGGATACCAGCCTGAATGCCCGGGTGTTCGGCGGAGGGACCAAG
CTGACCGTCCTA
> SBJ05-C11_light_kappa_chain constant domain
SEQ ID NO: 71
AGTCAGCCCAAGGCTGCCCCCTCGGTCACTCTGTTCCCGCCCTCGAGTGAGGA
GCTTCAAGCCAACAAGGCCACACTGGTGTGTCTCATAAGTGACTTCTACCCGG
GAGCCGTGACAGTGGCCTGGAAGGCAGATAGCAGCCCCGTCAAGGCGGGAGT
GGAGACCACCACACCCTCCAAACAAAGCAACAACAAGTACGCGGCCAGCAGC
TACCTGAGCCTGACGCCTGAGCAGTGGAAGTCCCACAGAAGCTACAGCTGCCA
GGTCACGCATGAAGGGAGCACCGTGGAGAAGACAGTGGCCCCTACAGAATGT
TCATAG
> SBJ05-C11_light_kappa_chain complete
SEQ ID NO: 72
ATGGGATGGTCATGTATCATCCTTTTTCTAGTAGCAACTGCAACCGGTTCCTGG
GCCCAGGCTGTGGTGACTCAGGAGCCCTCAATGTCTGCGGCCCCAGGACAGAA
GGTCACCATCTCCTGCTCTGGAATCAACTCCAACATTGGAAATAATCATGTTTC
CTGGTATCAGCAGCTCCCAGGAACAGCCCCCAAACTCCTCATTTATGACGATT
ATAAGCGACCCTCAGGGATTCCTGACCGATTCTCTGGCTCCAAGTCTGGCACGT
CAGCCACCCTGGGCATCACCGGACTCCAGACTGGGGACGAGGGCGATTATTAT
TGCGGGACATGGGATACCAGCCTGAATGCCCGGGTGTTCGGCGGAGGGACCA
AGCTGACCGTCCTAAGTCAGCCCAAGGCTGCCCCCTCGGTCACTCTGTTCCCGC
CCTCGAGTGAGGAGCTTCAAGCCAACAAGGCCACACTGGTGTGTCTCATAAGT
GACTTCTACCCGGGAGCCGTGACAGTGGCCTGGAAGGCAGATAGCAGCCCCGT
CAAGGCGGGAGTGGAGACCACCACACCCTCCAAACAAAGCAACAACAAGTAC
GCGGCCAGCAGCTACCTGAGCCTGACGCCTGAGCAGTGGAAGTCCCACAGAA
GCTACAGCTGCCAGGTCACGCATGAAGGGAGCACCGTGGAGAAGACAGTGGC
CCCTACAGAATGTTCATAG

### Sequence of the Antibody Herein Identified as SBJ05-D08

### Amino Acid Sequences of SBJ05-D08:

>CDR 1 of variable domain of Heavy chain of
SBJ05-D08
SEQ ID NO: 73
GFTFTKTW
>CDR 2 of variable domain of Heavy chain of
SBJ05-D08
SEQ ID NO: 74
IKSKIDGETT
>CDR 3 of variable domain of Heavy chain of
SBJ05-D08
SEQ ID NO: 75
TSRVLTTNDY
>CDR 1 of variable domain of Light chain of
SBJ05-D08
SEQ ID NO: 76
SSDVGGYDY
>CDR 2 of variable domain of Light chain of
SBJ05-D08 this sequence is not included in the
sequence listing because is less than 4 amino
acid
SEQ ID NO: 77
DVS (Asp-Val-Ser)
>CDR 3 of variable domain of Light chain of
SBJ05-D08
SEQ ID NO: 78
CSFTTSGTFV
>variable domain of Heavy chain of SBJ05-D08
SEQ ID NO: 79
EVQLVESGGGLVKPGGSLRLSCAASGFTFTKTWMNWVRQAPGKGLEWLGR
IKSKIDGETTDYAAPVKGRFTISRDDSKNTVYLQMNSLGTEDTALYYCTS
RVLTTNDYWGQGTLVTVSS
>variable domain of Light chain of SBJ05-D08
SEQ ID NO: 80
QSALTQPASVSGSPGQSIAISCTGTSSDVGGYDYVSWYQQHPGKVPKHMI
YDVSNRPSGVSNRFSGSKSGNTASLTISGLQPEDEADYYCCSFTTSGTFV
FGTGTKVTVL
>Heavy chain of SBJ05-D08
SEQ ID NO: 81
MGWSCIILFLVATATGVHSEVQLVESGGGLVKPGGSLRLSCAASGFTFTK
TWMNWVRQAPGKGLEWLGRIKSKIDGETTDYAAPVKGRFTISRDDSKNTV
YLQMNSLGTEDTALYYCTSRVLTTNDYWGQGTLVTVSSASPTSPKSVFPL
APSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPP
CPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVM
HGALHNHYTQKSLSLFPG*
>Light chain of SBJ05-D08
SEQ ID NO: 82
MGWSCIILFLVATATGSWAQSALTQPASVSGSPGQSIAISCTGTSSDVGG
YDYVSWYQQHPGKVPKHMIYDVSNRPSGVSNRFSGSKSGNTASLTISGLQ
PEDEADYYCCSFTTSGTFVFGTGTKVTVLGQPKANPTVTLFPPSSEELQA
NKATLVCLISDFYPGAVTVAWKADSSPVKAGVETTTPSKQSNNKYAASSY
LSLTPEQWKSHRSYSCQVTHEGSTVEKTVAPTECS*

### Nucleic Acid Sequences of SBJ05-D08:

> SBJ05-D08_heay_chain leader sequence
SEQ ID NO: 83
ATGGGATGGTCATGTATCATCCTTTTTCTAGTAGCAACTGCAACCGGTGTACAT
TCC
> SBJ05-D08_heavy_chain variable domain
SEQ ID NO: 84
GAGGTGCAGCTGGTGGAGTCTGGGGGAGGCTTGGTAAAGCCTGGGGGGTCCCT
TAGACTCTCCTGTGCGGCCTCTGGATTCACTTTCACTAAGACCTGGATGAACTG
GGTCCGCCAGGCTCCAGGGAAGGGGCTGGAGTGGCTTGGTCGTATTAAAAGCA
AAATTGATGGTGAGACAACAGACTACGCTGCACCCGTGAAAGGCAGATTCACC
ATCTCAAGAGATGATTCAAAAAACACGGTGTATCTGCAAATGAACAGCCTGGG
AACCGAGGACACAGCCCTATATTACTGTACCTCTCGAGTCCTGACTACGAATG
ACTACTGGGGCCAGGGAACCCTGGTCACCGTCTCCTCAG
> SBJ05-D08_heavy_chain constant domain
SEQ ID NO: 85
CATCCCCGACCAGCCCCAAGTCGGTCTTCCCCCTGGCACCCTCCTCCAAGAGC
ACCTCTGGGGGCACAGCGGCCCTGGGCTGCCTGGTCAAGGACTACTTCCCCGA
ACCTGTGACGGTCTCGTGGAACTCAGGCGCCCTGACCAGCGGCGTGCACACCT
TCCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAAG
CCCAGCAACACCAAGGTGGACAAGAGAGTTGAGCCCAAATCTTGTGACAAAA
CTCACACATGCCCACCGTGCCCAGCACCTGAACTCCTGGGGGGACCGTCAGTC
TTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAA
CTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCA
GGACTGGCTGAATGGCAAGGAGTACAAGTGCAAGGTCTCCAACAAAGCCCTCC
CAGCCCCCATCGAGAAAACCATCTCCAAAGCCAAAGGGCAGCCCCGAGAACC
ACAGGTGTACACCCTGCCCCCATCCCGGGAGGAGATGACCAAGAACCAGGTCA
GCCTGACCTGCCTGGTCAAAGGCTTCTATCCCAGCGACATCGCCGTGGAGTGG
GAGAGCAATGGGCAGCCGGAGAACAACTACAAGACCACGCCTCCCGTGCTGG
ACTCCGACGGCTCCTTCTTCCTCTATAGCAAGCTCACCGTGGACAAGAGCAGG
TGGCAGCAGGGGAACGTCTTCTCATGCTCCGTGATGCATGGCGCTCTGCACAA
CCACTACACGCAGAAGAGCCTCTCCCTGTTCCCCGGGTAA
> SBJ05-D08_heavy_chain complete
SEQ ID NO: 86
ATGGGATGGTCATGTATCATCCTTTTTCTAGTAGCAACTGCAACCGGTGTACAT
TCCGAGGTGCAGCTGGTGGAGTCTGGGGGAGGCTTGGTAAAGCCTGGGGGGTC
CCTTAGACTCTCCTGTGCGGCCTCTGGATTCACTTTCACTAAGACCTGGATGAA
CTGGGTCCGCCAGGCTCCAGGGAAGGGGCTGGAGTGGCTTGGTCGTATTAAAA
GCAAAATTGATGGTGAGACAACAGACTACGCTGCACCCGTGAAAGGCAGATTC
ACCATCTCAAGAGATGATTCAAAAAACACGGTGTATCTGCAAATGAACAGCCT
GGGAACCGAGGACACAGCCCTATATTACTGTACCTCTCGAGTCCTGACTACGA
ATGACTACTGGGGCCAGGGAACCCTGGTCACCGTCTCCTCAGCATCCCCGACC
AGCCCCAAGTCGGTCTTCCCCCTGGCACCCTCCTCCAAGAGCACCTCTGGGGG
CACAGCGGCCCTGGGCTGCCTGGTCAAGGACTACTTCCCCGAACCTGTGACGG
TCTCGTGGAACTCAGGCGCCCTGACCAGCGGCGTGCACACCTTCCCGGCTGTC
CTACAGTCCTCAGGACTCTACTCCCTCAGCAGCGTGGTGACCGTGCCCTCCAGC
AGCTTGGGCACCCAGACCTACATCTGCAACGTGAATCACAAGCCCAGCAACAC
CAAGGTGGACAAGAGAGTTGAGCCCAAATCTTGTGACAAAACTCACACATGCC
CACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTG
GTGGTGGACGTGAGCCACGAAGACCCTGAGGTCAAGTTCAACTGGTACGTGGA
CGGCGTGGAGGTGCATAATGCCAAGACAAAGCCGCGGGAGGAGCAGTACAAC
AGCACGTACCGTGTGGTCAGCGTCCTCACCGTCCTGCACCAGGACTGGCTGAA
TGGCAAGGAGTACAAGTGCAAGGTCTCCAACAAAGCCCTCCCAGCCCCCATCG
AGAAAACCATCTCCAAAGCCAAAGGGCAGCCCCGAGAACCACAGGTGTACAC
CCTGCCCCCATCCCGGGAGGAGATGACCAAGAACCAGGTCAGCCTGACCTGCC
TGGTCAAAGGCTTCTATCCCAGCGACATCGCCGTGGAGTGGGAGAGCAATGGG
CAGCCGGAGAACAACTACAAGACCACGCCTCCCGTGCTGGACTCCGACGGCTC
CTTCTTCCTCTATAGCAAGCTCACCGTGGACAAGAGCAGGTGGCAGCAGGGGA
ACGTCTTCTCATGCTCCGTGATGCATGGCGCTCTGCACAACCACTACACGCAGA
AGAGCCTCTCCCTGTTCCCCGGGTAA
> SBJ05-D08_light_kappa_chain leader sequence
SEQ ID NO: 87
ATGGGATGGTCATGTATCATCCTTTTTCTAGTAGCAACTGCAACCGGTTCCTGG
GCC
> SBJ05-D08_light_kappa_chain variable domain
SEQ ID NO: 88
CAGTCTGCCCTGACTCAGCCTGCCTCCGTGTCTGGGTCTCCTGGACAGTCGATC
GCCATCTCCTGCACTGGAACGAGCAGTGACGTTGGTGGTTATGACTATGTCTCC
TGGTACCAACAACACCCCGGCAAAGTCCCCAAACACATGATTTATGATGTCAG
TAATCGGCCCTCAGGGGTTTCTAATCGCTTCTCTGGCTCCAAGTCTGGCAACAC
GGCCTCCCTGACCATCTCTGGACTCCAGCCTGAGGACGAGGCTGATTATTACTG
CTGCTCATTTACAACCAGCGGAACTTTTGTCTTCGGAACTGGGACCAAGGTCAC
CGTCCT
> SBJ05-D08_light_kappa_chain constant domain
SEQ ID NO: 89
TGGTCAGCCCAAGGCCAACCCCACTGTCACTCTGTTCCCACCCTCGAGTGAGG
AGCTTCAAGCCAACAAGGCCACACTGGTGTGTCTCATAAGTGACTTCTACCCG
GGAGCCGTGACAGTGGCCTGGAAGGCAGATAGCAGCCCCGTCAAGGCGGGAG
TGGAGACCACCACACCCTCCAAACAAAGCAACAACAAGTACGCGGCCAGCAG
CTACCTGAGCCTGACGCCTGAGCAGTGGAAGTCCCACAGAAGCTACAGCTGCC
AGGTCACGCATGAAGGGAGCACCGTGGAGAAGACAGTGGCCCCTACAGAATG
TTCATAG
> SBJ05-D08_light_kappa_chain complete
SEQ ID NO: 90
ATGGGATGGTCATGTATCATCCTTTTTCTAGTAGCAACTGCAACCGGTTCCTGG
GCCCAGTCTGCCCTGACTCAGCCTGCCTCCGTGTCTGGGTCTCCTGGACAGTCG
ATCGCCATCTCCTGCACTGGAACGAGCAGTGACGTTGGTGGTTATGACTATGTC
TCCTGGTACCAACAACACCCCGGCAAAGTCCCCAAACACATGATTTATGATGT
CAGTAATCGGCCCTCAGGGGTTTCTAATCGCTTCTCTGGCTCCAAGTCTGGCAA
CACGGCCTCCCTGACCATCTCTGGACTCCAGCCTGAGGACGAGGCTGATTATT
ACTGCTGCTCATTTACAACCAGCGGAACTTTTGTCTTCGGAACTGGGACCAAG
GTCACCGTCCTTGGTCAGCCCAAGGCCAACCCCACTGTCACTCTGTTCCCACCC
TCGAGTGAGGAGCTTCAAGCCAACAAGGCCACACTGGTGTGTCTCATAAGTGA
CTTCTACCCGGGAGCCGTGACAGTGGCCTGGAAGGCAGATAGCAGCCCCGTCA
AGGCGGGAGTGGAGACCACCACACCCTCCAAACAAAGCAACAACAAGTACGC
GGCCAGCAGCTACCTGAGCCTGACGCCTGAGCAGTGGAAGTCCCACAGAAGCT
ACAGCTGCCAGGTCACGCATGAAGGGAGCACCGTGGAGAAGACAGTGGCCCC
TACAGAATGTTCATAG

### Sequence of the Antibody Herein Identified as SBJ05-D14

### Amino Acid Sequences of SBJ05-D14:

>CDR 1 of variable domain of Heavy chain of
SBJ05-D14
SEQ ID NO: 91
GFTFSNTW
>CDR 2 of variable domain of Heavy chain of
SBJ05-D14
SEQ ID NO: 92
IKRKVDGETT
>CDR 3 of variable domain of Heavy chain of
SBJ05-D14
SEQ ID NO: 93
TSRVLTTNDH
>CDR 1 of variable domain of Light chain of
SBJ05-D14
SEQ ID NO: 94
SSDVGRYDY
>CDR 2 of variable domain of Light chain of
SBJ05-D14 this sequence is not included in the
sequence listing because is less than 4 amino
acid
SEQ ID NO: 95
DVS (Asp-Val-Ser)
>CDR 3 of variable domain of Light chain of
SBJ05-D14
SEQ ID NO: 96
CSFTGGEIFV
>variable domain of Heavy chain of SBJ05-D14
SEQ ID NO: 97
EVQLVESGGGLVKPGGSLRLSCAASGFTFSNTWMNWVRQAPGKGLEYLGR
IKRKVDGETTHYAAPAKGRFTISRDDSKNTLYLQLSSLGTEDTALYYCTS
RVLTTNDHWGQGTLVTV
>variable domain of Light chain of SBJ05-D14
SEQ ID NO: 98
QSALTQPASVSGSPGQSITISCTGTSSDVGRYDYVSWYQQHPGKVPKLLI
YDVSNRPSGGSNRFSGSKSGNTASLTISGLQPEDEADYYCCSFTGGEIFV
FGTGTKVTVL
>Heavy chain of SBJ05-D14
SEQ ID NO: 99
MGWSCIILFLVATATGVHSEVQLVESGGGLVKPGGSLRLSCAASGFTFSN
TWMNWVRQAPGKGLEYLGRIKRKVDGETTHYAAPAKGRFTISRDDSKNTL
YLQLSSLGTEDTALYYCTSRVLTTNDHWGQGTLVTVHSSVRSTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSV
MHGALHNHYTQKSLSLFPG*
>Light chain of SBJ05-D14
SEQ ID NO: 100
MGWSCIILFLVATATGSWAQSALTQPASVSGSPGQSITISCTGTSSDVGR
YDYVSWYQQHPGKVPKLLIYDVSNRPSGGSNRFSGSKSGNTASLTISGLQ
PEDEADYYCCSFTGGEIFVFGTGTKVTVLGQPKANPTVTLFPPSSEELQA
NKATLVCLISDFYPGAVTVAWKADSSPVKAGVETTTPSKQSNNKYAASSY
LSLTPEQWKSHRSYSCQVTHEGSTVEKTVAPTECS*

### Nucleic Acid Sequences of SBJ05-D14:

> SBJ05-D14_heavy_chain leader sequence
SEQ ID NO: 101
ATGGGATGGTCATGTATCATCCTTTTTCTAGTAGCAACTGCAACCGGTGTACAT
TCC
> SBJ05-D14_heavy_chain variable domain
SEQ ID NO: 102
GAGGTGCAGCTGGTGGAGTCTGGGGGAGGCTTGGTAAAGCCTGGGGGGTCCCT
TAGACTCTCCTGTGCGGCCTCTGGATTCACTTTCAGTAACACCTGGATGAACTG
GGTCCGCCAGGCTCCAGGGAAGGGGCTGGAGTACCTTGGTCGTATTAAAAGGA
AAGTTGATGGTGAGACAACACACTACGCTGCACCCGCGAAAGGCAGATTCACC
ATCTCAAGAGATGATTCCAAAAACACTCTGTATCTGCAACTGAGCAGCCTGGG
AACCGAGGACACAGCCCTATATTATTGCACCTCTCGAGTCCTGACTACCAATG
ACCACTGGGGCCAGGGAACCCTGGTCACCGT
> SBJ05-D14_heavy_chain constant domain
SEQ ID NO: 103
ACACTCGAGCGTACGGTCGACCAAGGGCCCATCGGTCTTCCCCCTGGCACCCT
CCTCCAAGAGCACCTCTGGGGGCACAGCGGCCCTGGGCTGCCTGGTCAAGGAC
TACTTCCCCGAACCTGTGACGGTCTCGTGGAACTCAGGCGCCCTGACCAGCGG
CGTGCACACCTTCCCGGCTGTCCTACAGTCCTCAGGACTCTACTCCCTCAGCAG
CGTGGTGACCGTGCCCTCCAGCAGCTTGGGCACCCAGACCTACATCTGCAACG
TGAATCACAAGCCCAGCAACACCAAGGTGGACAAGAGAGTTGAGCCCAAATC
TTGTGACAAAACTCACACATGCCCACCGTGCCCAGCACCTGAACTCCTGGGGG
GACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAG
GTCAAGTTCAACTGGTACGTGGACGGCGTGGAGGTGCATAATGCCAAGACAAA
GCCGCGGGAGGAGCAGTACAACAGCACGTACCGTGTGGTCAGCGTCCTCACCG
TCCTGCACCAGGACTGGCTGAATGGCAAGGAGTACAAGTGCAAGGTCTCCAAC
AAAGCCCTCCCAGCCCCCATCGAGAAAACCATCTCCAAAGCCAAAGGGCAGCC
CCGAGAACCACAGGTGTACACCCTGCCCCCATCCCGGGAGGAGATGACCAAG
AACCAGGTCAGCCTGACCTGCCTGGTCAAAGGCTTCTATCCCAGCGACATCGC
CGTGGAGTGGGAGAGCAATGGGCAGCCGGAGAACAACTACAAGACCACGCCT
CCCGTGCTGGACTCCGACGGCTCCTTCTTCCTCTATAGCAAGCTCACCGTGGAC
AAGAGCAGGTGGCAGCAGGGGAACGTCTTCTCATGCTCCGTGATGCATGGCGC
TCTGCACAACCACTACACGCAGAAGAGCCTCTCCCTGTTCCCCGGGTAA
> SBJ05-D14_heavy_chain complete
SEQ ID NO: 104
ATGGGATGGTCATGTATCATCCTTTTTCTAGTAGCAACTGCAACCGGTGTACAT
TCCGAGGTGCAGCTGGTGGAGTCTGGGGGAGGCTTGGTAAAGCCTGGGGGGTC
CCTTAGACTCTCCTGTGCGGCCTCTGGATTCACTTTCAGTAACACCTGGATGAA
CTGGGTCCGCCAGGCTCCAGGGAAGGGGCTGGAGTACCTTGGTCGTATTAAAA
GGAAAGTTGATGGTGAGACAACACACTACGCTGCACCCGCGAAAGGCAGATT
CACCATCTCAAGAGATGATTCCAAAAACACTCTGTATCTGCAACTGAGCAGCC
TGGGAACCGAGGACACAGCCCTATATTATTGCACCTCTCGAGTCCTGACTACC
AATGACCACTGGGGCCAGGGAACCCTGGTCACCGTACACTCGAGCGTACGGTC
GACCAAGGGCCCATCGGTCTTCCCCCTGGCACCCTCCTCCAAGAGCACCTCTG
GGGGCACAGCGGCCCTGGGCTGCCTGGTCAAGGACTACTTCCCCGAACCTGTG
ACGGTCTCGTGGAACTCAGGCGCCCTGACCAGCGGCGTGCACACCTTCCCGGC
TGTCCTACAGTCCTCAGGACTCTACTCCCTCAGCAGCGTGGTGACCGTGCCCTC
CAGCAGCTTGGGCACCCAGACCTACATCTGCAACGTGAATCACAAGCCCAGCA
ACACCAAGGTGGACAAGAGAGTTGAGCCCAAATCTTGTGACAAAACTCACAC
ATGCCCACCGTGCCCAGCACCTGAACTCCTGGGGGGACCGTCAGTCTTCCTCTT
CCCCCCAAAACCCAAGGACACCCTCATGATCTCCCGGACCCCTGAGGTCACAT
GCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGT
ACAACAGCACGTACCGTGTGGTCAGCGTCCTCACCGTCCTGCACCAGGACTGG
CTGAATGGCAAGGAGTACAAGTGCAAGGTCTCCAACAAAGCCCTCCCAGCCCC
CATCGAGAAAACCATCTCCAAAGCCAAAGGGCAGCCCCGAGAACCACAGGTG
TACACCCTGCCCCCATCCCGGGAGGAGATGACCAAGAACCAGGTCAGCCTGAC
CTGCCTGGTCAAAGGCTTCTATCCCAGCGACATCGCCGTGGAGTGGGAGAGCA
ATGGGCAGCCGGAGAACAACTACAAGACCACGCCTCCCGTGCTGGACTCCGAC
GGCTCCTTCTTCCTCTATAGCAAGCTCACCGTGGACAAGAGCAGGTGGCAGCA
GGGGAACGTCTTCTCATGCTCCGTGATGCATGGCGCTCTGCACAACCACTACA
CGCAGAAGAGCCTCTCCCTGTTCCCCGGGTAA
> SBJ05-D14_light_kappa_chain leader sequence
SEQ ID NO: 105
ATGGGATGGTCATGTATCATCCTTTTTCTAGTAGCAACTGCAACCGGTTCCTGG
GCC
> SBJ05-D14_light_kappa_chain variable domain
SEQ ID NO: 106
CAGTCTGCCCTGACTCAGCCTGCCTCCGTGTCTGGGTCTCCTGGACAGTCGATC
ACCATCTCCTGCACTGGAACCAGCAGTGACGTTGGTCGTTATGACTATGTCTCC
TGGTACCAACAGCACCCAGGCAAAGTCCCCAAATTGTTGATTTATGATGTCAG
TAATCGGCCCTCAGGGGGATCCAATCGCTTCTCTGGCTCCAAGTCTGGCAACA
CGGCCTCCCTGACCATCTCTGGACTCCAGCCTGAGGACGAGGCCGATTATTACT
GCTGCTCATTTACTGGCGGCGAAATTTTTGTCTTCGGAACTGGGACCAAGGTCA
CTGTCCT
> SBJ05-D14_light_kappa_chain constant domain
SEQ ID NO: 107
TGGTCAGCCCAAGGCCAACCCCACTGTCACTCTGTTCCCACCCTCGAGTGAGG
AGCTTCAAGCCAACAAGGCCACACTGGTGTGTCTCATAAGTGACTTCTACCCG
GGAGCCGTGACAGTGGCCTGGAAGGCAGATAGCAGCCCCGTCAAGGCGGGAG
TGGAGACCACCACACCCTCCAAACAAAGCAACAACAAGTACGCGGCCAGCAG
CTACCTGAGCCTGACGCCTGAGCAGTGGAAGTCCCACAGAAGCTACAGCTGCC
AGGTCACGCATGAAGGGAGCACCGTGGAGAAGACAGTGGCCCCTACAGAATG
TTCATAG
> SBJ05-D14_light_kappa_chain complete
SEQ ID NO: 108
ATGGGATGGTCATGTATCATCCTTTTTCTAGTAGCAACTGCAACCGGTTCCTGG
GCCCAGTCTGCCCTGACTCAGCCTGCCTCCGTGTCTGGGTCTCCTGGACAGTCG
ATCACCATCTCCTGCACTGGAACCAGCAGTGACGTTGGTCGTTATGACTATGTC
TCCTGGTACCAACAGCACCCAGGCAAAGTCCCCAAATTGTTGATTTATGATGTC
AGTAATCGGCCCTCAGGGGGATCCAATCGCTTCTCTGGCTCCAAGTCTGGCAA
CACGGCCTCCCTGACCATCTCTGGACTCCAGCCTGAGGACGAGGCCGATTATT
ACTGCTGCTCATTTACTGGCGGCGAAATTTTTGTCTTCGGAACTGGGACCAAGG
TCACTGTCCTTGGTCAGCCCAAGGCCAACCCCACTGTCACTCTGTTCCCACCCT
CGAGTGAGGAGCTTCAAGCCAACAAGGCCACACTGGTGTGTCTCATAAGTGAC
TTCTACCCGGGAGCCGTGACAGTGGCCTGGAAGGCAGATAGCAGCCCCGTCAA
GGCGGGAGTGGAGACCACCACACCCTCCAAACAAAGCAACAACAAGTACGCG
GCCAGCAGCTACCTGAGCCTGACGCCTGAGCAGTGGAAGTCCCACAGAAGCTA
CAGCTGCCAGGTCACGCATGAAGGGAGCACCGTGGAGAAGACAGTGGCCCCT
ACAGAATGTTCATAG

### Sequence of the Antibody Herein Identified as SBJ05-K07

### Amino Acid Sequences of SBJ05-K07:

>CDR 1 of variable domain of Heavy chain of
SBJ05-K07
SEQ ID NO: 127
EFTFSSYA
>CDR 2 of variable domain of Heavy chain of
SBJ05-K07
SEQ ID NO: 128
ISTGGDRT
>CDR 3 of variable domain of Heavy chain of
SBJ05-K07
SEQ ID NO: 129
AKSLESGSIPTRVRALDY
>CDR 1 of variable domain of Light chain of
SBJ05-K07
SEQ ID NO: 130
QGITNF
>CDR 2 of variable domain of Light chain of
SBJ05-K07 this sequence is not included in the
sequence listing because is less than 4 amino
acid
SEQ ID NO: 131
AAS (Ala-Ala-Ser)
>CDR 3 of variable domain of Light chain of
SBJ05-K07
SEQ ID NO: 132
QKYNSAPWT
>variable domain of Heavy chain of SBJ05-K07
SEQ ID NO: 133
EVQLVESGGDLVQPGGSLRLSCAASEFTFSSYAMSWVRQAPGKGLEWVAV
ISTGGDRTYYADSVKGRFTISRDNSKNTLYLQMNSLRVEDTAVYYCAKSL
ESGSIPTRVRALDYWGQGTLVTVSS
>variable domain of Light chain of SBJ05-K07
SEQ ID NO: 134
AIQMTQSPSSLSASVGDRVTITCRASQGITNFLAWYQQKPGKVPKLLIYA
ASTLQSGVPSRFSGSGSGTDFTLTISSLQPEDVATYYCQKYNSAPWTFGQ
GTKVEIK
>Heavy chain of SBJ05-K07
SEQ ID NO: 135
MGWSCIILFLVATATGVHSEVQLVESGGDLVQPGGSLRLSCAASEFTFSS
YAMSWVRQAPGKGLEWVAVISTGGDRTYYADSVKGRFTISRDNSKNTLYL
QMNSLRVEDTAVYYCAKSLESGSIPTRVRALDYWGQGTLVTVSSASTKGP
SVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSCDKT
HTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVF
SCSVMHGALHNHYTQKSLSLFPG*
>Light chain of SBJ05-K07
SEQ ID NO: 136
MGWSCIILFLVATATGVHSAIQMTQSPSSLSASVGDRVTITCRASQGITN
FLAWYQQKPGKVPKLLIYAASTLQSGVPSRFSGSGSGTDFTLTISSLQPE
DVATYYCQKYNSAPWTFGQGTKVEIKRTVAAPSVFIFPPSDEQLKSGTAS
VVCLLNNFYPREAKVQWKVDNALQSGNSQESVTEQDSKDSTYSLSSTLTL
SKADYEKHKVYACEVTHQGLSSPVTKSFNRGEC*

### Nucleic Acid Sequences of SBJ05-K07:

> SBJ05-K07_heavy_chain leader sequence
SEQ ID NO: 137
ATGGGATGGTCATGTATCATCCTTTTTCTAGTAGCAACTGCAACCGGTGTACAT
TCC
> SBJ05-K07_heavy_chain variable domain
SEQ ID NO: 138
GAGGTGCAGCTGGTGGAGTCTGGGGGAGACTTGGTACAGCCTGGGGGGTCCCT
GAGACTCTCCTGTGCAGCCTCTGAATTCACCTTTAGCAGCTATGCCATGAGCTG
GGTCCGCCAGGCTCCAGGGAAGGGGCTGGAGTGGGTCGCAGTTATTAGTACTG
GTGGTGATAGGACATACTACGCAGACTCCGTGAAGGGCCGGTTCACCATCTCC
AGAGACAATTCCAAGAACACGTTGTATCTGCAAATGAACAGCCTGAGAGTCGA
GGACACGGCCGTATATTACTGTGCGAAATCCCTCGAAAGTGGTTCGATACCGA
CTCGGGTCCGCGCTTTGGACTACTGGGGCCAGGGAACCCTGGTCACCGTCTCCT
CAG
> SBJ05-K07_heavy_chain constant domain
SEQ ID NO: 139
CTTCCACCAAGGGCCCATCGGTCTTCCCCCTGGCACCCTCCTCCAAGAGCACCT
CTGGGGGCACAGCGGCCCTGGGCTGCCTGGTCAAGGACTACTTCCCCGAACCT
GTGACGGTCTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCC
CTCCAGCAGCTTGGGCACCCAGACCTACATCTGCAACGTGAATCACAAGCCCA
GCAACACCAAGGTGGACAAGAGAGTTGAGCCCAAATCTTGTGACAAAACTCA
CACATGCCCACCGTGCCCAGCACCTGAACTCCTGGGGGGACCGTCAGTCTTCC
TCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAG
CAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCACA
GGTGTACACCCTGCCCCCATCCCGGGAGGAGATGACCAAGAACCAGGTCAGCC
TGACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTC
CGACGGCTCCTTCTTCCTCTATAGCAAGCTCACCGTGGACAAGAGCAGGTGGC
AGCAGGGGAACGTCTTCTCATGCTCCGTGATGCATGGCGCTCTGCACAACCAC
TACACGCAGAAGAGCCTCTCCCTGTTCCCCGGGTAA
> SBJ05-K07_heavy_chain complete
SEQ ID NO: 140
ATGGGATGGTCATGTATCATCCTTTTTCTAGTAGCAACTGCAACCGGTGTACAT
TCCGAGGTGCAGCTGGTGGAGTCTGGGGGAGACTTGGTACAGCCTGGGGGGTC
CCTGAGACTCTCCTGTGCAGCCTCTGAATTCACCTTTAGCAGCTATGCCATGAG
CTGGGTCCGCCAGGCTCCAGGGAAGGGGCTGGAGTGGGTCGCAGTTATTAGTA
CTGGTGGTGATAGGACATACTACGCAGACTCCGTGAAGGGCCGGTTCACCATC
TCCAGAGACAATTCCAAGAACACGTTGTATCTGCAAATGAACAGCCTGAGAGT
CGAGGACACGGCCGTATATTACTGTGCGAAATCCCTCGAAAGTGGTTCGATAC
CGACTCGGGTCCGCGCTTTGGACTACTGGGGCCAGGGAACCCTGGTCACCGTC
TCCTCAGCTTCCACCAAGGGCCCATCGGTCTTCCCCCTGGCACCCTCCTCCAAG
AGCACCTCTGGGGGCACAGCGGCCCTGGGCTGCCTGGTCAAGGACTACTTCCC
CGAACCTGTGACGGTCTCGTGGAACTCAGGCGCCCTGACCAGCGGCGTGCACA
CCTTCCCGGCTGTCCTACAGTCCTCAGGACTCTACTCCCTCAGCAGCGTGGTGA
CCGTGCCCTCCAGCAGCTTGGGCACCCAGACCTACATCTGCAACGTGAATCAC
AAGCCCAGCAACACCAAGGTGGACAAGAGAGTTGAGCCCAAATCTTGTGACA
AAACTCACACATGCCCACCGTGCCCAGCACCTGAACTCCTGGGGGGACCGTCA
GTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTT
CAACTGGTACGTGGACGGCGTGGAGGTGCATAATGCCAAGACAAAGCCGCGG
GAGGAGCAGTACAACAGCACGTACCGTGTGGTCAGCGTCCTCACCGTCCTGCA
CCAGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAG
GTCAGCCTGACCTGCCTGGTCAAAGGCTTCTATCCCAGCGACATCGCCGTGGA
GTGGGAGAGCAATGGGCAGCCGGAGAACAACTACAAGACCACGCCTCCCGTG
CTGGACTCCGACGGCTCCTTCTTCCTCTATAGCAAGCTCACCGTGGACAAGAGC
AGGTGGCAGCAGGGGAACGTCTTCTCATGCTCCGTGATGCATGGCGCTCTGCA
CAACCACTACACGCAGAAGAGCCTCTCCCTGTTCCCCGGGTAA
> SBJ05-K07_light_kappa_chain leader sequence
SEQ ID NO: 141
ATGGGATGGTCATGTATCATCCTTTTTCTAGTAGCAACTGCAACCGGTGTACAT
TCC
> SBJ05-K07_light_kappa_chain variable domain
SEQ ID NO: 142
GCCATCCAGATGACCCAGTCTCCATCCTCCCTGTCTGCATCTGTAGGAGACCGA
GTCACCATCACTTGCCGGGCGAGTCAGGGCATTACCAATTTTTTAGCCTGGTAT
CAGCAGAAACCAGGGAAAGTTCCTAAGCTCCTGATCTATGCTGCATCCACTTT
GCAATCAGGGGTCCCATCTCGGTTCAGTGGCAGTGGATCTGGGACAGATTTCA
CTCTCACCATCAGCAGCCTGCAGCCTGAAGATGTTGCAACTTATTACTGTCAAA
AGTATAACAGTGCCCCGTGGACGTTCGGCCAAGGGACCAAGGTGGAAATCAA
AC
> SBJ05-K07_light_kappa_chain constant domain
SEQ ID NO: 143
GAACTGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGT
GTTAG
> SBJ05-K07_light_kappa_chain complete
SEQ ID NO: 144
ATGGGATGGTCATGTATCATCCTTTTTCTAGTAGCAACTGCAACCGGTGTACAT
TCCGCCATCCAGATGACCCAGTCTCCATCCTCCCTGTCTGCATCTGTAGGAGAC
CGAGTCACCATCACTTGCCGGGCGAGTCAGGGCATTACCAATTTTTTAGCCTGG
TATCAGCAGAAACCAGGGAAAGTTCCTAAGCTCCTGATCTATGCTGCATCCAC
TTTGCAATCAGGGGTCCCATCTCGGTTCAGTGGCAGTGGATCTGGGACAGATTT
CACTCTCACCATCAGCAGCCTGCAGCCTGAAGATGTTGCAACTTATTACTGTCA
AAAGTATAACAGTGCCCCGTGGACGTTCGGCCAAGGGACCAAGGTGGAAATC
AAACGAACTGTGGCTGCACCATCTGTCTTCATCTTCCCGCCATCTGATGAGCAG
TTGAAATCTGGAACTGCCTCTGTTGTGTGCCTGCTGAATAACTTCTATCCCAGA
GAGGCCAAAGTACAGTGGAAGGTGGATAACGCCCTCCAATCGGGTAACTCCCA
GGAGAGTGTCACAGAGCAGGACAGCAAGGACAGCACCTACAGCCTCAGCAGC
ACCCTGACGCTGAGCAAAGCAGACTACGAGAAACACAAAGTCTACGCCTGCG
AAGTCACCCATCAGGGCCTGAGCTCGCCCGTCACAAAGAGCTTCAACAGGGGA
GAGTGTTAG

### Sequence of the Antibody Herein Identified as SBJ05-M13

### Amino Acid Sequences of SBJ05-M13:

>CDR 1 of variable domain of Heavy chain of
SBJ05-M13
SEQ ID NO: 145
GFSFSDYA
>CDR 2 of variable domain of Heavy chain of
SBJ05-M13
SEQ ID NO: 146
IDNNGANT
>CDR 3 of variable domain of Heavy chain of
SBJ05-M13
SEQ ID NO: 147
VRGTTT
>CDR 1 of variable domain of Light chain of
SBJ05-M13
SEQ ID NO: 148
QSLVHSDGNTY
>CDR 2 of variable domain of Light chain of
SBJ05-M13 this sequence is not included in the
sequence listing because is less than 4 amino
acid
SEQ ID NO: 149
EIS (Glu-Ile-Ser)
>CDR 3 of variable domain of Light chain of
SBJ05-M13
SEQ ID NO: 150
LQATHFPHGT
>variable domain of Heavy chain of SBJ05-M13
SEQ ID NO: 151
QVQLVQSGGGLVQPGGSLRLSCSASGFSFSDYAVHWVRQPPGKGLEYLSA
IDNNGANTFYVDSVKGRFTISRDNSKNMLYLQMSGLRVDDTAVYYCVRGT
TTWGQGTLVTVSS
>variable domain of Light chain of SBJ05-M13
SEQ ID NO: 152
DIVMTQSPLSSSVTLGQPASISCRSSQSLVHSDGNTYLSWLQQRPGQPPR
LLISEISKRFSGVPDRFSGSGAGTDFTLKISRVEAEDVGVYYCLQATHFP
HGTFGQGTKLEIK
>Heavy chain of SBJ05-M13
SEQ ID NO: 153
MGWSCIILFLVATATGVHSQVQLVQSGGGLVQPGGSLRLSCSASGFSFSD
YAVHWVRQPPGKGLEYLSAIDNNGANTFYVDSVKGRFTISRDNSKNMLYL
QMSGLRVDDTAVYYCVRGTTTWGQGTLVTVSSASTKGPSVFPLAPSSKST
SGGTAALGCLVKDYFPEPVTVSWNSGALTSGVHTFPAVLQSSGLYSLSSV
VTVPSSSLGTQTYICNVNHKPSNTKVDKRVEPKSCDKTHTCPPCPAPELL
GGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHGALHNH
YTQKSLSLFPG*
>Light chain of SBJ05-M13
SEQ ID NO: 154
MGWSCIILFLVATATGVHSDIVMTQSPLSSSVTLGQPASISCRSSQSLVH
SDGNTYLSWLQQRPGQPPRLLISEISKRFSGVPDRFSGSGAGTDFTLKIS
RVEAEDVGVYYCLQATHFPHGTFGQGTKLEIKRTVAAPSVFIFPPSDEQL
KSGTASVVCLLNNFYPREAKVQWKVDNALQSGNSQESVTEQDSKDSTYSL
SSTLTLSKADYEKHKVYACEVTHQGLSSPVTKSFNRGEC*

### Nucleic Acid Sequences of SBJ05-M13:

> SBJ05-M13_heavy_chain leader sequence
SEQ ID NO: 155
ATGGGATGGTCATGTATCATCCTTTTTCTAGTAGCAACTGCAACCGGTGTACAT
TCC
> SBJ05-M13_heavy_chain variable domain
SEQ ID NO: 156
CAGGTGCAGCTGGTGCAGTCTGGGGGCGGCTTGGTCCAGCCGGGGGGGTCCCT
GAGACTCTCCTGTTCAGCCTCTGGATTCAGCTTCAGTGACTATGCTGTGCACTG
GGTCCGCCAGCCTCCAGGGAAGGGACTGGAATACCTTTCAGCTATTGATAATA
ATGGGGCTAACACATTCTACGTAGACTCCGTGAAGGGCAGATTCACCATCTCC
AGAGACAATTCCAAGAACATGTTGTATCTTCAGATGAGTGGGCTGAGAGTTGA
CGACACCGCTGTGTATTACTGTGTGAGGGGGACTACCACCTGGGGCCAGGGAA
CCCTAGTCACCGTCTCCTCAG
> SBJ05-M13_heavy_chain constant domain
SEQ ID NO: 157
CTTCCACCAAGGGCCCATCGGTCTTCCCCCTGGCACCCTCCTCCAAGAGCACCT
CTGGGGGCACAGCGGCCCTGGGCTGCCTGGTCAAGGACTACTTCCCCGAACCT
GTGACGGTCTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCC
CTCCAGCAGCTTGGGCACCCAGACCTACATCTGCAACGTGAATCACAAGCCCA
GCAACACCAAGGTGGACAAGAGAGTTGAGCCCAAATCTTGTGACAAAACTCA
CACATGCCCACCGTGCCCAGCACCTGAACTCCTGGGGGGACCGTCAGTCTTCC
TCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAG
CAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCACA
GGTGTACACCCTGCCCCCATCCCGGGAGGAGATGACCAAGAACCAGGTCAGCC
TGACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTC
CGACGGCTCCTTCTTCCTCTATAGCAAGCTCACCGTGGACAAGAGCAGGTGGC
AGCAGGGGAACGTCTTCTCATGCTCCGTGATGCATGGCGCTCTGCACAACCAC
TACACGCAGAAGAGCCTCTCCCTGTTCCCCGGGTAA
> SBJ05-M13_heavy_chain complete
SEQ ID NO: 158
ATGGGATGGTCATGTATCATCCTTTTTCTAGTAGCAACTGCAACCGGTGTACAT
TCCCAGGTGCAGCTGGTGCAGTCTGGGGGCGGCTTGGTCCAGCCGGGGGGGTC
CCTGAGACTCTCCTGTTCAGCCTCTGGATTCAGCTTCAGTGACTATGCTGTGCA
CTGGGTCCGCCAGCCTCCAGGGAAGGGACTGGAATACCTTTCAGCTATTGATA
ATAATGGGGCTAACACATTCTACGTAGACTCCGTGAAGGGCAGATTCACCATC
TCCAGAGACAATTCCAAGAACATGTTGTATCTTCAGATGAGTGGGCTGAGAGT
TGACGACACCGCTGTGTATTACTGTGTGAGGGGGACTACCACCTGGGGCCAGG
GAACCCTAGTCACCGTCTCCTCAGCTTCCACCAAGGGCCCATCGGTCTTCCCCC
TGGCACCCTCCTCCAAGAGCACCTCTGGGGGCACAGCGGCCCTGGGCTGCCTG
GTCAAGGACTACTTCCCCGAACCTGTGACGGTCTCGTGGAACTCAGGCGCCCT
GACCAGCGGCGTGCACACCTTCCCGGCTGTCCTACAGTCCTCAGGACTCTACTC
CCTCAGCAGCGTGGTGACCGTGCCCTCCAGCAGCTTGGGCACCCAGACCTACA
TCTGCAACGTGAATCACAAGCCCAGCAACACCAAGGTGGACAAGAGAGTTGA
GCCCAAATCTTGTGACAAAACTCACACATGCCCACCGTGCCCAGCACCTGAAC
TCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAA
GACCCTGAGGTCAAGTTCAACTGGTACGTGGACGGCGTGGAGGTGCATAATGC
CAAGACAAAGCCGCGGGAGGAGCAGTACAACAGCACGTACCGTGTGGTCAGC
GTCCTCACCGTCCTGCACCAGGACTGGCTGAATGGCAAGGAGTACAAGTGCAA
GGTCTCCAACAAAGCCCTCCCAGCCCCCATCGAGAAAACCATCTCCAAAGCCA
AAGGGCAGCCCCGAGAACCACAGGTGTACACCCTGCCCCCATCCCGGGAGGA
GATGACCAAGAACCAGGTCAGCCTGACCTGCCTGGTCAAAGGCTTCTATCCCA
GCGACATCGCCGTGGAGTGGGAGAGCAATGGGCAGCCGGAGAACAACTACAA
GACCACGCCTCCCGTGCTGGACTCCGACGGCTCCTTCTTCCTCTATAGCAAGCT
CACCGTGGACAAGAGCAGGTGGCAGCAGGGGAACGTCTTCTCATGCTCCGTGA
TGCATGGCGCTCTGCACAACCACTACACGCAGAAGAGCCTCTCCCTGTTCCCC
GGGTAA
> SBJ05-M13_light_kappa_chain leader sequence
SEQ ID NO: 159
ATGGGATGGTCATGTATCATCCTTTTTCTAGTAGCAACTGCAACCGGTGTACAT
TCC
> SBJ05-M13_light_kappa_chain variable domain
SEQ ID NO: 160
GATATTGTGATGACTCAGTCTCCACTCTCCTCATCTGTCACCCTTGGACAGCCG
GCCTCCATCTCCTGCAGGTCTAGTCAAAGCCTCGTACACAGTGATGGAAACAC
TTACTTGAGTTGGCTTCAGCAGAGGCCAGGCCAGCCACCAAGACTCCTAATTT
CTGAGATTTCTAAGCGGTTCTCTGGGGTCCCAGACAGATTCAGTGGCAGTGGG
GCAGGGACAGATTTCACACTGAAAATCAGCAGGGTGGAGGCTGAAGATGTCG
GGGTTTATTATTGCTTGCAAGCTACACATTTTCCTCATGGGACTTTTGGCCAGG
GGACCAAGCTGGAGATCAAAC
> SBJ05-M13_light_kappa_chain constant domain
SEQ ID NO: 161
GAACTGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGT
GTTAG
> SBJ05-M13_light_kappa_chain complete
SEQ ID NO: 162
ATGGGATGGTCATGTATCATCCTTTTTCTAGTAGCAACTGCAACCGGTGTACAT
TCCGATATTGTGATGACTCAGTCTCCACTCTCCTCATCTGTCACCCTTGGACAG
CCGGCCTCCATCTCCTGCAGGTCTAGTCAAAGCCTCGTACACAGTGATGGAAA
CACTTACTTGAGTTGGCTTCAGCAGAGGCCAGGCCAGCCACCAAGACTCCTAA
TTTCTGAGATTTCTAAGCGGTTCTCTGGGGTCCCAGACAGATTCAGTGGCAGTG
GGGCAGGGACAGATTTCACACTGAAAATCAGCAGGGTGGAGGCTGAAGATGT
CGGGGTTTATTATTGCTTGCAAGCTACACATTTTCCTCATGGGACTTTTGGCCA
GGGGACCAAGCTGGAGATCAAACGAACTGTGGCTGCACCATCTGTCTTCATCT
TCCCGCCATCTGATGAGCAGTTGAAATCTGGAACTGCCTCTGTTGTGTGCCTGC
TGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACA
GCACCTACAGCCTCAGCAGCACCCTGACGCTGAGCAAAGCAGACTACGAGAA
ACACAAAGTCTACGCCTGCGAAGTCACCCATCAGGGCCTGAGCTCGCCCGTCA
CAAAGAGCTTCAACAGGGGAGAGTGTTAG

### Sequence of the Antibody Herein Identified as SBJ05-N02

### Amino Acid Sequences of Sbj05-N02:

>CDR 1 of variable domain of Heavy chain of
SBJ05-N02
SEQ ID NO: 163
GFTFSNHW
>CDR 2 of variable domain of Heavy chain of
SBJ05-N02
SEQ ID NO: 164
INPDGTYT
>CDR 3 of variable domain of Heavy chain of
SBJ05-N02
SEQ ID NO: 165
ARDLPRSDPPGVGWGSGMDV
>CDR 1 of variable domain of Light chain of
SBJ05-N02
SEQ ID NO: 166
QTISTY
>CDR 2 of variable domain of Light chain of
SBJ05-N02 this sequence isnot included in the
sequence listing because is less than 4 amino
acid
SEQ ID NO: 167
ASS (Ala-Ser-Ser)
>CDR 3 of variable domain of Light chain of
SBJ05-N02
SEQ ID NO: 168
QQSYSTPPT
>variable domain of Heavy chain of SBJ05-N02
SEQ ID NO: 169
EVQLVESGGGLVQPGGSLRLSCAASGFTFSNHWMHWVRQGPGKGLVWVSR
INPDGTYTSYADSVSGRFTIARDNAKNTLYLHMNSLRDEDTAVYYCARDL
PRSDPPGVGWGSGMDVWGQGTRVTVSS
>variable domain of Light chain of SBJ05-N02
SEQ ID NO: 170
AIQMTQSPSSLSASAGDRVTITCRASQTISTYLSWYQQKPGKAPKMLIYA
SSSLQSGVPSRFSGSGSGTDFTLTISRLQPEDFATYYCQQSYSTPPTFGP
GTKVEIK
>Heavy chain of SBJ05-N02
SEQ ID NO: 171
MGWSCIILFLVATATGVHSEVQLVESGGGLVQPGGSLRLSCAASGFTFSN
HWMHWVRQGPGKGLVWVSRINPDGTYTSYADSVSGRFTIARDNAKNTLYL
HMNSLRDEDTAVYYCARDLPRSDPPGVGWGSGMDVWGQGTRVTVSSASTK
GPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSCD
KTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGN
VFSCSVMHGALHNHYTQKSLSLFPG*
>Light chain of SBJ05-N02
SEQ ID NO: 172
MGWSCIILFLVATATGVHSAIQMTQSPSSLSASAGDRVTITCRASQTIST
YLSWYQQKPGKAPKMLIYASSSLQSGVPSRFSGSGSGTDFTLTISRLQPE
DFATYYCQQSYSTPPTFGPGTKVEIKRTVAAPSVFIFPPSDEQLKSGTAS
VVCLLNNFYPREAKVQWKVDNALQSGNSQESVTEQDSKDSTYSLSSTLTL
SKADYEKHKVYACEVTHQGLSSPVTKSFNRGEC*

### Nucleic Acid Sequences of SBJ05-N02:

SBJ05-N02 heavy_chain leader sequence
> SEQ ID NO: 173
ATGGGATGGTCATGTATCATCCTTTTTCTAGTAGCAACTGCAACCGGTGTACAT
TCC
SBJ05-N02_heavy_chain variable domain
> SEQ ID NO: 174
GAGGTGCAGCTGGTGGAGTCTGGGGGAGGCTTAGTTCAGCCGGGGGGGTCCCT
GAGACTCTCCTGTGCAGCCTCTGGATTCACCTTCAGTAACCACTGGATGCACTG
GGTCCGCCAAGGTCCAGGGAAGGGACTGGTGTGGGTCTCACGAATTAATCCTG
ATGGGACTTACACAAGTTACGCGGACTCCGTGAGTGGCCGATTCACCATCGCC
AGAGACAACGCCAAGAACACCCTGTACCTGCACATGAACAGTCTGAGAGACG
AGGACACGGCTGTATATTACTGTGCAAGAGACTTGCCCAGATCTGACCCCCCC
GGTGTGGGATGGGGCTCCGGTATGGACGTCTGGGGCCAAGGGACCAGGGTCA
CCGTCTCCTCA
SBJ05-N02_heavy_chain constant domain
> SEQ ID NO: 175
GCCTCCACCAAGGGCCCATCGGTCTTCCCCCTGGCACCCTCCTCCAAGAGCAC
CTCTGGGGGCACAGCGGCCCTGGGCTGCCTGGTCAAGGACTACTTCCCCGAAC
CTGTGACGGTCTCGTGGAACTCAGGCGCCCTGACCAGCGGCGTGCACACCTTC
CCGGCTGTCCTACAGTCCTCAGGACTCTACTCCCTCAGCAGCGTGGTGACCGTG
CCCTCCAGCAGCTTGGGCACCCAGACCTACATCTGCAACGTGAATCACAAGCC
CAGCAACACCAAGGTGGACAAGAGAGTTGAGCCCAAATCTTGTGACAAAACT
CACACATGCCCACCGTGCCCAGCACCTGAACTCCTGGGGGGACCGTCAGTCTT
CCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACT
GGTACGTGGACGGCGTGGAGGTGCATAATGCCAAGACAAAGCCGCGGGAGGA
GCAGTACAACAGCACGTACCGTGTGGTCAGCGTCCTCACCGTCCTGCACCAGG
ACTGGCTGAATGGCAAGGAGTACAAGTGCAAGGTCTCCAACAAAGCCCTCCCA
GCCCCCATCGAGAAAACCATCTCCAAAGCCAAAGGGCAGCCCCGAGAACCAC
AGGTGTACACCCTGCCCCCATCCCGGGAGGAGATGACCAAGAACCAGGTCAGC
CTGACCTGCCTGGTCAAAGGCTTCTATCCCAGCGACATCGCCGTGGAGTGGGA
GAGCAATGGGCAGCCGGAGAACAACTACAAGACCACGCCTCCCGTGCTGGAC
TCCGACGGCTCCTTCTTCCTCTATAGCAAGCTCACCGTGGACAAGAGCAGGTG
GCAGCAGGGGAACGTCTTCTCATGCTCCGTGATGCATGGCGCTCTGCACAACC
ACTACACGCAGAAGAGCCTCTCCCTGTTCCCCGGGTAA
SBJ05-N02_heavy_chain complete
> SEQ ID NO: 176
ATGGGATGGTCATGTATCATCCTTTTTCTAGTAGCAACTGCAACCGGTGTACAT
TCCGAGGTGCAGCTGGTGGAGTCTGGGGGAGGCTTAGTTCAGCCGGGGGGGTC
CCTGAGACTCTCCTGTGCAGCCTCTGGATTCACCTTCAGTAACCACTGGATGCA
CTGGGTCCGCCAAGGTCCAGGGAAGGGACTGGTGTGGGTCTCACGAATTAATC
CTGATGGGACTTACACAAGTTACGCGGACTCCGTGAGTGGCCGATTCACCATC
GCCAGAGACAACGCCAAGAACACCCTGTACCTGCACATGAACAGTCTGAGAG
ACGAGGACACGGCTGTATATTACTGTGCAAGAGACTTGCCCAGATCTGACCCC
CCCGGTGTGGGATGGGGCTCCGGTATGGACGTCTGGGGCCAAGGGACCAGGGT
CACCGTCTCCTCAGCCTCCACCAAGGGCCCATCGGTCTTCCCCCTGGCACCCTC
CTCCAAGAGCACCTCTGGGGGCACAGCGGCCCTGGGCTGCCTGGTCAAGGACT
ACTTCCCCGAACCTGTGACGGTCTCGTGGAACTCAGGCGCCCTGACCAGCGGC
GTGCACACCTTCCCGGCTGTCCTACAGTCCTCAGGACTCTACTCCCTCAGCAGC
GTGGTGACCGTGCCCTCCAGCAGCTTGGGCACCCAGACCTACATCTGCAACGT
GAATCACAAGCCCAGCAACACCAAGGTGGACAAGAGAGTTGAGCCCAAATCT
TGTGACAAAACTCACACATGCCCACCGTGCCCAGCACCTGAACTCCTGGGGGG
ACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGG
TCAAGTTCAACTGGTACGTGGACGGCGTGGAGGTGCATAATGCCAAGACAAAG
CCGCGGGAGGAGCAGTACAACAGCACGTACCGTGTGGTCAGCGTCCTCACCGT
CCTGCACCAGGACTGGCTGAATGGCAAGGAGTACAAGTGCAAGGTCTCCAACA
AAGCCCTCCCAGCCCCCATCGAGAAAACCATCTCCAAAGCCAAAGGGCAGCCC
CGAGAACCACAGGTGTACACCCTGCCCCCATCCCGGGAGGAGATGACCAAGA
ACCAGGTCAGCCTGACCTGCCTGGTCAAAGGCTTCTATCCCAGCGACATCGCC
GTGGAGTGGGAGAGCAATGGGCAGCCGGAGAACAACTACAAGACCACGCCTC
CCGTGCTGGACTCCGACGGCTCCTTCTTCCTCTATAGCAAGCTCACCGTGGACA
AGAGCAGGTGGCAGCAGGGGAACGTCTTCTCATGCTCCGTGATGCATGGCGCT
CTGCACAACCACTACACGCAGAAGAGCCTCTCCCTGTTCCCCGGGTAA
SBJ05-N02_light_kappa_chain leader sequence
> SEQ ID NO: 177
ATGGGATGGTCATGTATCATCCTTTTTCTAGTAGCAACTGCAACCGGTGTACAT
TCC
SBJ05-N02_light_kappa_chain variable domain
> SEQ ID NO: 178
GCCATCCAGATGACCCAGTCTCCATCCTCCCTGTCTGCATCTGCAGGAGACAG
AGTCACCATCACTTGCCGGGCAAGTCAGACCATTAGCACCTATTTAAGTTGGT
ATCAGCAAAAACCAGGGAAAGCCCCTAAGATGTTGATCTATGCTTCATCCAGT
TTGCAAAGTGGGGTCCCATCAAGGTTCAGTGGCAGTGGATCTGGGACAGATTT
CACTCTCACCATCAGCCGTCTGCAACCTGAAGACTTTGCAACTTACTACTGTCA
ACAGAGTTACAGTACCCCTCCCACTTTCGGCCCTGGGACCAAAGTGGAGATCA
AA
SBJ05-N02_light_kappa_chain constant domain
> SEQ ID NO: 179
AGAACTGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAG
GCCAAAGTACAGTGGAAGGTGGATAACGCCCTCCAATCGGGTAACTCCCAGGA
GAGTGTCACAGAGCAGGACAGCAAGGACAGCACCTACAGCCTCAGCAGCACC
CTGACGCTGAGCAAAGCAGACTACGAGAAACACAAAGTCTACGCCTGCGAAG
TCACCCATCAGGGCCTGAGCTCGCCCGTCACAAAGAGCTTCAACAGGGGAGAG
TGTTAG
SBJ05-N02_light_kappa_chain complete
> SEQ ID NO: 180
ATGGGATGGTCATGTATCATCCTTTTTCTAGTAGCAACTGCAACCGGTGTACAT
TCCGCCATCCAGATGACCCAGTCTCCATCCTCCCTGTCTGCATCTGCAGGAGAC
AGAGTCACCATCACTTGCCGGGCAAGTCAGACCATTAGCACCTATTTAAGTTG
GTATCAGCAAAAACCAGGGAAAGCCCCTAAGATGTTGATCTATGCTTCATCCA
GTTTGCAAAGTGGGGTCCCATCAAGGTTCAGTGGCAGTGGATCTGGGACAGAT
TTCACTCTCACCATCAGCCGTCTGCAACCTGAAGACTTTGCAACTTACTACTGT
CAACAGAGTTACAGTACCCCTCCCACTTTCGGCCCTGGGACCAAAGTGGAGAT
CAAAAGAACTGTGGCTGCACCATCTGTCTTCATCTTCCCGCCATCTGATGAGCA
GTTGAAATCTGGAACTGCCTCTGTTGTGTGCCTGCTGAATAACTTCTATCCCAG
AGAGGCCAAAGTACAGTGGAAGGTGGATAACGCCCTCCAATCGGGTAACTCCC
AGGAGAGTGTCACAGAGCAGGACAGCAAGGACAGCACCTACAGCCTCAGCAG
CACCCTGACGCTGAGCAAAGCAGACTACGAGAAACACAAAGTCTACGCCTGC
GAAGTCACCCATCAGGGCCTGAGCTCGCCCGTCACAAAGAGCTTCAACAGGGG
AGAGTGTTAG

### Sequence of the Antibody Herein Identified as SBJ08-D18

### Amino Acid Sequences of SBJ08-D18:

CDR 1 of variable domain of Heavy chain of SBJ08-D18
>SEQ ID NO: 181
GFTFSNYV
CDR 2 of variable domain of Heavy chain of SBJ08-D18
>SEQ ID NO: 182
ISTTGYTT
CDR 3 of variable domain of Heavy chain of SBJ08-D18
>SEQ ID NO: 183
GTLGTTARDFDY
CDR 1 of variable domain of Light chain of SBJ08-D18
>SEQ ID NO: 184
QDIRND
CDR 2 of variable domain of Light chain of SBJ08-D18 this sequence is
not included in the sequence listing because is less than 4 amino acid
>SEQ ID NO: 185
AAS (Ala-Ala-Ser)
CDR 3 of variable domain of Light chain of SBJ08-D18
>SEQ ID NO: 186
LQHNSHPFA
variable domain of Heavy chain of SBJ08-D18
>SEQ ID NO: 187
EVQLVESGGGLVQPGGSLRLSCAASGFTFSNYVMSWVRQAPGKGLEWVSAISTTG
YTTPYANSVKGRFTISRDNSKNTLYLQMNSLRAEDTALYFCGTLGTTARDFDYWG
QGTLVTVSS
variable domain of Light chain of SBJ08-D18
>SEQ ID NO: 188
AIQMTQSPSSLSASVGDRVTITCRASQDIRNDLGWYQQKPGKAPKRLIYAASNLQS
GVPSRFSGSGSGTEFTLTISSLQPEDFATYYCLQHNSHPFAFGPGTKVDIK
Heavy chain of SBJ08-D18
>SEQ ID NO: 189
MGWSCIILFLVATATGVHSEVQLVESGGGLVQPGGSLRLSCAASGFTFSNYVMSW
VRQAPGKGLEWVSAISTTGYTTPYANSVKGRFTISRDNSKNTLYLQMNSLRAEDT
ALYFCGTLGTTARDFDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGN
VFSCSVMHGALHNHYTQKSLSLFPG*
Light chain of SBJ08-D18
>SEQ ID NO: 190
MGWSCIILFLVATATGVHSAIQMTQSPSSLSASVGDRVTITCRASQDIRNDLGWYQ
QKPGKAPKRLIYAASNLQSGVPSRFSGSGSGTEFTLTISSLQPEDFATYYCLQHNSH
PFAFGPGTKVDIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPV
TKSFNRGEC*

### Nucleic Acid Sequences of SBJ08-D18:

SBJ08-D18_heavy_chain leader sequence
> SEQ ID NO: 191
ATGGGATGGTCATGTATCATCCTTTTTCTAGTAGCAACTGCAACCGGTGTACAT
TCC
SBJ08-D18_heavy_chain variable domain
> SEQ ID NO: 192
GAGGTGCAGCTGGTGGAGTCTGGGGGAGGCTTGGTACAGCCTGGGGGGTCCCT
GAGACTCTCCTGTGCAGCCTCTGGTTTCACCTTTAGTAACTATGTCATGAGCTG
GGTCCGCCAGGCTCCAGGGAAGGGGCTGGAGTGGGTCTCAGCAATTAGTACTA
CTGGTTATACCACACCCTACGCAAACTCTGTGAAGGGCCGGTTCACCATCTCCA
GAGACAATTCCAAGAACACGCTGTATCTGCAAATGAACAGCCTGAGAGCCGA
GGACACGGCCCTTTATTTCTGTGGGACCCTGGGGACAACAGCACGCGATTTTG
ACTACTGGGGCCAGGGAACCCTGGTCACCGTCTCCTCAG
SBJ08-D18_heavy_chain constant domain
> SEQ ID NO: 193
CTTCCACCAAGGGCCCATCGGTCTTCCCCCTGGCACCCTCCTCCAAGAGCACCT
CTGGGGGCACAGCGGCCCTGGGCTGCCTGGTCAAGGACTACTTCCCCGAACCT
GTGACGGTCTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCC
CTCCAGCAGCTTGGGCACCCAGACCTACATCTGCAACGTGAATCACAAGCCCA
GCAACACCAAGGTGGACAAGAGAGTTGAGCCCAAATCTTGTGACAAAACTCA
CACATGCCCACCGTGCCCAGCACCTGAACTCCTGGGGGGACCGTCAGTCTTCC
TCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAG
CAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCACA
GGTGTACACCCTGCCCCCATCCCGGGAGGAGATGACCAAGAACCAGGTCAGCC
TGACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTC
CGACGGCTCCTTCTTCCTCTATAGCAAGCTCACCGTGGACAAGAGCAGGTGGC
AGCAGGGGAACGTCTTCTCATGCTCCGTGATGCATGGCGCTCTGCACAACCAC
TACACGCAGAAGAGCCTCTCCCTGTTCCCCGGGTAA
SBJ08-D18_heavy_chain complete
> SEQ ID NO: 194
ATGGGATGGTCATGTATCATCCTTTTTCTAGTAGCAACTGCAACCGGTGTACAT
TCCGAGGTGCAGCTGGTGGAGTCTGGGGGAGGCTTGGTACAGCCTGGGGGGTC
CCTGAGACTCTCCTGTGCAGCCTCTGGTTTCACCTTTAGTAACTATGTCATGAG
CTGGGTCCGCCAGGCTCCAGGGAAGGGGCTGGAGTGGGTCTCAGCAATTAGTA
CTACTGGTTATACCACACCCTACGCAAACTCTGTGAAGGGCCGGTTCACCATCT
CCAGAGACAATTCCAAGAACACGCTGTATCTGCAAATGAACAGCCTGAGAGCC
GAGGACACGGCCCTTTATTTCTGTGGGACCCTGGGGACAACAGCACGCGATTT
TGACTACTGGGGCCAGGGAACCCTGGTCACCGTCTCCTCAGCTTCCACCAAGG
GCCCATCGGTCTTCCCCCTGGCACCCTCCTCCAAGAGCACCTCTGGGGGCACA
GCGGCCCTGGGCTGCCTGGTCAAGGACTACTTCCCCGAACCTGTGACGGTCTC
GTGGAACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAGCAGCT
TGGGCACCCAGACCTACATCTGCAACGTGAATCACAAGCCCAGCAACACCAAG
GTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAA
ACCCAAGGACACCCTCATGATCTCCCGGACCCCTGAGGTCACATGCGTGGTGG
TGGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGCA
CGTACCGTGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAA
AACCATCTCCAAAGCCAAAGGGCAGCCCCGAGAACCACAGGTGTACACCCTGC
CCCCATCCCGGGAGGAGATGACCAAGAACCAGGTCAGCCTGACCTGCCTGGTC
AAAGGCTTCTATCCCAGCGACATCGCCGTGGAGTGGGAGAGCAATGGGCAGCC
GGAGAACAACTACAAGACCACGCCTCCCGTGCTGGACTCCGACGGCTCCTTCT
TCCTCTATAGCAAGCTCACCGTGGACAAGAGCAGGTGGCAGCAGGGGAACGTC
TTCTCATGCTCCGTGATGCATGGCGCTCTGCACAACCACTACACGCAGAAGAG
CCTCTCCCTGTTCCCCGGGTAA
SBJ08-D18_light_kappa_chain leader sequence
> SEQ ID NO: 195
ATGGGATGGTCATGTATCATCCTTTTTCTAGTAGCAACTGCAACCGGTGTACAT
TCC
SBJ08-D18_light_kappa_chain variable domain
> SEQ ID NO: 196
GCCATCCAGATGACCCAGTCTCCATCCTCCCTGTCTGCATCTGTCGGAGACAGA
GTCACCATCACTTGCCGGGCAAGTCAGGACATTAGAAATGACTTAGGCTGGTA
TCAGCAGAAACCAGGGAAAGCCCCTAAGCGCCTGATCTATGCTGCATCCAATT
TGCAAAGTGGGGTCCCATCAAGGTTCAGCGGCAGTGGATCTGGGACAGAATTC
ACTCTCACAATCAGCAGCCTGCAGCCTGAAGATTTTGCAACTTATTACTGTCTA
CAACATAATAGTCACCCATTCGCTTTCGGCCCTGGGACCAAAGTGGATATCAA
AC
SBJ08-D18_light_kappa_chain constant domain
> SEQ ID NO: 197
GAACTGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGT
GTTAG
SBJ08-D18_light_kappa_chain complete
> SEQ ID NO: 198
ATGGGATGGTCATGTATCATCCTTTTTCTAGTAGCAACTGCAACCGGTGTACAT
TCCGCCATCCAGATGACCCAGTCTCCATCCTCCCTGTCTGCATCTGTCGGAGAC
AGAGTCACCATCACTTGCCGGGCAAGTCAGGACATTAGAAATGACTTAGGCTG
GTATCAGCAGAAACCAGGGAAAGCCCCTAAGCGCCTGATCTATGCTGCATCCA
ATTTGCAAAGTGGGGTCCCATCAAGGTTCAGCGGCAGTGGATCTGGGACAGAA
TTCACTCTCACAATCAGCAGCCTGCAGCCTGAAGATTTTGCAACTTATTACTGT
CTACAACATAATAGTCACCCATTCGCTTTCGGCCCTGGGACCAAAGTGGATAT
CAAACGAACTGTGGCTGCACCATCTGTCTTCATCTTCCCGCCATCTGATGAGCA
GTTGAAATCTGGAACTGCCTCTGTTGTGTGCCTGCTGAATAACTTCTATCCCAG
AGAGGCCAAAGTACAGTGGAAGGTGGATAACGCCCTCCAATCGGGTAACTCCC
AGGAGAGTGTCACAGAGCAGGACAGCAAGGACAGCACCTACAGCCTCAGCAG
CACCCTGACGCTGAGCAAAGCAGACTACGAGAAACACAAAGTCTACGCCTGC
GAAGTCACCCATCAGGGCCTGAGCTCGCCCGTCACAAAGAGCTTCAACAGGGG
AGAGTGTTAG

### Sequence of the Antibody Herein Identified as SBJ08-F04

### Amino Acid Sequences of SBJ08-F04:

CDR 1 of variable domain of Heavy chain of SBJ08-F04
>SEQ ID NO: 199
GFTFSSYG
CDR 2 of variable domain of Heavy chain of SBJ08-F04
>SEQ ID NO: 200
IWYDGSNK
CDR 3 of variable domain of Heavy chain of SBJ08-F04
>SEQ ID NO: 201
ASEYYYGSSGHLPIDC
CDR 1 of variable domain of Light chain of SBJ08-F04
>SEQ ID NO: 202
QSVSSY
CDR 2 of variable domain of Light chain of SBJ08-F04 this sequence is
not included in the sequence listing because is less than 4 amino acid
>SEQ ID NO: 203
DAS (Asp-Ala-Ser)
CDR 3 of variable domain of Light chain of SBJ08-F04
>SEQ ID NO: 204
QQRSNWPRT
variable domain of Heavy chain of SBJ08-F04
>SEQ ID NO: 205
EVQLVESGGGVVQPGRSLRLSCVASGFTFSSYGMHWVRQAPGKGLEWVAIIWYD
GSNKYYADSVKGRFTISRDNSKNTLYLQMNSLRAEDTAVYYCASEYYYGSSGHLP
IDCWGQGTLVTVSS
variable domain of Light chain of SBJ08-F04
>SEQ ID NO: 206
EIVLTQSPATLSLSPGERATLSCRASQSVSSYLAWYQQKPGQAPRLVIYDASNRAT
GIPARFSGSGSGTDFTLTISSLEPEDFAVYYCQQRSNWPRTFGQGTK VEIK
Heavy chain of SBJ08-F04
>SEQ ID NO: 207
MGWSCIILFLVATATGVHSEVQLVESGGGVVQPGRSLRLSCVASGFTFSSYGMHW
VRQAPGKGLEWVAIIWYDGSNKYYADSVKGRFTISRDNSKNTLYLQMNSLRAED
TAVYYCASEYYYGSSGHLPIDCWGQGTLVTVSSASTKGPSVFPLAPSSKSTSGGTA
ALGCLVKDYFPEPVTVSWNSGALTSGVHTFPAVLQSSGLYSLSSVVTVPSSSLGTQ
TYICNVNHKPSNTKVDKR VEPKSCDKTHTCPPCPAPELLGGPSVFLFPPKPKDTLMI
SRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRW
QQGNVFSCSVMHGALHNHYTQKSLSLFPG*
Light chain of SBJ08-F04
>SEQ ID NO: 208
MGWSCIILFLVATATGVHSEIVLTQSPATLSLSPGERATLSCRASQSVSSYLAWYQ
QKPGQAPRLVIYDASNRATGIPARFSGSGSGTDFTLTISSLEPEDFAVYYCQQRSNW
PRTFGQGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPV
TKSFNRGEC*

### Nucleic Acid Sequences of SBJ08-F04:

SBJ08-F04_heavy_chain leader sequence
> SEQ ID NO: 209
ATGGGATGGTCATGTATCATCCTTTTTCTAGTAGCAACTGCAACCGGTGTACAT
TCC
SBJ08-F04_heavy chain variable domain
> SEQ ID NO: 210
GAGGTGCAGCTGGTGGAGTCTGGGGGAGGCGTGGTCCAGCCTGGGAGGTCCCT
GAGACTCTCCTGTGTAGCGTCTGGATTCACCTTCAGTAGTTATGGCATGCACTG
GGTCCGCCAGGCTCCAGGCAAGGGGCTGGAGTGGGTGGCAATTATATGGTATG
ATGGAAGTAATAAATACTATGCAGACTCCGTGAAGGGCCGATTCACCATCTCC
AGAGACAATTCCAAGAACACGCTGTATCTGCAAATGAACAGCCTGAGAGCCG
AGGACACGGCTGTCTATTATTGTGCGAGCGAGTATTACTATGGTAGTAGTGGT
CATTTACCCATTGACTGTTGGGGCCAGGGAACCCTGGTCACCGTCTCCTCAG
SBJ08-F04_heavy_chain constant domain
> SEQ ID NO: 211
CTTCCACCAAGGGCCCATCGGTCTTCCCCCTGGCACCCTCCTCCAAGAGCACCT
CTGGGGGCACAGCGGCCCTGGGCTGCCTGGTCAAGGACTACTTCCCCGAACCT
GTGACGGTCTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCC
CTCCAGCAGCTTGGGCACCCAGACCTACATCTGCAACGTGAATCACAAGCCCA
GCAACACCAAGGTGGACAAGAGAGTTGAGCCCAAATCTTGTGACAAAACTCA
CACATGCCCACCGTGCCCAGCACCTGAACTCCTGGGGGGACCGTCAGTCTTCC
TCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAG
CAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCACA
GGTGTACACCCTGCCCCCATCCCGGGAGGAGATGACCAAGAACCAGGTCAGCC
TGACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTC
CGACGGCTCCTTCTTCCTCTATAGCAAGCTCACCGTGGACAAGAGCAGGTGGC
AGCAGGGGAACGTCTTCTCATGCTCCGTGATGCATGGCGCTCTGCACAACCAC
TACACGCAGAAGAGCCTCTCCCTGTTCCCCGGGTAA
SBJ08-F04_heavy_chain complete
> SEQ ID NO: 212
ATGGGATGGTCATGTATCATCCTTTTTCTAGTAGCAACTGCAACCGGTGTACAT
TCCGAGGTGCAGCTGGTGGAGTCTGGGGGAGGCGTGGTCCAGCCTGGGAGGTC
CCTGAGACTCTCCTGTGTAGCGTCTGGATTCACCTTCAGTAGTTATGGCATGCA
CTGGGTCCGCCAGGCTCCAGGCAAGGGGCTGGAGTGGGTGGCAATTATATGGT
ATGATGGAAGTAATAAATACTATGCAGACTCCGTGAAGGGCCGATTCACCATC
TCCAGAGACAATTCCAAGAACACGCTGTATCTGCAAATGAACAGCCTGAGAGC
CGAGGACACGGCTGTCTATTATTGTGCGAGCGAGTATTACTATGGTAGTAGTG
GTCATTTACCCATTGACTGTTGGGGCCAGGGAACCCTGGTCACCGTCTCCTCAG
CTTCCACCAAGGGCCCATCGGTCTTCCCCCTGGCACCCTCCTCCAAGAGCACCT
CTGGGGGCACAGCGGCCCTGGGCTGCCTGGTCAAGGACTACTTCCCCGAACCT
GTGACGGTCTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCC
CTCCAGCAGCTTGGGCACCCAGACCTACATCTGCAACGTGAATCACAAGCCCA
GCAACACCAAGGTGGACAAGAGAGTTGAGCCCAAATCTTGTGACAAAACTCA
CACATGCCCACCGTGCCCAGCACCTGAACTCCTGGGGGGACCGTCAGTCTTCC
TCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAG
CAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCACA
GGTGTACACCCTGCCCCCATCCCGGGAGGAGATGACCAAGAACCAGGTCAGCC
TGACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTC
CGACGGCTCCTTCTTCCTCTATAGCAAGCTCACCGTGGACAAGAGCAGGTGGC
AGCAGGGGAACGTCTTCTCATGCTCCGTGATGCATGGCGCTCTGCACAACCAC
TACACGCAGAAGAGCCTCTCCCTGTTCCCCGGGTAA
SBJ08-F04_light_kappa_chain leader sequence
> SEQ ID NO: 213
ATGGGATGGTCATGTATCATCCTTTTTCTAGTAGCAACTGCAACCGGTGTACAT
TCC
SBJ08-F04_light_kappa_chain variable domain
> SEQ ID NO: 214
GAAATTGTGTTGACACAGTCTCCAGCCACCCTGTCTTTGTCTCCAGGGGAAAG
AGCCACCCTCTCCTGCAGGGCCAGTCAGAGTGTTAGCAGTTACTTAGCCTGGT
ACCAACAGAAACCTGGCCAGGCTCCCAGGCTCGTCATCTATGATGCATCCAAC
AGGGCCACTGGCATCCCAGCCAGGTTCAGTGGCAGTGGGTCTGGGACAGACTT
CACTCTCACCATCAGCAGCCTAGAGCCTGAAGATTTTGCAGTTTATTACTGTCA
GCAGCGTAGCAACTGGCCTCGGACGTTCGGCCAAGGGACCAAGGTGGAAATC
AAAC
SBJ08-F04_light_kappa_chain constant domain
> SEQ ID NO: 215
GAACTGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGT
GTTAG
SBJ08-F04_light_kappa_chain complete
> SEQ ID NO: 216
ATGGGATGGTCATGTATCATCCTTTTTCTAGTAGCAACTGCAACCGGTGTACAT
TCCGAAATTGTGTTGACACAGTCTCCAGCCACCCTGTCTTTGTCTCCAGGGGAA
AGAGCCACCCTCTCCTGCAGGGCCAGTCAGAGTGTTAGCAGTTACTTAGCCTG
GTACCAACAGAAACCTGGCCAGGCTCCCAGGCTCGTCATCTATGATGCATCCA
ACAGGGCCACTGGCATCCCAGCCAGGTTCAGTGGCAGTGGGTCTGGGACAGAC
TTCACTCTCACCATCAGCAGCCTAGAGCCTGAAGATTTTGCAGTTTATTACTGT
CAGCAGCGTAGCAACTGGCCTCGGACGTTCGGCCAAGGGACCAAGGTGGAAA
TCAAACGAACTGTGGCTGCACCATCTGTCTTCATCTTCCCGCCATCTGATGAGC
AGTTGAAATCTGGAACTGCCTCTGTTGTGTGCCTGCTGAATAACTTCTATCCCA
GAGAGGCCAAAGTACAGTGGAAGGTGGATAACGCCCTCCAATCGGGTAACTC
CCAGGAGAGTGTCACAGAGCAGGACAGCAAGGACAGCACCTACAGCCTCAGC
AGCACCCTGACGCTGAGCAAAGCAGACTACGAGAAACACAAAGTCTACGCCT
GCGAAGTCACCCATCAGGGCCTGAGCTCGCCCGTCACAAAGAGCTTCAACAGG
GGAGAGTGTTAG

### Sequence of the Antibody Herein Identified as SBJ08-H10

### Amino Acid Sequences of SBJ08-H10:

CDR 1 of variable domain of Heavy chain of SBJ08-H10
>SEQ ID NO: 217
GFTFSSHA
CDR 2 of variable domain of Heavy chain of SBJ08-H10
>SEQ ID NO: 218
ISGTGDRT
CDR 3 of variable domain of Heavy chain of SBJ08-H10
>SEQ ID NO: 219
ARAPIGTFYYDN
CDR 1 of variable domain of Light chain of SBJ08-H10
>SEQ ID NO: 220
QSVGTRN
CDR 2 of variable domain of Light chain of SBJ08-H10 this sequence is
not included in the sequence listing because is less than 4 amino acid
>SEQ ID NO: 221
GAS (Gly-Ala-Ser)
CDR 3 of variable domain of Light chain of SBJ08-H10
>SEQ ID NO: 222
QQYGISPRT
variable domain of Heavy chain of SBJ08-H10
>SEQ ID NO: 223
EVQLVESGGGLVQPGGSLRLSCAASGFTFSSHAMSWVRQAPGKGLEWVSAISGTG
DRTDDTGSVRGRFTISRDNSKNTVYLQMFFLTVEDTALYYCARAPIGTFYYDNWG
QGTLVTVSS
variable domain of Light chain of SBJ08-H10
>SEQ ID NO: 224
ETTLTQSPGTLSLSPGERATLSCRASQSVGTRNLAWYQQKPGQSPRLLIYGASSRA
TGIPDRFSGGGSGTDFTLTISRLEPEDFAVYYCQQYGISPRTFGQGTKVEIK
Heavy chain of SBJ08-H10
>SEQ ID NO: 225
HGMVMYHPFSSTTATGVHSEVQLVESGGGLVQPGGSLRLSCAASGFTFSSHAMS
WVRQAPGKGLEWVSAISGTGDRTDDTGSVRGRFTISRDNSKNTVYLQMFFLTVED
TALYYCARAPIGTFYYDNWGQGTLVTVSSASTKGPSVFPLAPSSKSTSGGTAALGC
LVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSL
TCL VKGFYPSDIA VEWESNGQPENNYKTTPPVLDSDGSFFLYSKLTVDKSRWQQG
NVFSCSVMHGALHNHYTQKSLSLFPG*
Light chain of SBJ08-H10
>SEQ ID NO: 226
MGWSCIILFLVATATGVHSETTLTQSPGTLSLSPGERATLSCRASQSVGTRNLAWY
QQKPGQSPRLLIYGASSRATGIPDRFSGGGSGTDFTLTISRLEPEDFAVYYCQQYGIS
PRTFGQGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPV
TKSFNRGEC*

### Nucleic Acid Sequences of SBJ08-H10:

SBJ08-H10 heavy chain leader sequence
> SEQ ID NO: 227
CATGGGATGGTCATGTATCATCCTTTTTCTAGTACAACTGCAACCGGTGTACAT
TCC
> SEQ ID NO: 228
SBJ08-H10_heavy_chain variable domain
GAGGTGCAGCTGGTGGAGTCTGGGGGAGGCTTGGTACAGCCTGGGGGGTCCCT
GAGACTCTCCTGTGCAGCCTCTGGATTCACCTTTAGTAGTCATGCCATGAGCTG
GGTCCGCCAGGCTCCAGGGAAGGGGCTGGAGTGGGTCTCAGCCATAAGTGGC
ACTGGTGATAGAACAGACGACACAGGCTCCGTGAGGGGCCGGTTCACCATCTC
CAGAGACAATTCCAAGAACACGGTGTATCTGCAAATGTTTTTTTTGACAGTCG
AGGACACGGCCCTATATTACTGTGCGAGAGCCCCTATCGGAACTTTCTACTATG
ACAACTGGGGCCAGGGAACCCTGGTCACCGTCTCATCAG
SBJ08-H10_heavy_chain constant domain
> SEQ ID NO: 229
CCTCCACCAAGGGCCCATCGGTCTTCCCCCTGGCACCCTCCTCCAAGAGCACCT
CTGGGGGCACAGCGGCCCTGGGCTGCCTGGTCAAGGACTACTTCCCCGAACCT
GTGACGGTCTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCC
CTCCAGCAGCTTGGGCACCCAGACCTACATCTGCAACGTGAATCACAAGCCCA
GCAACACCAAGGTGGACAAGAGAGTTGAGCCCAAATCTTGTGACAAAACTCA
CACATGCCCACCGTGCCCAGCACCTGAACTCCTGGGGGGACCGTCAGTCTTCC
TCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAG
CAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCACA
GGTGTACACCCTGCCCCCATCCCGGGAGGAGATGACCAAGAACCAGGTCAGCC
TGACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTC
CGACGGCTCCTTCTTCCTCTATAGCAAGCTCACCGTGGACAAGAGCAGGTGGC
AGCAGGGGAACGTCTTCTCATGCTCCGTGATGCATGGCGCTCTGCACAACCAC
TACACGCAGAAGAGCCTCTCCCTGTTCCCCGGGTAA
SBJ08-H10_heavy_chain complete
> SEQ ID NO: 230
CATGGGATGGTCATGTATCATCCTTTTTCTAGTACAACTGCAACCGGTGTACAT
TCCGAGGTGCAGCTGGTGGAGTCTGGGGGAGGCTTGGTACAGCCTGGGGGGTC
CCTGAGACTCTCCTGTGCAGCCTCTGGATTCACCTTTAGTAGTCATGCCATGAG
CTGGGTCCGCCAGGCTCCAGGGAAGGGGCTGGAGTGGGTCTCAGCCATAAGTG
GCACTGGTGATAGAACAGACGACACAGGCTCCGTGAGGGGCCGGTTCACCATC
TCCAGAGACAATTCCAAGAACACGGTGTATCTGCAAATGTTTTTTTTGACAGTC
GAGGACACGGCCCTATATTACTGTGCGAGAGCCCCTATCGGAACTTTCTACTAT
GACAACTGGGGCCAGGGAACCCTGGTCACCGTCTCATCAGCCTCCACCAAGGG
CCCATCGGTCTTCCCCCTGGCACCCTCCTCCAAGAGCACCTCTGGGGGCACAGC
GGCCCTGGGCTGCCTGGTCAAGGACTACTTCCCCGAACCTGTGACGGTCTCGT
GGAACTCAGGCGCCCTGACCAGCGGCGTGCACACCTTCCCGGCTGTCCTACAG
TCCTCAGGACTCTACTCCCTCAGCAGCGTGGTGACCGTGCCCTCCAGCAGCTTG
GGCACCCAGACCTACATCTGCAACGTGAATCACAAGCCCAGCAACACCAAGGT
GGACAAGAGAGTTGAGCCCAAATCTTGTGACAAAACTCACACATGCCCACCGT
GCCCAGCACCTGAACTCCTGGGGGGACCGTCAGTCTTCCTCTTCCCCCCAAAAC
CCAAGGACACCCTCATGATCTCCCGGACCCCTGAGGTCACATGCGTGGTGGTG
GACGTGAGCCACGAAGACCCTGAGGTCAAGTTCAACTGGTACGTGGACGGCGT
GGAGGTGCATAATGCCAAGACAAAGCCGCGGGAGGAGCAGTACAACAGCACG
TACCGTGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAA
CCATCTCCAAAGCCAAAGGGCAGCCCCGAGAACCACAGGTGTACACCCTGCCC
CCATCCCGGGAGGAGATGACCAAGAACCAGGTCAGCCTGACCTGCCTGGTCAA
AGGCTTCTATCCCAGCGACATCGCCGTGGAGTGGGAGAGCAATGGGCAGCCGG
AGAACAACTACAAGACCACGCCTCCCGTGCTGGACTCCGACGGCTCCTTCTTC
CTCTATAGCAAGCTCACCGTGGACAAGAGCAGGTGGCAGCAGGGGAACGTCTT
CTCATGCTCCGTGATGCATGGCGCTCTGCACAACCACTACACGCAGAAGAGCC
TCTCCCTGTTCCCCGGGTAA
SBJ08-H10_light_kappa_chain leader sequence
> SEQ ID NO: 231
ATGGGATGGTCATGTATCATCCTTTTTCTAGTAGCAACTGCAACCGGTGTACAT
TCC
SBJ08-H10_light_kappa_chain variable domain
> SEQ ID NO: 232
GAAACGACACTCACGCAGTCTCCAGGCACCCTGTCTCTGTCTCCAGGGGAAAG
AGCCACCCTCTCCTGCAGGGCCAGTCAGAGTGTTGGCACCAGGAACTTAGCCT
GGTACCAGCAGAAACCTGGCCAGTCTCCCAGGCTCCTCATCTATGGTGCATCC
AGCAGGGCCACTGGCATCCCAGACAGGTTCAGTGGCGGTGGGTCTGGGACAG
ACTTCACTCTCACCATCAGCAGACTGGAGCCTGAAGATTTTGCAGTGTATTACT
GTCAGCAGTATGGTATCTCACCTCGCACGTTCGGCCAAGGGACCAAGGTGGAA
ATCAAAC
SBJ08-H10_light_kappa_chain constant domain
> SEQ ID NO: 233
GAACTGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGT
GTTAG
SBJ08-H10_light_kappa_chain complete
> SEQ ID NO: 234
ATGGGATGGTCATGTATCATCCTTTTTCTAGTAGCAACTGCAACCGGTGTACAT
TCCGAAACGACACTCACGCAGTCTCCAGGCACCCTGTCTCTGTCTCCAGGGGA
AAGAGCCACCCTCTCCTGCAGGGCCAGTCAGAGTGTTGGCACCAGGAACTTAG
CCTGGTACCAGCAGAAACCTGGCCAGTCTCCCAGGCTCCTCATCTATGGTGCAT
CCAGCAGGGCCACTGGCATCCCAGACAGGTTCAGTGGCGGTGGGTCTGGGACA
GACTTCACTCTCACCATCAGCAGACTGGAGCCTGAAGATTTTGCAGTGTATTAC
TGTCAGCAGTATGGTATCTCACCTCGCACGTTCGGCCAAGGGACCAAGGTGGA
AATCAAACGAACTGTGGCTGCACCATCTGTCTTCATCTTCCCGCCATCTGATGA
GCAGTTGAAATCTGGAACTGCCTCTGTTGTGTGCCTGCTGAATAACTTCTATCC
CAGAGAGGCCAAAGTACAGTGGAAGGTGGATAACGCCCTCCAATCGGGTAAC
TCCCAGGAGAGTGTCACAGAGCAGGACAGCAAGGACAGCACCTACAGCCTCA
GCAGCACCCTGACGCTGAGCAAAGCAGACTACGAGAAACACAAAGTCTACGC
CTGCGAAGTCACCCATCAGGGCCTGAGCTCGCCCGTCACAAAGAGCTTCAACA
GGGGAGAGTGTTAG

### Sequence of the Antibody Herein Identified as SBJ08-J10

### Amino Acid Sequences of SBJ08-J10:

CDR 1 of variable domain of Heavy chain of SBJ08-J10
>SEQ ID NO: 235
GLTFSSHS
CDR 2 of variable domain of Heavy chain of SBJ08-J10
>SEQ ID NO: 236
ISGDSDHI
CDR 3 of variable domain of Heavy chain of SBJ08-J10
>SEQ ID NO: 237
TNWGKFAVVSY
CDR 1 of variable domain of Light chain of SBJ08-J10
>SEQ ID NO: 238
QSLVFRDGNIY
CDR 2 of variable domain of Light chain of SBJ08-J10 this sequence is
not included in the sequence listing because is less than 4 amino acid
>SEQ ID NO: 239
QIS (Gln-Ile-Ser)
CDR 3 of variable domain of Light chain of SBJ08-J10
>SEQ ID NO: 240
MQGSHWPYS
variable domain of Heavy chain of SBJ08-J10
>SEQ ID NO: 241
EVQLVESGGGLVQPGGSLRLSCAASGLTFSSHSMGWVRQAPGKGLEWVSIISGDS
DHIYYADSVKGRFTISRDNSQNTVDLLMSGLRAEDTAVYYCTNWGKFAVVSYCG
QGTLVTVSS
variable domain of Light chain of SBJ08-J10
>SEQ ID NO: 242
DIVMTQSPLSLPVTLGQPASISCRSSQSLVFRDGNIYLNWFQQRPGQSPRRLIYQISK
RDSGVPDRFSGSGSGTDFTLQISRVEAEDVGVYYCMQGSHWPYSFGQGTKLEIK
Heavy chain of SBJ08-J10
>SEQ ID NO: 243
MGWSCIILFLVATATGVHSEVQLVESGGGLVQPGGSLRLSCAASGLTFSSHSMGW
VRQAPGKGLEWVSIISGDSDHIYYADSVKGRFTISRDNSQNTVDLLMSGLRAEDTA
VYYCTNWGKFAVVSYCGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNV
NHKPSNTKVDKRVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNVF
SCSVMHGALHNHYTQKSLSLFPG*
Light chain of SBJ08-J10
>SEQ ID NO: 244
MGWSCIILFLVATATGVHSDIVMTQSPLSLPVTLGQPASISCRSSQSLVFRDGNIYL
NWFQQRPGQSPRRLIYQISKRDSGVPDRFSGSGSGTDFTLQISRVEAEDVGVYYCM
QGSHWPYSFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQ
GLSSPVTKSFNRGEC*

### Nucleic Acid Sequences of SBJ08-J10:

SBJ08-J10_heavy_chain leader sequence
> SEQ ID NO: 245
ATGGGATGGTCATGTATCATCCTTTTTCTAGTAGCAACTGCAACCGGTGTACAT
TCC
SBJ08-J10_heavy_chain variable domain
> SEQ ID NO: 246
GAGGTGCAGCTGGTGGAGTCTGGGGGAGGCTTGGTACAGCCTGGGGGGTCCCT
GAGACTCTCCTGTGCAGCCTCTGGACTCACCTTTAGCAGCCATAGCATGGGTTG
GGTCCGCCAGGCTCCAGGGAAGGGGCTGGAGTGGGTCTCAATTATTAGTGGAG
ATAGTGATCATATATACTACGCAGATTCCGTGAAGGGCCGATTCACCATCTCC
AGAGACAATTCCCAGAACACCGTGGATCTGCTAATGAGCGGCCTGAGAGCCGA
GGACACGGCCGTATATTACTGTACGAACTGGGGAAAGTTCGCAGTTGTATCAT
ACTGCGGCCAGGGAACCCTGGTCACCGTCTCTTCAG
SBJ08-J10_heavy_chain constant domain
> SEQ ID NO: 247
CCTCCACCAAGGGCCCATCGGTCTTCCCCCTGGCACCCTCCTCCAAGAGCACCT
CTGGGGGCACAGCGGCCCTGGGCTGCCTGGTCAAGGACTACTTCCCCGAACCT
GTGACGGTCTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCC
CTCCAGCAGCTTGGGCACCCAGACCTACATCTGCAACGTGAATCACAAGCCCA
GCAACACCAAGGTGGACAAGAGAGTTGAGCCCAAATCTTGTGACAAAACTCA
CACATGCCCACCGTGCCCAGCACCTGAACTCCTGGGGGGACCGTCAGTCTTCC
TCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAG
CAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCACA
GGTGTACACCCTGCCCCCATCCCGGGAGGAGATGACCAAGAACCAGGTCAGCC
TGACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTC
CGACGGCTCCTTCTTCCTCTATAGCAAGCTCACCGTGGACAAGAGCAGGTGGC
AGCAGGGGAACGTCTTCTCATGCTCCGTGATGCATGGCGCTCTGCACAACCAC
TACACGCAGAAGAGCCTCTCCCTGTTCCCCGGGTAA
SBJ08-J10_heavy_chain complete
> SEQ ID NO: 248
ATGGGATGGTCATGTATCATCCTTTTTCTAGTAGCAACTGCAACCGGTGTACAT
TCCGAGGTGCAGCTGGTGGAGTCTGGGGGAGGCTTGGTACAGCCTGGGGGGTC
CCTGAGACTCTCCTGTGCAGCCTCTGGACTCACCTTTAGCAGCCATAGCATGGG
TTGGGTCCGCCAGGCTCCAGGGAAGGGGCTGGAGTGGGTCTCAATTATTAGTG
GAGATAGTGATCATATATACTACGCAGATTCCGTGAAGGGCCGATTCACCATC
TCCAGAGACAATTCCCAGAACACCGTGGATCTGCTAATGAGCGGCCTGAGAGC
CGAGGACACGGCCGTATATTACTGTACGAACTGGGGAAAGTTCGCAGTTGTAT
CATACTGCGGCCAGGGAACCCTGGTCACCGTCTCTTCAGCCTCCACCAAGGGC
CCATCGGTCTTCCCCCTGGCACCCTCCTCCAAGAGCACCTCTGGGGGCACAGC
GGCCCTGGGCTGCCTGGTCAAGGACTACTTCCCCGAACCTGTGACGGTCTCGT
GGAACTCAGGCGCCCTGACCAGCGGCGTGCACACCTTCCCGGCTGTCCTACAG
TCCTCAGGACTCTACTCCCTCAGCAGCGTGGTGACCGTGCCCTCCAGCAGCTTG
GGCACCCAGACCTACATCTGCAACGTGAATCACAAGCCCAGCAACACCAAGGT
GGACAAGAGAGTTGAGCCCAAATCTTGTGACAAAACTCACACATGCCCACCGT
GCCCAGCACCTGAACTCCTGGGGGGACCGTCAGTCTTCCTCTTCCCCCCAAAAC
CCAAGGACACCCTCATGATCTCCCGGACCCCTGAGGTCACATGCGTGGTGGTG
GACGTGAGCCACGAAGACCCTGAGGTCAAGTTCAACTGGTACGTGGACGGCGT
GGAGGTGCATAATGCCAAGACAAAGCCGCGGGAGGAGCAGTACAACAGCACG
TACCGTGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAA
CCATCTCCAAAGCCAAAGGGCAGCCCCGAGAACCACAGGTGTACACCCTGCCC
CCATCCCGGGAGGAGATGACCAAGAACCAGGTCAGCCTGACCTGCCTGGTCAA
AGGCTTCTATCCCAGCGACATCGCCGTGGAGTGGGAGAGCAATGGGCAGCCGG
AGAACAACTACAAGACCACGCCTCCCGTGCTGGACTCCGACGGCTCCTTCTTC
CTCTATAGCAAGCTCACCGTGGACAAGAGCAGGTGGCAGCAGGGGAACGTCTT
CTCATGCTCCGTGATGCATGGCGCTCTGCACAACCACTACACGCAGAAGAGCC
TCTCCCTGTTCCCCGGGTAA
SBJ08-J10_light_kappa_chain leader sequence
> SEQ ID NO: 249
ATGGGATGGTCATGTATCATCCTTTTTCTAGTAGCAACTGCAACCGGTGTACAT
TCC
SBJ08-J10_light_kappa_chain variable domain
> SEQ ID NO: 250
GATATTGTGATGACTCAGTCTCCACTCTCCCTGCCCGTCACCCTTGGACAGCCG
GCCTCCATCTCCTGCAGGTCTAGTCAAAGCCTCGTATTCCGTGATGGAAACATC
TACTTAAATTGGTTTCAGCAGAGGCCAGGCCAATCTCCAAGGCGCCTAATTTAT
CAGATTTCTAAGCGGGACTCTGGGGTCCCAGACAGATTCAGCGGCAGTGGATC
AGGCACTGATTTCACACTGCAAATCAGCAGGGTGGAGGCTGAGGATGTTGGGG
TTTATTACTGCATGCAAGGTTCACACTGGCCGTACAGTTTTGGCCAGGGGACCA
AGCTGGAGATCAAAC
SBJ08-J10_light_kappa_chain constant domain
> SEQ ID NO: 251
GAACTGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGT
GTTAG
SBJ08-J10_light_kappa_chain complete
> SEQ ID NO: 252
ATGGGATGGTCATGTATCATCCTTTTTCTAGTAGCAACTGCAACCGGTGTACAT
TCCGATATTGTGATGACTCAGTCTCCACTCTCCCTGCCCGTCACCCTTGGACAG
CCGGCCTCCATCTCCTGCAGGTCTAGTCAAAGCCTCGTATTCCGTGATGGAAAC
ATCTACTTAAATTGGTTTCAGCAGAGGCCAGGCCAATCTCCAAGGCGCCTAAT
TTATCAGATTTCTAAGCGGGACTCTGGGGTCCCAGACAGATTCAGCGGCAGTG
GATCAGGCACTGATTTCACACTGCAAATCAGCAGGGTGGAGGCTGAGGATGTT
GGGGTTTATTACTGCATGCAAGGTTCACACTGGCCGTACAGTTTTGGCCAGGG
GACCAAGCTGGAGATCAAACGAACTGTGGCTGCACCATCTGTCTTCATCTTCCC
GCCATCTGATGAGCAGTTGAAATCTGGAACTGCCTCTGTTGTGTGCCTGCTGAA
TAACTTCTATCCCAGAGAGGCCAAAGTACAGTGGAAGGTGGATAACGCCCTCC
AATCGGGTAACTCCCAGGAGAGTGTCACAGAGCAGGACAGCAAGGACAGCAC
CTACAGCCTCAGCAGCACCCTGACGCTGAGCAAAGCAGACTACGAGAAACAC
AAAGTCTACGCCTGCGAAGTCACCCATCAGGGCCTGAGCTCGCCCGTCACAAA
GAGCTTCAACAGGGGAGAGTGTTAG

### Sequence of the Antibody Herein Identified as SBJ08-K19

### Amino Acid Sequences of SBJ08-K19:

CDR 1 of variable domain of Heavy chain of SBJ08-K19
>SEQ ID NO: 253
GFTFSSYS
CDR 2 of variable domain of Heavy chain of SBJ08-K19
>SEQ ID NO: 254
IDGSSTFI
CDR 3 of variable domain of Heavy chain of SBJ08-K19
>SEQ ID NO: 255
ATRGSHSITNGFDI
CDR 1 of variable domain of Light chain of SBJ08-K19
>SEQ ID NO: 256
QSVSSS
CDR 2 of variable domain of Light chain of SBJ08-K19 this sequence is
not included in the sequence listing because is less than 4 amino acid
>SEQ ID NO: 257
DAS (Asp-Ala-Ser)
CDR 3 of variable domain of Light chain of SBJ08-K19
>SEQ ID NO: 258
QQRSNWPPYS
variable domain of Heavy chain of SBJ08-K19
>SEQ ID NO: 259
EVQLVESGGGLVKPGGSLRLSCAASGFTFSSYSMHWVRQAPGKGLEWVSSIDGSS
TFIYYADSVKGRFTISRDNAKNSLYLQMNSLRAEDTAVFYCATRGSHSITNGFDIW
GHGTMVTVSS
variable domain of Light chain of SBJ08-K19
>SEQ ID NO: 260
EIVLTQSPATLSLSPGERATLSCRASQSVSSSLAWYQQKPGQAPRLLIYDASNRATG
IPARFSGSGSGTDFTLTISSLEPEDFAVYYCQQRSNWPPYSFGQGTKLEIK
Heavy chain of SBJ08-K19
>SEQ ID NO: 261
MGWSCIILFLVATATGVHSEVQLVESGGGLVKPGGSLRLSCAASGFTFSSYSMHW
VRQAPGKGLEWVSSIDGSSTFIYYADSVKGRFTISRDNAKNSLYLQMNSLRAEDTA
VFYCATRGSHSITNGFDIWGHGTMVTVSSASTKGPSVFPLAPSSKSTSGGTAALGC
LVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQG
NVFSCSVMHGALHNHYTQKSLSLFPG*
Light chain of SBJ08-K19
>SEQ ID NO: 262
MGWSCIILFLVATATGVHSEIVLTQSPATLSLSPGERATLSCRASQSVSSSLAWYQQ
KPGQAPRLLIYDASNRATGIPARFSGSGSGTDFTLTISSLEPEDFAVYYCQQRSNWP
PYSFGQGTKLEIKGTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPV
TKSFNRGEC*

### Nucleic Acid Sequences of SBJ08-K19:

SBJ08-K19_heavy_chain leader sequence
> SEQ ID NO: 263
ATGGGATGGTCATGTATCATCCTTTTTCTAGTAGCAACTGCAACCGGTGTACAT
TCC
SBJ08-K19_heavy_chain variable domain
> SEQ ID NO: 264
GAGGTGCAGCTGGTGGAGTCTGGGGGAGGCCTGGTCAAGCCTGGGGGGTCCCT
GAGACTCTCCTGTGCAGCCTCTGGATTCACCTTCAGTAGTTATAGCATGCACTG
GGTCCGCCAGGCTCCAGGGAAGGGGCTGGAGTGGGTCTCATCCATTGATGGTA
GTAGTACTTTCATATACTACGCAGACTCAGTGAAGGGCCGATTCACCATCTCCA
GAGACAACGCCAAGAACTCACTGTATCTGCAAATGAACAGCCTGAGGGCCGA
GGACACGGCTGTGTTTTACTGTGCGACTCGGGGTAGTCATTCGATCACAAATG
GTTTTGATATCTGGGGCCATGGGACAATGGTCACCGTCTCTTCAG
SBJ08-K19_heavy_chain constant domain
> SEQ ID NO: 265
CCTCCACCAAGGGCCCATCGGTCTTCCCCCTGGCACCCTCCTCCAAGAGCACCT
CTGGGGGCACAGCGGCCCTGGGCTGCCTGGTCAAGGACTACTTCCCCGAACCT
GTGACGGTCTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCC
CTCCAGCAGCTTGGGCACCCAGACCTACATCTGCAACGTGAATCACAAGCCCA
GCAACACCAAGGTGGACAAGAGAGTTGAGCCCAAATCTTGTGACAAAACTCA
CACATGCCCACCGTGCCCAGCACCTGAACTCCTGGGGGGACCGTCAGTCTTCC
TCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAG
CAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCACA
GGTGTACACCCTGCCCCCATCCCGGGAGGAGATGACCAAGAACCAGGTCAGCC
TGACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTC
CGACGGCTCCTTCTTCCTCTATAGCAAGCTCACCGTGGACAAGAGCAGGTGGC
AGCAGGGGAACGTCTTCTCATGCTCCGTGATGCATGGCGCTCTGCACAACCAC
TACACGCAGAAGAGCCTCTCCCTGTTCCCCGGGTAA
SBJ08-K19_heavy_chain complete
> SEQ ID NO: 266
ATGGGATGGTCATGTATCATCCTTTTTCTAGTAGCAACTGCAACCGGTGTACAT
TCCGAGGTGCAGCTGGTGGAGTCTGGGGGAGGCCTGGTCAAGCCTGGGGGGTC
CCTGAGACTCTCCTGTGCAGCCTCTGGATTCACCTTCAGTAGTTATAGCATGCA
CTGGGTCCGCCAGGCTCCAGGGAAGGGGCTGGAGTGGGTCTCATCCATTGATG
GTAGTAGTACTTTCATATACTACGCAGACTCAGTGAAGGGCCGATTCACCATCT
CCAGAGACAACGCCAAGAACTCACTGTATCTGCAAATGAACAGCCTGAGGGCC
GAGGACACGGCTGTGTTTTACTGTGCGACTCGGGGTAGTCATTCGATCACAAA
TGGTTTTGATATCTGGGGCCATGGGACAATGGTCACCGTCTCTTCAGCCTCCAC
CAAGGGCCCATCGGTCTTCCCCCTGGCACCCTCCTCCAAGAGCACCTCTGGGG
GCACAGCGGCCCTGGGCTGCCTGGTCAAGGACTACTTCCCCGAACCTGTGACG
GTCTCGTGGAACTCAGGCGCCCTGACCAGCGGCGTGCACACCTTCCCGGCTGT
CCTACAGTCCTCAGGACTCTACTCCCTCAGCAGCGTGGTGACCGTGCCCTCCAG
CAGCTTGGGCACCCAGACCTACATCTGCAACGTGAATCACAAGCCCAGCAACA
CCAAGGTGGACAAGAGAGTTGAGCCCAAATCTTGTGACAAAACTCACACATGC
CCACCGTGCCCAGCACCTGAACTCCTGGGGGGACCGTCAGTCTTCCTCTTCCCC
CCAAAACCCAAGGACACCCTCATGATCTCCCGGACCCCTGAGGTCACATGCGT
GGTGGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTACAA
CAGCACGTACCGTGTGGTCAGCGTCCTCACCGTCCTGCACCAGGACTGGCTGA
ATGGCAAGGAGTACAAGTGCAAGGTCTCCAACAAAGCCCTCCCAGCCCCCATC
GAGAAAACCATCTCCAAAGCCAAAGGGCAGCCCCGAGAACCACAGGTGTACA
CCCTGCCCCCATCCCGGGAGGAGATGACCAAGAACCAGGTCAGCCTGACCTGC
CTGGTCAAAGGCTTCTATCCCAGCGACATCGCCGTGGAGTGGGAGAGCAATGG
GCAGCCGGAGAACAACTACAAGACCACGCCTCCCGTGCTGGACTCCGACGGCT
CCTTCTTCCTCTATAGCAAGCTCACCGTGGACAAGAGCAGGTGGCAGCAGGGG
AACGTCTTCTCATGCTCCGTGATGCATGGCGCTCTGCACAACCACTACACGCAG
AAGAGCCTCTCCCTGTTCCCCGGGTAA
SBJ08-K19_light_kappa_chain leader sequence
> SEQ ID NO: 267
ATGGGATGGTCATGTATCATCCTTTTTCTAGTAGCAACTGCAACCGGTGTACAT
TCC
SBJ08-K19_light_kappa_chain variable domain
> SEQ ID NO: 268
GAAATTGTGTTGACACAGTCTCCAGCCACCCTGTCTTTGTCTCCAGGGGAAAG
AGCCACCCTCTCCTGCAGGGCCAGTCAGAGTGTTAGCAGCTCCTTAGCCTGGT
ACCAACAGAAACCTGGCCAGGCTCCCAGGCTCCTCATCTATGATGCATCCAAC
AGGGCCACTGGCATCCCAGCCAGGTTCAGTGGCAGTGGGTCTGGGACAGACTT
CACTCTCACCATCAGCAGCCTAGAGCCGGAAGATTTTGCAGTTTATTACTGTCA
GCAGCGTAGCAACTGGCCTCCGTACAGTTTTGGCCAGGGGACCAAGCTGGAGA
TCAAA
SBJ08-K19_light_kappa_chain constant domain
> SEQ ID NO: 269
GGAACTGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAG
GCCAAAGTACAGTGGAAGGTGGATAACGCCCTCCAATCGGGTAACTCCCAGGA
GAGTGTCACAGAGCAGGACAGCAAGGACAGCACCTACAGCCTCAGCAGCACC
CTGACGCTGAGCAAAGCAGACTACGAGAAACACAAAGTCTACGCCTGCGAAG
TCACCCATCAGGGCCTGAGCTCGCCCGTCACAAAGAGCTTCAACAGGGGAGAG
TGTTAG
SBJ08-K19_light_kappa_chain complete
> SEQ ID NO: 270
ATGGGATGGTCATGTATCATCCTTTTTCTAGTAGCAACTGCAACCGGTGTACAT
TCCGAAATTGTGTTGACACAGTCTCCAGCCACCCTGTCTTTGTCTCCAGGGGAA
AGAGCCACCCTCTCCTGCAGGGCCAGTCAGAGTGTTAGCAGCTCCTTAGCCTG
GTACCAACAGAAACCTGGCCAGGCTCCCAGGCTCCTCATCTATGATGCATCCA
ACAGGGCCACTGGCATCCCAGCCAGGTTCAGTGGCAGTGGGTCTGGGACAGAC
TTCACTCTCACCATCAGCAGCCTAGAGCCGGAAGATTTTGCAGTTTATTACTGT
CAGCAGCGTAGCAACTGGCCTCCGTACAGTTTTGGCCAGGGGACCAAGCTGGA
GATCAAAGGAACTGTGGCTGCACCATCTGTCTTCATCTTCCCGCCATCTGATGA
GCAGTTGAAATCTGGAACTGCCTCTGTTGTGTGCCTGCTGAATAACTTCTATCC
CAGAGAGGCCAAAGTACAGTGGAAGGTGGATAACGCCCTCCAATCGGGTAAC
TCCCAGGAGAGTGTCACAGAGCAGGACAGCAAGGACAGCACCTACAGCCTCA
GCAGCACCCTGACGCTGAGCAAAGCAGACTACGAGAAACACAAAGTCTACGC
CTGCGAAGTCACCCATCAGGGCCTGAGCTCGCCCGTCACAAAGAGCTTCAACA
GGGGAGAGTGTTAG

### Sequence of the Antibody Herein Identified as SBJ08-M13

### Amino Acid Sequences of SBJ08-M13:

> CDR 1 of variable domain of Heavy chain of SBJ08-M13
SEQ ID NO: 271
GFTFSTYA
> CDR 2 of variable domain of Heavy chain of SBJ08-M13
SEQ ID NO: 272
FSGNSDDT
> CDR 3 of variable domain of Heavy chain of SBJ08-M13
SEQ ID NO: 273
VRLWGGGTYMNRGDY
> CDR 1 of variable domain of Light chain of SBJ08-M13
SEQ ID NO: 274
QGISNY
> CDR 2 of variable domain of Light chain of SBJ08-M13 this sequence is
not included in the sequence listing because is less than 4 amino acid
SEQ ID NO: 275
AAS (Ala-Ala-Ser)
> CDR 3 of variable domain of Light chain of SBJ08-M13
SEQ ID NO: 276
QQYKSYPLT
> variable domain of Heavy chain of SBJ08-M13
SEQ ID NO: 277
EVQLVESGGDLVHPGGSLRLSCAASGFTFSTYAMSWVRQAPGKGLDWVSTFSGN
SDDTFYADSVKGRFTISRDNSKNTLYLQMNSLRAEDTAVYFCVRLWGGGTYMNR
GDYWGQGTLVTVSS
> variable domain of Light chain of SBJ08-M13
SEQ ID NO: 278
AIQMTQSPSSLSASVGDRVTITCRASQGISNYLAWFQQKPGKAPKSLIYAASSLQSG
VPSKFSGSGSGTDFTLTISSLEPEDFATYYCQQYKSYPLTFGGGTKVEIK
> Heavy chain of SBJ08-M13
SEQ ID NO: 279
MGWSCIILFLVATATGVHSEVQLVESGGDLVHPGGSLRLSCAASGFTFSTYAMSW
VRQAPGKGLDWVSTFSGNSDDTFYADSVKGRFTISRDNSKNTLYLQMNSLRAEDT
AVYFCVRLWGGGTYMNRGDYWGQGTLVTVSSASPTSPKSVFPLAPSSKSTSGGT
AALGCLVKDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSR
WQQGNVFSCSVMHGALHNHYTQKSLSLFPG*
> Light chain of SBJ08-M13
SEQ ID NO: 280
MGWSCIILFLVATATGVHSAIQMTQSPSSLSASVGDRVTITCRASQGISNYLAWFQ
QKPGKAPKSLIYAASSLQSGVPSKFSGSGSGTDFTLTISSLEPEDFATYYCQQYKSY
PLTFGGGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPV
TKSFNRGEC*

### Nucleic Acid Sequences of SBJ08-M13:

> SBJ08-M13_heavy_chain leader sequence
SEQ ID NO: 281
ATGGGATGGTCATGTATCATCCTTTTTCTAGTAGCAACTGCAACCGGTGTACAT
TCC
> SBJ08-M13_heavy_chain variable domain
SEQ ID NO: 282
GAGGTGCAGCTGGTGGAGTCTGGGGGAGACTTGGTACACCCTGGGGGGTCCCT
GAGACTCTCTTGTGCAGCCTCTGGATTCACCTTTAGTACCTATGCCATGAGCTG
GGTCCGCCAGGCTCCAGGGAAGGGGCTGGACTGGGTCTCAACTTTTAGTGGCA
ATAGTGATGACACATTCTACGCAGACTCCGTGAAGGGCCGGTTCACCATCTCC
AGAGACAATTCCAAGAACACGCTGTATCTGCAAATGAACAGCCTGAGAGCCG
AGGACACCGCCGTATATTTCTGTGTGAGATTGTGGGGTGGTGGGACCTACATG
AACCGGGGGGACTACTGGGGCCAGGGAACCCTGGTCACCGTCTCCTCAG
> SBJ08-M13_heavy_chain constant domain
SEQ ID NO: 283
CATCCCCGACCAGCCCCAAGTCGGTCTTCCCCCTGGCACCCTCCTCCAAGAGC
ACCTCTGGGGGCACAGCGGCCCTGGGCTGCCTGGTCAAGGACTACTTCCCCGA
ACCTGTGACGGTCTCGTGGAACTCAGGCGCCCTGACCAGCGGCGTGCACACCT
TCCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAAG
CCCAGCAACACCAAGGTGGACAAGAGAGTTGAGCCCAAATCTTGTGACAAAA
CTCACACATGCCCACCGTGCCCAGCACCTGAACTCCTGGGGGGACCGTCAGTC
TTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAA
CTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCA
GGACTGGCTGAATGGCAAGGAGTACAAGTGCAAGGTCTCCAACAAAGCCCTCC
CAGCCCCCATCGAGAAAACCATCTCCAAAGCCAAAGGGCAGCCCCGAGAACC
ACAGGTGTACACCCTGCCCCCATCCCGGGAGGAGATGACCAAGAACCAGGTCA
GCCTGACCTGCCTGGTCAAAGGCTTCTATCCCAGCGACATCGCCGTGGAGTGG
GAGAGCAATGGGCAGCCGGAGAACAACTACAAGACCACGCCTCCCGTGCTGG
ACTCCGACGGCTCCTTCTTCCTCTATAGCAAGCTCACCGTGGACAAGAGCAGG
TGGCAGCAGGGGAACGTCTTCTCATGCTCCGTGATGCATGGCGCTCTGCACAA
CCACTACACGCAGAAGAGCCTCTCCCTGTTCCCCGGGTAA
> SBJ08-M13_heavy_chain complete
SEQ ID NO: 284
ATGGGATGGTCATGTATCATCCTTTTTCTAGTAGCAACTGCAACCGGTGTACAT
TCCGAGGTGCAGCTGGTGGAGTCTGGGGGAGACTTGGTACACCCTGGGGGGTC
CCTGAGACTCTCTTGTGCAGCCTCTGGATTCACCTTTAGTACCTATGCCATGAG
CTGGGTCCGCCAGGCTCCAGGGAAGGGGCTGGACTGGGTCTCAACTTTTAGTG
GCAATAGTGATGACACATTCTACGCAGACTCCGTGAAGGGCCGGTTCACCATC
TCCAGAGACAATTCCAAGAACACGCTGTATCTGCAAATGAACAGCCTGAGAGC
CGAGGACACCGCCGTATATTTCTGTGTGAGATTGTGGGGTGGTGGGACCTACA
TGAACCGGGGGGACTACTGGGGCCAGGGAACCCTGGTCACCGTCTCCTCAGCA
TCCCCGACCAGCCCCAAGTCGGTCTTCCCCCTGGCACCCTCCTCCAAGAGCACC
TCTGGGGGCACAGCGGCCCTGGGCTGCCTGGTCAAGGACTACTTCCCCGAACC
TGTGACGGTCTCGTGGAACTCAGGCGCCCTGACCAGCGGCGTGCACACCTTCC
CGGCTGTCCTACAGTCCTCAGGACTCTACTCCCTCAGCAGCGTGGTGACCGTGC
CCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTC
ACACATGCCCACCGTGCCCAGCACCTGAACTCCTGGGGGGACCGTCAGTCTTC
CTCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAG
CAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCACA
GGTGTACACCCTGCCCCCATCCCGGGAGGAGATGACCAAGAACCAGGTCAGCC
TGACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTC
CGACGGCTCCTTCTTCCTCTATAGCAAGCTCACCGTGGACAAGAGCAGGTGGC
AGCAGGGGAACGTCTTCTCATGCTCCGTGATGCATGGCGCTCTGCACAACCAC
TACACGCAGAAGAGCCTCTCCCTGTTCCCCGGGTAA
> SBJ08-M13_light_kappa_chain leader sequence
SEQ ID NO: 285
ATGGGATGGTCATGTATCATCCTTTTTCTAGTAGCAACTGCAACCGGTGTACAT
TCC
> SBJ08-M13_light_kappa_chain variable domain
SEQ ID NO: 286
GCCATCCAGATGACCCAGTCTCCATCCTCACTGTCTGCATCTGTAGGAGACAG
AGTCACCATCACTTGTCGGGCGAGTCAGGGCATTAGCAATTATTTAGCCTGGTT
TCAGCAGAAACCAGGGAAAGCCCCTAAGTCCCTGATCTATGCTGCATCCAGTT
TGCAAAGTGGGGTCCCATCAAAATTCAGCGGCAGTGGATCTGGGACAGATTTC
ACTCTCACCATCAGCAGCCTGGAGCCTGAAGATTTTGCAACTTATTACTGCCAA
CAGTATAAAAGTTACCCGCTCACTTTCGGCGGAGGGACCAAGGTGGAGATCAA
AC
> SBJ08-M13_light_kappa_chain constant domain
SEQ ID NO: 287
GAACTGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGT
GTTAG
> SBJ08-M13_light_kappa_chain complete
SEQ ID NO: 288
ATGGGATGGTCATGTATCATCCTTTTTCTAGTAGCAACTGCAACCGGTGTACAT
TCCGCCATCCAGATGACCCAGTCTCCATCCTCACTGTCTGCATCTGTAGGAGAC
AGAGTCACCATCACTTGTCGGGCGAGTCAGGGCATTAGCAATTATTTAGCCTG
GTTTCAGCAGAAACCAGGGAAAGCCCCTAAGTCCCTGATCTATGCTGCATCCA
GTTTGCAAAGTGGGGTCCCATCAAAATTCAGCGGCAGTGGATCTGGGACAGAT
TTCACTCTCACCATCAGCAGCCTGGAGCCTGAAGATTTTGCAACTTATTACTGC
CAACAGTATAAAAGTTACCCGCTCACTTTCGGCGGAGGGACCAAGGTGGAGAT
CAAACGAACTGTGGCTGCACCATCTGTCTTCATCTTCCCGCCATCTGATGAGCA
GTTGAAATCTGGAACTGCCTCTGTTGTGTGCCTGCTGAATAACTTCTATCCCAG
AGAGGCCAAAGTACAGTGGAAGGTGGATAACGCCCTCCAATCGGGTAACTCCC
AGGAGAGTGTCACAGAGCAGGACAGCAAGGACAGCACCTACAGCCTCAGCAG
CACCCTGACGCTGAGCAAAGCAGACTACGAGAAACACAAAGTCTACGCCTGC
GAAGTCACCCATCAGGGCCTGAGCTCGCCCGTCACAAAGAGCTTCAACAGGGG
AGAGTGTTAG

### Sequence of the Antibody Herein Identified as SBJ08-N23

### Amino Acid Sequences of SBJ8N3

> CDR 1 of variable domain of Heavy chain of SBJ08-N23
SEQ ID NO: 289
GISVSNIY
> CDR 2 of variable domain of Heavy chain of SBJ08-N23
SEQ ID NO: 290
IYSSGGI
> CDR 3 of variable domain of Heavy chain of SBJ08-N23
SEQ ID NO: 291
VRDVRGTCGGDCYK
> CDR 1 of variable domain of Light chain of SBJ08-N23
SEQ ID NO: 292
QSL VHSDGNTY
> CDR 2 of variable domain of Light chain of SBJ08-N23 this sequence is
not included in the sequence listing because is less than 4 amino acid
SEQ ID NO: 293
KVS (Lys-Val-Ser)
> CDR 3 of variable domain of Light chain of SBJ08-N23
SEQ ID NO: 294
MQGAQWWT
> variable domain of Heavy chain of SBJ08-N23
SEQ ID NO: 295
EVQLVESGGGLVQPGGSLRLSCAVSGISVSNIYMTWVRQAPGRGLEWVSVIYSSG
GIEYPDSVKGRFTISRDNSKNTLYLQMNSLRAEDTAVYYCVRDVRGTCGGDCYK
WGQGTLVTVSS
> variable domain of Light chain of SBJ08-N23
SEQ ID NO: 296
DIVMTQTPLSLPVTLGQPASISCRSSQSLVHSDGNTYLSWFQQRPGQSPRRLIYKVS
NRDSGVPDRFSGSGSGSEFTLKISRVEAEDVGVYYCMQGAQWWTFGQGTRVEIK
> Heavy chain of SBJ08-N23
SEQ ID NO: 297
MGWSCIILFLVATATGVHSEVQLVESGGGLVQPGGSLRLSCAVSGISVSNIYMTWV
RQAPGRGLEWVSVIYSSGGIEYPDSVKGRFTISRDNSKNTLYLQMNSLRAEDTAVY
YCVRDVRGTCGGDCYKWGQGTLVTVSSASPTSPKSVFPLAPSSKSTSGGTAALGC
LVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQG
NVFSCSVMHGALHNHYTQKSLSLFPG*
> Light chain of SBJ08-N23
SEQ ID NO: 298
MGWSCIILFLVATATGVHSDIVMTQTPLSLPVTLGQPASISCRSSQSLVHSDGNTYL
SWFQQRPGQSPRRLIYKVSNRDSGVPDRFSGSGSGSEFTLKISRVEAEDVGVYYCM
QGAQWWTFGQGTRVEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQ
GLSSPVTKSFNRGEC*

### Nucleic Acid Sequences of SBJ08-N23:

> SBJ08-N23_heavy_chain leader sequence
SEQ ID NO: 299
ATGGGATGGTCATGTATCATCCTTTTTCTAGTAGCAACTGCAACCGGTGTACAT
TCC
> SBJ08-N23_heavy_chain variable domain
SEQ ID NO: 300
GAGGTGCAGCTGGTGGAGTCTGGGGGAGGCTTGGTCCAGCCGGGGGGGTCCCT
CAGACTCTCCTGTGCAGTCTCTGGAATCAGCGTCAGTAACATCTATATGACATG
GGTCCGCCAGGCTCCAGGGAGGGGGCTGGAGTGGGTCTCAGTGATTTACAGTA
GTGGTGGTATAGAGTACCCAGACTCCGTGAAGGGCAGATTCACCATCTCCAGA
GACAATTCCAAGAACACGCTGTATCTTCAAATGAACAGCCTGAGAGCCGAGGA
CACGGCTGTATATTACTGTGTGAGAGATGTGCGGGGGACGTGTGGTGGTGACT
GCTACAAGTGGGGCCAGGGAACCCTGGTCACCGTCTCCTCAG
> SBJ08-N23_heavy chain constant domain
SEQ ID NO: 301
CATCCCCGACCAGCCCCAAGTCGGTCTTCCCCCTGGCACCCTCCTCCAAGAGC
ACCTCTGGGGGCACAGCGGCCCTGGGCTGCCTGGTCAAGGACTACTTCCCCGA
ACCTGTGACGGTCTCGTGGAACTCAGGCGCCCTGACCAGCGGCGTGCACACCT
TCCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAAG
CCCAGCAACACCAAGGTGGACAAGAGAGTTGAGCCCAAATCTTGTGACAAAA
CTCACACATGCCCACCGTGCCCAGCACCTGAACTCCTGGGGGGACCGTCAGTC
TTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAA
CTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCA
GGACTGGCTGAATGGCAAGGAGTACAAGTGCAAGGTCTCCAACAAAGCCCTCC
CAGCCCCCATCGAGAAAACCATCTCCAAAGCCAAAGGGCAGCCCCGAGAACC
ACAGGTGTACACCCTGCCCCCATCCCGGGAGGAGATGACCAAGAACCAGGTCA
GCCTGACCTGCCTGGTCAAAGGCTTCTATCCCAGCGACATCGCCGTGGAGTGG
GAGAGCAATGGGCAGCCGGAGAACAACTACAAGACCACGCCTCCCGTGCTGG
ACTCCGACGGCTCCTTCTTCCTCTATAGCAAGCTCACCGTGGACAAGAGCAGG
TGGCAGCAGGGGAACGTCTTCTCATGCTCCGTGATGCATGGCGCTCTGCACAA
CCACTACACGCAGAAGAGCCTCTCCCTGTTCCCCGGGTAA
> SBJ08-N23_heavy_chain complete
SEQ ID NO: 302
ATGGGATGGTCATGTATCATCCTTTTTCTAGTAGCAACTGCAACCGGTGTACAT
TCCGAGGTGCAGCTGGTGGAGTCTGGGGGAGGCTTGGTCCAGCCGGGGGGGTC
CCTCAGACTCTCCTGTGCAGTCTCTGGAATCAGCGTCAGTAACATCTATATGAC
ATGGGTCCGCCAGGCTCCAGGGAGGGGGCTGGAGTGGGTCTCAGTGATTTACA
GTAGTGGTGGTATAGAGTACCCAGACTCCGTGAAGGGCAGATTCACCATCTCC
AGAGACAATTCCAAGAACACGCTGTATCTTCAAATGAACAGCCTGAGAGCCGA
GGACACGGCTGTATATTACTGTGTGAGAGATGTGCGGGGGACGTGTGGTGGTG
ACTGCTACAAGTGGGGCCAGGGAACCCTGGTCACCGTCTCCTCAGCATCCCCG
ACCAGCCCCAAGTCGGTCTTCCCCCTGGCACCCTCCTCCAAGAGCACCTCTGGG
GGCACAGCGGCCCTGGGCTGCCTGGTCAAGGACTACTTCCCCGAACCTGTGAC
GGTCTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCA
GCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATG
CCCACCGTGCCCAGCACCTGAACTCCTGGGGGGACCGTCAGTCTTCCTCTTCCC
CCCAAAACCCAAGGACACCCTCATGATCTCCCGGACCCCTGAGGTCACATGCG
TGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACA
ACAGCACGTACCGTGTGGTCAGCGTCCTCACCGTCCTGCACCAGGACTGGCTG
AATGGCAAGGAGTACAAGTGCAAGGTCTCCAACAAAGCCCTCCCAGCCCCCAT
CGAGAAAACCATCTCCAAAGCCAAAGGGCAGCCCCGAGAACCACAGGTGTAC
ACCCTGCCCCCATCCCGGGAGGAGATGACCAAGAACCAGGTCAGCCTGACCTG
CCTGGTCAAAGGCTTCTATCCCAGCGACATCGCCGTGGAGTGGGAGAGCAATG
GGCAGCCGGAGAACAACTACAAGACCACGCCTCCCGTGCTGGACTCCGACGGC
TCCTTCTTCCTCTATAGCAAGCTCACCGTGGACAAGAGCAGGTGGCAGCAGGG
GAACGTCTTCTCATGCTCCGTGATGCATGGCGCTCTGCACAACCACTACACGCA
GAAGAGCCTCTCCCTGTTCCCCGGGTAA
> SBJ08-N23_light_kappa_chain leader sequence
SEQ ID NO: 303
ATGGGATGGTCATGTATCATCCTTTTTCTAGTAGCAACTGCAACCGGTGTACAT
TCC
> SBJ08-N23_light_kappa_chain variable domain
SEQ ID NO: 304
GATATTGTGATGACCCAGACTCCACTCTCCCTGCCCGTCACCCTTGGACAGCCG
GCCTCCATCTCCTGCAGGTCTAGTCAAAGCCTCGTACACAGTGATGGAAACAC
CTACTTGAGTTGGTTTCAGCAGAGGCCAGGCCAATCTCCAAGGCGCCTAATTT
ATAAGGTGTCTAACCGGGACTCTGGGGTCCCAGACAGATTCAGCGGCAGTGGG
TCAGGCAGTGAATTCACACTGAAAATCAGCAGGGTGGAGGCTGAGGATGTTGG
GGTCTATTACTGCATGCAAGGCGCACAGTGGTGGACGTTCGGCCAAGGGACCA
GGGTGGAAATCAAAC
>SBJ08-N23_light_kappa_chain constant domain
SEQ ID NO: 305
GAACTGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGT
GTTAG
> SBJ08-N23_light_kappa_chain complete
SEQ ID NO: 306
ATGGGATGGTCATGTATCATCCTTTTTCTAGTAGCAACTGCAACCGGTGTACAT
TCCGATATTGTGATGACCCAGACTCCACTCTCCCTGCCCGTCACCCTTGGACAG
CCGGCCTCCATCTCCTGCAGGTCTAGTCAAAGCCTCGTACACAGTGATGGAAA
CACCTACTTGAGTTGGTTTCAGCAGAGGCCAGGCCAATCTCCAAGGCGCCTAA
TTTATAAGGTGTCTAACCGGGACTCTGGGGTCCCAGACAGATTCAGCGGCAGT
GGGTCAGGCAGTGAATTCACACTGAAAATCAGCAGGGTGGAGGCTGAGGATG
TTGGGGTCTATTACTGCATGCAAGGCGCACAGTGGTGGACGTTCGGCCAAGGG
ACCAGGGTGGAAATCAAACGAACTGTGGCTGCACCATCTGTCTTCATCTTCCC
GCCATCTGATGAGCAGTTGAAATCTGGAACTGCCTCTGTTGTGTGCCTGCTGAA
TAACTTCTATCCCAGAGAGGCCAAAGTACAGTGGAAGGTGGATAACGCCCTCC
AATCGGGTAACTCCCAGGAGAGTGTCACAGAGCAGGACAGCAAGGACAGCAC
CTACAGCCTCAGCAGCACCCTGACGCTGAGCAAAGCAGACTACGAGAAACAC
AAAGTCTACGCCTGCGAAGTCACCCATCAGGGCCTGAGCTCGCCCGTCACAAA
GAGCTTCAACAGGGGAGAGTGTTAG

### Sequence of the Antibody Herein Identified as SBJ08-003

### Amino Acid Sequences of SBJ08-003:

> CDR 1 of variable domain of Heavy chain of SBJ08-O03
SEQ ID NO: 307
GFTFNGFA
> CDR 2 of variable domain of Heavy chain of SBJ08-O03
SEQ ID NO: 308
ISDSGDHR
> CDR 3 of variable domain of Heavy chain of SBJ08-O03
SEQ ID NO: 309
AKHVVWGQWYFDS
> CDR 1 of variable domain of Light chain of SBJ08-O03
SEQ ID NO: 310
QTVTSNY
> CDR 2 of variable domain of Light chain of SBJ08-O03 this sequence is
not included in the sequence listing because is less than 4 amino acid
SEQ ID NO: 311
GAS (Gly-Ala-Ser)
> CDR 3 of variable domain of Light chain of SBJ08-O03
SEQ ID NO: 312
QQYGSSVTSGPA
> variable domain of Heavy chain of SBJ08-O03
SEQ ID NO: 313
EVQLVESGGGLVQPGGSLILSCAVSGFTFNGFAMTWVRRAPGKGLEWVSTISDSG
DHRFYADSVKGRFTISRDNSKNTLYLQMISLRAEDTAVYSCAKHVVWGQWYFDS
WGQGTLVTVSS
> variable domain of Light chain of SBJ08-O03
SEQ ID NO: 314
EIVMTQSPGTLSLSPGERATLSCRASQTVTSNYLAWYQQKPGQAPRLLISGASSRA
TGIPDRFSGSGSGTDFTLTISRLEPEDFVVYYCQQYGSSVTSGPAFGGGTKVEIK
> Heavy chain of SBJ08-O03
SEQ ID NO: 315
MGWSCIILFLVATATGVHSEVQLVESGGGLVQPGGSLILSCAVSGFTFNGFAMTW
VRRAPGKGLEWVSTISDSGDHRFYADSVKGRFTISRDNSKNTLYLQMISLRAEDTA
VYSCAKHVVWGQWYFDSWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQG
NVFSCSVMHGALHNHYTQKSLSLFPG*
> Light chain of SBJ08-O03
SEQ ID NO: 316
MGWSCIILFLVATATGVHSEIVMTQSPGTLSLSPGERATLSCRASQTVTSNYLAWY
QQKPGQAPRLLISGASSRATGIPDRFSGSGSGTDFTLTISRLEPEDFVVYYCQQYGS
SVTSGPAFGGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGL
SSPVTKSFNRGEC*

### Nucleic Acid Sequences of SBJ08-003:

> SBJ08-O03_heavy_chain leader sequence
SEQ ID NO: 317
ATGGGATGGTCATGTATCATCCTTTTTCTAGTAGCAACTGCAACCGGTGTACAT
TCC
> SBJ08-O03_heavy_chain variable domain
SEQ ID NO: 318
GAGGTGCAGCTGGTGGAGTCTGGGGGAGGCTTGGTACAGCCTGGGGGGTCCCT
GATACTCTCCTGTGCAGTCTCTGGATTCACCTTTAATGGCTTTGCCATGACCTG
GGTCCGCCGGGCTCCAGGGAAGGGGCTGGAGTGGGTCTCAACTATTAGTGATA
GTGGTGATCACAGATTCTACGCAGACTCCGTGAAGGGCCGGTTCACCATCTCC
AGAGACAATTCCAAGAACACCCTGTATCTGCAAATGATCAGCCTGAGAGCCGA
GGACACGGCCGTATATTCCTGTGCGAAACACGTAGTCTGGGGACAGTGGTACT
TTGACTCCTGGGGCCAGGGAACCCTGGTCACCGTCTCCTCAG
> SBJ08-O03_heavy_chain constant domain
SEQ ID NO: 319
CTTCCACCAAGGGCCCATCGGTCTTCCCCCTGGCACCCTCCTCCAAGAGCACCT
CTGGGGGCACAGCGGCCCTGGGCTGCCTGGTCAAGGACTACTTCCCCGAACCT
GTGACGGTCTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCC
CTCCAGCAGCTTGGGCACCCAGACCTACATCTGCAACGTGAATCACAAGCCCA
GCAACACCAAGGTGGACAAGAGAGTTGAGCCCAAATCTTGTGACAAAACTCA
CACATGCCCACCGTGCCCAGCACCTGAACTCCTGGGGGGACCGTCAGTCTTCC
TCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAG
CAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCACA
GGTGTACACCCTGCCCCCATCCCGGGAGGAGATGACCAAGAACCAGGTCAGCC
TGACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTC
CGACGGCTCCTTCTTCCTCTATAGCAAGCTCACCGTGGACAAGAGCAGGTGGC
AGCAGGGGAACGTCTTCTCATGCTCCGTGATGCATGGCGCTCTGCACAACCAC
TACACGCAGAAGAGCCTCTCCCTGTTCCCCGGGTAA
> SBJ08-O03_heavy_chain complete
SEQ ID NO: 320
ATGGGATGGTCATGTATCATCCTTTTTCTAGTAGCAACTGCAACCGGTGTACAT
TCCGAGGTGCAGCTGGTGGAGTCTGGGGGAGGCTTGGTACAGCCTGGGGGGTC
CCTGATACTCTCCTGTGCAGTCTCTGGATTCACCTTTAATGGCTTTGCCATGAC
CTGGGTCCGCCGGGCTCCAGGGAAGGGGCTGGAGTGGGTCTCAACTATTAGTG
ATAGTGGTGATCACAGATTCTACGCAGACTCCGTGAAGGGCCGGTTCACCATC
TCCAGAGACAATTCCAAGAACACCCTGTATCTGCAAATGATCAGCCTGAGAGC
CGAGGACACGGCCGTATATTCCTGTGCGAAACACGTAGTCTGGGGACAGTGGT
ACTTTGACTCCTGGGGCCAGGGAACCCTGGTCACCGTCTCCTCAGCTTCCACCA
AGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCTGTGACGGT
CTCGTGGAACTCAGGCGCCCTGACCAGCGGCGTGCACACCTTCCCGGCTGTCC
TACAGTCCTCAGGACTCTACTCCCTCAGCAGCGTGGTGACCGTGCCCTCCAGCA
GCTTGGGCACCCAGACCTACATCTGCAACGTGAATCACAAGCCCAGCAACACC
AAGGTGGACAAGAGAGTTGAGCCCAAATCTTGTGACAAAACTCACACATGCCC
ACCGTGCCCAGCACCTGAACTCCTGGGGGGACCGTCAGTCTTCCTCTTCCCCCC
AAAACCCAAGGACACCCTCATGATCTCCCGGACCCCTGAGGTCACATGCGTGG
TGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAACA
GCACGTACCGTGTGGTCAGCGTCCTCACCGTCCTGCACCAGGACTGGCTGAAT
GGCAAGGAGTACAAGTGCAAGGTCTCCAACAAAGCCCTCCCAGCCCCCATCGA
GAAAACCATCTCCAAAGCCAAAGGGCAGCCCCGAGAACCACAGGTGTACACC
CTGCCCCCATCCCGGGAGGAGATGACCAAGAACCAGGTCAGCCTGACCTGCCT
GGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCC
TTCTTCCTCTATAGCAAGCTCACCGTGGACAAGAGCAGGTGGCAGCAGGGGAA
CGTCTTCTCATGCTCCGTGATGCATGGCGCTCTGCACAACCACTACACGCAGAA
GAGCCTCTCCCTGTTCCCCGGGTAA
> SBJ08-O03_light_kappa_chain leader sequence
SEQ ID NO: 321
ATGGGATGGTCATGTATCATCCTTTTTCTAGTAGCAACTGCAACCGGTGTACAT
TCC
> SBJ08-O03_light_kappa_chain variable domain
SEQ ID NO: 322
GAAATTGTGATGACGCAGTCTCCAGGCACCCTGTCTTTGTCTCCAGGGGAAAG
AGCCACCCTCTCCTGCAGGGCCAGTCAGACTGTTACCAGCAACTACTTAGCCT
GGTACCAGCAGAAACCTGGCCAGGCTCCCAGGCTCCTCATCTCTGGTGCATCC
AGCAGGGCCACTGGCATCCCAGACAGGTTCAGTGGCAGTGGGTCTGGGACAG
ACTTCACTCTCACCATCAGCAGACTGGAGCCTGAAGATTTCGTAGTGTATTACT
GTCAGCAGTATGGTAGCTCAGTCACCTCTGGTCCCGCTTTCGGCGGAGGGACC
AAGGTGGAGATCAAAC
> SBJ08-O03_light_kappa_chain constant domain
SEQ ID NO: 323
GAACTGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGT
GTTAG
> SBJ08-O03_light_kappa_chain complete
SEQ ID NO: 324
ATGGGATGGTCATGTATCATCCTTTTTCTAGTAGCAACTGCAACCGGTGTACAT
TCCGAAATTGTGATGACGCAGTCTCCAGGCACCCTGTCTTTGTCTCCAGGGGAA
AGAGCCACCCTCTCCTGCAGGGCCAGTCAGACTGTTACCAGCAACTACTTAGC
CTGGTACCAGCAGAAACCTGGCCAGGCTCCCAGGCTCCTCATCTCTGGTGCAT
CCAGCAGGGCCACTGGCATCCCAGACAGGTTCAGTGGCAGTGGGTCTGGGACA
GACTTCACTCTCACCATCAGCAGACTGGAGCCTGAAGATTTCGTAGTGTATTAC
TGTCAGCAGTATGGTAGCTCAGTCACCTCTGGTCCCGCTTTCGGCGGAGGGAC
CAAGGIGGAGATCAAACGAACTGTGGCTGCACCATCTGTCTTCATCTTCCCGCC
ATCTGATGAGCAGTTGAAATCTGGAACTGCCTCTGTTGTGTGCCTGCTGAATAA
CTTCTATCCCAGAGAGGCCAAAGTACAGTGGAAGGTGGATAACGCCCTCCAAT
CGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAA
GTCTACGCCTGCGAAGTCACCCATCAGGGCCTGAGCTCGCCCGTCACAAAGAG
CTTCAACAGGGGAGAGTGTTAG

### Sequence of the Antibody Herein Identified as SBJ08-009

### Amino Acid Sequences of SBJ08-009:

> CDR 1 of variable domain of Heavy chain of SBJ08-O09
SEQ ID NO: 325
GFTFSSYS
> CDR 2 of variable domain of Heavy chain of SBJ08-O09
SEQ ID NO: 326
ISGTGGTK
> CDR 3 of variable domain of Heavy chain of SBJ08-O09
SEQ ID NO: 327
AKRTTNSGAFDV
> CDR 1 of variable domain of Light chain of SBJ08-O09
SEQ ID NO: 328
QSVTGSH
> CDR 2 of variable domain of Light chain of SBJ08-O09 this sequence is
not included in the sequence listing because is less than 4 amino acid
SEQ ID NO: 329
DAS (Asp-Ala-Ser)
> CDR 3 of variable domain of Light chain of SBJ08-O09
SEQ ID NO: 330
QQYGSSPLFS
> variable domain of Heavy chain of SBJ08-O09
SEQ ID NO: 331
EVQLVESGGGLVQPGGSLRLSCAASGFTFSSYSMIWVRQASGKGLEWVSHISGTG
GTKNYADSVKGRFTTSRDSSKNTLYLQMNSLRDEDTAVYYCAKRTTNSGAFDVW
GQGTMVTVSS
> variable domain of Light chain of SBJ08-O09
SEQ ID NO: 332
ETTLTQSPGTLSLSPGERATLSCRASQSVTGSHLAWYQQKPGQAPRLLIYDASSRA
TGIPDRFSGSGSGTDFTLTISRLEPEDFAVYYCQQYGSSPLFSFGQGTKLEIK
> Heavy chain of SBJ08-O09
SEQ ID NO: 333
MGWSCIILFLVATATGVHSEVQLVESGGGLVQPGGSLRLSCAASGFTFSSYSMIWV
RQASGKGLEWVSHISGTGGTKNYADSVKGRFTTSRDSSKNTLYLQMNSLRDEDT
AVYYCAKRTTNSGAFDVWGQGTMVTVSSASTKGPSVFPLAPSSKSTSGGTAALGC
LVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQG
NVFSCSVMHGALHNHYTQKSLSLFPG*
> Light chain of SBJ08-O09
SEQ ID NO: 334
MGWSCIILFLVATATGVHSETTLTQSPGTLSLSPGERATLSCRASQSVTGSHLAWY
QQKPGQAPRLLIYDASSRATGIPDRFSGSGSGTDFTLTISRLEPEDFAVYYCQQYGS
SPLFSFGQGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSP
VTKSFNRGEC*

### Nucleic Acid Sequences of SBJ08-009:

> SBJ08-O09 heavy chain leader sequence
SEQ ID NO: 335
ATGGGATGGTCATGTATCATCCTTTTTCTAGTAGCAACTGCAACCGGTGTACAT
TCC
> SBJ08-O09_heavy_chain variable domain
SEQ ID NO: 336
GAGGTGCAGCTGGTGGAGTCTGGGGGAGGCTTGGTGCAGCCTGGGGGGTCCCT
GAGACTCTCCTGTGCAGCCTCTGGATTCACCTTCAGCAGCTATTCCATGATCTG
GGTCCGCCAGGCTTCAGGGAAGGGGCTGGAGTGGGTCTCACATATTAGTGGTA
CTGGTGGTACCAAAAACTACGCAGACTCCGTGAAGGGCCGCTTCACCACCTCC
AGAGACAGCTCCAAGAACACGCTGTATCTGCAAATGAACAGCCTGAGAGACG
AGGACACGGCCGTCTATTACTGTGCGAAACGGACGACGAATTCTGGTGCTTTT
GATGTTTGGGGCCAAGGGACAATGGTCACCGTCTCTTCAG
> SBJ08-O09_heavy_chain constant domain
SEQ ID NO: 337
CCTCCACCAAGGGCCCATCGGTCTTCCCCCTGGCACCCTCCTCCAAGAGCACCT
CTGGGGGCACAGCGGCCCTGGGCTGCCTGGTCAAGGACTACTTCCCCGAACCT
GTGACGGTCTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCC
CTCCAGCAGCTTGGGCACCCAGACCTACATCTGCAACGTGAATCACAAGCCCA
GCAACACCAAGGTGGACAAGAGAGTTGAGCCCAAATCTTGTGACAAAACTCA
CACATGCCCACCGTGCCCAGCACCTGAACTCCTGGGGGGACCGTCAGTCTTCC
TCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAG
CAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCACA
GGTGTACACCCTGCCCCCATCCCGGGAGGAGATGACCAAGAACCAGGTCAGCC
TGACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTC
CGACGGCTCCTTCTTCCTCTATAGCAAGCTCACCGTGGACAAGAGCAGGTGGC
AGCAGGGGAACGTCTTCTCATGCTCCGTGATGCATGGCGCTCTGCACAACCAC
TACACGCAGAAGAGCCTCTCCCTGTTCCCCGGGTAA
> SBJ08-O09_heavy_chain complete
SEQ ID NO: 338
ATGGGATGGTCATGTATCATCCTTTTTCTAGTAGCAACTGCAACCGGTGTACAT
TCCGAGGTGCAGCTGGTGGAGTCTGGGGGAGGCTTGGTGCAGCCTGGGGGGTC
CCTGAGACTCTCCTGTGCAGCCTCTGGATTCACCTTCAGCAGCTATTCCATGAT
CTGGGTCCGCCAGGCTTCAGGGAAGGGGCTGGAGTGGGTCTCACATATTAGTG
GTACTGGTGGTACCAAAAACTACGCAGACTCCGTGAAGGGCCGCTTCACCACC
TCCAGAGACAGCTCCAAGAACACGCTGTATCTGCAAATGAACAGCCTGAGAGA
CGAGGACACGGCCGTCTATTACTGTGCGAAACGGACGACGAATTCTGGTGCTT
TTGATGTTTGGGGCCAAGGGACAATGGTCACCGTCTCTTCAGCCTCCACCAAG
GGCCCATCGGTCTTCCCCCTGGCACCCTCCTCCAAGAGCACCTCTGGGGGCAC
AGCGGCCCTGGGCTGCCTGGTCAAGGACTACTTCCCCGAACCTGTGACGGTCT
CGTGGAACTCAGGCGCCCTGACCAGCGGCGTGCACACCTTCCCGGCTGTCCTA
CAGTCCTCAGGACTCTACTCCCTCAGCAGCGTGGTGACCGTGCCCTCCAGCAG
CTTGGGCACCCAGACCTACATCTGCAACGTGAATCACAAGCCCAGCAACACCA
AGGTGGACAAGAGAGTTGAGCCCAAATCTTGTGACAAAACTCACACATGCCCA
CCGTGCCCAGCACCTGAACTCCTGGGGGGACCGTCAGTCTTCCTCTTCCCCCCA
AAACCCAAGGACACCCTCATGATCTCCCGGACCCCTGAGGTCACATGCGTGGT
GGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAG
CACGTACCGTGTGGTCAGCGTCCTCACCGTCCTGCACCAGGACTGGCTGAATG
GCAAGGAGTACAAGTGCAAGGTCTCCAACAAAGCCCTCCCAGCCCCCATCGAG
AAAACCATCTCCAAAGCCAAAGGGCAGCCCCGAGAACCACAGGTGTACACCC
TGCCCCCATCCCGGGAGGAGATGACCAAGAACCAGGTCAGCCTGACCTGCCTG
GTCAAAGGCTTCTATCCCAGCGACATCGCCGTGGAGTGGGAGAGCAATGGGCA
GCCGGAGAACAACTACAAGACCACGCCTCCCGTGCTGGACTCCGACGGCTCCT
TCTTCCTCTATAGCAAGCTCACCGTGGACAAGAGCAGGTGGCAGCAGGGGAAC
GTCTTCTCATGCTCCGTGATGCATGGCGCTCTGCACAACCACTACACGCAGAA
GAGCCTCTCCCTGTTCCCCGGGTAA
> SBJ08-O09_light_kappa_chain leader sequence
SEQ ID NO: 339
ATGGGATGGTCATGTATCATCCTTTTTCTAGTAGCAACTGCAACCGGTGTACAT
TCC
> SBJ08-O09_light_kappa_chain variable domain
SEQ ID NO: 340
GAAACGACACTCACGCAGTCTCCAGGCACCCTGTCTTTGTCTCCAGGGGAAAG
AGCCACCCTCTCCTGCAGGGCCAGTCAGAGTGTTACCGGCAGCCACTTAGCCT
GGTACCAGCAGAAACCTGGCCAGGCTCCCAGGCTCCTCATCTATGATGCATCC
AGCAGGGCCACTGGCATCCCAGACAGGTTCAGTGGCAGTGGGTCTGGGACAG
ACTTCACTCTCACCATCAGCAGACTGGAGCCTGAAGATTTTGCAGTGTATTACT
GTCAGCAGTATGGTAGCTCACCTCTGTTCAGTTTTGGCCAGGGGACCAAGCTG
GAGATCAAAC
> SBJ08-O09_light_kappa_chain constant domain
SEQ ID NO: 341
GAACTGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGT
GTTAG
> SBJ08-O09_light_kappa_chain complete
SEQ ID NO: 342
ATGGGATGGTCATGTATCATCCTTTTTCTAGTAGCAACTGCAACCGGTGTACAT
TCCGAAACGACACTCACGCAGTCTCCAGGCACCCTGTCTTTGTCTCCAGGGGA
AAGAGCCACCCTCTCCTGCAGGGCCAGTCAGAGTGTTACCGGCAGCCACTTAG
CCTGGTACCAGCAGAAACCTGGCCAGGCTCCCAGGCTCCTCATCTATGATGCA
TCCAGCAGGGCCACTGGCATCCCAGACAGGTTCAGTGGCAGTGGGTCTGGGAC
AGACTTCACTCTCACCATCAGCAGACTGGAGCCTGAAGATTTTGCAGTGTATTA
CTGTCAGCAGTATGGTAGCTCACCTCTGTTCAGTTTTGGCCAGGGGACCAAGCT
GGAGATCAAACGAACTGTGGCTGCACCATCTGTCTTCATCTTCCCGCCATCTGA
TGAGCAGTTGAAATCTGGAACTGCCTCTGTTGTGTGCCTGCTGAATAACTTCTA
TCCCAGAGAGGCCAAAGTACAGTGGAAGGTGGATAACGCCCTCCAATCGGGT
AACTCCCAGGAGAGTGTCACAGAGCAGGACAGCAAGGACAGCACCTACAGCC
TCAGCAGCACCCTGACGCTGAGCAAAGCAGACTACGAGAAACACAAAGTCTA
CGCCTGCGAAGTCACCCATCAGGGCCTGAGCTCGCCCGTCACAAAGAGCTTCA
ACAGGGGAGAGTGTTAG

### Sequence of the Antibody Herein Identified as SBJ09-G14

### Amino Acid Sequences of SBJ09-G14:

> CDR 1 of variable domain of Heavy chain of SBJ09-G14
SEQ ID NO: 343
GFSVSRSW
> CDR 2 of variable domain of Heavy chain of SBJ09-G14
SEQ ID NO: 344
IQSDDNKI
> CDR 3 of variable domain of Heavy chain of SBJ09-G14
SEQ ID NO: 345
ARDRGWGLFDI
> CDR 1 of variable domain of Light chain of SBJ09-G14
SEQ ID NO: 346
QSLLQSDGKTY
> CDR 2 of variable domain of Light chain of SBJ09-G14 this sequence is
not included in the sequence listing because is less than 4 amino acid
SEQ ID NO: 347
EVS (Glu-Val-Ser)
> CDR 3 of variable domain of Light chain of SBJ09-G14
SEQ ID NO: 348
VHHLPLPLT
> variable domain of Heavy chain of SBJ09-G14
SEQ ID NO: 349
EVQLVESGGGLVQPGGSLRLSCAASGFSVSRSWMNWVRQAPGKGLVWVSSIQSD
DNKIGYADSVRGRFAVSGDNAKNTVFLQMNSLRAEDTAVYYCARDRGWGLFDI
WGQGTLVTVSS
> variable domain of Light chain of SBJ09-G14
SEQ ID NO: 350
DIVMTQSPLSLSVTPGQPASISCKSSQSLLQSDGKTYLHWYLQKPGQSPQPLIYEVS
NRFSGVPDRFSGSGSGTDFTLKISRVEAEDVGVYYCVHHLPLPLTFGGGTKVEIK
> Heavy chain of SBJ09-G14
SEQ ID NO: 351
MGWSCIILFLVATATGVHSEVQLVESGGGLVQPGGSLRLSCAASGFSVSRSWMNW
VRQAPGKGLVWVSSIQSDDNKIGYADSVRGRFAVSGDNAKNTVFLQMNSLRAED
TAVYYCARDRGWGLFDIWGQGTLVTVSSASPTSPKSVFPLAPSSKSTSGGTAALG
CLVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQG
NVFSCSVMHGALHNHYTQKSLSLFPG*
> Light chain of SBJ09-G14
SEQ ID NO: 352
MGWSCIILFLVATATGVHSDIVMTQSPLSLSVTPGQPASISCKSSQSLLQSDGKTYL
HWYLQKPGQSPQPLIYEVSNRFSGVPDRFSGSGSGTDFTLKISRVEAEDVGVYYCV
HHLPLPLTFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQG
LSSPVTKSFNRGEC*

### Nucleic Acid Sequences of SBJ09-G14:

> SBJ09-G14_heavy_chain leader sequence
SEQ ID NO: 353
ATGGGATGGTCATGTATCATCCTTTTTCTAGTAGCAACTGCAACCGGTGTACAT
TCC
> SBJ09-G14_heavy_chain variable domain
SEQ ID NO: 354
GAGGTGCAGCTGGTGGAGTCTGGGGGAGGCTTAGTTCAGCCGGGGGGGTCCCT
GAGACTCTCCTGTGCAGCCTCTGGATTCAGCGTCAGTAGGTCCTGGATGAATTG
GGTCCGCCAAGCTCCAGGGAAGGGGCTGGTGTGGGTCTCATCTATTCAAAGTG
ATGACAATAAGATAGGCTACGCGGACTCCGTGAGGGGCCGATTCGCCGTCTCC
GGAGACAACGCCAAGAATACGGTGTTTCTGCAAATGAACAGTCTGAGAGCCG
AGGATACGGCTGTCTATTACTGCGCAAGAGATCGGGGCTGGGGACTTTTTGAT
ATCTGGGGCCAAGGGACATTGGTCACCGTCTCTTCAG
> SBJ09-G14_heavy_chain constant domain
SEQ ID NO: 355
CATCCCCGACCAGCCCCAAGTCGGTCTTCCCCCTGGCACCCTCCTCCAAGAGC
ACCTCTGGGGGCACAGCGGCCCTGGGCTGCCTGGTCAAGGACTACTTCCCCGA
ACCTGTGACGGTCTCGTGGAACTCAGGCGCCCTGACCAGCGGCGTGCACACCT
TCCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAAG
CCCAGCAACACCAAGGTGGACAAGAGAGTTGAGCCCAAATCTTGTGACAAAA
CTCACACATGCCCACCGTGCCCAGCACCTGAACTCCTGGGGGGACCGTCAGTC
TTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAA
CTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCA
GGACTGGCTGAATGGCAAGGAGTACAAGTGCAAGGTCTCCAACAAAGCCCTCC
CAGCCCCCATCGAGAAAACCATCTCCAAAGCCAAAGGGCAGCCCCGAGAACC
ACAGGTGTACACCCTGCCCCCATCCCGGGAGGAGATGACCAAGAACCAGGTCA
GCCTGACCTGCCTGGTCAAAGGCTTCTATCCCAGCGACATCGCCGTGGAGTGG
GAGAGCAATGGGCAGCCGGAGAACAACTACAAGACCACGCCTCCCGTGCTGG
ACTCCGACGGCTCCTTCTTCCTCTATAGCAAGCTCACCGTGGACAAGAGCAGG
TGGCAGCAGGGGAACGTCTTCTCATGCTCCGTGATGCATGGCGCTCTGCACAA
CCACTACACGCAGAAGAGCCTCTCCCTGTTCCCCGGGTAA
> SBJ09-G14_heavy_chain complete
SEQ ID NO: 356
ATGGGATGGTCATGTATCATCCTTTTTCTAGTAGCAACTGCAACCGGTGTACAT
TCCGAGGTGCAGCTGGTGGAGTCTGGGGGAGGCTTAGTTCAGCCGGGGGGGTC
CCTGAGACTCTCCTGTGCAGCCTCTGGATTCAGCGTCAGTAGGTCCTGGATGAA
TTGGGTCCGCCAAGCTCCAGGGAAGGGGCTGGTGTGGGTCTCATCTATTCAAA
GTGATGACAATAAGATAGGCTACGCGGACTCCGTGAGGGGCCGATTCGCCGTC
TCCGGAGACAACGCCAAGAATACGGTGTTTCTGCAAATGAACAGTCTGAGAGC
CGAGGATACGGCTGTCTATTACTGCGCAAGAGATCGGGGCTGGGGACTTTTTG
ATATCTGGGGCCAAGGGACATTGGTCACCGTCTCTTCAGCATCCCCGACCAGC
CCCAAGTCGGTCTTCCCCCTGGCACCCTCCTCCAAGAGCACCTCTGGGGGCAC
AGCGGCCCTGGGCTGCCTGGTCAAGGACTACTTCCCCGAACCTGTGACGGTCT
CGTGGAACTCAGGCGCCCTGACCAGCGGCGTGCACACCTTCCCGGCTGTCCTA
CAGTCCTCAGGACTCTACTCCCTCAGCAGCGTGGTGACCGTGCCCTCCAGCAG
CTTGGGCACCCAGACCTACATCTGCAACGTGAATCACAAGCCCAGCAACACCA
AGGTGGACAAGAGAGTTGAGCCCAAATCTTGTGACAAAACTCACACATGCCCA
CCGTGCCCAGCACCTGAACTCCTGGGGGGACCGTCAGTCTTCCTCTTCCCCCCA
AAACCCAAGGACACCCTCATGATCTCCCGGACCCCTGAGGTCACATGCGTGGT
GGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAG
CACGTACCGTGTGGTCAGCGTCCTCACCGTCCTGCACCAGGACTGGCTGAATG
GCAAGGAGTACAAGTGCAAGGTCTCCAACAAAGCCCTCCCAGCCCCCATCGAG
AAAACCATCTCCAAAGCCAAAGGGCAGCCCCGAGAACCACAGGTGTACACCC
TGCCCCCATCCCGGGAGGAGATGACCAAGAACCAGGTCAGCCTGACCTGCCTG
GTCAAAGGCTTCTATCCCAGCGACATCGCCGTGGAGTGGGAGAGCAATGGGCA
GCCGGAGAACAACTACAAGACCACGCCTCCCGTGCTGGACTCCGACGGCTCCT
TCTTCCTCTATAGCAAGCTCACCGTGGACAAGAGCAGGTGGCAGCAGGGGAAC
GTCTTCTCATGCTCCGTGATGCATGGCGCTCTGCACAACCACTACACGCAGAA
GAGCCTCTCCCTGTTCCCCGGGTAA
> SBJ09-G14_light_kappa_chain leader sequence
SEQ ID NO: 357
ATGGGATGGTCATGTATCATCCTTTTTCTAGTAGCAACTGCAACCGGTGTACAT
TCC
> SBJ09-G14_light_kappa_chain variable domain
SEQ ID NO: 358
GATATTGTGATGACTCAGTCTCCACTCTCTCTGTCCGTCACCCCTGGACAGCCG
GCCTCCATCTCCTGCAAGTCTAGTCAGAGCCTCCTGCAAAGTGATGGAAAGAC
CTATTTGCATTGGTACCTGCAGAAGCCAGGCCAGTCTCCACAGCCCCTGATCTA
TGAAGTTTCCAACCGGTTCTCTGGAGTGCCAGATAGGTTCAGTGGCAGCGGGT
CAGGGACAGATTTCACACTGAAAATCAGCCGGGTGGAGGCTGAGGATGTTGG
GGTTTATTACTGCGTGCACCATTTGCCCCTTCCTCTCACATTCGGCGGAGGGAC
CAAGGTTGAGATCAAAC
> SBJ09-G14_light_kappa_chain constant domain
SEQ ID NO: 359
GAACTGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGT
GTTAG
> SBJ09-G14_light_kappa_chain complete
SEQ ID NO: 360
ATGGGATGGTCATGTATCATCCTTTTTCTAGTAGCAACTGCAACCGGTGTACAT
TCCGATATTGTGATGACTCAGTCTCCACTCTCTCTGTCCGTCACCCCTGGACAG
CCGGCCTCCATCTCCTGCAAGTCTAGTCAGAGCCTCCTGCAAAGTGATGGAAA
GACCTATTTGCATTGGTACCTGCAGAAGCCAGGCCAGTCTCCACAGCCCCTGA
TCTATGAAGTTTCCAACCGGTTCTCTGGAGTGCCAGATAGGTTCAGTGGCAGC
GGGTCAGGGACAGATTTCACACTGAAAATCAGCCGGGTGGAGGCTGAGGATG
TTGGGGTTTATTACTGCGTGCACCATTTGCCCCTTCCTCTCACATTCGGCGGAG
GGACCAAGGTTGAGATCAAACGAACTGTGGCTGCACCATCTGTCTTCATCTTCC
CGCCATCTGATGAGCAGTTGAAATCTGGAACTGCCTCTGTTGTGTGCCTGCTGA
ATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCA
CCTACAGCCTCAGCAGCACCCTGACGCTGAGCAAAGCAGACTACGAGAAACA
CAAAGTCTACGCCTGCGAAGTCACCCATCAGGGCCTGAGCTCGCCCGTCACAA
AGAGCTTCAACAGGGGAGAGTGTTAG

### Sequence of the Antibody Herein Identified as SBJ09-I10

### Amino Acid Sequences of SBJ09-I10:

> CDR 1 of variable domain of Heavy chain of SBJ09-I10
SEQ ID NO: 361
GITFNTHS
> CDR 2 of variable domain of Heavy chain of SBJ09-I10
SEQ ID NO: 362
IDGSSSFI
> CDR 3 of variable domain of Heavy chain of SBJ09-I10
SEQ ID NO: 363
ATGCPSVVNQGLEN
> CDR 1 of variable domain of Light chain of SBJ09-I10
SEQ ID NO: 364
QGIGND
> CDR 2 of variable domain of Light chain of SBJ09-I10 this sequence is
not included in the sequence listing because is less than 4 amino acid
SEQ ID NO: 365
GAS (Gly-Ala-Ser)
> CDR 3 of variable domain of Light chain of SBJ09-I10
SEQ ID NO: 366
LQHHSYPIT
> variable domain of Heavy chain of SBJ09-I10
SEQ ID NO: 367
EVQLVESGGGFVQPGGSLRLSCAASGITFNTHSMAWVRQAPGKGLEWVSSIDGSS
SFIYYTDSVRGRFTISRDNSKNTLYLQLTSLRAEDTAVYYCATGCPSVVNQGLENW
GQGSLVTVSS
> variable domain of Light chain of SBJ09-I10
SEQ ID NO: 368
AIQMTQSPSSLSASAGDRVTITCRASQGIGNDLGWYQQKPGKAPKRLIYGASSLQS
GVPSRFSGSGSETEFTLTISSLQPEDVATYYCLQHHSYPITFGQGTRLEIK
> Heavy chain of SBJ09-I10
SEQ ID NO: 369
MGWSCIILFLVATATGVHSEVQLVESGGGFVQPGGSLRLSCAASGITENTHSMAW
VRQAPGKGLEWVSSIDGSSSFIYYTDSVRGRFTISRDNSKNTLYLQLTSLRAEDTAV
YYCATGCPSVVNQGLENWGQGSLVTVSSASPTSPKSVFPLAPSSKSTSGGTAALGC
LVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQG
NVFSCSVMHGALHNHYTQKSLSLFPG*
> Light chain of SBJ09-I10
SEQ ID NO: 370
MGWSCIILFLVATATGVHSAIQMTQSPSSLSASAGDRVTITCRASQGIGNDLGWYQ
QKPGKAPKRLIYGASSLQSGVPSRFSGSGSETEFTLTISSLQPEDVATYYCLQHHSY
PITFGQGTRLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVT
KSFNRGEC*

### Nucleic Acid Sequences of SBJ09-I10:

> SBJ09-110_heavy_chain leader sequence
SEQ ID NO: 371
ATGGGATGGTCATGTATCATCCTTTTTCTAGTAGCAACTGCAACCGGTGTACATTCC
> SBJ09-110_heavy_chain variable domain
SEQ ID NO: 372
GAGGTGCAGCTGGTGGAGTCTGGGGGAGGCTTTGTACAGCCAGGGGGGAGCC
TGCGACTCTCCTGTGCAGCCTCAGGAATCACTTTTAACACCCATTCCATGGCCT
GGGTCCGCCAGGCTCCAGGGAAGGGGCTGGAGTGGGTCTCGTCTATTGATGGC
AGTAGTAGTTTCATTTACTACACAGACTCCGTGAGGGGCCGGTTCACCATCTCC
AGAGACAATTCCAAGAACACCCTGTATCTGCAACTGACCAGCCTGAGAGCCGA
GGACACGGCCGTATATTACTGTGCGACAGGTTGTCCTTCGGTGGTAAATCAGG
GGTTGGAGAACTGGGGCCAGGGATCCCTGGTCACCGTCTCCTCAG
> SBJ09-110_heavy_chain constant domain
SEQ ID NO: 373
CATCCCCGACCAGCCCCAAGTCGGTCTTCCCCCTGGCACCCTCCTCCAAGAGC
ACCTCTGGGGGCACAGCGGCCCTGGGCTGCCTGGTCAAGGACTACTTCCCCGA
ACCTGTGACGGTCTCGTGGAACTCAGGCGCCCTGACCAGCGGCGTGCACACCT
TCCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAAG
CCCAGCAACACCAAGGTGGACAAGAGAGTTGAGCCCAAATCTTGTGACAAAA
CTCACACATGCCCACCGTGCCCAGCACCTGAACTCCTGGGGGGACCGTCAGTC
TTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAA
CTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCA
GGACTGGCTGAATGGCAAGGAGTACAAGTGCAAGGTCTCCAACAAAGCCCTCC
CAGCCCCCATCGAGAAAACCATCTCCAAAGCCAAAGGGCAGCCCCGAGAACC
ACAGGTGTACACCCTGCCCCCATCCCGGGAGGAGATGACCAAGAACCAGGTCA
GCCTGACCTGCCTGGTCAAAGGCTTCTATCCCAGCGACATCGCCGTGGAGTGG
GAGAGCAATGGGCAGCCGGAGAACAACTACAAGACCACGCCTCCCGTGCTGG
ACTCCGACGGCTCCTTCTTCCTCTATAGCAAGCTCACCGTGGACAAGAGCAGG
TGGCAGCAGGGGAACGTCTTCTCATGCTCCGTGATGCATGGCGCTCTGCACAA
CCACTACACGCAGAAGAGCCTCTCCCTGTTCCCCGGGTAA
> SBJ09-110_heavy_chain complete
SEQ ID NO: 374
ATGGGATGGTCATGTATCATCCTTTTTCTAGTAGCAACTGCAACCGGTGTACAT
TCCGAGGTGCAGCTGGTGGAGTCTGGGGGAGGCTTTGTACAGCCAGGGGGGA
GCCTGCGACTCTCCTGTGCAGCCTCAGGAATCACTTTTAACACCCATTCCATGG
CCTGGGTCCGCCAGGCTCCAGGGAAGGGGCTGGAGTGGGTCTCGTCTATTGAT
GGCAGTAGTAGTTTCATTTACTACACAGACTCCGTGAGGGGCCGGTTCACCAT
CTCCAGAGACAATTCCAAGAACACCCTGTATCTGCAACTGACCAGCCTGAGAG
CCGAGGACACGGCCGTATATTACTGTGCGACAGGTTGTCCTTCGGTGGTAAAT
CAGGGGTTGGAGAACTGGGGCCAGGGATCCCTGGTCACCGTCTCCTCAGCATC
CCCGACCAGCCCCAAGTCGGTCTTCCCCCTGGCACCCTCCTCCAAGAGCACCTC
TGGGGGCACAGCGGCCCTGGGCTGCCTGGTCAAGGACTACTTCCCCGAACCTG
TGACGGTCTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCC
TCCAGCAGCTTGGGCACCCAGACCTACATCTGCAACGTGAATCACAAGCCCAG
CAACACCAAGGTGGACAAGAGAGTTGAGCCCAAATCTTGTGACAAAACTCAC
ACATGCCCACCGTGCCCAGCACCTGAACTCCTGGGGGGACCGTCAGTCTTCCT
CTTCCCCCCAAAACCCAAGGACACCCTCATGATCTCCCGGACCCCTGAGGTCA
CATGCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGAGC
AGTACAACAGCACGTACCGTGTGGTCAGCGTCCTCACCGTCCTGCACCAGGAC
TGGCTGAATGGCAAGGAGTACAAGTGCAAGGTCTCCAACAAAGCCCTCCCAGC
CCCCATCGAGAAAACCATCTCCAAAGCCAAAGGGCAGCCCCGAGAACCACAG
GTGTACACCCTGCCCCCATCCCGGGAGGAGATGACCAAGAACCAGGTCAGCCT
GACCTGCCTGGTCAAAGGCTTCTATCCCAGCGACATCGCCGTGGAGTGGGAGA
GCAATGGGCAGCCGGAGAACAACTACAAGACCACGCCTCCCGTGCTGGACTCC
GACGGCTCCTTCTTCCTCTATAGCAAGCTCACCGTGGACAAGAGCAGGTGGCA
GCAGGGGAACGTCTTCTCATGCTCCGTGATGCATGGCGCTCTGCACAACCACT
ACACGCAGAAGAGCCTCTCCCTGTTCCCCGGGTAA
> SBJ09-110_light_kappa_chain leader sequence
SEQ ID NO: 375
ATGGGATGGTCATGTATCATCCTTTTTCTAGTAGCAACTGCAACCGGTGTACAT
TCC
> SBJ09-110_light_kappa_chain variable domain
SEQ ID NO: 376
GCCATCCAGATGACCCAGTCTCCATCCTCCCTGTCTGCATCTGCAGGAGACAG
AGTCACCATCACTTGCCGGGCAAGTCAGGGCATTGGAAATGATTTAGGCTGGT
ATCAGCAGAAACCAGGGAAAGCCCCTAAGCGCCTGATCTATGGTGCATCCAGT
TTGCAAAGTGGGGTCCCATCAAGGTTCAGCGGCAGTGGATCTGAGACTGAATT
CACTCTCACAATCAGCAGCCTGCAGCCTGAAGATGTTGCAACTTATTACTGTCT
ACAGCATCATAGTTACCCGATCACCTTCGGCCAAGGGACACGACTGGAGATTA
AAC
> SBJ09-110_light_kappa_chain constant domain
SEQ ID NO: 377
GAACTGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGT
GTTAG
> SBJ09-110_light_kappa_chain complete
SEQ ID NO: 378
ATGGGATGGTCATGTATCATCCTTTTTCTAGTAGCAACTGCAACCGGTGTACAT
TCCGCCATCCAGATGACCCAGTCTCCATCCTCCCTGTCTGCATCTGCAGGAGAC
AGAGTCACCATCACTTGCCGGGCAAGTCAGGGCATTGGAAATGATTTAGGCTG
GTATCAGCAGAAACCAGGGAAAGCCCCTAAGCGCCTGATCTATGGTGCATCCA
GTTTGCAAAGTGGGGTCCCATCAAGGTTCAGCGGCAGTGGATCTGAGACTGAA
TTCACTCTCACAATCAGCAGCCTGCAGCCTGAAGATGTTGCAACTTATTACTGT
CTACAGCATCATAGTTACCCGATCACCTTCGGCCAAGGGACACGACTGGAGAT
TAAACGAACTGTGGCTGCACCATCTGTCTTCATCTTCCCGCCATCTGATGAGCA
GTTGAAATCTGGAACTGCCTCTGTTGTGTGCCTGCTGAATAACTTCTATCCCAG
AGAGGCCAAAGTACAGTGGAAGGTGGATAACGCCCTCCAATCGGGTAACTCCC
AGGAGAGTGTCACAGAGCAGGACAGCAAGGACAGCACCTACAGCCTCAGCAG
CACCCTGACGCTGAGCAAAGCAGACTACGAGAAACACAAAGTCTACGCCTGC
GAAGTCACCCATCAGGGCCTGAGCTCGCCCGTCACAAAGAGCTTCAACAGGGG
AGAGTGTTAG

### Sequence of the Antibody Herein Identified as SBJ09-M06

### Amino Acid Sequences of SBJ09-M06:

> CDR 1 of variable domain of Heavy chain of SBJ09-M06
SEQ ID NO: 379
GFSVSRSW
> CDR 2 of variable domain of Heavy chain of SBJ09-M06
SEQ ID NO: 380
IQSDDNKI
> CDR 3 of variable domain of Heavy chain of SBJ09-M06
SEQ ID NO: 381
ARDRGWGLFDI
> CDR 1 of variable domain of Light chain of SBJ09-M06
SEQ ID NO: 382
QSLLQSDGKTY
> CDR 2 of variable domain of Light chain of SBJ09-M06 this sequence is
not included in the sequence listing because is less than 4 amino acid
SEQ ID NO: 383
EVS (Glu-Val-Ser)
> CDR 3 of variable domain of Light chain of SBJ09-M06
SEQ ID NO: 384
VHHLPLPLT
> variable domain of Heavy chain of SBJ09-M06
SEQ ID NO: 385
EVQLVESGGGLVQPGGSLRLSCAASGFSVSRSWMNWVRQAPGKGLVWVSSIQSD
DNKIGYADSVRGRFAVSGDNAKNTVFLQMNSLRAEDTAVYYCARDRGWGLFDI
WGQGTLVTVSS
> variable domain of Light chain of SBJ09-M06
SEQ ID NO: 386
DIVMTQTPLSLSVTPGQPASISCKSSQSLLQSDGKTYLHWYLQKPGQSPQPLIYEVS
NRFSGVPDRFSGNGSGTDFTLKISR VEAEDVGVYYCVHHLPLPLTFGGGTKVEIK
>Heavy chain of SBJ09-M06
SEQ ID NO: 387
MGWSCIILFLVATATGVHSEVQLVESGGGLVQPGGSLRLSCAASGFSVSRSWMNW
VRQAPGKGLVWVSSIQSDDNKIGYADSVRGRFAVSGDNAKNTVFLQMNSLRAED
TAVYYCARDRGWGLFDIWGQGTLVTVSSASPTSPKSVFPLAPSSKSTSGGTAALG
CLVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSL
TCLVKGFYPSDIA VEWESNGQPENNYKTTPPVLDSDGSFFLYSKLTVDKSRWQQG
NVFSCSVMHGALHNHYTQKSLSLFPG*
> Light chain of SBJ09-M06
SEQ ID NO: 388
MGWSCIILFLVATATGVHSDIVMTQTPLSLSVTPGQPASISCKSSQSLLQSDGKTYL
HWYLQKPGQSPQPLIYEVSNRFSGVPDRFSGNGSGTDFTLKISRVEAEDVGVYYCV
HHLPLPLTFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQG
LSSPVTKSFNRGEC*

### Nucleic Acid Sequences of SBJ09-M06:

> SBJ09-M06_heavy_chain leader sequence
SEQ ID NO: 389
ATGGGATGGTCATGTATCATCCTTTTTCTAGTAGCAACTGCAACCGGTGTACAT
TCC
> SBJ09-M06_heavy_chain variable domain
SEQ ID NO: 390
GAGGTGCAGCTGGTGGAGTCTGGGGGAGGCTTAGTTCAGCCGGGGGGGTCCCT
GAGACTCTCCTGTGCAGCCTCTGGATTCAGCGTCAGTAGGTCCTGGATGAATTG
GGTCCGCCAAGCTCCAGGGAAGGGGCTGGTGTGGGTCTCATCTATTCAAAGTG
ATGACAATAAGATAGGCTACGCGGACTCCGTGAGGGGCCGATTCGCCGTCTCC
GGAGACAACGCCAAGAATACGGTGTTTCTGCAAATGAACAGTCTGAGAGCCG
AGGATACGGCTGTCTATTACTGCGCAAGAGATCGGGGCTGGGGACTTTTTGAT
ATCTGGGGCCAAGGGACATTGGTCACCGTCTCTTCAG
> SBJ09-M06_heavy_chain constant domain
SEQ ID NO: 391
CATCCCCGACCAGCCCCAAGTCGGTCTTCCCCCTGGCACCCTCCTCCAAGAGC
ACCTCTGGGGGCACAGCGGCCCTGGGCTGCCTGGTCAAGGACTACTTCCCCGA
ACCTGTGACGGTCTCGTGGAACTCAGGCGCCCTGACCAGCGGCGTGCACACCT
TCCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAAG
CCCAGCAACACCAAGGTGGACAAGAGAGTTGAGCCCAAATCTTGTGACAAAA
CTCACACATGCCCACCGTGCCCAGCACCTGAACTCCTGGGGGGACCGTCAGTC
TTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAA
CTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCA
GGACTGGCTGAATGGCAAGGAGTACAAGTGCAAGGTCTCCAACAAAGCCCTCC
CAGCCCCCATCGAGAAAACCATCTCCAAAGCCAAAGGGCAGCCCCGAGAACC
ACAGGTGTACACCCTGCCCCCATCCCGGGAGGAGATGACCAAGAACCAGGTCA
GCCTGACCTGCCTGGTCAAAGGCTTCTATCCCAGCGACATCGCCGTGGAGTGG
GAGAGCAATGGGCAGCCGGAGAACAACTACAAGACCACGCCTCCCGTGCTGG
ACTCCGACGGCTCCTTCTTCCTCTATAGCAAGCTCACCGTGGACAAGAGCAGG
TGGCAGCAGGGGAACGTCTTCTCATGCTCCGTGATGCATGGCGCTCTGCACAA
CCACTACACGCAGAAGAGCCTCTCCCTGTTCCCCGGGTAA
> SBJ09-M06_heavy_chain complete
SEQ ID NO: 392
ATGGGATGGTCATGTATCATCCTTTTTCTAGTAGCAACTGCAACCGGTGTACAT
TCCGAGGTGCAGCTGGTGGAGTCTGGGGGAGGCTTAGTTCAGCCGGGGGGGTC
CCTGAGACTCTCCTGTGCAGCCTCTGGATTCAGCGTCAGTAGGTCCTGGATGAA
TTGGGTCCGCCAAGCTCCAGGGAAGGGGCTGGTGTGGGTCTCATCTATTCAAA
GTGATGACAATAAGATAGGCTACGCGGACTCCGTGAGGGGCCGATTCGCCGTC
TCCGGAGACAACGCCAAGAATACGGTGTTTCTGCAAATGAACAGTCTGAGAGC
CGAGGATACGGCTGTCTATTACTGCGCAAGAGATCGGGGCTGGGGACTTTTTG
ATATCTGGGGCCAAGGGACATTGGTCACCGTCTCTTCAGCATCCCCGACCAGC
CCCAAGTCGGTCTTCCCCCTGGCACCCTCCTCCAAGAGCACCTCTGGGGGCAC
AGCGGCCCTGGGCTGCCTGGTCAAGGACTACTTCCCCGAACCTGTGACGGTCT
CGTGGAACTCAGGCGCCCTGACCAGCGGCGTGCACACCTTCCCGGCTGTCCTA
CAGTCCTCAGGACTCTACTCCCTCAGCAGCGTGGTGACCGTGCCCTCCAGCAG
CTTGGGCACCCAGACCTACATCTGCAACGTGAATCACAAGCCCAGCAACACCA
AGGTGGACAAGAGAGTTGAGCCCAAATCTTGTGACAAAACTCACACATGCCCA
CCGTGCCCAGCACCTGAACTCCTGGGGGGACCGTCAGTCTTCCTCTTCCCCCCA
AAACCCAAGGACACCCTCATGATCTCCCGGACCCCTGAGGTCACATGCGTGGT
GGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAG
CACGTACCGTGTGGTCAGCGTCCTCACCGTCCTGCACCAGGACTGGCTGAATG
GCAAGGAGTACAAGTGCAAGGTCTCCAACAAAGCCCTCCCAGCCCCCATCGAG
AAAACCATCTCCAAAGCCAAAGGGCAGCCCCGAGAACCACAGGTGTACACCC
TGCCCCCATCCCGGGAGGAGATGACCAAGAACCAGGTCAGCCTGACCTGCCTG
GTCAAAGGCTTCTATCCCAGCGACATCGCCGTGGAGTGGGAGAGCAATGGGCA
GCCGGAGAACAACTACAAGACCACGCCTCCCGTGCTGGACTCCGACGGCTCCT
TCTTCCTCTATAGCAAGCTCACCGTGGACAAGAGCAGGTGGCAGCAGGGGAAC
GTCTTCTCATGCTCCGTGATGCATGGCGCTCTGCACAACCACTACACGCAGAA
GAGCCTCTCCCTGTTCCCCGGGTAA
> SBJ09-M06_light_kappa_chain leader sequence
SEQ ID NO: 393
ATGGGATGGTCATGTATCATCCTTTTTCTAGTAGCAACTGCAACCGGTGTACAT
TCC
> SBJ09-M06_light_kappa_chain variable domain
SEQ ID NO: 394
GATATTGTGATGACCCAGACTCCACTCTCTCTGTCCGTCACCCCTGGACAGCCG
GCCTCCATCTCCTGCAAGTCTAGTCAGAGCCTCCTGCAAAGTGATGGAAAGAC
CTATTTGCATTGGTACCTGCAGAAGCCAGGCCAGTCTCCACAGCCCCTGATCTA
TGAAGTTTCCAACCGGTTCTCTGGAGTGCCAGATAGGTTCAGTGGCAACGGGT
CAGGGACAGATTTCACACTGAAAATCAGCCGGGTGGAGGCTGAGGATGTTGG
GGTTTATTACTGCGTGCACCATTTGCCCCTTCCTCTCACATTCGGCGGAGGGAC
CAAGGTTGAGATCAAAC
> SBJ09-M06_light_kappa_chain constant domain
SEQ ID NO: 395
GAACTGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGT
GTTAG
> SBJ09-M06_light_kappa_chain complete
SEQ ID NO: 396
ATGGGATGGTCATGTATCATCCTTTTTCTAGTAGCAACTGCAACCGGTGTACAT
TCCGATATTGTGATGACCCAGACTCCACTCTCTCTGTCCGTCACCCCTGGACAG
CCGGCCTCCATCTCCTGCAAGTCTAGTCAGAGCCTCCTGCAAAGTGATGGAAA
GACCTATTTGCATTGGTACCTGCAGAAGCCAGGCCAGTCTCCACAGCCCCTGA
TCTATGAAGTTTCCAACCGGTTCTCTGGAGTGCCAGATAGGTTCAGTGGCAAC
GGGTCAGGGACAGATTTCACACTGAAAATCAGCCGGGTGGAGGCTGAGGATG
TTGGGGTTTATTACTGCGTGCACCATTTGCCCCTTCCTCTCACATTCGGCGGAG
GGACCAAGGTTGAGATCAAACGAACTGTGGCTGCACCATCTGTCTTCATCTTCC
CGCCATCTGATGAGCAGTTGAAATCTGGAACTGCCTCTGTTGTGTGCCTGCTGA
ATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCA
CCTACAGCCTCAGCAGCACCCTGACGCTGAGCAAAGCAGACTACGAGAAACA
CAAAGTCTACGCCTGCGAAGTCACCCATCAGGGCCTGAGCTCGCCCGTCACAA
AGAGCTTCAACAGGGGAGAGTGTTAG

### Sequence of the Antibody Herein Identified as SBJ10-H18

### Amino Acid Sequences of SBJ10-1118:

> CDR 1 of variable domain of Heavy chain of SBJ10-H18
SEQ ID NO: 397
GFTFSSYT
> CDR 2 of variable domain of Heavy chain of SBJ10-H18
SEQ ID NO: 398
ISSSRSSI
> CDR 3 of variable domain of Heavy chain of SBJ10-H18
SEQ ID NO: 399
ARGGRSDSFFDH
> CDR 1 of variable domain of Light chain of SBJ10-H18
SEQ ID NO: 400
QSL VFSDGDTY
> CDR 2 of variable domain of Light chain of SBJ10-H18 this sequence is
not included in the sequence listing because is less than 4 amino acid
SEQ ID NO: 401
KVS (Lys-Val-Ser)
> CDR 3 of variable domain of Light chain of SBJ10-H18
SEQ ID NO: 402
MQGALWPLT
> variable domain of Heavy chain of SBJ10-H18
SEQ ID NO: 403
EVQLVESGGGLVKSGGSLRLSCAASGFTFSSYTMNWVRQAPGKGLEWVSSISSSR
SSIFYAGSVKGRFTISRDNAKNSLYLQMNSLRVEDTAVYFCARGGRSDSFFDHWG
QGTLVTVSS
> variable domain of Light chain of SBJ10-H18
SEQ ID NO: 404
DIVMTQTPLSLPVTLGQPASISCRSSQSLVFSDGDTYLNWFQQRPGQSPRRLIYKVS
NRDSGVPDRFSGSGSGTDFTLKISRVEAEDVGVYYCMQGALWPLTFGQGTKVEIK
> Heavy chain of SBJ10-H18
SEQ ID NO: 405
MGWSCIILFLVATATGVHSEVQLVESGGGLVKSGGSLRLSCAASGFTFSSYTMNW
VRQAPGKGLEWVSSISSSRSSIFYAGSVKGRFTISRDNAKNSLYLQMNSLRVEDTA
VYFCARGGRSDSFFDHWGQGTLVTVSSASPTSPKSVFPLAPSSKSTSGGTAALGCL
VKDYFPEPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGN
VFSCSVMHGALHNHYTQKSLSLFPG*
> Light chain of SBJ10-H18
SEQ ID NO: 406
MGWSCIILFLVATATGVHSDIVMTQTPLSLPVTLGQPASISCRSSQSLVFSDGDTYL
NWFQQRPGQSPRRLIYKVSNRDSGVPDRFSGSGSGTDFTLKISRVEAEDVGVYYC
MQGALWPLTFGQGTKVEIKRTVAAPSVFIFPPSDEQLKSGTASVVCLLNNFYPREA
KVQWKVDNALQSGNSQESVTEQDSKDSTYSLSSTLTLSKADYEKHKVYACEVTH
QGLSSPVTKSFNRGEC*

### Nucleic Acid Sequences of SBJ10-H18:

> SBJ10-H18_heavy_chain leader sequence
SEQ ID NO: 407
ATGGGATGGTCATGTATCATCCTTTTTCTAGTAGCAACTGCAACCGGTGTACAT
TCC
> SBJ10-H18_heavy_chain variable domain
SEQ ID NO: 408
GAGGTGCAGCTGGTGGAGTCTGGGGGAGGCCTGGTCAAGTCTGGGGGGTCCCT
GAGACTCTCCTGTGCAGCCTCTGGATTCACCTTCAGTAGTTATACCATGAATTG
GGTCCGCCAGGCTCCAGGGAAGGGGCTGGAGTGGGTCTCATCCATCAGTAGTA
GCCGTAGTTCCATATTCTACGCAGGCTCAGTGAAGGGCCGATTCACCATCTCCA
GAGACAATGCCAAGAACTCACTGTATCTGCAAATGAACAGCCTGAGAGTCGAG
GACACGGCTGTATATTTCTGTGCGAGAGGAGGGCGGTCAGATTCCTTCTTTGAC
CACTGGGGCCAGGGAACCCTGGTCACCGTCTCCTCAG
> SBJ10-H18_heavy_chain constant domain
SEQ ID NO: 409
CATCCCCGACCAGCCCCAAGTCGGTCTTCCCCCTGGCACCCTCCTCCAAGAGC
ACCTCTGGGGGCACAGCGGCCCTGGGCTGCCTGGTCAAGGACTACTTCCCCGA
ACCTGTGACGGTCTCGTGGAACTCAGGCGCCCTGACCAGCGGCGTGCACACCT
TCCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAAG
CCCAGCAACACCAAGGTGGACAAGAGAGTTGAGCCCAAATCTTGTGACAAAA
CTCACACATGCCCACCGTGCCCAGCACCTGAACTCCTGGGGGGACCGTCAGTC
TTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAA
CTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCA
GGACTGGCTGAATGGCAAGGAGTACAAGTGCAAGGTCTCCAACAAAGCCCTCC
CAGCCCCCATCGAGAAAACCATCTCCAAAGCCAAAGGGCAGCCCCGAGAACC
ACAGGTGTACACCCTGCCCCCATCCCGGGAGGAGATGACCAAGAACCAGGTCA
GCCTGACCTGCCTGGTCAAAGGCTTCTATCCCAGCGACATCGCCGTGGAGTGG
GAGAGCAATGGGCAGCCGGAGAACAACTACAAGACCACGCCTCCCGTGCTGG
ACTCCGACGGCTCCTTCTTCCTCTATAGCAAGCTCACCGTGGACAAGAGCAGG
TGGCAGCAGGGGAACGTCTTCTCATGCTCCGTGATGCATGGCGCTCTGCACAA
CCACTACACGCAGAAGAGCCTCTCCCTGTTCCCCGGGTAA
> SBJ10-H18_heavy_chain complete
SEQ ID NO: 410
ATGGGATGGTCATGTATCATCCTTTTTCTAGTAGCAACTGCAACCGGTGTACAT
TCCGAGGTGCAGCTGGTGGAGTCTGGGGGAGGCCTGGTCAAGTCTGGGGGGTC
CCTGAGACTCTCCTGTGCAGCCTCTGGATTCACCTTCAGTAGTTATACCATGAA
TTGGGTCCGCCAGGCTCCAGGGAAGGGGCTGGAGTGGGTCTCATCCATCAGTA
GTAGCCGTAGTTCCATATTCTACGCAGGCTCAGTGAAGGGCCGATTCACCATCT
CCAGAGACAATGCCAAGAACTCACTGTATCTGCAAATGAACAGCCTGAGAGTC
GAGGACACGGCTGTATATTTCTGTGCGAGAGGAGGGCGGTCAGATTCCTTCTT
TGACCACTGGGGCCAGGGAACCCTGGTCACCGTCTCCTCAGCATCCCCGACCA
GCCCCAAGTCGGTCTTCCCCCTGGCACCCTCCTCCAAGAGCACCTCTGGGGGC
ACAGCGGCCCTGGGCTGCCTGGTCAAGGACTACTTCCCCGAACCTGTGACGGT
CTCGTGGAACTCAGGCGCCCTGACCAGCGGCGTGCACACCTTCCCGGCTGTCC
TACAGTCCTCAGGACTCTACTCCCTCAGCAGCGTGGTGACCGTGCCCTCCAGCA
GCTTGGGCACCCAGACCTACATCTGCAACGTGAATCACAAGCCCAGCAACACC
AAGGTGGACAAGAGAGTTGAGCCCAAATCTTGTGACAAAACTCACACATGCCC
ACCGTGCCCAGCACCTGAACTCCTGGGGGGACCGTCAGTCTTCCTCTTCCCCCC
AAAACCCAAGGACACCCTCATGATCTCCCGGACCCCTGAGGTCACATGCGTGG
TGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAACA
GCACGTACCGTGTGGTCAGCGTCCTCACCGTCCTGCACCAGGACTGGCTGAAT
GGCAAGGAGTACAAGTGCAAGGTCTCCAACAAAGCCCTCCCAGCCCCCATCGA
GAAAACCATCTCCAAAGCCAAAGGGCAGCCCCGAGAACCACAGGTGTACACC
CTGCCCCCATCCCGGGAGGAGATGACCAAGAACCAGGTCAGCCTGACCTGCCT
GGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCC
TTCTTCCTCTATAGCAAGCTCACCGTGGACAAGAGCAGGTGGCAGCAGGGGAA
CGTCTTCTCATGCTCCGTGATGCATGGCGCTCTGCACAACCACTACACGCAGAA
GAGCCTCTCCCTGTTCCCCGGGTAA
> SBJ10-H18_light_kappa_chain leader sequence
SEQ ID NO: 411
ATGGGATGGTCATGTATCATCCTTTTTCTAGTAGCAACTGCAACTGGTGTACAT
TCC
> SBJ10-H18_light_kappa_chain variable domain
SEQ ID NO: 412
GATATTGTGATGACCCAGACTCCACTCTCCCTGCCCGTCACCCTTGGACAGCCG
GCCTCCATCTCCTGCAGGTCTAGTCAAAGCCTCGTATTCAGTGATGGAGACACC
TACTTGAATTGGTTTCAGCAGAGGCCAGGCCAATCTCCAAGGCGCCTAATTTAT
AAGGTTTCTAACCGGGACTCTGGGGTCCCAGACAGATTCAGCGGCAGTGGGTC
AGGCACTGATTTCACACTGAAAATCAGCAGGGTGGAGGCTGAGGATGTTGGGG
TTTATTACTGCATGCAAGGTGCACTCTGGCCTCTAACGTTCGGCCAAGGGACCA
AGGTAGAAATCAAAC
> SBJ10-H18_light_kappa_chain constant domain
SEQ ID NO: 413
GAACTGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGT
GTTAG
> SBJ10-H18_light_kappa_chain complete
SEQ ID NO: 414
ATGGGATGGTCATGTATCATCCTTTTTCTAGTAGCAACTGCAACTGGTGTACAT
TCCGATATTGTGATGACCCAGACTCCACTCTCCCTGCCCGTCACCCTTGGACAG
CCGGCCTCCATCTCCTGCAGGTCTAGTCAAAGCCTCGTATTCAGTGATGGAGAC
ACCTACTTGAATTGGTTTCAGCAGAGGCCAGGCCAATCTCCAAGGCGCCTAAT
TTATAAGGTTTCTAACCGGGACTCTGGGGTCCCAGACAGATTCAGCGGCAGTG
GGTCAGGCACTGATTTCACACTGAAAATCAGCAGGGTGGAGGCTGAGGATGTT
GGGGTTTATTACTGCATGCAAGGTGCACTCTGGCCTCTAACGTTCGGCCAAGG
GACCAAGGTAGAAATCAAACGAACTGTGGCTGCACCATCTGTCTTCATCTTCC
CGCCATCTGATGAGCAGTTGAAATCTGGAACTGCCTCTGTTGTGTGCCTGCTGA
ATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCA
CCTACAGCCTCAGCAGCACCCTGACGCTGAGCAAAGCAGACTACGAGAAACA
CAAAGTCTACGCCTGCGAAGTCACCCATCAGGGCCTGAGCTCGCCCGTCACAA
AGAGCTTCAACAGGGGAGAGTGTTAG

### Sequence of the Antibody Herein Identified as SBJ11-C06

### Amino Acid Sequences of SBJ11-C06:

> CDR 1 of variable domain of Heavy chain of SBJ11-C06
SEQ ID NO: 415
GFTFSSYS
> CDR 2 of variable domain of Heavy chain of SBJ11-C06
SEQ ID NO: 416
ISETSYHI
> CDR 3 of variable domain of Heavy chain of SBJ11-C06
SEQ ID NO: 417
AAHVPCGGTCNGIDY
> CDR 1 of variable domain of Light chain of SBJ11-C06
SEQ ID NO: 418
QGIGND
> CDR 2 of variable domain of Light chain of SBJ11-C06 this sequence is
not included in the sequence listing because is less than 4 amino acid
SEQ ID NO: 419
GAS (Gly-Ala-Ser)
> CDR 3 of variable domain of Light chain of SBJ11-C06
SEQ ID NO: 420
LQHNAYPRT
> variable domain of Heavy chain of SBJ11-C06
SEQ ID NO: 421
EVQLVESGGGLVKPGGSLRLSCAASGFTFSSYSMNWVRQAPGKGLEWVSSISETS
YHIYYADSVKGRFTISRDNARNSLYLQMNSLRAEDTGIYFCAAHVPCGGTCNGID
YWGQGTLVTVSS
> variable domain of Light chain of SBJ11-C06
SEQ ID NO: 422
AIQMTQSPSSLSASVGDRVTITCRASQGIGNDLGWYQHKPGMAPKRLIYGASTLQS
GVPSRISGSGSGTEFTLTISSLQPEDFATYYCLQHNAYPRTFGQGTRLEI
> Heavy chain of SBJ11-C06
SEQ ID NO: 423
MGWSCIILFLVATATGVHSEVQLVESGGGLVKPGGSLRLSCAASGFTFSSYSMNW
VRQAPGKGLEWVSSISETSYHIYYADSVKGRFTISRDNARNSLYLQMNSLRAEDTG
IYFCAAHVPCGGTCNGIDYWGQGTLVTVSSASPTSPKSVFPLAPSSKSTSGGTAAL
GCLVKDYFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPKDTLMISR
TPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQ
GNVFSCSVMHGALHNHYTQKSLSLFPG*
> Light chain of SBJ11-C06
SEQ ID NO: 424
MGWSCIILFLVATATGVHSAIQMTQSPSSLSASVGDRVTITCRASQGIGNDLGWYQ
HKPGMAPKRLIYGASTLQSGVPSRISGSGSGTEFTLTISSLQPEDFATYYCLQHNAY
PRTFGQGTRLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPV
TKSFNRGEC*

### Nucleic Acid Sequences of SBJ11-C06:

> SBJ11-C06_heavy_chain leader sequence
SEQ ID NO: 425
ATGGGATGGTCATGTATCATCCTTTTTCTAGTAGCAACTGCAACCGGTGTACAT
TCC
> SBJ11-C06_heavy_chain variable domain
SEQ ID NO: 426
GAGGTGCAGCTGGTGGAGTCTGGGGGAGGCCTGGTCAAGCCTGGGGGGTCCCT
GAGACTCTCCTGTGCAGCCTCTGGATTCACGTTCAGTAGCTACAGCATGAACTG
GGTCCGCCAGGCTCCAGGGAAGGGACTGGAGTGGGTCTCATCCATCAGTGAGA
CTAGTTATCACATATACTACGCAGACTCAGTGAAGGGCCGATTCACCATCTCC
AGAGACAACGCCAGGAACTCACTTTATCTACAAATGAACAGCCTGAGAGCCGA
GGACACGGGTATATATTTCTGTGCGGCACATGTACCGTGTGGCGGCACCTGTA
ACGGGATTGACTACTGGGGCCAGGGAACCCTGGTCACCGTCTCCTCAG
> SBJ11-C06_heavy_chain constant domain
SEQ ID NO: 427
CATCCCCGACCAGCCCCAAGTCGGTCTTCCCCCTGGCACCCTCCTCCAAGAGC
ACCTCTGGGGGCACAGCGGCCCTGGGCTGCCTGGTCAAGGACTACTTCCCCGA
ACCTGTGACGGTCTCGTGGAACTCAGGCGCCCTGACCAGCGGCGTGCACACCT
TCCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAAG
CCCAGCAACACCAAGGTGGACAAGAGAGTTGAGCCCAAATCTTGTGACAAAA
CTCACACATGCCCACCGTGCCCAGCACCTGAACTCCTGGGGGGACCGTCAGTC
TTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAA
CTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCA
GGACTGGCTGAATGGCAAGGAGTACAAGTGCAAGGTCTCCAACAAAGCCCTCC
CAGCCCCCATCGAGAAAACCATCTCCAAAGCCAAAGGGCAGCCCCGAGAACC
ACAGGTGTACACCCTGCCCCCATCCCGGGAGGAGATGACCAAGAACCAGGTCA
GCCTGACCTGCCTGGTCAAAGGCTTCTATCCCAGCGACATCGCCGTGGAGTGG
GAGAGCAATGGGCAGCCGGAGAACAACTACAAGACCACGCCTCCCGTGCTGG
ACTCCGACGGCTCCTTCTTCCTCTATAGCAAGCTCACCGTGGACAAGAGCAGG
TGGCAGCAGGGGAACGTCTTCTCATGCTCCGTGATGCATGGCGCTCTGCACAA
CCACTACACGCAGAAGAGCCTCTCCCTGTTCCCCGGGTAA
> SBJ11-C06_heavy_chain complete
SEQ ID NO: 428
ATGGGATGGTCATGTATCATCCTTTTTCTAGTAGCAACTGCAACCGGTGTACAT
TCCGAGGTGCAGCTGGTGGAGTCTGGGGGAGGCCTGGTCAAGCCTGGGGGGTC
CCTGAGACTCTCCTGTGCAGCCTCTGGATTCACGTTCAGTAGCTACAGCATGAA
CTGGGTCCGCCAGGCTCCAGGGAAGGGACTGGAGTGGGTCTCATCCATCAGTG
AGACTAGTTATCACATATACTACGCAGACTCAGTGAAGGGCCGATTCACCATC
TCCAGAGACAACGCCAGGAACTCACTTTATCTACAAATGAACAGCCTGAGAGC
CGAGGACACGGGTATATATTTCTGTGCGGCACATGTACCGTGTGGCGGCACCT
GTAACGGGATTGACTACTGGGGCCAGGGAACCCTGGTCACCGTCTCCTCAGCA
TCCCCGACCAGCCCCAAGTCGGTCTTCCCCCTGGCACCCTCCTCCAAGAGCACC
TCTGGGGGCACAGCGGCCCTGGGCTGCCTGGTCAAGGACTACTTCCCCGAACC
TGTGACGGTCTCGTGGAACTCAGGCGCCCTGACCAGCGGCGTGCACACCTTCC
CGGCTGTCCTACAGTCCTCAGGACTCTACTCCCTCAGCAGCGTGGTGACCGTGC
CCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTC
ACACATGCCCACCGTGCCCAGCACCTGAACTCCTGGGGGGACCGTCAGTCTTC
CTCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAG
CAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCACA
GGTGTACACCCTGCCCCCATCCCGGGAGGAGATGACCAAGAACCAGGTCAGCC
TGACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTC
CGACGGCTCCTTCTTCCTCTATAGCAAGCTCACCGTGGACAAGAGCAGGTGGC
AGCAGGGGAACGTCTTCTCATGCTCCGTGATGCATGGCGCTCTGCACAACCAC
TACACGCAGAAGAGCCTCTCCCTGTTCCCCGGGTAA
> SBJ11-C06_light_kappa_chain leader sequence
SEQ ID NO: 429
ATGGGATGGTCATGTATCATCCTTTTTCTAGTAGCAACTGCAACCGGTGTACAT
TCC
> SBJ11-C06_light_kappa_chain variable domain
SEQ ID NO: 430
GCCATCCAGATGACCCAGTCTCCATCCTCCCTGTCTGCATCTGTAGGAGACAGA
GTCACCATCACTTGCCGGGCAAGTCAGGGCATAGGAAATGATTTAGGCTGGTA
TCAGCACAAACCAGGGATGGCCCCTAAGCGCCTGATCTATGGTGCATCCACTT
TGCAAAGTGGGGTCCCATCAAGGATCAGCGGCAGTGGGTCTGGGACAGAATTC
ACTCTCACAATCAGCAGCCTGCAGCCTGAGGATTTTGCAACTTATTACTGTCTA
CAACATAATGCTTACCCTCGAACGTTCGGCCAAGGGACCAGGCTGGAAATCAA
> SBJ11-C06_light_kappa_chain constant domain
SEQ ID NO: 431
GCGAACTGTGGCTGCACCATCTGTCTTCATCTTCCCGCCATCTGATGAGCAGTT
GAAATCTGGAACTGCCTCTGTTGTGTGCCTGCTGAATAACTTCTATCCCAGAGA
GGCCAAAGTACAGTGGAAGGTGGATAACGCCCTCCAATCGGGTAACTCCCAGG
AGAGTGTCACAGAGCAGGACAGCAAGGACAGCACCTACAGCCTCAGCAGCAC
CCTGACGCTGAGCAAAGCAGACTACGAGAAACACAAAGTCTACGCCTGCGAA
GTCACCCATCAGGGCCTGAGCTCGCCCGTCACAAAGAGCTTCAACAGGGGAGA
GTGTTAG
> SBJ11-C06_light_kappa_chain complete
SEQ ID NO: 432
ATGGGATGGTCATGTATCATCCTTTTTCTAGTAGCAACTGCAACCGGTGTACAT
TCCGCCATCCAGATGACCCAGTCTCCATCCTCCCTGTCTGCATCTGTAGGAGAC
AGAGTCACCATCACTTGCCGGGCAAGTCAGGGCATAGGAAATGATTTAGGCTG
GTATCAGCACAAACCAGGGATGGCCCCTAAGCGCCTGATCTATGGTGCATCCA
CTTTGCAAAGTGGGGTCCCATCAAGGATCAGCGGCAGTGGGTCTGGGACAGAA
TTCACTCTCACAATCAGCAGCCTGCAGCCTGAGGATTTTGCAACTTATTACTGT
CTACAACATAATGCTTACCCTCGAACGTTCGGCCAAGGGACCAGGCTGGAAAT
CAAGCGAACTGTGGCTGCACCATCTGTCTTCATCTTCCCGCCATCTGATGAGCA
GTTGAAATCTGGAACTGCCTCTGTTGTGTGCCTGCTGAATAACTTCTATCCCAG
AGAGGCCAAAGTACAGTGGAAGGTGGATAACGCCCTCCAATCGGGTAACTCCC
AGGAGAGTGTCACAGAGCAGGACAGCAAGGACAGCACCTACAGCCTCAGCAG
CACCCTGACGCTGAGCAAAGCAGACTACGAGAAACACAAAGTCTACGCCTGC
GAAGTCACCCATCAGGGCCTGAGCTCGCCCGTCACAAAGAGCTTCAACAGGGG
AGAGTGTTAG

### Sequence of the Antibody Herein Identified as SBJ11-J13

### Amino Acid Sequences of SBJ11-J13:

> CDR 1 of variable domain of Heavy chain of SBJ11-J13
SEQ ID NO: 433
GFAFSNYF
> CDR 2 of variable domain of Heavy chain of SBJ11-J13
SEQ ID NO: 434
INQDGSDK
> CDR 3 of variable domain of Heavy chain of SBJ11-J13
SEQ ID NO: 435
ARASLSSAADY
> CDR 1 of variable domain of Light chain of SBJ11-J13
SEQ ID NO: 436
QSLVYSDGNTN
> CDR 2 of variable domain of Light chain of SBJ11-J13 this sequence is
not included in the sequence listing because is less than 4 amino acid
SEQ ID NO: 437
RVS (Arg-Val-Ser)
> CDR 3 of variable domain of Light chain of SBJ11-J13
SEQ ID NO: 438
MQSTHWPHT
> variable domain of Heavy chain of SBJ11-J13
SEQ ID NO: 439
EVQLVESGGGLVQPEGSLRLSCAASGFAFSNYFMNWVRQAPGKGLEWVANINQD
GSDKRYVDSVKGRFTISRDNAKNSLYLEMNSLRADDTAVYYCARASLSSAADYW
GQGTLVTVSS
> variable domain of Light chain of SBJ11-J13
SEQ ID NO: 440
EIVLTQSPLSLPVTLGQPASISCWSSQSLVYSDGNTNLNWYQQRPGQSPRRLIYRVS
NRDSGVPDRFSGSGSGSDFTLKISRVEAEDVGIYYCMQSTHWPHTFGQGTKLEIK
> Heavy chain of SBJ11-J13
SEQ ID NO: 441
MGWSCIILFLVATATGVHSEVQLVESGGGLVQPEGSLRLSCAASGFAFSNYFMNW
VRQAPGKGLEWVANINQDGSDKRYVDSVKGRFTISRDNAKNSLYLEMNSLRADD
TAVYYCARASLSSAADYWGQGTLVTVSSASPTSPKSVFPLAPSSKSTSGGTAALGC
LVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQG
NVFSCSVMHGALHNHYTQKSLSLFPG*
> Light chain of SBJ11-J13
SEQ ID NO: 442
MGWSCIILFLVATATGVHSEIVLTQSPLSLPVTLGQPASISCWSSQSLVYSDGNTNL
NWYQQRPGQSPRRLIYRVSNRDSGVPDRFSGSGSGSDFTLKISRVEAEDVGIYYCM
QSTHWPHTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQ
GLSSPVTKSFNRGEC*
NUCLEIC ACID SEQUENCES OF SBJ11-J13:
> SBJ11-J13_heavy_chain leader sequence
SEQ ID NO: 443
ATGGGATGGTCATGTATCATCCTTTTTCTAGTAGCAACTGCAACCGGTGTACAT
TCC
> SBJ11-J13_heavy_chain variable domain
SEQ ID NO: 444
GAGGTGCAGCTGGTGGAGTCTGGGGGAGGCTTGGTCCAGCCTGAAGGGTCCCT
GAGACTCTCCTGTGCAGCCTCTGGATTCGCCTTTAGTAACTATTTTATGAACTG
GGTCCGCCAGGCTCCAGGGAAGGGGCTGGAGTGGGTGGCCAACATAAACCAA
GATGGAAGTGACAAACGTTATGTGGACTCGGTGAAGGGCCGATTCACCATCTC
CAGAGACAACGCCAAGAACTCACTGTACTTGGAAATGAACAGCCTGAGAGCC
GATGACACGGCTGTGTATTACTGTGCGAGAGCCTCCCTTTCATCCGCCGCTGAC
TACTGGGGCCAGGGAACCCTGGTCACCGTCTCCTCAG
> SBJ11-J13_heavy_chain constant domain
SEQ ID NO: 445
CATCCCCGACCAGCCCCAAGTCGGTCTTCCCCCTGGCACCCTCCTCCAAGAGC
ACCTCTGGGGGCACAGCGGCCCTGGGCTGCCTGGTCAAGGACTACTTCCCCGA
ACCTGTGACGGTCTCGTGGAACTCAGGCGCCCTGACCAGCGGCGTGCACACCT
TCCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAAG
CCCAGCAACACCAAGGTGGACAAGAGAGTTGAGCCCAAATCTTGTGACAAAA
CTCACACATGCCCACCGTGCCCAGCACCTGAACTCCTGGGGGGACCGTCAGTC
TTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAA
CTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCA
GGACTGGCTGAATGGCAAGGAGTACAAGTGCAAGGTCTCCAACAAAGCCCTCC
CAGCCCCCATCGAGAAAACCATCTCCAAAGCCAAAGGGCAGCCCCGAGAACC
ACAGGTGTACACCCTGCCCCCATCCCGGGAGGAGATGACCAAGAACCAGGTCA
GCCTGACCTGCCTGGTCAAAGGCTTCTATCCCAGCGACATCGCCGTGGAGTGG
GAGAGCAATGGGCAGCCGGAGAACAACTACAAGACCACGCCTCCCGTGCTGG
ACTCCGACGGCTCCTTCTTCCTCTATAGCAAGCTCACCGTGGACAAGAGCAGG
TGGCAGCAGGGGAACGTCTTCTCATGCTCCGTGATGCATGGCGCTCTGCACAA
CCACTACACGCAGAAGAGCCTCTCCCTGTTCCCCGGGTAA
> SBJ11-J13_heavy_chain complete
SEQ ID NO: 446
ATGGGATGGTCATGTATCATCCTTTTTCTAGTAGCAACTGCAACCGGTGTACAT
TCCGAGGTGCAGCTGGTGGAGTCTGGGGGAGGCTTGGTCCAGCCTGAAGGGTC
CCTGAGACTCTCCTGTGCAGCCTCTGGATTCGCCTTTAGTAACTATTTTATGAA
CTGGGTCCGCCAGGCTCCAGGGAAGGGGCTGGAGTGGGTGGCCAACATAAAC
CAAGATGGAAGTGACAAACGTTATGTGGACTCGGTGAAGGGCCGATTCACCAT
CTCCAGAGACAACGCCAAGAACTCACTGTACTTGGAAATGAACAGCCTGAGAG
CCGATGACACGGCTGTGTATTACTGTGCGAGAGCCTCCCTTTCATCCGCCGCTG
ACTACTGGGGCCAGGGAACCCTGGTCACCGTCTCCTCAGCATCCCCGACCAGC
CCCAAGTCGGTCTTCCCCCTGGCACCCTCCTCCAAGAGCACCTCTGGGGGCAC
AGCGGCCCTGGGCTGCCTGGTCAAGGACTACTTCCCCGAACCTGTGACGGTCT
CGTGGAACTCAGGCGCCCTGACCAGCGGCGTGCACACCTTCCCGGCTGTCCTA
CAGTCCTCAGGACTCTACTCCCTCAGCAGCGTGGTGACCGTGCCCTCCAGCAG
CTTGGGCACCCAGACCTACATCTGCAACGTGAATCACAAGCCCAGCAACACCA
AGGTGGACAAGAGAGTTGAGCCCAAATCTTGTGACAAAACTCACACATGCCCA
CCGTGCCCAGCACCTGAACTCCTGGGGGGACCGTCAGTCTTCCTCTTCCCCCCA
AAACCCAAGGACACCCTCATGATCTCCCGGACCCCTGAGGTCACATGCGTGGT
GGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAG
CACGTACCGTGTGGTCAGCGTCCTCACCGTCCTGCACCAGGACTGGCTGAATG
GCAAGGAGTACAAGTGCAAGGTCTCCAACAAAGCCCTCCCAGCCCCCATCGAG
AAAACCATCTCCAAAGCCAAAGGGCAGCCCCGAGAACCACAGGTGTACACCC
TGCCCCCATCCCGGGAGGAGATGACCAAGAACCAGGTCAGCCTGACCTGCCTG
GTCAAAGGCTTCTATCCCAGCGACATCGCCGTGGAGTGGGAGAGCAATGGGCA
GCCGGAGAACAACTACAAGACCACGCCTCCCGTGCTGGACTCCGACGGCTCCT
TCTTCCTCTATAGCAAGCTCACCGTGGACAAGAGCAGGTGGCAGCAGGGGAAC
GTCTTCTCATGCTCCGTGATGCATGGCGCTCTGCACAACCACTACACGCAGAA
GAGCCTCTCCCTGTTCCCCGGGTAA
> SBJ11-J13_light_kappa_chain leader sequence
SEQ ID NO: 447
ATGGGATGGTCATGTATCATCCTTTTTCTAGTAGCAACTGCAACCGGTGTACAT
TCC
> SBJ11-J13_light_kappa_chain variable domain
SEQ ID NO: 448
GAAATTGTGTTGACACAGTCTCCACTCTCCCTGCCCGTCACCCTTGGACAGCCG
GCCTCCATCTCCTGCTGGTCTAGCCAAAGCCTCGTATATAGTGATGGAAACACT
AACTTGAATTGGTATCAGCAGAGGCCAGGCCAATCTCCAAGGCGCCTAATTTA
TAGGGTTTCTAACCGGGACTCTGGGGTCCCAGACAGATTCAGCGGCAGTGGGT
CAGGCTCTGATTTCACACTGAAAATCAGCAGGGTGGAGGCTGAGGATGTTGGG
ATTTATTACTGCATGCAAAGTACACACTGGCCGCACACTTTTGGCCAGGGGAC
CAAGCTGGAGATCAAAC
> SBJ11-J13_light_kappa_chain constant domain
SEQ ID NO: 449
GAACTGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGT
GTTAG
> SBJ11-J13_light_kappa_chain complete
SEQ ID NO: 450
ATGGGATGGTCATGTATCATCCTTTTTCTAGTAGCAACTGCAACCGGTGTACAT
TCCGAAATTGTGTTGACACAGTCTCCACTCTCCCTGCCCGTCACCCTTGGACAG
CCGGCCTCCATCTCCTGCTGGTCTAGCCAAAGCCTCGTATATAGTGATGGAAAC
ACTAACTTGAATTGGTATCAGCAGAGGCCAGGCCAATCTCCAAGGCGCCTAAT
TTATAGGGTTTCTAACCGGGACTCTGGGGTCCCAGACAGATTCAGCGGCAGTG
GGTCAGGCTCTGATTTCACACTGAAAATCAGCAGGGTGGAGGCTGAGGATGTT
GGGATTTATTACTGCATGCAAAGTACACACTGGCCGCACACTTTTGGCCAGGG
GACCAAGCTGGAGATCAAACGAACTGTGGCTGCACCATCTGTCTTCATCTTCCC
GCCATCTGATGAGCAGTTGAAATCTGGAACTGCCTCTGTTGTGTGCCTGCTGAA
TAACTTCTATCCCAGAGAGGCCAAAGTACAGTGGAAGGTGGATAACGCCCTCC
AATCGGGTAACTCCCAGGAGAGTGTCACAGAGCAGGACAGCAAGGACAGCAC
CTACAGCCTCAGCAGCACCCTGACGCTGAGCAAAGCAGACTACGAGAAACAC
AAAGTCTACGCCTGCGAAGTCACCCATCAGGGCCTGAGCTCGCCCGTCACAAA
GAGCTTCAACAGGGGAGAGTGTTAG
> L-VH1_VH7 fw primer
SEQ ID NO: 451
CACTCCCAGGTGCAGCTGGTGCAG
> L-VH2 fw primer
SEQ ID NO: 452
TGGGTCTTRTCCCAGGTCACCTTG
> L-VH 3fw primer
SEQ ID NO: 453
AAGGTGTCCAGTGTSAGGTGCAG
> L-VH4_6 fw primer
SEQ ID NO: 454
GTCCTGTCCCAGGTGCAGCTGCAG
> L-VH5 fw primer
SEQ ID NO: 455
GAGTCTGTTCCGAGGTGCAGCTGG
> IgG CH rev primer
SEQ ID NO: 456
GTGCCAGGGGGAAGACCGATG
> IgA CH rev primer
SEQ ID NO: 457
GCMGAGGCTCAGCGGGAAGAC
> IgM CH rev primer
SEQ ID NO: 458
GAGACGAGGGGGAAAAGGGTTG
> L-VK1 fw primer
SEQ ID NO: 459
CAGGTGCCAGATGTGHCATCCAG
> L-VK2 fw primer
SEQ ID NO: 460
CTGGATCCAGTGSGGATATTGTGATG
> L-VK3 fw primer
SEQ ID NO: 461
CCCAGATACCACCGGAGAAATTGTG
> L-VK4 fw primer
SEQ ID NO: 462
CTCTGGTGCCTACGGGGACATCGTG
> L-VK5 fw primer
SEQ ID NO: 463
CTGATACCAGGGCAGAAACGACAC
> CK rev 1st primer
SEQ ID NO: 464
GAACACTCTCCCCTGTTGAAGCTCTTTG
> L-VL1 fw primer
SEQ ID NO: 465
GGTCCTGGGCCCAGTCTGTGCTG
> L-VL2 fw primer
SEQ ID NO: 466
GGTCCTGGGCCCAGTCTGCCCTG
> L-VL3 fw primer
SEQ ID NO: 467
TCTGTGRCCTCCTATGAGCTGAC
> L-VL4_VL5_VL9 fw primer
SEQ ID NO: 468
CTCTCGCAGCCTGTGCTGACTCA
> L-VL6 fw primer
SEQ ID NO: 469
GTTCTTGGGCCAATTTTATGCTG
> L-VL7 fw primer
SEQ ID NO: 470
GGTCCAATTCTCAGGCTGTGGTG
> L-VL8 fw primer
SEQ ID NO: 471
GAGTGGATTCTCAGACTGTGGTG
> L-VL10 fw primer
SEQ ID NO: 472
GTCAGTGGTCCAGGCAGGGCTGAC
> CL rev 1st primer
SEQ ID NO: 473
GTGCTCCCTTCATGCGTGACC
> HIFI*_C134_VH1_5_7
SEQ ID NO: 474
GT ATC ATC CTT TTT CTA GTA GCA ACT GCA ACC GGT GTACATTCC CAG GTG
CAG CTG GTG CAG TCT G
> HIFI*_C134_VH2
SEQ ID NO: 475
GT ATC ATC CTT TTT CTA GTA GCA ACT GCA ACC GGT GTACATTCC CAG GTC
ACC TTG AAG GAG TCT GGT C
> HIFI*_C134_VH3
SEQ ID NO: 476
GT ATC ATC CTT TTT CTA GTA GCA ACT GCA ACC GGT GTACATTCC GAG GTG
CAG CTG GTG GAG TCT GGG GGA G
> HIFI*_C134_VH4_6_a
SEQ ID NO: 477
GT ATC ATC CTT TTT CTA GTA GCA ACT GCA ACC GGT GTACATTCC CAG GTG
CAG CTG CAG GAG TCG GG
> HIFI*_C134_VH4_6_b
SEQ ID NO: 478
GT ATC ATC CTT TTT CTA GTA GCA ACT GCA ACC GGT GTACATTCC CAG GTG
CAG CTG CAG CAG TGG GG
> HIFI*_IgG_CH_rev
SEQ ID NO: 479
CTT GGA GGA GGG TGC CAG GGG GAA GAC CGA TGG GCC CTT GGT GGA RGC
> HIFI*_IgA_CH_rev
SEQ ID NO: 480
CTT GGA GGA GGG TGC CAG GGG GAA GAC CGA CTT GGG GCT GGT CGG GGA
> HIFI*_IgM_CH_rev
SEQ ID NO: 481
CTT GGA GGA GGG TGC CAG GGG GAA G?C CGA TGG GGC GGA TGC ACT CCC
> HIFI*_C135_VK1
SEQ ID NO: 482
GT ATC ATC CTT TTT CTA GTA GCA ACT GCA ACC GGT GTACATTCC GCC ATC
CAG ATG ACC CAG TCT CCA TC
> HIFI*_C135_VK2_a
SEQ ID NO: 483
GT ATC ATC CTT TTT CTA GTA GCA ACT GCA ACC GGT GTACATTCC GAT ATT
GTG ATG ACC CAG ACT CCA CTC TC
> HIFI*_C135_VK2_b
SEQ ID NO: 484
GT ATC ATC CTT TTT CTA GTA GCA ACT GCA ACC GGT GTACATTCC GAT ATT
GTG ATG ACT CAG TCT CCA CTC TC
> HIFI*_C135_VK3_a
SEQ ID NO: 485
GT ATC ATC CTT TTT CTA GTA GCA ACT GCA ACC GGT GTACATTCC GAA ATT
GTG TTG ACA CAG TCT CCA G
> HIFI*_C135_VK3_b
SEQ ID NO: 486
GT ATC ATC CTT TTT CTA GTA GCA ACT GCA ACC GGT GTACATTCC GAA ATT
GTG ATG ACG CAG TCT CCA G
> HIFI*_C135_VK4
SEQ ID NO: 487
GT ATC ATC CTT TTT CTA GTA GCA ACT GCA ACC GGT GTACATTCC GAC ATC
GTG ATG ACC CAG TCT CCA G
> HIFI*_C135_VK5
SEQ ID NO: 488
GT ATC ATC CTT TTT CTA GTA GCA ACT GCA ACC GGT GTACATTCC GAA ACG
ACA CTC ACG CAG TCT CCA G
> HIFI*_C080_VK_Rev
SEQ ID NO: 489
GA TTT CAA CTG CTC ATC AGA TGG CGG GAA GAT GAA GAC AGA TGG TGC
AGC CAC AGT TC
> HIFI*_C080_VL1
SEQ ID NO: 490
GT ATC ATC CTT TTT CTA GTA GCA ACT GCA ACC GGT TCCTGGGCC CAG TCT
GTG CTG ACT CAG CCG CCC TCA G
> HIFI*_C080_VL2
SEQ ID NO: 491
GT ATC ATC CTT TTT CTA GTA GCA ACT GCA ACC GGT TCCTGGGCC CAG TCT
GCC CTG ACT CAG CCT GCC TCC G
> HIFI*_C080_VL3_a
SEQ ID NO: 492
GT ATC ATC CTT TTT CTA GTA GCA ACT GCA ACC GGT TCCTGGGCC TCC TAT
GAG CTG ACA CAG CCA C
> HIFI*_C080_VL3_b
SEQ ID NO: 493
GT ATC ATC CTT TTT CTA GTA GCA ACT GCA ACC GGT TCCTGGGCC TCC TAT
GAG CTG ACT CAG GAC C
> HIFI*_C080_VL4
SEQ ID NO: 494
GT ATC ATC CTT TTT CTA GTA GCA ACT GCA ACC GGT TCCTGGGCC CAG CCT
GTG CTG ACT CAA TCG TCC TCT G
> HIFI*_C080_VL5-9
SEQ ID NO: 495
GT ATC ATC CTT TTT CTA GTA GCA ACT GCA ACC GGT TCCTGGGCC CAG CCT
GTG CTG ACT CAG CCR ACT TC
> HIFI*_C080_VL6
SEQ ID NO: 496
GT ATC ATC CTT TTT CTA GTA GCA ACT GCA ACC GGT TCCTGGGCC AAT TTT
ATG CTG ACT CAG CCC CAC TC
> HIFI*_C080_VL7
SEQ ID NO: 497
GT ATC ATC CTT TTT CTA GTA GCA ACT GCA ACC GGT TCCTGGGCC CAG GCT
GTG GTG ACT CAG GAG CCC TC
> HIFI*_C080_VL8
SEQ ID NO: 498
GT ATC ATC CTT TTT CTA GTA GCA ACT GCA ACC GGT TCCTGGGCC CAG ACT
GTG GTG ACC CAG GAG CCA TC
> HIFI*_C080_VL10
SEQ ID NO: 499
GT ATC ATC CTT TTT CTA GTA GCA ACT GCA ACC GGT TCCTGGGCC CAG GCA
GGG CTG ACT CAG CCA CCC TCG G
> HIFI*_CL_generic
SEQ ID NO: 500
G TGT GGC CTT GTT GGC TTG AAG CTC CTC ACT CGA GGG YGG GAA CAG
AGT G
> CMV_TAP_FW
SEQ ID NO: 501
TTAGGCACCCCAGGCTTTAC
> poly A_TAP_Rev
SEQ ID NO: 502
AGATGGTTCTTTCCGCCTCA

1. A human monoclonal antibody or an antigen-binding portion thereof that specifically binds to a surface antigen of Klebsiellapneumoniae.

2. The human monoclonal antibody or an-antigen-binding portion thereof of claim 1, which specifically binds to a surface antigen of at least one drug-resistant or multi-drug resistant strain of Klebsiella pneumoniae.

3. The human monoclonal antibody or an-antigen-binding portion thereof of claim 2, which specifically binds to a surface antigen of at least one New Delhi Metallo-β-lactamase (NDM)-producing strain of Klebsiella pneumoniae.

4. The human monoclonal antibody or an-antigen-binding portion thereof of claim 2, wherein the at least one strain of Klebsiella pneumoniae is a Klebsiella pneumoniae capsular type K64 strain.

5. The human monoclonal antibody or an-antigen-binding portion thereof of claim 2, wherein the at least one strain of Klebsiella pneumoniae is selected from the group consisting of. Klebsiella pneumoniae Sequence Type 147 (ST147), Klebsiella pneumoniae Sequence Type 258 (ST258), Klebsiella pneumoniae Sequence Type 493 (ST493), Klebsiella pneumoniae Sequence Type 307 (ST307), and Klebsiellapneumoniae Sequence Type 13 (ST13).

6. The human monoclonal antibody or an-antigen-binding portion thereof of claim 5, wherein the at least one strain of Klebsiella pneumoniae is Klebsiella pneumoniae Sequence Type 147 (ST147).

7. The human monoclonal antibody or an-antigen-binding portion thereof of claim 2, wherein the at least one strain of Klebsiellapneumoniae is selected from NDM-1 positive Klebsiellapneumoniae Sequence Type 147 (ST147) strain and NDM-9 positive Klebsiella pneumoniae Sequence Type 147 (ST147) strain.

8. The human monoclonal antibody or an-antigen-binding portion thereof of claim 2, which specifically binds to capsular-antigen of and is bactericidal against at least one of the following strains: NDM-1 positive Klebsiella pneumoniae Sequence Type 147 (ST147) strain and NDM-9 positive Klebsiella pneumoniae Sequence Type 147 (ST147) strain.

9. The human monoclonal antibody or an--antigen-binding portion thereof of claim 2, which specifically binds to capsular-antigen of and is bactericidal against NDM-1 positive Klebsiella pneumoniae Sequence Type 147 (ST147) strain.

10. The human monoclonal antibody or an-antigen-binding portion thereof of claim 1, wherein the surface antigen is selected from capsular-antigen and O-antigen.

11. The human monoclonal antibody or an--antigen-binding portion thereof claim 1, wherein the surface antigen is a capsular-antigen.

12. The human monoclonal antibody or an--- antigen-binding portion thereof of claim 1, wherein the surface antigen is capsular polysaccharide.

13. The human monoclonal antibody or an-antigen-binding portion thereof of claim 1, wherein the surface antigen is capsular polysaccharide of K64 type.

14. The human monoclonal antibody or an-antigen-binding portion thereof of claim 1, wherein the antibody or an-antigen-binding portion thereof shows 50% inhibitory concentration (IC50) of less than 100 ng/ml when tested by an in vitro fluorescence-based serum bactericidal assay (F-SBA).

15. The human monoclonal antibody or an-antigen-binding portion thereof of claim 14, wherein the antibody or antigen-binding portion is tested in an in vitro F-SBA assay against the ST147 NDM-1 strain, the ST147 NDM-9 strain, and/or the ST307 NDM-5 strain of Klebsiella pneumoniae.

16. The human monoclonal antibody or an-antigen-binding portion thereof of claim 14, wherein the antibody or antigen-binding portion is tested in an in vitro F-SBA assay against the ST147 _{NDM-1} strain; and/or the ST147 _{NDM-9} strain of Klebsiella pneumoniae.

17. The human monoclonal antibody or an--antigen-binding portion thereof of claim 1, comprising a heavy chain variable domain (V_{H}) and a light chain variable domain (VL), wherein the V_{H} and VL comprise the following complementarity-determining regions (CDRs):
CDR1 of V_{H} having SEQ ID NO: 325,
CDR2 of V_{H} having SEQ ID NO: 326,
CDR3 of V_{H} having SEQ ID NO: 327,
CDR1 of VL having SEQ ID NO: 328,
CDR2 of VL having the sequence DAS (Asp-Ala-Ser), and
CDR3 of VL having SEQ ID NO: 330;
or
CDR1 of V_{H} having SEQ ID NO: 181,
CDR2 of V_{H} having SEQ ID NO: 182,
CDR3 of V_{H} having SEQ ID NO: 183,
CDR1 of VL having SEQ ID NO: 184,
CDR2 of VL having the sequence AAS (Ala-Ala-Ser), and
CDR3 of VL having SEQ ID NO: 186;
or
CDR1 of V_{H} having SEQ ID NO: 1,
CDR2 of V_{H} having SEQ ID NO: 2,
CDR3 of V_{H} having SEQ ID NO: 3,
CDR1 of VL having SEQ ID NO: 4,
CDR2 of VL having the sequence AAS (Ala-Ala-Ser)ₜ and
CDR3 of VL having SEQ ID NO: 6;
or
CDR1 of V_{H} having SEQ ID NO: 19,
CDR2 of V_{H} having SEQ ID NO: 20,
CDR3 of V_{H} having SEQ ID NO: 21,
CDR1 of VL having SEQ ID NO: 22,
CDR2 of VL having the sequence GAS (Gly-Ala-Ser), and
CDR3 of VL having SEQ ID NO: 24;
or
CDR1 of V_{H} having SEQ ID NO: 37,
CDR2 of V_{H} having SEQ ID NO: 38,
CDR3 of V_{H} having SEQ ID NO: 39,
CDR1 of VL having SEQ ID NO: 40,
CDR2 of VL having the sequence AAS (Ala-Ala-Ser)ₜ and
CDR3 of VL having SEQ ID NO: 42:
or
CDR1 of V_{H} having SEQ ID NO: 55,
CDR2 of V_{H} having SEQ ID NO: 56,
CDR3 of V_{H} having SEQ ID NO: 57,
CDR1 of VL having SEQ ID NO: 58,
CDR2 of VL having the sequence DDY (Asp-Asp-Tyr), and
CDR3 of VL having SEQ ID NO: 60;
or
CDR1 of V_{H} having SEQ ID NO: 73,
CDR2 of V_{H} having SEQ ID NO: 74,
CDR3 of V_{H} having SEQ ID NO: 75,
CDR1 of VL having SEQ ID NO: 76,
CDR2 of VL having the sequence DVS (Asp-Val-Ser), and
CDR3 of VL having SEQ ID NO: 78;
or
CDR1 of V_{H} having SEQ ID NO: 91,
CDR2 of V_{H} having SEQ ID NO: 92,
CDR3 of V_{H} having SEQ ID NO: 93,
CDR1 of VL having SEQ ID NO: 94,
CDR2 of VL having the sequence DVS (Asp-Val-Ser), and
CDR3 of VL having SEQ ID NO: 96;
or
CDR1 of V_{H} having SEQ ID NO: 127,
CDR2 of V_{H} having SEQ ID NO: 128,
CDR3 of V_{H} having SEQ ID NO: 129,
CDR1 of VL having SEQ ID NO: 130,
CDR2 of VL having the sequence AAS (Ala-Ala-Ser), and
CDR3 of VL having SEQ ID NO: 132;
or
CDR1 of V_{H} having SEQ ID NO: 145,
CDR2 of V_{H} having SEQ ID NO: 146,
CDR3 of V_{H} having SEQ ID NO: 147,
CDR1 of VL having SEQ ID NO: 148,
CDR2 of VL having the sequence EIS (Glu-Ile-Ser), and
CDR3 of VL having SEQ ID NO: 150;
or
CDR1 of V_{H} having SEQ ID NO: 163,
CDR2 of V_{H} having SEQ ID NO: 164,
CDR3 of V_{H} having SEQ ID NO: 165,
CDR1 of VL having SEQ ID NO: 166,
CDR2 of VL having the sequence ASS (Ala-Ser-Ser), and
CDR3 of VL having SEQ ID NO: 168;
or
CDR1 of V_{H} having SEQ ID NO: 199,
CDR2 of V_{H} having SEQ ID NO: 200,
CDR3 of V_{H} having SEQ ID NO: 201,
CDR1 of VL having SEQ ID NO: 202,
CDR2 of VL having the sequence DAS (Asp-Ala-Ser), and
CDR3 of VL having SEQ ID NO: 204;
or
CDR1 of V_{H} having SEQ ID NO: 217,
CDR2 of V_{H} having SEQ ID NO: 218,
CDR3 of V_{H} having SEQ ID NO: 219,
CDR1 of VL having SEQ ID NO: 220,
CDR2 of VL having the sequence GAS (Gly-Ala-Ser), and
CDR3 of VL having SEQ ID NO: 222;
or
CDR1 of V_{H} having SEQ ID NO: 235,
CDR2 of V_{H} having SEQ ID NO: 236,
CDR3 of V_{H} having SEQ ID NO: 237,
CDR1 of VL having SEQ ID NO: 238,
CDR2 of VL having the sequence QIS (Gln-Ile-Ser), and
CDR3 of VL having SEQ ID NO: 240;
or
CDR1 of V_{H} having SEQ ID NO: 253,
CDR2 of V_{H} having SEQ ID NO: 254,
CDR3 of V_{H} having SEQ ID NO: 255,
CDR1 of VL having SEQ ID NO: 256,
CDR2 of VL having the sequence DAS (Asp-Ala-Ser), and
CDR3 of VL having SEQ ID NO: 258;
or
CDR1 of V_{H} having SEQ ID NO: 271,
CDR2 of V_{H} having SEQ ID NO: 272,
CDR3 of V_{H} having SEQ ID NO: 273,
CDR1 of VL having SEQ ID NO: 274,
CDR2 of VL having the sequence AAS (Ala-Ala-Ser), and
CDR3 of VL having SEQ ID NO: 276;
or
CDR1 of V_{H} having SEQ ID NO: 289,
CDR2 of V_{H} having SEQ ID NO: 290,
CDR3 of V_{H} having SEQ ID NO: 291,
CDR1 of VL having SEQ ID NO: 292,
CDR2 of VL having the sequence KVS (Lys-Val-Ser), and
CDR3 of VL having SEQ ID NO: 294;
or
CDR1 of V_{H} having SEQ ID NO: 307,
CDR2 of V_{H} having SEQ ID NO: 308,
CDR3 of V_{H} having SEQ ID NO: 309,
CDR1 of VL having SEQ ID NO: 310,
CDR2 of VL having the sequence GAS (Gly-Ala-Ser), and
CDR3 of VL having SEQ ID NO: 312;
or
CDR1 of V_{H} having SEQ ID NO: 343,
CDR2 of V_{H} having SEQ ID NO: 344,
CDR3 of V_{H} having SEQ ID NO: 345,
CDR1 of VL having SEQ ID NO: 346,
CDR2 of VL having the sequence EVS (Glu-Val-Ser), and
CDR3 of VL having SEQ ID NO: 348;
or
CDR1 of V_{H} having SEQ ID NO: 361,
CDR2 of V_{H} having SEQ ID NO: 362,
CDR3 of V_{H} having SEQ ID NO: 363,
CDR1 of VL having SEQ ID NO: 364,
CDR2 of VL having the sequence GAS (Gly-Ala-Ser), and
CDR3 of VL having SEQ ID NO: 366;
or
CDR1 of V_{H} having SEQ ID NO: 379,
CDR2 of V_{H} having SEQ ID NO: 380,
CDR3 of V_{H} having SEQ ID NO: 381,
CDR1 of VL having SEQ ID NO: 382,
CDR2 of VL having the sequence EVS (Glu-Val-Ser), and
CDR3 of VL having SEQ ID NO: 383;
or
CDR1 of V_{H} having SEQ ID NO: 397,
CDR2 of V_{H} having SEQ ID NO: 398,
CDR3 of V_{H} having SEQ ID NO: 399,
CDR1 of VL having SEQ ID NO: 400,
CDR2 of VL having the sequence KVS (Lys-Val-Ser), and
CDR3 of VL having SEQ ID NO: 402;
or
CDR1 of V_{H} having SEQ ID NO: 415,
CDR2 of V_{H} having SEQ ID NO: 416,
CDR3 of V_{H} having SEQ ID NO: 417,
CDR1 of VL having SEQ ID NO: 418,
CDR2 of VL having the sequence GAS (Gly-Ala-Ser), and
CDR3 of VL having SEQ ID NO: 420;
or
CDR1 of V_{H} having SEQ ID NO: 433,
CDR2 of V_{H} having SEQ ID NO: 434,
CDR3 of V_{H} having SEQ ID NO: 435,
CDR1 of VL having SEQ ID NO: 436,
CDR2 of VL having the sequence RVS (Arg-Val-Ser), and
CDR3 of VL having SEQ ID NO: 438.

18. The human monoclonal antibody or antigen-binding portion thereof of claim 1, comprising a heavy chain variable domain (V_{H}) and a light chain variable domain (VL), wherein:
the V_{H} has SEQ ID NO: 331 and the VL has SEQ ID NO: 332; or
the V_{H} has SEQ ID NO: 187 and the VL has SEQ ID NO: 188, or
the V_{H} has SEQ ID NO: 7 and the VL has SEQ ID NO: 8; or
the V_{H} has SEQ ID NO: 25 and the VL has SEQ ID NO: 26; or
the V_{H} has SEQ ID NO: 43 and the VL has SEQ ID NO: 44; or
the V_{H} has SEQ ID NO: 61 and the VL has SEQ ID NO: 62; or
the V_{H} has SEQ ID NO: 79 and the VL has SEQ ID NO: 80; or
the V_{H} has SEQ ID NO: 97 and the VL has SEQ ID NO: 98; or
the V_{H} has SEQ ID NO: 133 and the VL has SEQ ID NO: 134, or
the V_{H} has SEQ ID NO: 151 and the VL has SEQ ID NO: 152, or
the V_{H} has SEQ ID NO: 169 and the VL has SEQ ID NO: 170, or
the V_{H} has SEQ ID NO: 205 and the VL has SEQ ID NO: 206, or
the V_{H} has SEQ ID NO: 223 and the VL has SEQ ID NO: 224; or
the V_{H} has SEQ ID NO: 241 and the VL has SEQ ID NO: 242; or
the V_{H} has SEQ ID NO: 259 and the VL has SEQ ID NO: 260; or
the V_{H} has SEQ ID NO: 277 and the VL has SEQ ID NO: 278; or
the V_{H} has SEQ ID NO: 295 and the VL has SEQ ID NO: 296; or
the V_{H} has SEQ ID NO: 313 and the VL has SEQ ID NO: 314, or
the V_{H} has SEQ ID NO: 349 and the VL has SEQ ID NO: 350, or
the V_{H} has SEQ ID NO: 367 and the VL has SEQ ID NO: 368; or
the V_{H} has SEQ ID NO: 385 and the VL has SEQ ID NO: 386, or
the V_{H} has SEQ ID NO: 403 and the VL has SEQ ID NO: 404; or
the V_{H} has SEQ ID NO: 421 and the VL has SEQ ID NO: 422; or
the V_{H} has SEQ ID NO: 439 and the VL has SEQ ID NO: 440.

19. The human monoclonal antibody or antigen-binding portion thereof of claim 1, comprising a heavy-chain variable domain (V_{H}) and a light chain variable domain (VL)ₜwherein the VL and the VH are each at least 85% identical in amino acid sequence, respectively, to
a VH having SEQ ID NO: 331 and a VL having SEQ ID NO: 332 or
a VH having SEQ ID NO: 187 and a VL having SEQ ID NO: 188 or
a VH having SEQ ID NO: 7 and a VL having SEQ ID NO: 8; or
a VH having SEQ ID NO: 25 and a VL having SEQ ID NO: 26; or
a VH having SEQ ID NO: 43 and a VL having SEQ ID NO: 44 or
a VH having SEQ ID NO: 61 and a VL having SEQ ID NO: 62 or
a VH having SEQ ID NO: 79 and a VL having SEQ ID NO: 80 or
a VH having SEQ ID NO: 97 and a VL having SEQ ID NO: 98 or
a VH having SEQ ID NO: 133 and a VL having SEQ ID NO: 134 or
a VH having SEQ ID NO: 151 and a VL having SEQ ID NO: 152 or
a VH having SEQ ID NO: 169 and a VL having SEQ ID NO: 170 or
a VH having SEQ ID NO: 205 and a VL having SEQ ID NO: 206 or
a VH having SEQ ID NO: 223 and a VL having SEQ ID NO: 224 or
a VH having SEQ ID NO: 241 and a VL having SEQ ID NO: 242 or
a VH having SEQ ID NO: 259 and a VL having SEQ ID NO: 260 or
a VH having SEQ ID NO: 277 and a VL having SEQ ID NO: 278 or
a VH having SEQ ID NO: 295 and a VL having SEQ ID NO: 296 or
a VH having SEQ ID NO: 313 and a VL having SEQ ID NO: 314 or
a VH having SEQ ID NO: 349 and a VL having SEQ ID NO: 350 or
a VH having SEQ ID NO: 367 and a VL having SEQ ID NO: 368 or
a VH having SEQ ID NO: 385 and a VL having SEQ ID NO: 386 or
a VH having SEQ ID NO: 403 and a VL having SEQ ID NO: 404 or
a VH having SEQ ID NO: 421 and a VL having SEQ ID NO: 422 or
a VH having SEQ ID NO: 439 and a VL having SEQ ID NO: 440.

20. The human monoclonal antibody of claim 1, comprising a heavy chain and a light chain, wherein:
the heavy chain of the antibody has SEQ ID NO: 333 and the light chain of the antibody has SEQ ID NO: 334; or
the heavy chain of the antibody has SEQ ID NO: 189 and the light chain of the antibody has SEQ ID NO: 190; or
the heavy chain of the antibody has SEQ ID NO: 9 and the light chain of the antibody has SEQ ID NO: 10; or
the heavy chain of the antibody has SEQ ID NO: 27 and the light chain of the antibody has SEQ ID NO: 28; or
the heavy chain of the antibody has SEQ ID NO: 45 and the light chain of the antibody has SEQ ID NO: 46;or
the heavy chain of the antibody has SEQ ID NO: 63 and the light chain of the antibody has SEQ ID NO: 64; or
or the heavy chain of the antibody has SEQ ID NO: 81 and the light chain of the antibody has SEQ ID NO: 82; or
or the heavy chain of the antibody has SEQ ID NO: 99 and the light chain of the antibody has SEQ ID NO: 100 or
the heavy chain of the antibody has SEQ ID NO: 135 and the light chain of the antibody has SEQ ID NO: 136; or
the heavy chain of the antibody has SEQ ID NO: 153 and the light chain of the antibody has SEQ ID NO: 154; or
the heavy chain of the antibody has SEQ ID NO: 171 and the light chain of the antibody has SEQ ID NO: 172;or
the heavy chain of the antibody has SEQ ID NO: 207 and the light chain of the antibody has SEQ ID NO: 208; or
the heavy chain of the antibody has SEQ ID NO: 225 and the light chain of the antibody has SEQ ID NO: 226; or
the heavy chain of the antibody has SEQ ID NO: 243 and the light chain of the antibody has SEQ ID NO: 244 to
the heavy chain of the antibody has SEQ ID NO: 261 and the light chain of the antibody has SEQ ID NO: 262; or
the heavy chain of the antibody has SEQ ID NO: 279 and the light chain of the antibody has SEQ ID NO: 280; or
the heavy chain of the antibody has SEQ ID NO: 297 and the light chain of the antibody has SEQ ID NO: 298; or
the heavy chain of the antibody has SEQ ID NO: 315 and the light chain of the antibody has SEQ ID NO: 316; or
the heavy chain of the antibody has SEQ ID NO: 351 and the light chain of the antibody has SEQ ID NO: 352; or
the heavy chain of the antibody has SEQ ID NO: 369 and the light chain of the antibody has SEQ ID NO: 370; or
the heavy chain of the antibody has SEQ ID NO: 387 and the light chain of the antibody has SEQ ID NO: 388; or
the heavy chain of the antibody has SEQ ID NO: 405 and the light chain of the antibody has SEQ ID NO: 406; or
the heavy chain of the antibody has SEQ ID NO: 423 and the light chain of the antibody has SEQ ID NO: 424; or
the heavy chain of the antibody has SEQ ID NO: 441 and the light chain of the antibody has SEQ ID NO: 442.

21-36. (canceled)
